(19) United States
(12) Patent Application Publication
(10) Pub. No.: US 2026/0265348 A1
(43) Pub. Date: Sep. 10, 2026

(54) Novel Anti-HSV Antibody

(71) Applicant: Heidelberg ImmunoTherapeutics GMBH, Heidelberg (DE)

(72) Inventors: Torsten SCHALLER, Heidelberg (DE); Narges SEYFIZADEH, Heidelberg (DE); Michaela ARNDT, Heidelberg (DE)

(73) Assignee: Heidelberg ImmunoTherapeutics GMBH, Heidelberg (DE)

(21) Appl. No.: 19/493,831

(22) PCT Filed: Jun. 14, 2024
(86) PCT No.: PCT/EP2024/066523

§ 371 Date: Jan. 22, 2026

(30) Foreign Application Priority Data
Jun. 16, 2023 (EP) 23179700.2

(51) Int. Cl. C07K 16/087; A61K 47/68; A61K 49/00

(52) U.S. Cl. CPC C07K 16/087; A61K 47/6839; A61K 49/0002; C07K 2317/21; C07K 2317/24; C07K 2317/31

(57) ABSTRACT

Described is a (first) anti-HSV antibody or an antigen-binding fragment thereof binding to the glycoprotein B (gB) of HSV-1 and/or HSV-2, wherein said antibody comprises the complementarity determining regions V_{H}CDR1, V_{H}CDR3, V_{L}CDR1, V_{L}CDR2, and V_{L}CDR3, each comprising the sequences as defined in the claims, wherein said antibody or antigen-binding fragment has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹. Moreover, described is a combination of (A) said (first) anti-HSV antibody or an antigen-binding fragment thereof; and (B) a second anti-HSV antibody or an antigen-binding fragment thereof recognizing/binding to the glycoprotein B (gB) of the HSV-1 and/or HSV-2, wherein said antibody comprises the complementarity determining regions V_{H}CDR1, V_{H}CDR2, V_{H}CDR3, V_{L}CDR1, V_{L}CDR2, and V_{L}CDR3, each comprising the sequences as defined in the claims, wherein said second antibody has a dissociation constant Kd of at most 40 nM, preferably at most 30 nM, more preferably at most 20 nM, even more preferably at most 15 nM, at most 13 nM and at most 10 nM. Further, described is a pharmaceutical composition comprising an effective amount of said anti-HSV antibody or the antigen-binding fragment thereof or the combination of said antibodies and at least one pharmaceutically acceptable excipient. Further, described is an anti-HSV antibody or the antigen-binding fragment thereof or said combination of said antibodies for use in a method for the prophylactical or therapeutical treatment of a disorder or disease as defined in the claims. Further, described is a bispecific antibody binding to the glycoprotein B (gB) of the HSV-1 and/or HSV-2 which comprises: (A) a first binding domain comprising: the complementarity determining regions V_{H}CDR1, V_{H}CDR2, V_{H}CDR3, V_{L}CDR1, V_{L}CDR2, and V_{L}CDR3 of the above first antibody; and (B) a second binding domain comprising: the complementarity determining regions V_{H}CDR1, V_{H}CDR2, V_{H}CDR3, V_{L}CDR1, V_{L}CDR2, and V_{L}CDR3 of the above second antibody; wherein said bispecific antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹. Finally, described is a trispecific antibody which, in addition to the first and second binding domain as described for the bispecific antibody, comprises a third binding domain.

### CROSS-REFERENCE TO RELATED APPLICATIONS

This application is a § 371 National Stage Application of PCT/EP2024/066523 filed on Jun. 14, 2024, which claims priority to EP 23179700.2 filed on Jun. 16, 2023, both of these documents are hereby incorporated by reference in their entirety.

The present invention relates to a (first) anti-HSV antibody or an antigen-binding fragment thereof binding to the glycoprotein B (gB) of HSV-1 and/or HSV-2, wherein said antibody comprises the complementarity determining regions V_{H}CDR1, V_{H}CDR3, V_{L}CDR1, V_{L}CDR2, and V_{L}CDR3, each comprising the sequences as defined in the claims, wherein said antibody or antigen-binding fragment has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹. Moreover, the present invention relates to a combination of (A) said (first) anti-HSV antibody or an antigen-binding fragment thereof; and (B) a second anti-HSV antibody or an antigen-binding fragment thereof recognizing/binding to the glycoprotein B (gB) of the HSV-1 and/or HSV-2, wherein said antibody comprises the complementarity determining regions V_{H}CDR1, V_{H}CDR2, V_{H}CDR3, V_{L}CDR1, V_{L}CDR2, and V_{L}CDR3, each comprising the sequences as defined in the claims, wherein said second antibody has a dissociation constant Kd of at most 40 nM, preferably at most 30 nM, more preferably at most 20 nM, even more preferably at most 15 nM, at most 13 nM and at most 10 nM. Further, the present invention relates to a pharmaceutical composition comprising an effective amount of said anti-HSV antibody or the antigen-binding fragment thereof or the combination of said antibodies and at least one pharmaceutically acceptable excipient. Further, the present invention relates to said anti-HSV antibody or the antigen-binding fragment thereof or said combination of said antibodies for use in a method for the prophylactical or therapeutical treatment of a disorder or disease as defined in the claims. Finally, the present invention relates to a bispecific antibody binding to the glycoprotein B (gB) of the HSV-1 and/or HSV-2 which comprises: (A) a first binding domain comprising: the complementarity determining regions V_{H}CDR1, V_{H}CDR2, V_{H}CDR3, V_{L}CDR1, V_{L}CDR2, and V_{L}CDR3 of the above first antibody; and (B) a second binding domain comprising: the complementarity determining regions V_{H}CDR1, V_{H}CDR2, V_{H}CDR3, V_{L}CDR1, V_{L}CDR2, and V_{L}CDR3 of the above second antibody; wherein said bispecific antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹. Finally, the present invention relates to a trispecific antibody which, in addition to the first and second binding domain as described for the bispecific antibody, comprises a third binding domain.

Herpes simplex virus (HSV) refers to two closely related members of the herpesviridae family, Herpes simplex virus type 1 (HSV-1) and Herpes simplex virus type 2 (HSV-2), which in 2016 were worldwide prevalent in about 67%, or 13% of people aged 15-49 years, respectively [1]. HSV-1 and HSV-2 are among the most common viral infections in the world, are transmitted through close contact resulting in a life-long persistence. HSV-1 infections are often acquired in early childhood as subclinical infections while a subset present with severe disease. HSV-2 is usually acquired through sexual activity and can cause recurrent chronic lesions in the genital area. Infection with herpes simplex viruses is categorized into one of several distinct disorders based on the site of infection. Oral herpes (Herpes simplex labialis), the visible symptoms of which are colloquially called cold sores or fever blisters, is an infection of the face or mouth. Oral herpes is the most common form of infection. Genital herpes (Herpes simplex genitalis) is the second most common form of herpes. Other disorders such as herpetic whitlow, herpes gladiatorum, eczema herpeticum, herpetic sycosis, ocular herpes (herpes simplex retinitis, herpes simplex conjunctivitis, herpes simplex corneae or herpes simplex keratitis), cerebral herpes infection encephalitis, Mollaret's meningitis, neonatal herpes simplex, and possibly Bell's palsy are all caused by HSV-1 or HSV-2 infection, but both viruses can also infect and affect organs, such as lungs, kidneys and liver. HSV-1 infection and serology has been correlated in some studies with a higher risk for the development of Alzheimer Disease (AD) or dementia, suggesting that persistent HSV-1 infection could impact the development of neurological disorders and that anti-HSV therapies could potentially be used to prevent or prolong the time until onset of these neurological disorders.

The outcome of an infection with HSV-1 or HSV-2 is mostly asymptomatic but can be associated with mild symptoms or even manifest in a life-threatening condition, the spectrum of pathology is large. After primary infection HSV spreads from infected epithelial cells to axons of sensory neurons mainly of the peripheral nervous system innervating the site of the primary infection followed by retrograde transport to the respective dorsal root ganglia, where the virus establishes a latent reservoir for life. In case of infection of the central nervous system this is associated with high morbidity and mortality rates, while also in brain organoids HSV has been found to be able to establish latency in vitro, which could suggest the possibility that similar processes could happen in vivo. Like all herpesviruses, HSV-1 and HSV-2 have a lytic and a latent phase, reactivation from latency to lytic replication can occur upon diverse triggers, e.g., immunosuppression and lead to retrograde transport of viruses from the neurons to epithelial cells inducing the formation of blisters and lesions. Intermittent HSV reactivations hence result in the production of infectious HSV from latently infected neurons. It is possible that early reactivation events from latency to lytic replication occur more frequently than thought and that only occasionally these events lead to symptomatic disease. Suppression of HSV reactivation under immunocompetent conditions and spread may be a result of tissue-resident immune cells that may act immediately at the site and time of HSV reactivation, thereby preventing the spread. Reactivation of the virus can be triggered by a wide range of stress stimuli (e.g., immunodeficiency, illness, trauma, fever, menstruation, UV light and sexual intercourse) that act on the neuron, or at a peripheral site innervated by the infected ganglion, or systemically.

The pathogenesis of HSV infections can be caused by a direct cytopathic effect of the virus, resulting in cellular lysis and focal necrosis of the infected area, or by indirect immune responses, e.g. in the case of HSV stromal keratitis or encephalitis [2]. Herpes simplex is mostly transmitted by direct contact with a lesion or the body fluid of an infected individual. Oral herpes is easily diagnosed if the patient presents with visible sores or ulcers. Transmission may also occur through skin-to-skin contact during periods of asymptomatic viral shedding. Barrier protection methods are the most reliable method of preventing transmission of herpes, but they merely reduce rather than eliminate risk.

A cure for herpes has not yet been developed. Once infected, the virus remains in the body for life. Symptomatic recurrences (outbreaks) may occur from time to time. However, after several years, outbreaks become less severe and more sporadic, and some people will become perpetually asymptomatic and will no longer experience outbreaks, though they may still be contagious to others. Treatments with antivirals can reduce viral shedding and alleviate the severity of symptomatic episodes. Herpes simplex labialis (also called cold sores, herpes simplex labialis, recurrent herpes labialis, or orolabial herpes) is a type of herpes simplex disease occurring on the lip, i.e., an infection caused by HSV-1 or HSV-2. An outbreak typically causes small blisters or sores on or around the mouth commonly known as cold sores or fever blisters. The sores typically heal within 2 to 3 weeks, but the herpes virus remains dormant in the facial nerves, following orofacial infection, periodically reactivating (in symptomatic people) to create sores in the same area of the mouth or face at the site of the original infection. Cold sore has a rate of frequency that varies from rare episodes to 12 or more recurrences per year. People with the condition typically experience one to three outbreaks annually. The frequency and severity of outbreaks generally decreases over time.

Herpes simplex genitalis (or genital herpes) is a genital infection caused by HSV-1 or HSV-2. A 1998 study indicated HSV-infection was the most common sexually transmitted infection by the number of cases. Most individuals carrying the herpes simplex virus are unaware they have been infected and many will never suffer an outbreak, which involves blisters similar to cold sores. Although most individuals infected with genital herpes infections are asymptomatic, severe clinical manifestations, especially in populations with underlying immune compromising conditions, can occur. Surprisingly, increasing numbers of primary genital herpes infections by HSV-1 have been noted, while infection does not seem to result in chronically recurrent genital HSV-1 outbreaks, in contrast to HSV-2. Oral sex is thought to be the main way of HSV-1 transmission causing primary genital herpes lesions. When symptomatic, the typical manifestation of a primary herpes genital infection is clusters of genital sores consisting of inflamed papules and vesicles on the outer surface of the genitals, resembling cold sores. These usually appear 4-7 days after exposure to HSV for the first time. HSV-2 is usually the cause of chronically recurrent genital herpes disease but the differences to HSV-1 in terms of chronic persistence and reactivation are not understood. While there is no cure for herpes genitalis disease, over time usually symptoms are increasingly mild and outbreaks are decreasingly frequent in the elderly population. HSV-2 infection increases the risk of HIV acquisition as well as HIV transmission in dually infected individuals by two to three-fold [3].

Herpetic simplex keratitis is an inflammation of the eye predominantly caused by HSV infection of the cornea. Ocular infection with HSV can cause eye disease of different severity, ranging from conjunctivitis and dendritic keratitis to stromal edema and necrotizing stromal keratitis. HSV-1 causes more than 90% of ocular HSV infections and is the leading cause of virus-induced blindness in developed countries.

Moreover, there are other, rather rare HSV infections of mucosal or epidermal tissue which will be briefly addressed in the following. Chronic or disseminated cutaneous herpes simplex infections are known which are not restricted to labial or genital tract. Mostly, immunodeficient patients are affected with this disease like, e.g., patients with hypogammaglobulinema or patients with cutaneous T-cell lymphomas. Chronic cutaneous herpes simplex is a distinctive clinical presentation of the HSV in a compromised host. This infection can be defined as chronically active destructive skin lesions that potentially may progress into the disseminated (systemic) form. While most HSV infections display episodes that show healing in one or two weeks, the lesions of chronic cutaneous herpes simplex have an indolent course that may last for several months. Chronic cutaneous herpes simplex, which is common in immunosuppressed patients, is characterized by atypical, chronic, and persistent lesions, which complicate and delay the diagnosis. This may lead to death caused by associated complications. It is of vital importance that when evaluating chronic ulcers of long duration, especially in children, the possibility of a chronic herpes simplex virus infection be considered.

Herpes gladiatorum refers to a herpes skin infection that occurs in adolescence among wrestlers but it is also common in other contact sports. It usually occurs on the head, most commonly the jaw area, the neck, chest, face, stomach, and legs. Eczema herpeticum, also known as a form of Kaposi varicelliform eruption caused by viral infection, usually with the herpes simplex virus (HSV), is an extensive cutaneous vesicular eruption that arises from pre-existing skin disease, usually atopic dermatitis. Children with atopic dermatitis have a higher risk of developing eczema herpeticum, in which HSV type 1 (HSV-1) is the most common pathogen. Eczema herpeticum can be severe, progressing to disseminated infection and death if untreated. Diseases caused by HSV, in particular Herpes simplex labialis and Herpes simplex genitalis represent the most common infectious diseases of the skin.

Genital herpes can be perinatally transmitted and cause life-threatening neonatal HSV infection. Neonatal herpes infection is a rare event, estimated to occur in about ten cases of 100.000 livebirths [4]. Neonatal HSV infection can be caused by HSV-1, which is the predominant cause for neonatal infections in the Americas, Europe and Western Pacific, as well as HSV-2, the predominant cause for neonatal Herpes in Africa, South East Asia and Eastern Mediterranean [4]. In total, approximately 14.000 cases occur annually, of which 10.000 are HSV-2 and 4.000 HSV-1 induced [4].

HSV-1 seroprevalence has also been connected with the development of neurological disorders such as dementia and Alzheimer's Disease (AD). While the contribution of subclinical viral reactivations in the central nervous system remain controversial, the idea of the increased aggregation of Abeta protein as a defense mechanism to viral reactivation preventing viral spread is attractive.

At present, it is standard to use virostatic agents in anti-HSV therapies. The most common virostatic agents (e.g. acyclovir, penciclovir, foscarnet, idoxuridin) are nucleoside or pyrophosphate analogues whose common active principle is based on the inhibition of DNA synthesis in virus-infected cells. One of the most important therapeutic agents for the treatment of HSV infections is the purine nucleoside analogue acyclovir. It is phosphorylated by the viral thymidine kinase and then interferes with the viral DNA replication. In contrast, the human DNA polymerase is less susceptible to acyclovir by factor 30-50, for which reason merely marginal side effects are observed. In double-blind placebo-controlled studies with patients suffering from herpes simplex labialis infection, it has been demonstrated that acyclovir (in the form of Zovirax Creme) is capable of reducing the infection only by 0.5 days (e.g., from 4.8 days to 4.3 days) compared to placebo-treated patients [5].

Presently, the pyrophosphate analogue foscarnet is particularly employed in immunosuppressed patients against acyclovir-resistant herpes virus. This agent causes a direct inhibition of the viral DNA polymerase and has no influence on the thymidine kinase. However, the use of foscarnet leads to severe undesirable side effects such as renal failure, cardiac problems, has toxicity on the bone marrow, and may also cause cutaneous ulceration. Because of its teratogenic effects, foscarnet may also not be administered during pregnancy. Further, the formation of cross-resistant strains is observed, which makes the development of alternative therapeutic agents highly necessary. A passive or active immunoprophylaxis is currently not available. The currently used virostatic agents are only effective in infected cells in which the virus is actively replicating. Moreover, the treatment with current virostatics suffers the disadvantage that the risks to develop recurrences is not reduced or even prevented, but merely presents a treatment of symptoms with minimal clinical effects.

Humoral immunity plays an important role in controlling HSV and other viral infections. Circulating serum antibodies, which can bind viral envelope glycoproteins necessary for viral entry, develop during infection. It has been shown that the presence of maternal serum antibodies specific to HSV reduces neonatal transmission of HSV-2 [6]. People who are HSV-1 seropositive, have lower occurrences of symptomatic genital HSV-2 infections, suggesting a certain degree of cross-protection [6]. Indeed, an HSV-2 subunit vaccine targeting glycoprotein D (gD) induced 82% protection against culture-positive HSV-1, but showed no protection against HSV-2, acquisition in young women participating in the HerpeVac trial [7, 8]. The discrepancy in protection from HSV-1 and HSV-2 infection by this vaccine trial remains unexplained to date. Several attempts to generate a protective vaccine against chronic recurrent HSV-2 infection have failed to date [7-9]. The therapeutic vaccine candidate GEN-003, containing a deletion mutant of HSV-2 gD, HSV-2 ICP4.2 and Matrix-M2 adjuvant was tested in HSV-2 seropositive persons with genital herpes at different doses and was shown to reduce viral shedding, lesion and recurrence rates, as well as duration of recurrences at a specific dose however was not driven forward in clinical development [10][11]. Antibody as well as cellular immune responses were stimulated however long-term persistence was questionable [12][13]. A phase 1 clinical study with the replication-defective HSV-2 vaccine HSV529 showed safety and elucidation of serum neutralizing antibody responses in 78% of seronegative vaccine recipients, as well as modest CD4+ T cell responses [14]. This vaccine also induced antibodies that mediated HSV-2-specific natural killer cell activation and vaccine-induced antibodies were detectable in cervicovaginal fluid [15]. However, long-term prevention of recurrences was uncertain.

Neutralizing serum antibodies are capable of binding HSV in the gap between neuron endings and epithelial cells and limit bidirectional viral transfer between these tissues in vitro [16]. Evidently, serum antibodies orsystemically applied antibodies limit the extent of HSV infection and may prevent transmission. Several approaches for antibody-based treatment of HSV infections have been proposed and investigated [17]. Of note, some anti-HSV antibodies mediating antibody-dependent cellular cytotoxicity (ADCC) have been connected to superior protection. Low levels of ADCC-mediating antibodies were suggested to play an important role for the absent protection by vaccines against HSV-2 since in preclinical studies a gD-deleted HSV-2 vaccine candidate elicited potently protecting ADCC-mediating antibodies, with little neutralizing capacity [18, 19]. However, the role of antibody-mediated cellular phagocytosis (ADCP) and subsequent immune cell activation, in particular T-cell activation, has been neglected [17].

E317 is the original clone of the fully human antibody drug product UB-621, targeting HSV glycoprotein D (gD) [20], which was shown to reduce mortality in an intraperitoneal HSV-1 challenge model of adult mice. A phase 1 clinical trial for subcutaneous administration proved safety and tolerability in healthy individuals and phase 2 clinical trials for the treatment of recurring genital HSV infections were approved in the US and China.

HSV8, a fully human gD-specific IgG1 isolated using a phage display library, reduced the mortality in an intraperitoneal HSV-1 adult mouse model as well as in intranasal HSV-1/HSV-2 neonatal mouse models [21]. HSV8 was also tested in combination with a broadly neutralizing anti-HIV antibody in a phase 1 clinical trial and demonstrated safety to use the antibody in reducing vaginal transmission of HSV and HIV using the MB66 vaginal film [22]. Topically applied HSV8 also protected mice from vaginal transmission of HSV-2 [23].

As gD mediates the interaction of the virus with its cellular receptor(s), gD is an attractive target. However, other glycoproteins are essential for viral entry, hence impose also attractive targets for therapeutic intervention. The glycoprotein B (gB) plays a pivotal role for virus entry into target cells, since it constitutes the viral fusion protein. There are currently no approved anti-HSV gB monoclonal antibodies for therapy.

Recently, the murine monoclonal antibody (MAb)2c has been described which specifically recognizes the glycoprotein B (gB) of HSV-1 and HSV-2. HSV-gB is an integral part of the multicomponent fusion machinery required for virus entry and cell-cell fusion. It constitutes the fusogenic protein of the virus that enables fusion of virus and target cell membranes during the initial infection steps. While the exact mechanism underlying the triggering of viral and cellular membrane fusion is still not solved, it is envisioned that glycoprotein D binding to a receptor on target cells triggers, via glycoprotein H/L, the activation of gB to mediate fusion and gB-transition from a prefusion into a postfusion conformation. MAb2c has been demonstrated to neutralize cell-free virus and to abrogate viral cell-to-cell spread, a key mechanism by which HSV-1 or HSV-2 escapes humoral immune surveillance independent from antibody-dependent cellular cytotoxicity (ADCC) and/or complement-dependent cytotoxicity (CDC) [24-27], WO2011/038933, WO2015/197763. MAb2c was derived from mice immunized with inactivated HSV-1 strain 342 hv [24].

The corresponding humanized IgG1 MAb2c (HDIT101) is a novel therapeutically tested antibody. HDIT101 protected immunocompetent mice from a lethal intravaginal challenge with HSV-1 or HSV-2 and protected mice from developing a herpetic stromal keratitis [26-28]. In immunodeficient mice, HDIT101 protects better from a lethal infection by HSV-1 than HSV-2 [29]. HDIT101 prevents cell-to-cell spread, a feature of anti-HSV antibodies that was recently demonstrated to correlate with an increased protection from HSV-1 reactivation [30]. HDIT101 does not exert measurable effects in vitro via CDC or ADCC induction but is capable of mediating antibody-dependent cellular phagocytosis (ADCP) via binding of its Fc fragment to antigen presenting cells (APCs), resulting in subsequent cellular internalization of the antibody-HSV complex and induction of a robust anti-HSV T-cell response. This function may play an important role in the prevention of recurrent HSV reactivations [31].

In a recently conducted clinical phase I dose escalation study in healthy volunteers intravenous HDIT101 has shown an excellent safety profile even at excessively high doses of >12 g [32]. A clinical phase II study, MATCH-2 (‘Monoclonal Antibody Therapy for Chronic Herpes Virus 2 Infection’, ClinicalTrials.gov identifier NCT04165122), that compared the efficacy of a single intravenous dose of HDIT101 with episodic standard of care treatment with valacyclovir in patients with chronic recurrent anogenital HSV-2 infection, demonstrated that patients treated with HDIT101 had prolonged recurrence-free times after administration as compared to the control group.

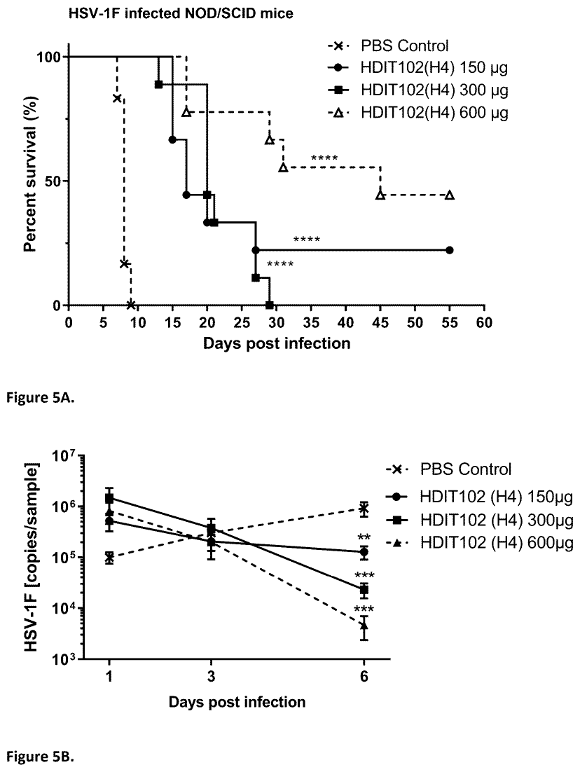

Moreover, WO 2023/003951 recently described some anti-HSV gB antibodies with neutralizing activity isolated from seropositive human subjects (see, e.g., FIG. 5A and FIG. 5B of WO 2023/003951).

Although the above antibody which specifically recognizes the glycoprotein B (gB) of HSV-1 and HSV-2 has been proven to have beneficial effects allowing the treatment and/or prevention of diseases caused by HSV-1 or HSV-2, there is still a need for improvements, in particular as regards specificity and/or binding capabilities so as to provide further means and methods to the known “toolbox” of known anti-HSV therapies.

Moreover, despite the positive observations with HDIT101 in the MATCH-2 study, in vitro emergence of HDIT101-resistant HSV-1 and HSV-2 escape mutants under suboptimal HDIT101 concentrations suggests that a long-term monotherapy of recurrent HSV infections with HDIT101 could potentially lead to the evolution of HDIT101-resistant mutant virus strains.

Hence, in light of the prior art, there is a need to provide further means and/or improvements for the effective treatment or prevention of infectious diseases caused by HSV.

The present invention is, in part, based on the surprising finding that a newly identified antibody that specifically recognizes the glycoprotein B (gB) of HSV-1 and HSV-2 exhibits unexpected properties as shown in in the appended Examples.

More specifically, the present invention provides a new antibody (HDIT102(H4)) which is derived from a phage library of head and neck cancer patients and, hence, is a fully human IgG1. HDIT102(H4) Fab has the unique feature of a very low dissociation-rate (k_{dis} of 2.9×10⁻⁵ s⁻¹), making binding of HDIT102(H4) to its target protein gB extremely strong and specific, which is an advancement over the currently closest comparator HDIT101, making its antiviral effects unique and very efficient. HDIT102(H4) showed no tissue cross-reactivity in human tissue immunohistochemistry experiments using a diverse set of tissues from three independent donors performed according to current drug development guidelines.

Moreover, while WO 2023/003951 discloses some anti-HSV gB antibodies with neutralizing activity isolated from seropositive human subjects (see, e.g., FIG. 5A and FIG. 5B of WO 2023/003951), as shown in in the appended Examples, the present invention is, in part, based on the surprising finding that the newly identified antibody that specifically recognizes the glycoprotein B (gB) of HSV-1 and HSV-2 exhibits unexpected properties over the antibodies disclosed in WO 2023/003951. More specifically, the binding characteristics of the antibody of the present invention vis-à-vis the binding characteristics of the antibodies of WO 2023/003951 have been tested. As is shown in Example 26, the antibody of the present invention binds to a different epitope compared to the antibodies of WO 2023/003951. Moreover, as also shown in Example 26, the antibodies of WO 2023/003951 showed slower association rates compared to the antibody of the present invention. Thus, taking into account the low dissociation rate of the antibody of the present invention, the antibody of the present invention has unexpected properties over the prior art antibodies in that, overall, the dissociation constant KD of the antibody of the present invention is superior over the prior art antibodies.

Further, while the antibodies tested in WO 2023/003951 had been shown to have neutralizing capability which, however, comes along with the activity of inducing antibody-dependent cellular cytotoxicity (ADCC), the antibody of the present invention has unique properties which is, in particular, relevant for its therapeutic use, as it potently neutralizes HSV-1 and HSV-2 without activating ADCC or CDC, thus avoiding non-specific toxicities in patients.

Furthermore, the present invention defined the epitope of HDIT102(H4) on the gB protein of HSV-1. The epitope is located in close proximity and partially overlapping with the epitope of HDIT101. In fact, in vitro competition studies demonstrated that HDIT102(H4) could compete with HDIT101 binding to recombinant gB protein in post-fusion state as well as ectopically expressed post-fusion gB on cells.

This finding, in part, paves the way for addressing the above-mentioned disadvantages in (long-term) monotherapy of recurrent HSV infections (wherein there is a danger of emerging HDIT101-resistant HSV-1 and HSV-2 escape mutants, potentially leading to the evolution of HDIT101-resistant mutant virus strains, in particular, under suboptimal HDIT101 concentrations). Indeed, the present invention is, in part, based on the surprising finding that while, as expected for competing antibodies, no synergistic effects of a combination using HDIT101 and HDIT102(H4) were observed in vitro, synergistic effects were observed on the survival of immunocompetent Balb/c mice after intravaginally infection with a lethal dose of HSV-2G when using the combination as compared to a monotherapy with equal total amount of IgG. This is especially surprising and unexpected given that both antibodies compete for binding to recombinant gB. This finding lead to the proposal that the formation of irregularly shaped immune complexes by asymmetrical cross-linking with two competing antibodies provides an improved signal to recognize the antibody-opsonized immune complexes. In an Example described below, individual gB protomers within the gB trimer are bound either by HDIT101 or by HDIT102(H4) in the combination cocktail and this leads to immune complexes that are asymmetrically cross-linked. The structural binding analysis of the HDIT101 and HDIT102(H4) Fab demonstrated perpendicular orientation of HDIT101 and HDIT102(H4) Fab when bound to recombinant gB.

Thus, in one aspect of this invention, the opposing orientations of two or more antibodies that compete for one binding site in an antigen, as part of a combinational therapeutic drug product, will lead to the formation of irregularly shaped immune complexes of a multimeric antigen (in case of gB, trimeric) opsonized with the antibodies and this will lead to a greater immune response. In case of the combination of HDIT101 and HDIT102(H4), this leads to a better protection of mice infected with a lethal dose of HSV-2G presumably by enhanced immune stimulation.

Indeed, surprisingly, the present invention demonstrates that a combination of two therapeutic anti-HSV antibodies which compete for binding to recombinant gB in vitro show a significantly better therapeutic response in an in vivo infection model at equal doses to the monotherapy with the individual antibodies. The present invention also demonstrates that a bispecific molecule with HDIT101 and HDIT102(H4) gB-targeting scFvs fused carboxyterminal with an IgG Fc domain has improved characteristics for neutralizing cell-free HSV while pertaining the low k_{dis} observed for HDIT102(H4) IgG. This compound thereby provides the superior characteristics of a combination of HDIT101 and HDIT102(H4) in one molecule.

Thus, the present invention, in part, solves the problem of emerging HDIT101-resistant HSV-1 and HSV-2 escape mutants in (long-term) monotherapy of recurrent HSV infections by providing a combination of the prior art antibody HDIT101 with HDIT102(H4) (a fully human antibody with superior characteristics), as described below either in combination therapy or in a single bispecific molecule. In fact, while in vitro propagation of HSV-1 or HSV-2 in the presence of suboptimal concentrations of HDIT101 or HDIT102(H4) alone let to the emergence of respective resistant virus strains by a single amino acid substitution, the combination of HDIT101 and HDIT102(H4) prevented the emergence of resistant virus. As beneficially shown in the appended Examples, no double-resistant virus could be propagated in vitro when applying a combination of HDIT101 and HDIT102(H4). In addition, when either HDIT101-resistant, or HDIT102(H4)-resistant virus was propagated in the presence of the respective other antibody, no development of resistance to the antibody used was observed, i.e., HDIT101-resistant virus did not become resistant to HDIT102(H4) and HDIT102(H4)-resistant virus did not become resistant to HDIT101. This is, in particular, surprising, because both antibodies target partially overlapping epitopes and compete for binding to recombinant gB and ectopically expressed gB on cells in vitro.

As a matter of fact, a combination of monoclonal anti-HSV antibodies to treat HSV infection has never been suggested. The combination of monoclonal antibodies has been described as possible treatment strategy for other viral infectious diseases. For example, two monoclonal antibodies, AX290 and AX677, targeting non-overlapping epitopes on the SARS-CoV-2 Spike protein, were shown to efficiently neutralize virus and prevented the emergence of resistant escape mutants in vitro [33]. Likewise, Baum et al. described that a cocktail of non-competing antibodies against SARS-CoV-2 did not cause the emergence of resistant mutants, however a cocktail with competing antibodies did [34]. Hence the results provided in the presented invention, that two competing antibodies against HSV-1/2 gB with partially overlapping epitopes did prevent the emergence of resistant virus and moreover worked synergistically in an in vivo infection model is an unexpected and novel finding. Synergistic effects in vivo, but not in vitro, were also observed for a combination of two neutralizing antibodies, however these were targeting distinct epitopes on Chikungunya virus E1 and E2 structural proteins [35]. Human broadly neutralizing monoclonal antibodies against hepatitis C virus have also been discovered to work synergistically when applied in combination, however also in this case antibodies with overlapping epitopes did not work synergistically in vitro when combined [36]. Combination therapy approaches with monoclonal antibodies targeting different non-overlapping sites on the Env protein have also been tested for human immunodeficiency virus (HIV) [37, 38]. A combination of antibodies has also been proposed in the therapeutics of non-infectious diseases, e.g. in cancer treatment [39].

However, synergistic therapeutic effects by the combination of two or more monoclonal antibodies that compete for binding to their target in vitro, i.e., have at least partially overlapping epitopes, is surprising in light of the prior art. In the present invention we show in an experimental in vivo model enhanced, synergistic therapeutic effects on survival using two antibodies targeting HSV gB with partially overlapping epitopes that compete for binding to recombinant gB in vitro. A combination of the two antibodies protected significantly better at equal doses than the individual antibodies alone.

In view of the prior art, the technical problem underlying the present invention is the provision of further means and methods for the treatment and/or prevention of infectious diseases caused by HSV, in particular, the provision of improved means and methods for the treatment of Herpes simplex infections which facilitates administration regimens known in the art and prevents local spreading of the infection and emergence of resistance mutants.

The technical problem is solved by provision of the embodiments characterized in the claims.

Thus, in a first aspect, the present invention relates to an anti-HSV antibody or an antigen-binding fragment thereof binding to the glycoprotein B (gB) of HSV-1 and/or HSV-2, wherein said antibody comprises:
the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6;
wherein said antibody or antigen-binding fragment has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹,
preferably, wherein said antibody or antigen-binding fragment thereof is capable of neutralizing HSV.

Moreover, in a second aspect, which is related to the above first aspect, the present invention relates to a combination of
(A) an anti-HSV antibody or an antigen-binding fragment thereof as defined in the context of the first aspect; and
(B) an anti-HSV antibody or an antigen-binding fragment thereof binding to the glycoprotein B (gB) of the HSV-1 and/or HSV-2, wherein said antibody comprises:
   the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO: 16; wherein said antibody has a dissociation constant Kd of at most 40 nM, preferably at most 30 nM, more preferably at most 20 nM, even more preferably at most 15 nM, at most 13 nM and at most 10 nM.

Further, in a third aspect, which is related to the above second aspect, the present invention relates to a bispecific antibody or an antigen-binding fragment thereof binding to the glycoprotein B (gB) of the HSV-1 and/or HSV-2 which comprises:
(A) a first binding domain comprising:
   the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6; and
(B) a second binding domain comprising:
   the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16;
wherein said bispecific antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹.

Further, in a fourth aspect, the present invention relates to a trispecific antibody or an antigen-binding fragment thereof binding to the glycoprotein B (gB) of the HSV-1 and/or HSV-2 which comprises:
(A) a first binding domain comprising:
   the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6; and
(B) a second binding domain comprising:
   the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16; and
(C) a third binding domain comprising:
   the complementarity determining regions V_{H}CDR1 comprising SEQ ID NO: 31, V_{H}CDR2 comprising SEQ ID NO: 32, V_{H}CDR3 comprising SEQ ID NO: 33, V_{L}CDR1 comprising SEQ ID NO: 34, V_{L}CDR2 comprising SEQ ID NO: 35, and V_{L}CDR3 comprising SEQ ID NO:36;
wherein said trispecific antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹; and wherein said trispecific antibody in a concentration of at most 10 nM, preferably of at most 5 nM, more preferably of at most 2 nM, of at most 1 nM, of at most 0.8 nM, of at most 0.6 nM, and most preferably of at most 0.5 nM, is capable of neutralising a defined amount of HSV of 100 TCID50.

In the following, the first aspect, is described in more detail.

The antibody or antigen-binding fragment thereof as used in accordance with the first aspect of the present invention is not particularly limited as long as it is an “anti-HSV antibody or an antigen-binding fragment thereof” and as long as it specifically binds to or interacts with the glycoprotein B (gB) of HSV-1 and/or HSV-2. The glycoprotein B (gB) of HSV-1 and/or HSV-2 is a domain or an antigen of HSV-1 and/or HSV-2 as described in more detail further below.

The term “binding to” as used in the context of the present invention defines a binding (interaction) of at least two “antigen-interaction-sites” with each other. The term “antigen-interaction-site”defines, in accordance with the present invention, a motif of a polypeptide, i.e., a part of the antibody or antigen-binding fragment of the present invention, which shows the capacity of specific interaction with a specific antigen or a specific group of antigens of the HSV, in particular, a specific antigen or group of antigens of the glycoprotein B (gB) of HSV-1 and/or HSV-2. The term “binding to” means in accordance with this invention that the antibody is capable of specifically interacting with and/or binding to at least two amino acids of the glycoprotein B (gB) of HSV-1 and/or HSV-2 as defined herein.

In preferred embodiments, in particular, when the binding to a specific epitope is concerned as described in more detail further below, said binding/interaction is preferably also understood to define a “specific recognition”. The term “specifically recognizing” means in accordance with this invention that the antibody is capable of specifically interacting with and/or binding to at least two amino acids of a defined epitope of the glycoprotein B (gB) of HSV-1 and/or HSV-2.

Antibodies can recognize to different epitopes on HSV gB. This term “recognizing”, in particular, relates to the specificity of the antibody molecule, i.e., to its ability to discriminate between the specific regions, in particular, its specific epitopes of the glycoprotein B (gB) of HSV-1 and/or HSV-2.

The term “specific interaction” as used in accordance with the present invention means that the antibody or antigen-binding fragment thereof of the invention does not or does not essentially cross-react with (poly) peptides of similar structures. Accordingly, the antibody or antigen-binding fragment thereof of the invention specifically binds to/interacts with structures of the glycoprotein B (gB), preferably the glycoprotein B (gB) of HSV-1 and/or HSV-2. Specific examples of such molecules are given herein below.

Cross-reactivity of a panel of antibody or antigen-binding fragment thereof under investigation may be tested, for example, by assessing binding of said panel of antibody or antigen-binding fragment thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to the certain structure of the HSV, e.g., a specific epitope or (poly) peptide/protein of the HSV (preferably, a specific epitope of the glycoprotein B (gB) of HSV-1 and/or HSV-2) but do not or do not essentially bind to any of the other epitope or (poly) peptides of the same glycoprotein B (gB) of HSV-1 and/or HSV-2, are considered specific for the epitope or (poly) peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g., with BIAcore®), analytical ultracentrifugation, isothermal titration calorimetry, fluorescence anisotropy, fluorescence spectroscopy or by radiolabeled ligand binding assays.

The term “binding to” does not only relate to a linear epitope but may also relate to a conformational epitope, a structural epitope or a discontinuous epitope consisting of two regions of the human target molecules or parts thereof. In the context of this invention, a conformational epitope is defined by two or more discrete amino acid sequences separated in the primary sequence which comes together on the surface of the molecule when the polypeptide folds to the native protein (Sela, Science 166 (1969), 1365 and Laver, Cell 61 (1990), 553-536).

Accordingly, specificity can be determined experimentally by methods known in the art and methods as described herein. Such methods comprise, but are not limited to Western Blots, ELISA-, RIA-, ECL-, IRMA-tests and peptide scans.

In a preferred embodiment, the anti-HSV antibody or the antigen-binding fragment thereof according the first aspect of the present invention is a monoclonal or a polyclonal antibody.

In a further preferred embodiment, the anti-HSV antibody or the antigen-binding fragment thereof according the first aspect of the present invention is a humanized or a fully human antibody.

In a further preferred embodiment, the anti-HSV antibody or the antigen-binding fragment thereof according the first aspect of the present invention is a murine antibody.

As already outlined above in the context of the first aspect of the present invention, the term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein in the context of the present disclosure, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Fully-human antibodies may also be produced, for example, by phage display which is a widely used screening technology which enables production and screening of fully human antibodies. Also phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

The term antibody also relates to recombinant human antibodies, heterologous antibodies and heterohybrid antibodies. The term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g., murine or rabbit) antibodies are chimeric immunoglobulins, immunoglobulin chains which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: JonesNature 321 (1986), 522-525; Reichmann Nature 332 (1998), 323-327 and Presta Curr Op Struct Biol 2 (1992), 593-596.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in the context of the anti-HSV antibody or the antigen-binding fragment thereof according the first aspect of the present invention, the term “antibody” relates to full immunoglobulin molecules as well as to parts of such immunoglobulin molecules (i.e., “antigen-binding fragment thereof”). Furthermore, the term relates, in general terms, to modified and/or altered antibody molecules. The term also relates to recombinantly or synthetically generated/synthesized antibodies. The term also relates to intact antibodies. The term antibody also comprises but is not limited to fully-human antibodies, chimeric antibodies, humanized antibodies, CDR-grafted antibodies and antibody constructs, like single chain Fvs (scFv) or antibody-fusion proteins.

The term “antigen-binding fragment thereof” in the context of the first aspect of the present invention is not particularly limited as long as said “antigen-binding fragment thereof” referred to has the above-mentioned capability of having a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹ which is described in more detail further below.

As mentioned, the anti-HSV antibody or the antigen-binding fragment thereof binding to the glycoprotein B (gB) of HSV-1 and/or HSV-2 and comprising the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6 according to the first aspect of the present invention has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹.

“Low dissociation rate k_{dis}” in this context means that the binding of the antibody to its target is extraordinarily strong and specific. More specifically, “low” means in this context that the antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹.

In further preferred embodiments, said anti-HSV antibody or antigen-binding fragment thereof has a low dissociation rate k_{dis} of at most 5.0×10⁻⁴ s⁻¹, preferably at most 4.0×10⁻⁴ s⁻¹, more preferably at most 3.0×10⁻⁴ s⁻¹, even more preferably at most 2.0×10⁻⁴ s⁻¹, at most 1.0×10⁻⁴ s⁻¹, at most 9.0×10⁻⁵ s⁻¹, at most 8.0×10⁻⁵ s⁻¹, at most 7.0×10⁻⁵ s⁻¹, at most 6.0×10⁻⁵ s⁻¹, at most 5.0×10⁻⁵ s⁻¹, at most 4.0×10⁻⁵ s⁻¹, at most 3.0×10⁻⁵ s⁻¹ and most preferably at most 2.9×10⁻⁵ s⁻¹, at most 2.0×10⁻⁵ s⁻¹, at most 1.5×10⁻⁵ s⁻¹, at most 1.0×10⁻⁵ s⁻¹, at most 5.0×10⁻⁶ s⁻¹, at most 2.0×10⁻⁶ s⁻¹, at most 1.0×10⁻⁶ s⁻¹, at most 5.0×10⁻⁷ s⁻¹, at most 2.0×10⁻⁷ s⁻¹, at most 1.0×10⁻⁷ s⁻¹, at most 1.0×10⁻⁸ s⁻¹, and at most 1.0×10⁻⁹ s⁻¹.

As shown herein above and below, the anti-HSV antibody of the present invention surprisingly has a very low dissociation rate (k_{dis}) of most preferably equal or lower than k_{dis} of 2.9×10⁻⁵ s⁻¹ when bound to recombinant gB when using bio-layer interferometry (BLI).

The dissociation rate k_{dis} may be determined by methods known to the person skilled in the art. In one embodiment, this dissociation rate k_{dis} is determined as described in the Examples appended hereto. In a particular embodiment, this dissociation rate k_{dis} is determined by using recombinant gB and bio-layer interferometry (Octet).

In a preferred embodiment this can be determined as outlined in the following:

To produce recombinant HSV-1 gB protein a codon-optimized DNA sequence encoding for HSV-1 gB ectodomain (aa 30-729; UniProtKB P06436.1) or HSV-2 gB ectodomain (aa 22-724; GenBank: QAU10948.1) including a signal peptide and a C-terminal tag can be cloned into a mammalian expression vector. HSV gB recombinant protein can then transiently be expressed in HEK293-E6 suspension cells cultured in culture media. HEK293-E6 cells are transfected with plasmid encoding gB. On day 5 after the transfection the supernatant is harvested by centrifugation steps. Next, the pH of the supernatant is adjusted by the addition of 1 ml 2 M Tris buffer pH 9/100 ml supernatant. Afterwards the gB protein is purified tag-affinity gravity flow purification. The tag of gB protein is then removed by thrombin digestion and dialyzed against 50 mM Tris, 150 mM NaCl, pH 8. The thrombin digested gB protein is then purified three times by tag-specific affinity chromatography to deplete the sample from any remaining tagged gB protein. Next, the gB protein is concentrated and is further purified via size-exclusion chromatography. The peek fractions are pooled and concentrated.

Anti-HSV gB antibody Fab can be generated either by papain digest and subsequent purification (as done for HDIT101), or generated recombinantly by transfection of light and truncated heavy chain encoding plasmids into 293T-E6 cells (as done for HDIT102(H4)). HSV gB recombinant protein is biotinylated and afterwards, the residual biotin is removed. An initial loading scout is performed to find out the best biosensor loading concentration. The streptavidin biosensors (of the Octet) are loaded with different concentrations of biotinylated gB and the absorption kinetics of the test antibody Fab fragments are measured. A dilution series of Fab fragments is then analysed to determine the dissociation rate (k_{dis}).

More specifically, the dissociation rate k_{dis} can be determined as outlined in the following: To produce recombinant HSV-1 gB protein a codon-optimized DNA sequence encoding for HSV-1F gB ectodomain (aa 30-729; UniProtKB P06436.1) or HSV-2G gB ectodomain (aa 22-724; GenBank: QAU10948.1) including a BM40 signal peptide and a C-terminal double Strep-tag can be cloned into a pCAGGS mammalian expression vector. HSV gB recombinant protein can then transiently be expressed in HEK293-E6 suspension cells cultured in serum-free media, e.g., F17 medium (ThermoFisher) supplemented with 0.1% Kolliphor (Sigma) and 4 mM Glutamine. HEK293-E6 cells are transfected with PeiMax (Polysciences) at a cell density of 1.5-2.0×10⁶ cells/ml with 1 μg plasmid encoding gB and 2 μg PeiMax/ml culture media. Twenty-four hours after the transfection Tryptone N1 feeder (Organi Technie) is added to the cultures. On day 5 after the transfection the supernatant is harvested by two centrifugation steps, first 1200 rpm to remove the cells and then 3600 rpm to remove cell debris. Next, the pH of the supernatant is adjusted by the addition of 1 ml 2 M Tris buffer pH 9/100 ml supernatant. Afterwards the gB protein is purified by Strep-Tactin XT (IBA, Germany) gravity flow purification according to the manufacturer protocol. The Strep-tag of gB protein is then removed by thrombin digestion (Serva) and dialyzed against 50 mM Tris, 150 mM NaCl, pH 8. The thrombin digested gB protein is then purified three times by Strep-Tactin XT affinity chromatography to deplete the sample from any remaining Strep-tagged gB protein. Next, the gB protein is concentrated with Amicon spin columns (cut-off 30 k) and is further purified with a Superdex 200 10/300 GL SEC column and an Äkta Pure FPLC. The peek fractions are pooled and concentrated with Amicon spin columns.

Anti-HSV gB antibody Fab can be generated either by papain digest and subsequent purification (as done for HDIT101), or generated recombinantly by transfection of light and truncated heavy chain encoding plasmids into 293T-E6 cells (as done for HDIT102(H4)). HSV gB recombinant protein is biotinylated (NHS-PEG4-Biotin (Thermo Fischer Scientific, A39259)) in ratio 3:1 for 30 min at room temperature and afterwards, the residual biotin is removed by using desalting columns and centrifugation at 1000 g for 2 min (Zeba Spin Desalting Columns; 7K MWCO, 2 ml (Thermo Scientific UE285726). An initial loading scout is performed to find out the best biosensor loading concentration. The streptavidin biosensors (of the Octet) are loaded with different concentrations of biotinylated gB and the absorption kinetics of the test antibody Fab fragments are measured. The optimal gB concentration for loading the biosensor was determined with 5 μg/ml. Biotinylated gB (wt) [5 μg/ml] is used to load the biosensor and the binding kinetics of antibody Fab fragments (100 nM) against immobilized gB can be analyzed using global 1:1 fit model. A dilution series of Fab fragments is then analysed to determine the dissociation rate (k_{dis}).

The anti-HSV antibody or the antigen-binding fragment thereof according the first aspect of the present invention is not particularly limited as long as it is an “anti-HSV antibody or an antigen-binding fragment thereof that binds to the glycoprotein B (gB) of HSV-1 and/or HSV-2”. Thus, the antibody may be any antibody which specifically binds to or specifically recognizes or interacts with a glycoprotein B (gB) of HSV-1 and/or HSV-2, i.e., a domain, an antigen of a glycoprotein B (gB) of HSV-1 and/or HSV-2. The skilled person is readily in a position to generate such an antibody directed to a given domain (i.e., an antigen, preferably a surface-antigen of a glycoprotein B (gB) of HSV-1 and/or HSV-2) and determine whether a respective antibody is capable of detecting/binding to a given domain, an antigen, preferably a surface-antigen of glycoprotein B (gB) of HSV-1 and/or HSV-2 based on the skilled person's common general knowledge and the methods described above.

The anti-HSV antibody or the antigen-binding fragment thereof according the first aspect of the present invention binds to/recognizes the “glycoprotein B (gB) of HSV-1 and/or HSV-2”. The viral antigen glycoproteins D, B, C, H, L, E or I (i.e., gD, gB, gC, gH, gL, gE, gI) are well-characterized and known in the art surface or envelope proteins of HSV-1 and/or HSV-2. These proteins may not only be found on the surface or in the envelope structure of HSV-1 and/or HSV-2, i.e., on the surface of released infectious particles (i.e., the envelope of free virions) but they may also be present on the surface of infected cells, i.e., on the surface of cells. Yet, in a more preferred embodiment, the antibody of the invention binds to/recognizes the viral surface antigen glycoprotein B (i.e., gB,) of the HSV-1 and/or HSV-2 envelope.

Thus, in a preferred embodiment, the anti-HSV antibody or the antigen-binding fragment thereof for use according to the present invention binds to the surface glycoprotein B (gB) of the HSV-1 and/or HSV-2 envelope in terms of the present invention as defined above, and preferably, recognizes an epitope thereof in terms of the present invention and as defined above.

The glycoprotein B of HSV-1 and/or HSV-2 is well-characterized and, without being bound to specific sequences, examples sequences of various HSV-1 and HSV-2 strains, respectively, are shown in SEQ ID NOs:9, 10 and 21 to 24 and 40. SEQ ID NO:9 shows the sequence of the glycoprotein B of HSV-1 strain F, SEQ ID NO:10 shows the sequence of the glycoprotein B of HSV-1 strain KOS, SEQ ID NO:21 shows the sequence of the glycoprotein B of HSV-1 strain gC-39-R6, SEQ ID NO:22 shows the sequence of the glycoprotein B of HSV-2 strain HG52, SEQ ID NO:23 shows the sequence of the glycoprotein B of HSV-2 strain 333, SEQ ID NO:24 shows the sequence of the glycoprotein B of HSV-2 strain MMA and SEQ ID NO:40 shows the sequence of glycoprotein B of HSV-2 strain G. A sequence alignment of these glycoprotein B amino acid sequences shows that the overall amino acid homology, preferably, identity of gB of HSV-1 and HSV-2 is 85% while the sequences are least conserved at the N- and C-terminal regions of the protein.

As mentioned, the anti-HSV antibody or the antigen-binding fragment thereof according the first aspect of the present invention comprises the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6.

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. CDR regions of an Ig-derived region may be determined by different means and methods known in the art.

For example, the CDR regions of an Ig-derived region may be determined as described in Kabat “Sequences of Proteins of Immunological Interest”, 5th edit. NIH Publication no. 91-3242 U.S. Department of Health and Human Services (1991); Chothia J. Mol. Biol. 196 (1987), 901-917 or Chothia Nature 342 (1989), 877-883.

In the context of the present invention, the CDR regions (as well as the framework regions (FR)) are determined according to the numbering scheme of Martin as described in Norman, R. A., F. Ambrosetti, A. Bonvin, L. J. Colwell, S. Kelm, S. Kumar and K. Krawczyk (2020). “Computational approaches to therapeutic antibody design: established methods and emerging trends.” Brief Bioinform 21(5): 1549-1567.

Hence, in the context of the present invention, reference to amino acid residues is according to the numbering scheme of Martin.

In a further preferred embodiment, the anti-HSV antibody or the antigen-binding fragment thereof according the first aspect of the present invention comprises the following framework regions:
an amino acid sequence with at least 70% sequence identity to each one of the amino acid residues shown in positions 1 to 25 (V_{H}FR1), 36 to 49 (V_{H}FR2), 67 to 98 (V_{H}FR3), 112 to 122 (V_{H}FR4) of SEQ ID NO: 7, 1 to 22 (V_{L}FR1), 34 to 48 (V_{L}FR2), 56 to 87 (V_{L}FR3), and 97 to 106 (V_{L}FR4) of SEQ ID NO: 8.

As mentioned above, in the context of the present invention, the CDR regions (as well as the framework regions (FR)) are determined according to the numbering scheme of Martin as described in Norman, R. A., F. Ambrosetti, A. Bonvin, L. J. Colwell, S. Kelm, S. Kumar and K.

Krawczyk (2020). “Computational approaches to therapeutic antibody design: established methods and emerging trends.” Brief Bioinform 21(5): 1549-1567.

Hence, in the context of the present invention, reference to amino acid residues is according to the numbering scheme of Martin.

In a further, preferred embodiment, the anti-HSV antibody or the antigen-binding fragment thereof according the first aspect of the present invention comprises an amino acid sequence with at least 75%, at least 80%, more preferably at least 85%, at least 90%, even more preferably at least 95%, and most preferably 98% or 99% overall sequence identity in the framework regions compared to each one of the amino acid residues shown in positions 1 to 30, 38 to 51, 68 to 99, and 112 to 122 of SEQ ID NO: 7 and in positions 1 to 23, 41 to 55, 63 to 94, and 104 to 114 of SEQ ID NO: 8. Such antibodies are suitable for the purposes of the present invention as long as the antibody or antigen-binding fragment thereof binds to gB of HSV-1 or HSV-2 and a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹ as described herein above and below and/or being capable of inhibiting the spreading of HSV from an infected cell to an adjacent second non-infected cell (cell-to-cell spread) and/or being capable of inhibiting cell-to-cell spread independent from antibody-dependent cellular cytotoxicity (ADCC) and/or complement-dependent cytotoxicity (CDC) as described herein above and below.

Thus, in a preferred embodiment, the anti-HSV antibody or the antigen-binding fragment thereof according the first aspect of the present invention comprises an amino acid sequence having the above variable regions of the light and heavy chains (i.e., the CDRs defined above, i.e., V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6) while the amino acid sequence have a variability in the framework region with at least 75%, at least 80%, more preferably at least 85%, at least 90%, even more preferably at least 95%, and most preferably 98% or 99% overall sequence identity in the framework regions compared to each one of the amino acid residues shown in positions 1 to 30, 38 to 51, 68 to 99, and 112 to 122 of SEQ ID NO: 7 and in positions 1 to 23, 41 to 55, 63 to 94, and 104 to 114 of SEQ ID NO:8.

In this context, a polypeptide has “at least X % sequence identity” in the framework regions to SEQ ID NO: 7 or 8 if SEQ ID NO: 7 or SEQ ID NO:8 is aligned with the best matching sequence of a polypeptide of interest and the amino acid identity between those two aligned sequences is at least X % over positions 1 to 30, 38 to 51, 68 to 99, and 112 to 122 of SEQ ID NO: 7 and positions 1 to 23, 41 to 55, 63 to 94, and 104 to 114 of SEQ ID NO: 8. As outlined in more detail further below, such an alignment of amino acid sequences can be performed using, for example, publicly available computer homology programs such as the “BLAST” program provided on the National Centre for Biotechnology Information (NCBI) homepage using default settings provided therein. Further methods of calculating sequence identity percentages of sets of amino acid sequences or nucleic acid sequences are known in the art.

In a further preferred embodiment, the anti-HSV antibody or the antigen-binding fragment thereof according the first aspect of the present invention comprises the V_{H} of SEQ ID NO:7 and the V_{L} of SEQ ID NO:8.

Thus, in a preferred embodiment, the anti-HSV antibody or the antigen-binding fragment thereof according the first aspect of the present invention is an antibody that binds to the glycoprotein B (gB) of HSV-1 and/or HSV-2 which comprises or consists of VH domain (heavy chain variable region) and VL domain (light chain variable region), i.e., the amino acid sequence of the variable region of the heavy chain of an antibody as depicted in SEQ ID NO:7 and the amino acid sequence of the variable region of the light chain of an antibody as depicted in SEQ ID NO:8.

However, the antibody as used in the present invention is not particularly limited to such variable heavy and light chain variable regions but may also be an antibody or antigen-binding fragment thereof that binds to the glycoprotein B (gB) of HSV-1 and/or HSV-2 envelope which comprises or consists of VH domain and VL domain with most preferably, 100%, with at least 99%, 98%, 97%, 96%, 95%, 90%, 85%, 75%, 70%, 65%, 60%, 55% or 50% sequence homology, preferably identity with the sequences of SEQ ID NOs: 7 and 8, respectively, as long as the antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹ as described herein above and below. Furthermore, the antibody or antigen-binding fragment thereof is a molecule that comprises VH and VL domains having up to 0, 1, 2, 3, 4, 5, 6, 7, 8, 9, 10 or more conservative amino acid substitutions with reference to the sequences of SEQ ID NOs: 7 and 8 as long as the antibody a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹ as described herein above and below. Moreover, the antibody or antigen-binding fragment thereof is an antibody fragment selected from the group consisting of Fab, Fab′, Fab′-SH, Fv, scFV, F(ab′)2, and a diabody.

In order to determine whether an amino acid sequence has a certain degree of identity to the sequences of SEQ ID NOs: 7 and 8, the skilled person can use means and methods well known in the art, e.g., alignments, either manually or by using computer programs known to the person skilled in the art. Such an alignment can, e.g., be done with means and methods known to the skilled person, e.g., by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences. In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the nucleic acid sequences or with the amino acid sequences as described above which are capable of binding to gB of HSV-1 or HSV-2 and having a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹ as described herein above and below and/or being capable of inhibiting the spreading of HSV from an infected cell to an adjacent second non-infected cell (cell-to-cell spread) or being capable of inhibiting cell-to-cell spread independent from antibody-dependent cellular cytotoxicity (ADCC) and/or complement-dependent cytotoxicity (CDC) as described herein above and below), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

Preferably, the amino acid substitution(s) are “conservative substitution(s)” which refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson Molecular Biology of the Gene, The Benjamin/Cummings Pub. Co. 4th Ed. (1987), 224. In addition, substitutions of structurally or functionally similar amino acids are less likely to disrupt biological activity. Within the context of the present invention the binding compounds/antibodies of the present invention comprise polypeptide chains with sequences that include up to 0 (no changes), 1, 2, 3, 4, 5, 6, 7, 8, 9, 10, 12, 15, 20 or more conservative amino acid substitutions when compared with the specific amino acid sequences disclosed herein, for example, SEQ ID NO: 7 (referring to the variable region of the antibody heavy chain of the antibody) and 8 (referring to the variable of the light chain of the antibody). As used herein, the phrase “up to X” conservative amino acid substitutions includes 0 substitutions and any number of substitutions up to 10 and including 0, 1, 2, 3, 4, 5, 6, 7, 8, 9, 10 substitutions.

Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The specificity of the antibody or antigen-binding fragment of the first aspect of the present invention may not only be expressed by the nature of the amino acid sequence of the antibody or the antigen-binding fragment as defined above but also by the epitope to which the antibody is capable of binding to. Thus, the present invention utilizes in a preferred embodiment an anti-HSV antibody or an antigen-binding fragment thereof in accordance with the present invention which recognizes the same epitope as the antibody as described above. Thus, in a further preferred embodiment, the anti-HSV antibody or the antigen-binding fragment thereof according the first aspect of the present invention is not limited to the above-described anti-HSV antibody or antigen-binding fragment thereof but may also be an anti-HSV antibody or an antigen-binding fragment thereof which recognizes the same epitope as said antibody as defined above.

More specifically, in a preferred embodiment, the present invention relates to an anti-HSV antibody or antigen-binding fragment thereof which recognizes the same epitope as said antibody as defined herein-above, wherein said epitope is located at the amino acids D199, A203, K204, Y303, R304, K320, Q321, V322, D323, Y326, R335 and T337 of glycoprotein B of HSV-1 strain F and contact amino acids residues D191, A195, K196, Y295, R296, K312, Q313, V314, D315, Y318, R327 and T329 of glycoprotein B of HSV-2 strain G, respectively, preferably, wherein said epitope consists of the contact amino acid residues D199, A203, K204, Y303, R304, K320, Q321, V322, D323, Y326, R335 and T337 of glycoprotein B of HSV-1 strain F (SEQ ID NO:9) and of the contact amino acids residues D191, A195, K196, Y295, R296, K312, Q313, V314, D315, Y318, R327 and T329 of glycoprotein B of HSV-2 strain G (SEQ ID NO:40), respectively.

In a particular embodiment, the epitope is determined by cryo-electron microscopy (Cryo-EM).

Whether an anti-HSV antibody or antigen-binding fragment thereof recognizes the same epitope as an (reference) antibody can be determined by routine methods known in the art. In the following, suitable methods are described in more general terms while further below, a more detailed method is described that lead to the identification of the residues of the amino acids D199, A203, K204, Y303, R304, K320, Q321, V322, D323, Y326, R335 and T337 of glycoprotein B of HSV-1 strain F and contact amino acids residues D191, A195, K196, Y295, R296, K312, Q313, V314, D315, Y318, R327 and T329 of glycoprotein B of HSV-2 strain G, respectively in accordance with the present invention.

In general, it may be understood by a person skilled in the art that the epitopes may be comprised in the gB protein, but may also be comprised in a degradation product thereof or may be a chemically synthesized peptide. The amino acid positions are only indicated to demonstrate the position of the corresponding amino acid sequence in the sequence of the gB protein. The invention encompasses all peptides comprising the epitope. The peptide may be a part of a polypeptide of more than 100 amino acids in length or may be a small peptide of less than 100, preferably less than 50, more preferably less than 25 amino acids, even more preferably less than 16 amino acids. The amino acids of such peptide may be natural amino acids or nonnatural amino acids (e.g., beta-amino acids, gamma-amino acids, D-amino acids) or a combination thereof. Further, the present invention may encompass the respective retro-inverso peptides of the epitopes. The peptide may be unbound or bound. It may be bound, e.g., to a small molecule (e.g., a drug or a fluorophor), to a high-molecular weight polymer (e.g., polyethylene glycol (PEG), polyethylene imine (PEI), hydroxypropylmethacrylate (HPMA), etc.) or to a protein, a fatty acid, a sugar moiety or may be inserted in a membrane. In order to test whether an antibody in question and the antibody of the present invention recognize the same epitope, the following competition study may be carried out: Vero cells infected with 3 moi (multiplicity of infection) are incubated after 20 h with varying concentrations of the antibody in question as the competitor for 1 hour. In a second incubation step, the antibody of the present invention is applied in a constant concentration of 100 nM and its binding is flow-cytometrically detected using a fluorescence-labelled antibody directed against the constant domains of the antibody of the invention. Binding that conducts anti-proportional to the concentration of the antibody in question is indicative for that both antibodies recognize the same epitope. However, many other assays are known in the art which may be used.

This antibody or the antigen-binding fragment of the present invention is not limited to the antibody detecting the above epitope of glycoprotein B of HSV-1 and HSV-2. In fact, also other antibodies which detect another epitope of glycoprotein B or even an epitope of another protein or polypeptide of HSV-1 and HSV-2 is envisaged as long as such an antibody a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹ as described herein above and below and/or being capable of inhibiting the spreading of HSV from an infected cell to an adjacent second non-infected cell (cell-to-cell spread) and/or being capable of inhibiting cell-to-cell spread independent from antibody-dependent cellular cytotoxicity (ADCC) and/or complement-dependent cytotoxicity (CDC) as described herein above and below.

With the normal skill of the person skilled in the art and by routine methods, the person skilled in the art can easily deduce from the sequences provided herein relevant epitopes (also functional fragments) of the polypeptides of HSV which are useful in the generation of antibodies like polyclonal and monoclonal antibodies. However, the person skilled in the art is readily in a position to also provide for engineered antibodies like CDR-grafted antibodies or also humanized and fully human antibodies and the like.

Particularly preferred in the context of the present invention are monoclonal antibodies. For the preparation of monoclonal antibodies, any technique which provides antibodies produced by continuous cell line cultures can be used. Examples for such techniques include the hybridoma technique, the trioma technique, the human B-cell hybridoma technique and the EBV-hybridoma technique to produce human monoclonal antibodies (Shepherd and Dean (2000), Monoclonal Antibodies: A Practical Approach, Oxford University Press, Goding and Goding (1996), Monoclonal Antibodies: Principles and Practice—Production and Application of Monoclonal Antibodies in Cell Biology, Biochemistry and Immunology, Academic Pr Inc, USA).

The antibody derivatives can also be produced by peptidomimetics. Further, techniques described for the production of single chain antibodies (see, inter alia, U.S. Pat. No. 4,946,778) can be adapted to produce single chain antibodies specifically recognizing an antigen of HSV. Also, transgenic animals may be used to express humanized antibodies to the polypeptide of HSV.

The present invention also envisages the production of specific antibodies against native polypeptides and recombinant polypeptides of glycoprotein B or any another protein or polypeptide of HSV-1 and HSV-2. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of HSV-1 or HSV-2 can be subcloned into an appropriated vector, wherein the recombinant polypeptide is to be expressed in an organism being able for an expression, for example in bacteria. Thus, the expressed recombinant protein can be intra-peritoneally injected into a mouse and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of HSV-1 and HSV-2 can be obtained by directly immunizing the animal by directly injecting intramuscularly the vector expressing the desired polypeptide or a protein or an epitope of HSV-1 and HSV-2, in particular an epitope of gB. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

The term “specifically binds”, as used herein, refers to a binding reaction that is determinative of the presence of the desired polypeptide or a protein or an epitope of HSV-1 and HSV-2, in particular an epitope of gB, and an antibody in the presence of a heterogeneous population of proteins and other biologics.

Thus, under designated assay conditions, the specified antibodies and a corresponding polypeptide or a protein or an epitope of HSV-1 and HSV-2, in particular an epitope of gB, bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The term “anti-HSV antibody or antigen-binding fragment thereof” means in accordance with this invention that the antibody molecule or antigen-binding fragment thereof is capable of specifically recognizing or specifically interacting with and/or binding to at least two amino acids of the desired polypeptide or a protein or an epitope of HSV-1 and HSV-2, in particular an epitope of gB. Said term relates to the specificity of the antibody molecule, i.e. to its ability to discriminate between the specific regions a desired polypeptide or a protein or an epitope of HSV-1 and HSV-2, in particular an epitope of gB. Accordingly, specificity can be determined experimentally by methods known in the art and methods as disclosed and described herein. Such methods comprise, but are not limited to Western blots, ELISA-, RIA-, ECL-, IRMA-tests and peptide scans. Such methods also comprise the determination of Kd-values as, inter alia, illustrated in the appended examples. The peptide scan (pepspot assay) is used routinely employed to map linear epitopes in a polypeptide antigen. The primary sequence of the polypeptide is synthesized successively on activated cellulose with peptides overlapping one another. The recognition of certain peptides by the antibody to be tested for its ability to detect or recognize a specific antigen/epitope is scored by routine colour development (secondary antibody with horseradish peroxide and 4-chloronaphtol and hydrogenperoxide), by a chemoluminescence reaction or similar means known in the art. In the case of, inter alia, chemoluminescence reactions, the reaction can be quantified. If the antibody reacts with a certain set of overlapping peptides one can deduce the minimum sequence of amino acids that are necessary for reaction. The same assay can reveal two distant clusters of reactive peptides, which indicate the recognition of a discontinuous, i.e. conformational epitope in the antigenic polypeptide (Geysen (1986), Mol. Immunol. 23, 709-715).

In a preferred embodiment, the present invention relates to an anti-HSV antibody or antigen-binding fragment thereof which recognizes the same epitope as said antibody as defined herein-above, wherein the recognition of said epitope is determined by cryo-electron microscopy (Cryo-EM).

As mentioned, the antibody of the present invention binds to a specific epitope on gB comprising the following contact amino acid residues in HSV-1 gB D199, A203, K204, Y303, R304, Q321, V322, D323, K320, Y326, R335 and T337 or the following in HSV-2 gB D191, A195, K196, Y295, R296, K312, Q313, V314, D315, Y318, R327 and T329. This epitope has been determined by cryo-electron microscopy (CryoEM) analysis.

In a preferred embodiment, the antibody of the present invention binds to a specific epitope on gB consisting of the following contact amino acid residues in HSV-1 gB D199, A203, K204, Y303, R304, Q321, V322, D323, K320, Y326, R335 and T337 or the following in HSV-2 gB D191, A195, K196, Y295, R296, K312, Q313, V314, D315, Y318, R327 and T329. This epitope has been determined by cryo-electron microscopy (CryoEM) analysis. Thus, in a particular embodiment, the epitope is determined by cryo-electron microscopy (Cryo-EM).

As outlined above, whether an antibody binds to the same epitope as a reference antibody can be determined by methods known to the person skilled in the art. Yet, in one embodiment, this is determined as described in the Examples appended hereto. In a particular embodiment, the epitope is determined by cryo-electron microscopy (Cryo-EM).

In a preferred embodiment the epitope can be determined as outlined in the following: HSV-1 gB and HDIT102(H4) Fab is mixed in an appropriate ratio. An aliquot of the mixture is adsorbed onto appropriate grids, blotted with filter paper and vitrified into liquid ethane at −180° C. Data of HSV-1 gB and HDIT102(H4)-Fab complex is acquired on a transmission electron microscope. Micrograph movies are recorded in counting mode at an appropriate magnification and with appropriate dose.

Data processing and model building is done using specific software for image processing steps, dose weighting and motion correction of dose-fractionated and gain-corrected movies and contrast transfer function (CTF) parameter estimation. Micrographs displaying strong drift, astigmatism and maximum CTF resolution worse than 8 Å are excluded from further processing. A total of 1-10 million particles are picked. The particle dataset is cleaned through three rounds of reference-free 2D classification. Stochastic algorithms are used to generate a de novo 3D initial model from the 2D particles. The particle dataset is further cleaned through three rounds of unsupervised 3D classification. The remaining particles are subjected to Bayesian particle polishing, CTF and aberration refinement, and a final high-resolution 3D refinement, which results in a final map.

The HSV-1 gB X-ray structure (PDB-ID: 2GUM) can manually be mutated at positions T313S, Q443L and V553A and placed into the final map using specific software. For the HDIT102(H4) Fab, the crystal structure of a humanized recombinant Fab fragment (PDB-ID 7PHU) is mutated in silico based on a sequence alignment. Three HDIT102(H4) Fabs are placed into the final map in silico. Specialized software is used for the initial fitting of HSV-1 gB and the three HDIT102(H4) Fabs into the final map. The final protein model is obtained by several iterations of manual model building, refinement and model validation. Data collection, refinement and validation statistics can be done as summarized in Table 1 and the data processing workflow in FIG. 8D.

In an even more preferred embodiment, more specifically, the epitope can be determined as outlined in the following:

HSV-1 gB and HDIT102(H4) Fab is mixed in a ratio of 1 to 3.5. A 4 μl aliquot of the mixture is adsorbed onto glow-discharged Quantifoil Cu-R2/1-300 mesh holey carbon-coated grids (Quantifoil, Germany), blotted with Whatman 1 filter paper and vitrified into liquid ethane at −180° C. using a Leica EM GP2 plunger (Leica microsystems, Austria) operated at 10° C. and 85% humidity. Data of HSV-1 gB and HDIT102(H4)-Fab complex is acquired on a Titan Krios G1 TEM (ThermoFisher, USA) operated at 300 kV and equipped with a Gatan Energy Filter (Ametek, USA) and a K2 Summit direct electron detector (Ametek, USA). Micrograph movies of 40 frames are recorded in counting mode at a magnification of 165,000× (pixel size 0.82 Å) with a dose of 1.15 e⁻/Å²/frame, resulting in a total accumulated dose on the specimen level of approximately 46 e⁻/Å² per exposure.

Data processing and model building is done as follows. All image processing steps are performed with Relion v4.0 (Kimanius, Dong et al., 2021, Biochem J, Vol. 478 (24)). Dose weighting and motion correction of dose-fractionated and gain-corrected movies are performed using Relion's implementation of the UCSF motioncor2 program. Contrast transfer function (CTF) parameters are estimated using ctffind 4.1.14 (Rohou and Grigorieff, 2015, J Struct Biol, Vol. 192 (2)). Micrographs displaying strong drift, astigmatism greater than 1000 Å and maximum CTF resolution worse than 8 Å are excluded from further processing. A total of 1 million particles are picked using the Laplacian-of-Gaussian (LoG) filter in Relion 4.0 (Kimanius, Dong et al., 2021, Biochem J, Vol. 478 (24)). The particle dataset is cleaned through three rounds of reference-free 2D classification resulting in 277′376 particles. Relion's Stochastic Gradient Desecnt (SGD) algorithm is used to generate a de novo 3D initial model from the 2D particles. The particle dataset is further cleaned through three rounds of unsupervised 3D classification. The remaining 208′280 particles are subjected to Bayesian particle polishing, CTF and aberration refinement, and a final high-resolution 3D refinement, which results in a final map with an overall resolution of 3.44 Å according to the gold standard Fourier shell correlation (FSC) at FSC=0.143.

The HSV-1 gB X-ray structure (PDB-ID: 2GUM) can manually be mutated at positions T313S, Q443L and V553A and placed into the final map using coot (Casanal, Lohkamp et al., 2020, Protein Sci, Vol. 29 (4)). For the HDIT102(H4) Fab, the crystal structure of a humanized recombinant Fab fragment (PDB-ID 7PHU) is mutated in coot (Casanal, Lohkamp et al., 2020, Protein Sci, Vol. 29 (4)) based on a sequence alignment generated by Needle EMBOSS (Rice, Longden et al., 2000, Trends Genet, Vol. 16 (6)). Three HDIT102(H4) Fabs are placed into the final map using coot (Casanal, Lohkamp et al., 2020, Protein Sci, Vol. 29 (4)). Molrep of the CCP-EM software suite v1.6 (Nicholls, Tykac et al., 2018, Acta Crystallogr D Struct Biol, Vol. 74 (Pt 6)) is used for the initial fitting of HSV-1 gB and the three HDIT102(H4) Fabs into the final map. The final protein model is obtained by several iterations of manual model building in coot (Casanal, Lohkamp et al., 2020, Protein Sci, Vol. 29 (4)), Refmac-Servalcat refinement and model validation in the CCP-EM software suite v1.6 (Nicholls, Tykac et al., 2018, Acta Crystallogr D Struct Biol, Vol. 74 (Pt 6)). Data collection, refinement and validation statistics can be done as summarized in Table 1 and the data processing workflow in FIG. 8D.

In a preferred embodiment, the present invention relates to an anti-HSV antibody or antigen-binding fragment thereof as defined herein above and below, wherein said anti-HSV antibody is a humanized or a fully human antibody.

“Humanization approaches” are well known in the art and in particular described for antibody molecules, e.g., Ig-derived molecules. The term “humanized” refers to humanized forms of non-human (e.g., murine) antibodies or fragments thereof (such as Fv, Fab, Fab′, F(ab′), scFvs, or other antigen-binding partial sequences of antibodies) which contain some portion of the sequence derived from non-human antibodies. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity.

Methods for humanizing non-human antibodies are well known in the art. Generally, a humanized antibody has one or more amino acids introduced into it from a source which is non-human in an attempt to retain the original binding activity of the antibody or to even improve it. Methods for humanization of antibodies/antibody molecules are further detailed in Jones et al., Nature 321 (1986), 522-525; Reichmann et al., Nature 332 (1988), 323-327; and Verhoeyen et al., Science 239 (1988), 1534-1536 and in patent literature, e.g., in EP-B1 451216. Specific examples of humanized antibodies, e.g., antibodies directed against EpCAM, are known in the art, see e.g. (LoBuglio, Proceedings of the American Society of Clinical Oncology Abstract (1997), 1562 and Khor, Proceedings of the American Society of Clinical Oncology Abstract (1997), 847).

In the context of humanization, the working principle is to improve the match between CDRs and FRs in an antibody. For improving this match, amino acid substitutions can be made either in the FRs or in the CDRs.

Humanized antibodies can be based on “chimeric antibodies” or on “CDR”-grafted antibodies. The term “chimeric” refers, in general terms, to an antibody in which a portion of the heavy and/or light chain is derived from a particular source or species, while the remainder of the heavy and/or light chain is derived from a different source or species.

Although the creation of an antibody chimera is normally undertaken to achieve a more human-like antibody (by replacing constant region of the mouse antibody with that from human) simple chimeras are not usually referred to as “humanized”. Rather, the amino acid sequence of a humanized antibody has usually undergone additional refinement so that the protein sequence of a humanized antibody is essentially identical to that of a human variant, despite the non-human origin of some of its complementarity-determining region (CDR) segments responsible for the ability of the antibody to bind to its target antigen.

More specifically, “humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (i.e., a CDR) of the recipient are replaced by residues from a hypervariable region of a non-human species (donor antibody) such as mouse, rat, rabbit or non-human primate having the desired antibody specificity, affinity, and capability. Thus, in certain embodiments, a humanized antibody will comprise substantially all of at least one, and typically two, variable domains, in which all or substantially all of the CDRs correspond to those of a non-human antibody, and all or substantially all of the frameworks correspond to those of a human antibody.

When “humanizing” an antibody, additional refinements, i.e., modifications to the antibody's amino acid sequence can be carried out that result, e.g., in a reduced immunogenicity in humans while retaining or substantially retaining the antigen-binding properties of the parent antibody.

Various modifications and adaptions in order to accommodate these variations are known to the person of skill in the art.

Thus, “humanizing” an antibody may in certain embodiments also include an additional refinement resulting in humanized antibodies that can comprise residues that are neither found in the recipient antibody nor in the donor antibody. These modifications are made to further refine antibody performance.

In some instances, framework region (FR) residues of the human immunoglobulin are replaced by corresponding non-human residues.

The humanized antibody optionally also will comprise at least a portion of an antibody/immunoglobulin constant region (Fc), typically that of a human immunoglobulin. A “humanized form” of an antibody, e.g., a non-human antibody, refers to an antibody that has undergone humanization. For further details, see Jones et al., Nature 321:522-525 (1986); Reichmann et al., Nature 332:323-329 (1988); and Presta, Curr. Op. Struct. Biol. 2:593-596 (1992).

Accordingly, in the context of this invention and in preferred embodiments, antibody molecules are provided, which are humanized and can successfully be employed in pharmaceutical compositions.

In a preferred embodiment, the anti-HSV antibody according the first aspect of the present invention is a full-length antibody, i.e., a full immunoglobulin molecule which is often also referred to as complete antibody.

The invention also provides anti-HSV antigen-binding fragments of the anti-HSV antibody according the first aspect of the present invention. Preferred antigen-binding fragments are described in the following:

“Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Pluckthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook (1989), loc. cit. The term “Ig-derived domain” particularly relates to (poly) peptide constructs comprising at least one CDR. Fragments or derivatives of the recited Ig-derived domains define (poly) peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

Accordingly, in the context of the present invention, the antibody molecule described herein above is selected from the group consisting of a full length antibody (immunoglobulin, like an IgG1, an IgG2, an IgG2a, an IgG2b, an IgA1, an IgGA2, an IgG3, an IgG4, an IgA, an IgM, an IgD or an IgE)), a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody.

Moreover, as already outlined above, the present invention also relates to antigen-binding fragments of the antibody of the present invention, preferably, selected from the group consisting of F(ab)-, Fab′-SH-, Fv-, Fab′-, F(ab′)2-fragments.

Thus, in a preferred embodiment, the anti-HSV antibody according the first aspect of the present invention is a human IgG1, an IgG2, an IgG2a, an IgG2b, an IgA1, an IgGA2, an IgG3, an IgG4, an IgA, an IgM, an IgD or an IgE antibody.

Moreover, in a preferred embodiment, the antigen-binding fragment of said anti-HSV antibody according the first aspect of the present invention is a human F(ab)-, Fab′-SH-, Fv-, Fab′-, or a F(ab′)2-fragment.

In in a further preferred embodiment, the anti-HSV antibody or the antigen-binding fragment thereof according the first aspect of the present invention has a dissociation constant Kd of at most 10 nM, preferably at most 8 nM, more preferably at most 4 nM, even more preferably at most 2 nM, at most 1 nM, at most 0.8 nM, at most 0.4 nM, at most 0.2 nM, at most 0.1 nM, at most 0.09 nM, at most 0.08 nM, at most 0.07 nM, at most 0.06 nM, at most 0.05 nM, at most 0.04 nM and most preferably at most 0.03 nM.

The Kd represents the dissociation constant as a measure of the propensity of a complex to dissociate reversibly into its components (i.e., the affinity of the antibody for the antigen) and is the inverse of the association constant. The Kd may be calculated from the Scatchard equation and methods for determining Kd are well known in the art.

As shown herein above and below, the anti-HSV antibody of the present invention provides a fully human antibody or antigen-binding fragment (HDIT102(H4)) that binds the glycoprotein B (gB) of Herpes Simplex Virus type 1 and type 2 at a conserved epitope with a Kd of approximately 0.09 to 0.03 nM.

The dissociation constant Kd may be determined by methods known to the person skilled in the art. In one embodiment, this dissociation constant Kd is determined as described in the Examples appended hereto.

Thus, in a preferred embodiment the Kd can be determined as outlined in the following: To produce recombinant HSV-1 gB protein a codon-optimized DNA sequence encoding for HSV-1 gB ectodomain (aa 30-729; UniProtKB P06436.1) or HSV-2 gB ectodomain (aa 22-724; GenBank: QAU10948.1) including a signal peptide and a C-terminal tag can be cloned into a mammalian expression vector. HSV gB recombinant protein can then transiently be expressed in HEK293-E6 suspension cells cultured in culture media. HEK293-E6 cells are transfected with plasmid encoding gB. On day 5 after the transfection the supernatant is harvested by centrifugation steps. Next, the pH of the supernatant is adjusted by the addition of 1 ml 2 M Tris buffer pH 9/100 ml supernatant. Afterwards the gB protein is purified tag-affinity gravity flow purification. The tag of gB protein is then removed by thrombin digestion and dialyzed against 50 mM Tris, 150 mM NaCl, pH 8. The thrombin digested gB protein is then purified three times by tag-specific affinity chromatography to deplete the sample from any remaining tagged gB protein. Next, the gB protein is concentrated and is further purified via size-exclusion chromatography. The peek fractions are pooled and concentrated.

Anti-HSV gB antibody Fab can be generated either by papain digest and subsequent purification (as done for HDIT101), or generated recombinantly by transfection of light and truncated heavy chain encoding plasmids into 293T-E6 cells (as done for HDIT102(H4)). HSV gB recombinant protein is biotinylated and afterwards, the residual biotin is removed. An initial loading scout is performed to find out the best biosensor loading concentration. The streptavidin biosensors (of the Octet) are loaded with different concentrations of biotinylated gB and the absorption kinetics of the test antibody Fab fragments are measured. A dilution series of Fab fragments is then analysed to determine the association rate (ka), the dissociation rate (kdis) and the dissociation constant Kd (Kd=kdis/ka).

More specifically, the Kd can be determined as outlined in the following:

To produce recombinant HSV-1 gB protein a codon-optimized DNA sequence encoding for HSV-1F gB ectodomain (aa 30-729; UniProtKB P06436.1) or HSV-2G gB ectodomain (aa 22-724; GenBank: QAU10948.1) including a BM40 signal peptide and a C-terminal double Strep-tag can be cloned into a pCAGGS mammalian expression vector. HSV gB recombinant protein can then transiently be expressed in HEK293-E6 suspension cells cultured in serum-free media, e.g. F17 medium (ThermoFisher) supplemented with 0.1% Kolliphor (Sigma) and 4 mM Glutamine. HEK293-E6 cells are transfected with PeiMax (Polysciences) at a cell density of 1.5-2.0×10⁶ cells/ml with 1 μg plasmid encoding gB and 2 μg PeiMax/ml culture media. Twenty-four hours after the transfection Tryptone N1 feeder (Organi Technie) is added to the cultures. On day 5 after the transfection the supernatant is harvested by two centrifugation steps, first 1200 rpm to remove the cells and then 3600 rpm to remove cell debris. Next, the pH of the supernatant is adjusted by the addition of 1 ml 2 M Tris buffer pH 9/100 ml supernatant. Afterwards the gB protein is purified by Strep-Tactin XT (IBA, Germany) gravity flow purification according to the manufacturer protocol. The Strep-tag of gB protein is then removed by thrombin digestion (Serva) and dialyzed against 50 mM Tris, 150 mM NaCl, pH 8. The thrombin digested gB protein is then purified three times by Strep-Tactin XT affinity chromatography to deplete the sample from any remaining Strep-tagged gB protein. Next, the gB protein is concentrated with Amicon spin columns (cut-off 30 k) and is further purified with a Superdex 200 10/300 GL SEC column and an Äkta Pure FPLC. The peek fractions are pooled and concentrated with Amicon spin columns.

Anti-HSV gB antibody Fab can be generated either by papain digest and subsequent purification (as done for HDIT101), or generated recombinantly by transfection of light and truncated heavy chain encoding plasmids into 293T-E6 cells (as done for HDIT102(H4)). HSV gB recombinant protein is biotinylated (NHS-PEG4-Biotin (Thermo Fischer Scientific, A39259)) in ratio 3:1 for 30 min at room temperature and afterwards, the residual biotin is removed by using desalting columns and centrifugation at 1000 g for 2 min (Zeba Spin Desalting Columns; 7K MWCO, 2 ml (Thermo Scientific UE285726). An initial loading scout is performed to find out the best biosensor loading concentration. The streptavidin biosensors (of the Octet) are loaded with different concentrations of biotinylated gB and the absorption kinetics of the test antibody Fab fragments are measured. The optimal gB concentration for loading the biosensor was determined with 5 μg/ml. Biotinylated gB (wt) [5 μg/ml] is used to load the biosensor and the binding kinetics of antibody Fab fragments (100 nM) against immobilized gB can be analyzed using global 1:1 fit model. A dilution series of Fab fragments is then analysed to determine the association rate (ka), the dissociation rate (kdis) and Kd (Kd=kdis/ka).

Other methods for determining the dissociation constant Kd are also known to the person skilled in the art. Without being bound to theory, as a further example, the dissociation constant Kd can generally also be determined by, e.g., using infected cells.

Accordingly, the dissociation constant Kd can be determined as outlined in the following: To determine the dissociation constant Kd, Vero cells (~80% confluent) are infected with a multiplicity of infection (MOI) of 1 with either HSV-1F or HSV-2G. After an incubation period of 16-20 hours, the virus-containing medium is discarded, and the cells are washed once with PBS, resuspended and harvested. Afterwards, the cells are pelleted at 300×g for 5 min and resuspended at a cell density of 5.0×10⁶ cells/ml in PBS. The suspension is evenly distributed in a volume of 100 μl/well on a 96-well plate. To determine the equilibrium dissociation constant, the HSV infected Vero cells are incubated in triplets with a 1:2 dilution series (0.03-500 nM) of the unconjugated primary antibody for 45 min at room temperature. After two times washing, a FITC-conjugated anti human Fc detection antibody (Fcγ IgG, polyclonal Rabbit anti-Human FITC, Jackson ImmunoResearch (309-096-008)) is added, targeting the Fc domain of the primary antibody. After an incubation period of 30 min cells are washed twice and taken up in 500 μl PBS. Following this, the mean fluorescence intensity (MFI) is determined for each sample by flow cytometry. As a negative control for staining, infected cells are incubated with only secondary antibodies. As a negative control for specificity, uninfected Vero cells are stained in the same way. The equilibrium dissociation constant (half maximal binding or half maximal saturation of antibody) is calculated under application of the one site specific binding method for nonlinear regression with Graph Pad Prism. The calculation principal follows as: Y=Bmax×X/(KD+X), based on a curve which is known as a rectangular hyperbola, binding isotherm, or saturation binding curve. Y is zero initially and increases to a maximum plateau value Bmax. The equation describes the equilibrium binding of a ligand to a receptor as a function of increasing ligand concentration. X is the concentration of the ligand, Y is the specific binding, Bmax is the maximum number of binding sites, expressed in the same units as the Y-axis. KD is the equilibrium dissociation constant, expressed in the same units as the X-axis (concentration). When the drug concentration equals KD, half the binding sites are occupied at equilibrium, i.e., for the normalized Y-values 50% of binding is observed (EC50).

In in a further preferred embodiment, the anti-HSV antibody or the antigen-binding fragment thereof according the first aspect of the present invention is capable of neutralizing HSV.

In a further more preferred embodiment, the antibody or antigen-binding fragment thereof is capable of neutralizing HSV-1 and/or HSV-2.

Accordingly, in a further preferred embodiment, the anti-HSV antibody or the antigen-binding fragment thereof according the first aspect of the present invention in a concentration of at most 20 nM, preferably of at most 16 nM, more preferably of at most 12 nM, of at most 10 nM, of at most 8 nM, of at most 6 nM, and most preferably of at most 4 nM, is capable of neutralising a defined amount of HSV of 100 TCID50.

“Neutralizing” herein means that the antibody opsonizes the virus so that it cannot infect any further cell. An assay for testing whether an antibody in a concentration of, e.g., at most 20 nM is capable of neutralizing a defined amount of HSV of, e.g., 100 TCID₅₀ Eis-Hübinger et al., Intervirology 32:351-360 (1991); Eis-Hübinger et al., Journal of General Virology 74:379-385 (1993) and in Examples 1 and 2 of WO2011/038933 A2. Thus, in a preferred embodiment, the antibody of the invention in a concentration of at most 20 nM, preferably of at most 16 nM, more preferably of at most 12 nM, of at most 10 nM, of at most 8 nM, of at most 6 nM, and most preferably of at most 4 nM, is capable of neutralizing a defined amount of HSV of 100 TCID₅₀ to more than 50%, preferably by more than 60%, more preferably by more than 80%, more preferably by more than 90%, such as more than 95%, more preferably 96%, e.g., more than 97%, and most preferably more than 98%, e.g., more than 99% or even 100%.

As shown herein above and below, the anti-HSV antibody of the present invention inhibits cell-free HSV-1 infection of Vero cells with an IC50 concentrations of at most 30 nM, at most 20 nM, more preferably at most 10 nM, at most 8 nM and most preferably at most 6 nM.

Preferably, the IC50 is determined as follows:

The IC50 may be determined by methods known to the person skilled in the art. In one embodiment, this IC50 is determined as described in the Examples appended hereto. In a particular embodiment, this IC50 is determined by using infected cells.

In a preferred embodiment the IC50 is determined as follows:

To examine the antiviral activity of anti-HSV gB antibodies towards cell-free virus the following method can be used. Different antibody dilutions are incubated with a constant viral dose (100 TCID50 HSV-1 or HSV-2). The antibody-virus mixtures are applied to 80-90% confluent Vero cells in 96-well plates in a volume of 100 μl per well. As a control, Vero cells are infected with a viral dose of 100 TCID50 without prior incubation with neutralizing antibody. The extent of the cytopathic effect is examined by light microscopy three days after infection. The neutralization concentration is determined to be the highest antibody dilution at which the virus is still completely neutralized and the formation of a CPE in the inoculated cell cultures is completely prevented. In addition, the neutralizing antibody concentration at which 50% of the cell culture wells are protected from infection (IC50) are calculated.

More specifically, the IC50 can be determined as outlined in the following:

To examine the antiviral activity of anti-HSV gB antibodies towards cell-free virus the following method can be used. Different antibody dilutions are incubated with a constant viral dose (100 TCID50 HSV-1F or HSV-2G) for 1 h at 37° C. The antibody-virus mixtures are applied to 80-90% confluent Vero cells in 96-well plates (2.0×10⁴ cells per well) in a volume of 100 μl per well. As a control, Vero cells are infected with a viral dose of 100 TCID50 without prior incubation with neutralizing antibody. The extent of the cytopathic effect is examined by light microscopy three days after infection. The neutralization titer is determined to be the highest antibody dilution at which the virus is still completely neutralized and the formation of a CPE in the inoculated cell cultures is completely prevented. In addition, the neutralizing antibody concentration at which 50% of the cell culture wells are protected from infection (IC50) are calculated using the 50% neutralization titer formula (Krawczyk, Krauss et al., 2011, J Virol, Vol. 85 (4)):

T=x+((b/10)·x), T=neutralizing antibody titer at which 50% of the infected cell cultures are protected from infection. x=Lowest antibody dilution for which at least 50% of cell cultures are infected. b=number of infected cell cultures, which at dilution x exceed the 50% infection rate.

In a further preferred embodiment, the anti-HSV antibody or the antigen-binding fragment thereof according the first aspect of the present invention is capable of inhibiting the spreading of HSV from an infected cell to an adjacent second non-infected cell (cell-to-cell spread).

The ability of antibodies to block cell-to-cell spread was recently correlated with the ability of antibodies to block recurrences of orolabial HSV-1 outbreaks (Alt, Wolf et al., 2023, Front Immunol, Vol. 14)).

Cell-to-cell spread is the ability of the herpes virus to spread to an adjacent second non-infected cell without releasing cell-free particles. Reducing or eliminating the ability of the herpes virus to spread to an adjacent cell has the beneficial effect that the generation of lesions is avoided. In order to examine whether an antibody is capable of inhibiting the spread of HSV from an infected cell to an adjacent second non-infected cell (cell-to-cell spread), methods well-known to the person skilled in the art can be used.

As an example, the following assay can be used: Vero cells grown to confluency on glass cover slips in 24-well tissue culture plates are infected for 4 h at 37° C. with a constant virus amount of 400 TCID₅₀/well. One median tissue culture infective dose (1 TCID₅₀) is the mount of a cytopathogenic agent, such as a virus, that will produce a cytopathic effect in 50% of the cell cultures inoculated. The virus inoculum is subsequently removed, the cells washed twice with PBS and further incubated for 2 days at 37° C. in 1 ml DMEM, 2% FCS, Pen/Strep containing an excess of either different anti-HSV antibodies or polyclonal anti-HSV control serum in order to prevent viral spreading via the supernatant. Viral antigens of HSV-infected cells are detected with a fluorescence labelled polyclonal goat-anti-HSV-serum (e.g. from BETHYL Laboratories, Montgomery, TX USA, Catalog No. A190-136F, Lot No. A190-136F-2). Preferably, an antibody is inhibiting cell-to-cell spread if less than 20% of the adjacent cells are infected, preferably wherein less than 15%, less than 10%, less than 5%, more preferably less than 3% and most preferably less than 1% of the adjacent cells are infected in the above assay.

Cell-to-cell spread may also be assayed as follows: The presence of neutralizing antibodies does not necessarily prevent cell-to-cell spread of herpesviridae. To compare antibodies on disruption of HSV-1 and HSV-2 cell-to-cell spread this particular dissemination mode can be mimicked in vitro using standard test methods. E.g.: To infect individual cells, confluent Vero cell monolayers are initially incubated with either HSV-1 or HSV-2 at low MOI (e.g. 100 TCID50), respectively. After 4 h of adsorption at 37° C., the viral inoculum is removed. To promote direct cell-to-cell transmission from individually infected cells but prevent viral spread through viral particles across the cell culture supernatant, Vero cell monolayers are treated with an excess of neutralizing anti-gB antibodies, controls, or medium alone. After 48 h virus spread can be detected by immunostaining with a mouse monoclonal antibody specific for a common epitope on glycoprotein D of HSV-1 and HSV-2 (e.g. Acris Antibodies, San Diego, CA, USA) and fluorescence-conjugated secondary antibody. Immunofluorescence images can be acquired with a fluorescence microscope at a 100- or 400-fold magnification.

The capability of inhibiting the spreading of HSV from an infected cell to an adjacent second non-infected cell (cell-to-cell spread) may also be determined as described in the Examples appended hereto. In a particular embodiment, this cell-to-cell spread is determined by using infected cells.

In in a further preferred embodiment, the anti-HSV antibody or the antigen-binding fragment thereof according the first aspect of the present invention exerts its antiviral or neutralizing activity independent from antibody-dependent cellular cytotoxicity (ADCC) and/or complement-dependent cytotoxicity (CDC), preferably, said antibody is capable of inhibiting cell-to-cell spread independent from antibody-dependent cellular cytotoxicity (ADCC) and/or complement-dependent cytotoxicity (CDC).

As the above-described assays for testing the capability whether an antibody is capable of inhibiting cell-to-cell spread do not contain complement and/or cytotoxic effector cells, the same assays may be used in order to determine whether an antibody is capable of inhibiting cell-to-cell spread independent from antibody-dependent cellular cytotoxicity (ADCC) and/or complement-dependent cytotoxicity (CDC).

In in a further preferred embodiment, the anti-HSV antibody or the antigen-binding fragment thereof according the first aspect of the present invention is conjugated to an effector moiety, a therapeutic moiety, or a detectable label.

In this context, the term “conjugated” refers to any method known in the art for functionally connecting protein domains, including without limitation recombinant fusion with or without intervening domains, intein-mediated fusion, non-covalent association, and covalent bonding, e.g., disulfide bonding peptide bonding, hydrogen bonding, electrostatic bonding, and conformational bonding, e.g., biotin-avidin associations. The conjugation to an effector moiety can be either by chemical or recombinant means. Chemical means refers to a reaction between the antibody and the effector moiety such that there is a covalent bond formed between the two molecules to form one molecule.

The term “effector moiety” means a compound intended to have an effect on a cell targeted by the antibody. The effector moiety may be, for example, a therapeutic moiety or a detectable moiety.

A “therapeutic moiety” is a compound intended to act as a therapeutic agent, such as a cytotoxic agent or drug. Examples of compounds are given below for the pharmaceutical composition.

A “detectable label” includes any compound or protein-tag detectable by spectroscopic, photochemical, biochemical, immunochemical, electrical, optical or Chemical means, such as a fluorescent label.

In the following, the second aspect, is described in more detail.

As mentioned above, in a second aspect, which is related to the above first aspect, the present invention relates to combination of
(A) an anti-HSV antibody or an antigen-binding fragment thereof according the first aspect of the present invention as defined above; and
(B) an anti-HSV antibody or an antigen-binding fragment thereof binding to the glycoprotein B (gB) of the HSV-1 and/or HSV-2, wherein said antibody comprises: the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO: 16; wherein said antibody has a dissociation constant Kd of at most 40 nM, preferably at most 30 nM, more preferably at most 20 nM, even more preferably at most 15 nM, at most 13 nM and at most 10 nM.

The first component (A), i.e., the anti-HSV antibody or the antigen-binding fragment thereof according the first aspect of the present invention has already been described above. As regards the preferred embodiments of the “the anti-HSV antibody or the antigen-binding fragment thereof according the first aspect of the present invention” the same applies, mutatis mutandis, to this first component (A) as has been set forth above in the context of the first aspect of the present invention as defined above.

The second component (B), is an anti-HSV antibody or an antigen-binding fragment thereof binding to the glycoprotein B (gB) of the HSV-1 and/or HSV-2, wherein said antibody comprises:
the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO: 16;
wherein said antibody has a dissociation constant Kd of at most 40 nM, preferably at most 30 nM, more preferably at most 20 nM, even more preferably at most 15 nM, at most 13 nM and at most 10 nM.

Methods for the determination of the dissociation constant Kd to be of at most 40 nM, preferably at most 30 nM, more preferably at most 20 nM, even more preferably at most 15 nM, at most 13 nM and at most 10 nM has already been described above in the context of the first aspect of the present invention. The same applies, mutatis mutandis, to the second component (B) of the second aspect of the present invention.

The term “CDR” as employed in relation to the second component (B) of the second aspect of the present invention relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J. Mol. Biol. 196 (1987), 901-917 or Chothia Nature 342 (1989), 877-883.

In the context of the present invention, the CDR regions (as well as the framework regions (FR)) are determined according to the numbering scheme of Martin as described in Norman, R. A., F. Ambrosetti, A. Bonvin, L. J. Colwell, S. Kelm, S. Kumar and K. Krawczyk (2020). “Computational approaches to therapeutic antibody design: established methods and emerging trends.” Brief Bioinform 21(5): 1549-1567.

Hence, in the context of the present invention, reference to amino acid residues is according to the numbering scheme of Martin.

Accordingly, in the context of the second component (B) of the second aspect of the present invention, the antibody molecule described herein above is selected from the group consisting of a full length antibody (immunoglobulin, like an IgG1, an IgG2, an IgG2a, an IgG2b, an IgA1, an IgGA2, an IgG3, an IgG4, an IgA, an IgM, an IgD or an IgE), a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody. Moreover, also antigen-binding fragments of said antibody molecule according to the second component (B) of the second aspect of the present invention are envisaged, preferably, selected from the group consisting of F(ab)-, Fab′-SH-, Fv-, Fab′-, F(ab′)2-fragments.

Moreover, the antibody of the second component (B) of the second aspect of the present invention is an antibody or antigen-binding fragment thereof that binds to the glycoprotein B (gB) of HSV-1 and/or HSV-2.

In a preferred embodiment, the antibody of the second component (B) of the second aspect of the present invention is an antibody or antigen-binding fragment thereof comprises or consists of VH domain (heavy chain variable region) and VL domain (light chain variable region), i.e., the amino acid sequence of the variable region of the heavy chain of an antibody as depicted in SEQ ID NO:19 and the amino acid sequence of the variable region of the light chain of an antibody as depicted in SEQ ID NO:20.

Thus, in a preferred embodiment, the antibody or antigen-binding fragment of the second component (B) of the second aspect of the present invention is an antibody or antigen-binding fragment thereof which comprises the V_{H} of SEQ ID NO:19 and the V_{L} of SEQ ID NO:20.

However, the antibody or antigen-binding fragment thereof as used in the present invention is not particularly limited to such variable heavy and light chain variable regions but may also be an antibody or antigen-binding fragment thereof that binds to the glycoprotein B (gB) of HSV-1 and/or HSV-2 envelope which comprises or consists of VH domain and VL domain with at least 95%, 90%, 85%, 75%, 70%, 65%, 60%, 55% or 50% sequence homology with the sequences of SEQ ID NOs: 19 and 20, respectively, as long as the antibody or antigen-binding fragment has a dissociation constant Kd of at most 40 nM, preferably at most 30 nM, more preferably at most 20 nM, even more preferably at most 15 nM, at most 13 nM and at most 10 nM, or is capable of inhibiting the spreading of HSV from an infected cell to an adjacent second non-infected cell (cell-to-cell spread) or is capable of inhibiting cell-to-cell spread independent from antibody-dependent cellular cytotoxicity (ADCC) and/or complement-dependent cytotoxicity (CDC) as described herein above and below.

Furthermore, the antibody or antigen-binding fragment thereof is a molecule that comprises VH and VL domains having up to 0, 1, 2, 3, 4, 5, 6, 7, 8, 9, 10 or more conservative amino acid substitutions with reference to the sequences of SEQ ID NOs: 19 and 20. Moreover, the antibody or antigen-binding fragment thereof is an antibody fragment selected from the group consisting of Fab, Fab′, Fab′-SH, FV, scFV, F(ab′)2, and a diabody.

In order to determine whether an amino acid sequence has a certain degree of identity to the sequences of SEQ ID NOs: 19 and 20, the skilled person can use means and methods well known in the art, e.g., alignments, either manually or by using computer programs known to the person skilled in the art. Such an alignment can, e.g., be done with means and methods known to the skilled person, e.g., by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences. In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the nucleic acid sequences or with the amino acid sequences as described above which are capable of binding to gB of HSV-1 or HSV-2 and having a dissociation constant Kd of at most 40 nM, preferably at most 30 nM, more preferably at most 20 nM, even more preferably at most 15 nM, at most 13 nM and at most 10 nM, or being capable of inhibiting the spreading of HSV from an infected cell to an adjacent second non-infected cell (cell-to-cell spread) or being capable of inhibiting cell-to-cell spread independent from antibody-dependent cellular cytotoxicity (ADCC) and/or complement-dependent cytotoxicity (CDC) as described herein above and below, when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

Preferably, the amino acid substitution(s) are “conservative substitution(s)” which refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson Molecular Biology of the Gene, The Benjamin/Cummings Pub. Co. 4th Ed. (1987), 224. In addition, substitutions of structurally or functionally similar amino acids are less likely to disrupt biological activity. Within the context of the present invention the binding compounds/antibodies of the present invention comprise polypeptide chains with sequences that include up to 0 (no changes), 1, 2, 3, 4, 5, 6, 7, 8, 9, 10, 12, 15, 20 or more conservative amino acid substitutions when compared with the specific amino acid sequences disclosed herein, for example, SEQ ID NO: 19 (referring to the variable region of the antibody heavy chain of the antibody) and 20 (referring to the variable of the light chain of the antibody). As used herein, the phrase “up to X” conservative amino acid substitutions includes 0 substitutions and any number of substitutions up to 10 and including 0, 1, 2, 3, 4, 5, 6, 7, 8, 9, 10 substitutions.

Such exemplary substitutions are preferably made in accordance with those set forth in Table 1 as already displayed above in the context of the first aspect of the present invention. The same applies, mutatis mutandis, to the second aspect of the present invention.

Moreover, in a preferred embodiment, the antibody or antigen-binding fragment of the second component (B) of the second aspect of the present invention is an antibody or antigen-binding fragment thereof that comprises the following framework regions:
an amino acid sequence with at least 70% sequence identity to each one of the amino acid residues shown in positions 1 to 30 (V_{H}FR1), 38 to 51 (V_{H}FR2), 68 to 99 (V_{H}FR3), and 112 to 122 (V_{H}FR4) of SEQ ID NO: 17, 1 to 23 (V_{L}FR1), 41 to 55 (V_{L}FR2), 63 to 94 (V_{L}FR3), and 104 to 114 (V_{L}FR4) of SEQ ID NO: 18.

As mentioned above, in the context of the present invention, the CDR regions (as well as the framework regions (FR)) are determined according to the numbering scheme of Martin as described in Norman, R. A., F. Ambrosetti, A. Bonvin, L. J. Colwell, S. Kelm, S. Kumar and K. Krawczyk (2020). “Computational approaches to therapeutic antibody design: established methods and emerging trends.” Brief Bioinform 21(5): 1549-1567.

Hence, in the context of the present invention, reference to amino acid residues is according to the numbering scheme of Martin.

In a further, preferred embodiment, the antibody or antigen-binding fragment of the second component (B) of the second aspect of the present invention comprises an amino acid sequence with at least 75%, at least 80%, more preferably at least 85%, at least 90%, even more preferably at least 95%, and most preferably 98% or 99% overall sequence identity in the framework regions compared to each one of the amino acid residues shown in positions 1 to 30, 38 to 51, 68 to 99, and 112 to 122 of SEQ ID NO: 17 and in positions 1 to 23, 41 to 55, 63 to 94, and 104 to 114 of SEQ ID NO: 18. Such antibodies are suitable for the medical uses of the present invention as long as the antibody or antigen-binding fragment binds to gB of HSV-1 or HSV-2 and has a dissociation constant Kd of at most 40 nM, preferably at most 30 nM, more preferably at most 20 nM, even more preferably at most 15 nM, at most 13 nM and at most 10 nM, or is capable of inhibiting the spreading of HSV from an infected cell to an adjacent second non-infected cell (cell-to-cell spread) or is capable of inhibiting cell-to-cell spread independent from antibody-dependent cellular cytotoxicity (ADCC) and/or complement-dependent cytotoxicity (CDC) as described herein above and below.

Thus, in a preferred embodiment, the antibody or antigen-binding fragment of the second component (B) of the second aspect of the present invention comprises an amino acid sequence having the above variable regions of the light and heavy chains (i.e., the CDRs defined above, i.e., V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16) while the amino acid sequence have a variability in the framework region with at least 75%, at least 80%, more preferably at least 85%, at least 90%, even more preferably at least 95%, and most preferably 98% or 99% overall sequence identity in the framework regions compared to each one of the amino acid residues shown in positions 1 to 30, 38 to 51, 68 to 99, and 112 to 122 of SEQ ID NO: 17 and in positions 1 to 23, 41 to 55, 63 to 94, and 104 to 114 of SEQ ID NO: 18.

In this context, a polypeptide has “at least X % sequence identity” in the framework regions to SEQ ID NO:17 or 18 if SEQ ID NO:17 or SEQ ID NO:18 is aligned with the best matching sequence of a polypeptide of interest and the amino acid identity between those two aligned sequences is at least X % over positions 1 to 30, 38 to 51, 68 to 99, and 112 to 122 of SEQ ID NO: 17 and positions 1 to 23, 41 to 55, 63 to 94, and 104 to 114 of SEQ ID NO: 18. As mentioned above, such an alignment of amino acid sequences can be performed using, for example, publicly available computer homology programs such as the “BLAST” program provided on the National Centre for Biotechnology Information (NCBI) homepage using default settings provided therein. Further methods of calculating sequence identity percentages of sets of amino acid sequences or nucleic acid sequences are known in the art.

Moreover, in a preferred embodiment, the antibody or antigen-binding fragment of the second component (B) of the second aspect of the present invention comprises the V_{H} of SEQ ID NO:19 and the V_{L} of SEQ ID NO:20.

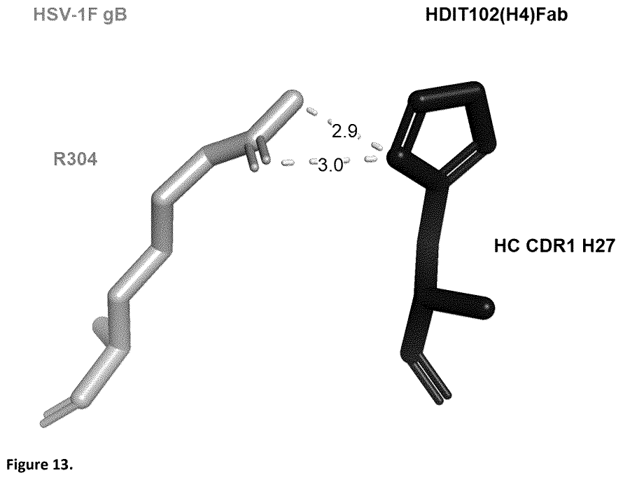

The specificity of the antibody or antigen-binding fragment of the second component (B) of the second aspect of the present invention may not only be expressed by the nature of the amino acid sequence of the antibody or the antigen-binding fragment as defined above but also by the epitope to which the antibody is capable of binding to. Thus, the present invention utilizes in a preferred embodiment an antibody or antigen-binding fragment which recognizes the same epitope as the antibody or antigen-binding fragment of the second component (B) of the second aspect of the present invention as described above. As shown in the Examples section and as illustrated in FIGS. 13A and 13B of WO2011/038933 A2, this epitope is a discontinuous or rather a pseudocontinuous epitope partially resistant to denaturation located at the amino acids 172-195 and 295-313 of glycoprotein B of HSV-1 and HSV-2. In the context of the present application, the epitope of the mAb 2c antibody may be located within the first 487 amino-terminal residues of the gB protein. Preferably, the epitope may comprise at least one amino acid sequence located within the amino acid sequence between position 172 and 307 of the gB protein.

In a preferred embodiment, the antibody or antigen-binding fragment of the second component (B) of the second aspect of the present invention is an antibody that recognizes the same epitope as said above-defined antibody or antigen-binding fragment of the second component (B) of the second aspect of the present invention.

In a more preferred embodiment, said epitope is located at the amino acids Y301-E305 and H308, K320, D323, Y326, P339, T341, W356 and P358 of glycoprotein B of HSV-1 and corresponding sites Y293-E297 and H300, K312, D315, Y318, P331, T333, W348 and P350 of glycoprotein B of HSV-2, preferably,
wherein said epitope consists of the contact amino acid residues Y301-E305 and H308, K320, D323, Y326, P339, T341, W356 and P358 of glycoprotein B of HSV-1 strain F (SEQ ID NO:9) and of the contact amino acids residues Y293-E297 and H300, K312, D315, Y318, P331, T333, W348 and P350 of glycoprotein B of HSV-2 strain G (SEQ ID NO:40), respectively.

In a particular embodiment, the epitope is determined by cryo-electron microscopy (Cryo-EM).

Previously, the epitope that is bound by the antibody or antigen-binding fragment of the second component (B) of the second aspect of the present invention has been described in, e.g., WO2011/038933. Said epitope has been determined by using overlapping 15-mer peptides spanning the gB region from amino acid 31 to 505 as it has also been described in Daumer et al., Med Microbiol Immunol 2011 (200):85-97. Using high-resolution 13-mer peptide microarrays Krawczyk et al., Journal of Virology 2011 (85):1793-1803 this epitope recognized by mAb 2c (i.e., the epitope that is bound by the antibody or antigen-binding fragment of the second component (B) of the second aspect of the present invention) has previously been further mapped.

Yet, in the context of the present invention and as shown in the appended Examples, the epitope that is bound by the antibody or antigen-binding fragment of the second component (B) of the second aspect of the present invention has been characterized in more detail by using cryo-electron microscopy (Cryo-EM). As outlined in more detail herein above and below, said epitope is located at the amino acids Y301-E305 and H308, K320, D323, Y326, P339, T341, W356 and P358 of glycoprotein B of HSV-1 and corresponding sites Y293-E297 and H300, K312, D315, Y318, P331, T333, W348 and P350 of glycoprotein B of HSV-2. While this characterization of the epitope is in line with the previously indicated one as described in the literature cited above, the present invention uses this more refined characterization of the epitope.

In general, before describing the epitope of the present invention in more detail as derived by Cryo-EM, it may be understood by a person skilled in the art that the epitopes may be comprised in the gB protein but may also be comprised in a degradation product thereof or may be a chemically synthesized peptide. The amino acid positions are only indicated to demonstrate the position of the corresponding amino acid sequence in the sequence of the gB protein. The invention encompasses all peptides comprising the epitope. The peptide may be a part of a polypeptide of more than 100 amino acids in length or may be a small peptide of less than 100, preferably less than 50, more preferably less than 25 amino acids, even more preferably less than 16 amino acids. The amino acids of such peptide may be natural amino acids or nonnatural amino acids (e.g., beta-amino acids, gamma-amino acids, D-amino acids) or a combination thereof. Further, the present invention may encompass the respective retro-inverso peptides of the epitopes. The peptide may be unbound or bound. It may be bound, e.g., to a small molecule (e.g., a drug or a fluorophor), to a high-molecular weight polymer (e.g., polyethylene glycol (PEG), polyethylene imine (PEI), hydroxypropylmethacrylate (HPMA), etc.) or to a protein, a fatty acid, a sugar moiety or may be inserted in a membrane. In general, as outlined above in the context of the first aspect of the present invention, whether an antibody binds to the same epitope as a reference antibody can be determined by methods known to the person skilled in the art. This applies, mutatis mutandis, to the antibody or antigen-binding fragment of the second component (B) of the second aspect of the present invention as it has been described above in the context of the first aspect of the present invention.

Thus, in order to test in the context of the antibody or antigen-binding fragment of the second component (B) of the second aspect of the present invention whether an antibody in question and the antibody of the present invention recognize the same epitope, the following competition study may be carried out: Vero cells infected with 3 moi (multiplicity of infection) are incubated after 20 h with varying concentrations of the antibody in question as the competitor for 1 hour. In a second incubation step, the antibody of the present invention is applied in a constant concentration of 100 nM and its binding is flow-cytometrically detected using a fluorescence-labelled antibody directed against the constant domains of the antibody of the invention. Binding that conducts anti-proportional to the concentration of the antibody in question is indicative for that both antibodies recognize the same epitope. However, many other assays are known in the art which may be used.

Thus, it is generally possible to determine an epitope bound by an antibody by using overlapping 15-mer peptides spanning the gB region from amino acid 31 to 505 as it has been described in Daumer et al., Med Microbiol Immunol 2011 (200):85-97. Moreover, using high-resolution 13-mer peptide microarrays as described in Krawczyk et al., Journal of Virology 2011 (85):1793-1803, an epitope of glycoprotein B of HSV-1 and HSV-2 recognized by mAb 2c can also be determined.

The sequence of the glycoprotein B of HSV-1 and/or HSV-2 is well-characterized and, as defined above, without being bound to specific sequences, examples sequences of various HSV-1 and HSV-2 strains, respectively, are shown in SEQ ID NOs:9, 10 and 21 to 24. The epitope recognized by the mAb 2c antibody as previously described is highly conserved among various HSV-strains as well as between HSV-1 and HSV-2.

With the normal skill of the person skilled in the art and by routine methods, the person skilled in the art can easily deduce from the sequences provided herein relevant epitopes (also functional fragments) of the polypeptides of HSV which are useful in the generation of antibodies like polyclonal and monoclonal antibodies. However, the person skilled in the art is readily in a position to also provide for engineered antibodies like CDR-grafted antibodies or also humanized and fully human antibodies and the like.

Particularly preferred in the context of the present invention (and, in particular, also in the context of the antibody or antigen-binding fragment of the second component (B) of the second aspect of the present invention) are monoclonal antibodies. For the preparation of monoclonal antibodies, any technique which provides antibodies produced by continuous cell line cultures can be used. Examples for such techniques include the hybridoma technique, the trioma technique, the human B-cell hybridoma technique and the EBV-hybridoma technique to produce human monoclonal antibodies (Shepherd and Dean (2000), Monoclonal Antibodies: A Practical Approach, Oxford University Press, Goding and Goding (1996), Monoclonal Antibodies: Principles and Practice—Production and Application of Monoclonal Antibodies in Cell Biology, Biochemistry and Immunology, Academic Pr Inc, USA).

The antibody derivatives of the antibody or antigen-binding fragment of the second component (B) of the second aspect of the present invention can also be produced by peptidomimetics. Further, techniques described for the production of single chain antibodies (see, inter alia, U.S. Pat. No. 4,946,778) can be adapted to produce single chain antibodies specifically recognizing an antigen of HSV. Also, transgenic animals may be used to express humanized antibodies to the polypeptide of HSV.

The present invention also envisages in the context of the antibody or antigen-binding fragment of the second component (B) of the second aspect of the present invention the production of specific antibodies against native polypeptides and recombinant polypeptides of glycoprotein B or any another protein or polypeptide of HSV-1 and HSV-2. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of HSV-1 or HSV-2 can be subcloned into an appropriated vector, wherein the recombinant polypeptide is to be expressed in an organism being able for an expression, for example in bacteria. Thus, the expressed recombinant protein can be intra-peritoneally injected into a mice and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of HSV-1 and HSV-2 can be obtained by directly immunizing the animal by directly injecting intramuscularly the vector expressing the desired polypeptide or a protein or an epitope of HSV-1 and HSV-2, in particular an epitope of gB. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

The term “specifically binds”, as used herein, refers to a binding reaction that is determinative of the presence of the desired polypeptide or a protein or an epitope of HSV-1 and HSV-2, in particular an epitope of gB, and an antibody in the presence of a heterogeneous population of proteins and other biologics.

Thus, under designated assay conditions, the specified antibodies and a corresponding polypeptide or a protein or an epitope of HSV-1 and HSV-2, in particular an epitope of gB, bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically, a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The term “anti-HSV antibody or antigen-binding fragment thereof” in the context of the antibody or antigen-binding fragment of the second component (B) of the second aspect of the present invention that the antibody molecule or antigen-binding fragment thereof is capable of specifically recognizing or specifically interacting with and/or binding to at least two amino acids of the desired polypeptide or a protein or an epitope of HSV-1 and HSV-2, in particular an epitope of gB. Said term relates to the specificity of the antibody molecule, i.e., to its ability to discriminate between the specific regions a desired polypeptide or a protein or an epitope of HSV-1 and HSV-2, in particular an epitope of gB. Accordingly, specificity can be determined experimentally by methods known in the art and methods as disclosed and described herein. Such methods comprise, but are not limited to Western blots, ELISA-, RIA-, ECL-, IRMA-tests and peptide scans. Such methods also comprise the determination of Kd-values as, inter alia, illustrated in the appended examples. The peptide scan (pepspot assay) is used routinely employed to map linear epitopes in a polypeptide antigen. The primary sequence of the polypeptide is synthesized successively on activated cellulose with peptides overlapping one another. The recognition of certain peptides by the antibody to be tested for its ability to detect or recognize a specific antigen/epitope is scored by routine colour development (secondary antibody with horseradish peroxide and 4-chloronaphtol and hydrogenperoxide), by a chemoluminescence reaction or similar means known in the art. In the case of, inter alia, chemoluminescence reactions, the reaction can be quantified. If the antibody reacts with a certain set of overlapping peptides one can deduce the minimum sequence of amino acids that are necessary for reaction. The same assay can reveal two distant clusters of reactive peptides, which indicate the recognition of a discontinuous, i.e., conformational epitope in the antigenic polypeptide (Geysen (1986), Mol. Immunol. 23, 709-715).

In a preferred embodiment, the antibody or antigen-binding fragment of the second component (B) of the second aspect of the present invention is the mAb 2c antibody (or an antigen-binding fragment thereof). This monoclonal antibody MAb 2c has been described elsewhere and has been demonstrated to neutralize virus by abrogating viral cell-to-cell spread, a key mechanism by which HSV-1/2 escapes humoral immune surveillance independent from antibody-dependent cellular cytotoxicity (ADCC) and/or complement-dependent cytotoxicity (CDC); Eis-Hübinger et al., Intervirology 32:351-360 (1991); Eis-Hübinger et al., Journal of General Virology 74:379-385 (1993); WO2011/038933 A2; Krawczyk A, et al., Journal of virology (2011); 85(4):1793-1803; Krawczyk A, et al., Proc Natl Acad Sci USA (2013); 110(17):6760-6765.

As mentioned above, in the context of the present invention and as shown in the appended Examples, the epitope that is bound by the antibody or antigen-binding fragment of the second component (B) of the second aspect of the present invention has been characterized in more detail by using cryo-electron microscopy (Cryo-EM). As outlined in more detail herein above and below, said epitope is located at the amino acids Y301-E305 and H308, K320, D323, Y326, P339, T341, W356 and P358 of glycoprotein B of HSV-1 and corresponding sites Y293-E297 and H300, K312, D315, Y318, P331, T333, W348 and P350 of glycoprotein B of HSV-2.

While this characterization of the epitope is in line with the previously indicated one as described in the literature cited above, the present invention uses this more refined characterization of the epitope.

Thus, in a preferred embodiment, the antibody or antigen-binding fragment of the second component (B) of the second aspect of the present invention is an antibody that recognizes the same epitope as said above-defined antibody or antigen-binding fragment of the second component (B) of the second aspect of the present invention, wherein said epitope is located at the amino acids Y301-E305 and H308, K320, D323, Y326, P339, T341, W356 and P358 of glycoprotein B of HSV-1 and corresponding sites Y293-E297 and H300, K312, D315, Y318, P331, T333, W348 and P350 of glycoprotein B of HSV-2, wherein the recognition of said epitope is determined by cryo-electron microscopy (Cryo-EM).
preferably,
wherein said epitope consists of the contact amino acid residues Y301-E305 and H308, K320, D323, Y326, P339, T341, W356 and P358 of glycoprotein B of HSV-1 strain F (SEQ ID NO:9) and of the contact amino acids residues Y293-E297 and H300, K312, D315, Y318, P331, T333, W348 and P350 of glycoprotein B of HSV-2 strain G (SEQ ID NO:40), respectively.

In a particular embodiment, the epitope is determined by cryo-electron microscopy (Cryo-EM).

Accordingly, the antibody or antigen-binding fragment of the second component (B) of the second aspect of the present invention binds to a specific epitope on gB comprising the following contact amino acid residues Y301-E305 and H308, K320, D323, Y326, P339, T341, W356 and P358 of glycoprotein B of HSV-1 and corresponding sites Y293-E297 and H300, K312, D315, Y318, P331, T333, W348 and P350 of glycoprotein B of HSV-2. This epitope has been determined by cryo-electron microscopy (CryoEM) analysis.

As outlined above in the context of the first aspect of the present invention, whether an antibody binds to the same epitope as a reference antibody can be determined by methods known to the person skilled in the art. Yet, in one embodiment, this is determined as described in the Examples appended hereto. In a particular embodiment, the epitope is determined by cryo-electron microscopy (Cryo-EM). This applies, mutatis mutandis, to the antibody or antigen-binding fragment of the second component (B) of the second aspect of the present invention as it has been described above in the context of the first aspect of the present invention.

Thus, more specifically, the epitope can be determined as outlined in the following: Cryo-EM grid preparation and data collection is done as follows: HSV-1 gB and HDIT101 Fab are mixed in an optimal ratio. An aliquot of the mixture is adsorbed onto appropriate grids, blotted with filter paper and vitrified into liquid ethane at −180° C. using a plunger operated at 8 to 12° C. and 75 to 95% humidity. Data of HSV-1-gB and HDIT101-Fab complex is acquired on an appropriate transmission electron microscope. Micrograph movies of 40 frames are recorded in counting mode at an appropriate magnification with an appropriate dose. For data processing and model building the following is done: All image processing steps are performed with appropriate software. Dose weighting and motion correction of dose-fractionated and gain-corrected movies are performed using appropriate software. Contrast transfer function (CTF) parameters were estimated using appropriate software. Micrographs displaying strong drift, astigmatism greater than 1000 Å and maximum CTF resolution worse than 8 Å are excluded from further processing. A total of approximately 1 to 10 million particles are picked. The particle dataset is cleaned through three rounds of reference-free 2D classification. Appropriate algorithms are used to generate a de novo 3D initial model from the 2D particles. The particle dataset is further cleaned through three rounds of unsupervised 3D classification. The remaining particles are subjected to Bayesian particle polishing, CTF and aberration refinement, and a final high-resolution 3D refinement, resulting in a final map. The HSV1 gB X-ray structure (PDB-ID: 2GUM) is manually mutated at positions T313S, Q443L and V553A and placed into the final map using appropriate software. For the HDIT101 Fab, the crystal structure of a humanized recombinant Fab fragment of a murine antibody (PDB-ID 3AAZ) is mutated in appropriate software based on a sequence alignment. Three HDIT101 Fabs are placed into the final map. Appropriate software is used for the initial fitting of HSV-1 gB and the three HDIT101 Fabs into the final map. The final protein model is obtained by several iterations of manual model building, refinement and model validation.

In an even more preferred embodiment, more specifically, the epitope can be determined as outlined in the following:

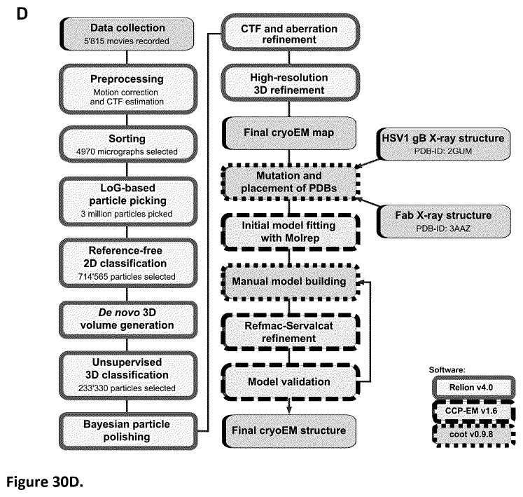

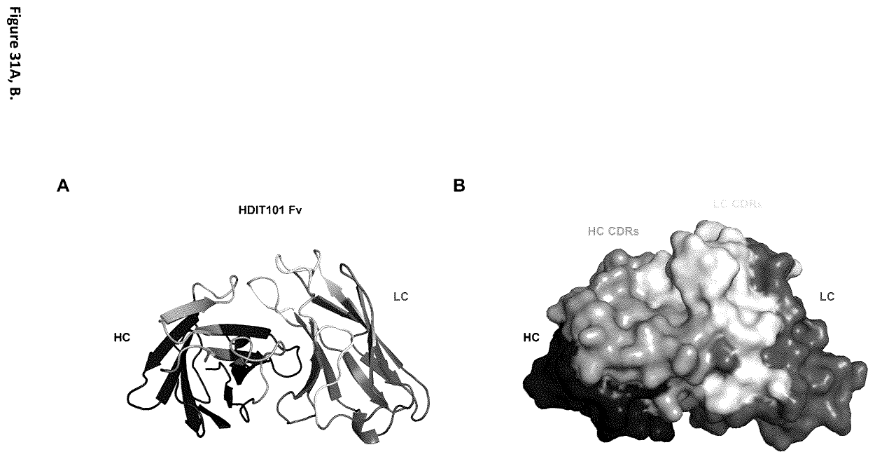

Cryo-EM grid preparation and data collection is done as follows: HSV-1 gB and HDIT101 Fab are mixed in a ratio of 1 to 3.5. A 4 μl aliquot of the mixture is adsorbed onto glow-discharged Quantifoil Cu-R1.2/1.3-300 mesh holey carbon-coated grids (Quantifoil, Germany), blotted with Whatman 1 filter paper and vitrified into liquid ethane at −180° C. using a Leica EM GP2 plunger (Leica microsystems, Austria) operated at 10° C. and 85% humidity. Data of HSV-1-gB and HDIT101-Fab complex is acquired on a Glacios TEM (ThermoFisher) operated at 200 kV and equipped with a Quantum K3 direct electron detector (Gatan). Micrograph movies of 40 frames are recorded in counting mode at a magnification of 45,000× (pixel size 0.878 Å) with a dose of 1.25 e⁻/Å²/frame, resulting in a total accumulated dose on the specimen level of approximately 50 e⁻/Å² per exposure. For data processing and model building the following is done: All image processing steps are performed with Relion v4.0 (Kimanius, Dong et al., 2021, Biochem J, Vol. 478 (24)). Dose weighting and motion correction of dose-fractionated and gain-corrected movies are performed using Relion's implementation of the UCSF motioncor2 program. Contrast transfer function (CTF) parameters were estimated using ctffind 4.1.14 (Rohou and Grigorieff, 2015, J Struct Biol, Vol. 192 (2)). Micrographs displaying strong drift, astigmatism greater than 1000 Å and maximum CTF resolution worse than 8 Å are excluded from further processing. A total of 3 million particles are picked using the Laplacian-of-Gaussian (LoG) filter in Relion 4.0 (Kimanius, Dong et al., 2021, Biochem J, Vol. 478 (24)). The particle dataset is cleaned through three rounds of reference-free 2D classification resulting in 714′565 particles. Relion's Stochastic Gradient Desecnt (SGD) algorithm was used to generate a de novo 3D initial model from the 2D particles. The particle dataset is further cleaned through three rounds of unsupervised 3D classification. The remaining 233′330 particles are subjected to Bayesian particle polishing, CTF and aberration refinement, and a final high-resolution 3D refinement, which resulted in a final map with an overall resolution of 3.27 Å according to the gold standard Fourier shell correlation (FSC) at FSC=0.143. The HSV1 gB X-ray structure (PDB-ID: 2GUM) is manually mutated at positions T313S, Q443L and V553A and placed into the final map using coot (Casanal, Lohkamp et al., 2020, Protein Sci, Vol. 29 (4)). For the HDIT101 Fab, the crystal structure of a humanized recombinant Fab fragment of a murine antibody (PDB-ID 3AAZ) is mutated in coot (Casanal, Lohkamp et al., 2020, Protein Sci, Vol. 29 (4)) based on a sequence alignment generated by Needle EMBOSS (Rice, Longden et al., 2000, Trends Genet, Vol. 16 (6)). Three HDIT101 Fabs are placed into the final map using coot (Casanal, Lohkamp et al., 2020, Protein Sci, Vol. 29 (4)). Molrep of the CCP-EM software suite v1.6 (Nicholls, Tykac et al., 2018, Acta Crystallogr D Struct Biol, Vol. 74 (Pt 6)) are used for the initial fitting of HSV1 gB and the three HDIT101 Fabs into the final map. The final protein model is obtained by several iterations of manual model building in coot (Casanal, Lohkamp et al., 2020, Protein Sci, Vol. 29 (4)), Refmac-Servalcat refinement and model validation in the CCP-EM software suite v1.6 (Nicholls, Tykac et al., 2018, Acta Crystallogr D Struct Biol, Vol. 74 (Pt 6)). Data collection, refinement and validation statistics are performed as summarized in Table 2 and the data processing workflow is shown in FIG. 30D.

The term “combination” in the context of the second aspect of the present invention, i.e., the combination of the above two components (A) and (B), relates to the following:

In a preferred embodiment, a simultaneous application is envisaged. Yet, the combination also encompasses a subsequent application of the two components. Thus, one of these components may be administered before, simultaneously with or after the other one of the combination, or vice versa.

Accordingly, “in combination” as used herein in the context of the second aspect of the present invention does not restrict the timing between the administration of the two components. Thus, when the two components are not administered simultaneously with/concurrently, the administrations may be separated by 1 minute, 5 minutes, 15 minutes, 30 minutes, 45 minutes, 1 hour, 2 hours, 4 hours, 6 hours, 12 hours, 24 hours, 48 hours or 72 hours or by any suitable time differential readily determined by one of skill in art and/or described herein. In a preferred embodiment, when the two components are not administered simultaneously with/concurrently, the administrations may be separated by 1 hour, 2 hours, 4 hours, 6 hours, 12 hours, 24 hours, 48 hours or 72 hours or by any suitable time differential readily determined by one of skill in art and/or described herein.

In a preferred embodiment, in the context of the second aspect of the present invention, the present invention relates to the combination of the anti-HSV antibodies or antigen-binding fragments thereof as defined above, wherein said anti-HSV antibody according to (B) is a humanized or fully human antibody.

The term “humanized or fully human antibody” has already been defined above in the context of the first aspect of the present invention. As regards this definition as well as preferred embodiments of the term “humanized or fully human antibody”, the same applies, mutatis mutandis, to the second component (B) as has been set forth above in the context of the first aspect of the present invention as defined above.

In a preferred embodiment, in the context of the second aspect of the present invention, the present invention relates to the combination of the anti-HSV antibodies or antigen-binding fragments thereof as defined above, wherein said anti-HSV antibody according to (B) is a full-length antibody.

The term “full-length antibody” has already been defined above in the context of the first aspect of the present invention. As regards this definition as well as preferred embodiments of the term “full-length antibody”, the same applies, mutatis mutandis, to the second component (B) as has been set forth above in the context of the first aspect of the present invention as defined above.

In a preferred embodiment, in the context of the second aspect of the present invention, the present invention relates to the combination of the anti-HSV antibodies or antigen-binding fragments thereof as defined above, wherein said anti-HSV antibody according to (B) is a human IgG1, an IgG2, an IgG2a, an IgG2b, an IgA1, an IgGA2, an IgG3, an IgG4, an IgA, an IgM, an IgD or an IgE antibody.

The terms “human IgG1, IgG2, IgG2a, IgG2b, IgA1, IgGA2, IgG3, IgG4, IgA, IgM, IgD and IgE antibody” have already been defined above in the context of the first aspect of the present invention. As regards this definition as well as preferred embodiments of the terms “human IgG1, IgG2, IgG2a, IgG2b, IgA1, IgGA2, IgG3, IgG4, IgA, IgM, IgD and IgE antibody”, the same applies, mutatis mutandis, to the second component (B) as has been set forth above in the context of the first aspect of the present invention as defined above.

In a preferred embodiment, in the context of the second aspect of the present invention, the present invention relates to the combination of the anti-HSV antibodies or antigen-binding fragments thereof as defined above, wherein said antigen-binding fragment according to (B) is a F(ab)-, Fab′-SH-, Fv-, Fab′-, or a F(ab′)2-fragment.

The terms “F(ab)-, Fab′-SH-, Fv-, Fab′-, and F(ab′)2-fragment” have already been defined above in the context of the first aspect of the present invention. As regards this definition as well as preferred embodiments of the terms “F(ab)-, Fab′-SH-, Fv-, Fab′-, and F(ab′)2-fragment”, the same applies, mutatis mutandis, to the second component (B) as has been set forth above in the context of the first aspect of the present invention as defined above.

In a preferred embodiment, in the context of the second aspect of the present invention, the present invention relates to the combination of the anti-HSV antibodies or antigen-binding fragments thereof as defined above, wherein the antibody according to (B) in a concentration of at most 20 nM, preferably of at most 16 nM, more preferably of at most 12 nM, of at most 10 nM, of at most 8 nM, of at most 6 nM, and most preferably of at most 4 nM, is capable of neutralising a defined amount of HSV of 100 TCID50.

The term “capable of neutralising a defined amount of HSV of 100 TCID50” has already been defined above in the context of the first aspect of the present invention. As regards this definition as well as preferred embodiments of the term “capable of neutralising a defined amount of HSV of 100 TCID50”, the same applies, mutatis mutandis, to the second component (B) as has been set forth above in the context of the first aspect of the present invention as defined above.

In a preferred embodiment, in the context of the second aspect of the present invention, the present invention relates to the combination of the anti-HSV antibodies or antigen-binding fragments thereof as defined above, wherein said anti-HSV antibody according to (B) is capable of inhibiting the spreading of HSV from an infected cell to an adjacent second non-infected cell (cell-to-cell spread).

The term “capable of inhibiting the spreading of HSV from an infected cell to an adjacent second non-infected cell (cell-to-cell spread)” has already been defined above in the context of the first aspect of the present invention. As regards this definition as well as preferred embodiments of the term “capable of inhibiting the spreading of HSV from an infected cell to an adjacent second non-infected cell (cell-to-cell spread)”, the same applies, mutatis mutandis, to the second component (B) as has been set forth above in the context of the first aspect of the present invention as defined above.

In a preferred embodiment, in the context of the second aspect of the present invention, the present invention relates to the combination of the anti-HSV antibodies or antigen-binding fragments thereof as defined above, wherein said anti-HSV antibody according to claim (B) exerts its antiviral or neutralizing activity independent from antibody-dependent cellular cytotoxicity (ADCC) and/or complement-dependent cytotoxicity (CDC), preferably, said antibody is capable of inhibiting cell-to-cell spread independent from antibody-dependent cellular cytotoxicity (ADCC) and/or complement-dependent cytotoxicity (CDC).

The term “capable of inhibiting cell-to-cell spread independent from antibody-dependent cellular cytotoxicity (ADCC) and/or complement-dependent cytotoxicity (CDC)” has already been defined above in the context of the first aspect of the present invention. As regards this definition as well as preferred embodiments of the term “capable of inhibiting cell-to-cell spread independent from antibody-dependent cellular cytotoxicity (ADCC) and/or complement-dependent cytotoxicity (CDC)”, the same applies, mutatis mutandis, to the second component (B) as has been set forth above in the context of the first aspect of the present invention as defined above.

In a preferred embodiment, in the context of the second aspect of the present invention, the present invention relates to the combination of the anti-HSV antibodies or antigen-binding fragments thereof as defined above, wherein said anti-HSV antibody according to (B) is conjugated to an effector moiety, a therapeutic moiety, or a detectable label.

The term “conjugated to an effector moiety, a therapeutic moiety, or a detectable label” has already been defined above in the context of the first aspect of the present invention. As regards this definition as well as preferred embodiments of the term “conjugated to an effector moiety, a therapeutic moiety, or a detectable label”, the same applies, mutatis mutandis, to the second component (B) as has been set forth above in the context of the first aspect of the present invention as defined above.

In the following, the third aspect, is described in more detail.

Thus, as mentioned above, in a third aspect, the present invention relates to a bispecific antibody or an antigen-binding fragment thereof binding to the glycoprotein B (gB) of the HSV-1 and/or HSV-2 which comprises:
(A) a first binding domain comprising:
   the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6; and
(B) a second binding domain comprising:
   the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16;
wherein said bispecific antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹.

The sequences of part (A) of the bispecific antibody's first binding domain comprising: the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6 correspond to the CDR sequences of the antibody according to the first aspect of the present invention and have already described in detail in the contest of the first aspect of the present invention.

As regards this definition as well as preferred embodiments, the same applies, mutatis mutandis, to the bispecific antibody as has been set forth above in the context of the first aspect of the present invention as defined above.

The sequences of part (B) of the bispecific antibody's binding domain comprising: the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16 correspond to the CDR sequences of the antibody according to the second aspect (part (B) therein) of the present invention and have already described in detail in the contest of the second aspect (part (B) therein) of the present invention.

As regards this definition as well as preferred embodiments, the same applies, mutatis mutandis, to the bispecific antibody as has been set forth above in the context of the second aspect (part (B) therein) of the present invention as defined above.

As regards the bispecific antibody's capability of having a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹ as well as preferred embodiments, the same applies, mutatis mutandis, to the bispecific antibody as has been set forth above in the context of the first aspect of the present invention (as regards part (A)) and the second aspect of the present invention (as regards part (A)), respectively, as defined above.

Thus, in further preferred embodiments, said bispecific antibody has a low dissociation rate k_{dis} of at most 5.0×10⁻⁴ s⁻¹, preferably at most 4.0×10⁻⁴ s⁻¹, more preferably at most 3.0×10⁻⁴ s⁻¹, even more preferably at most 2.0×10⁻⁴ s⁻¹, at most 1.0×10⁻⁴ s⁻¹, at most 9.0×10⁻⁵ s⁻¹, at most 8.0×10⁻⁵ s⁻¹, at most 7.0×10⁻⁵ s⁻¹, at most 6.0×10⁻⁵ s⁻¹, at most 5.0×10⁻⁵ s⁻¹, at most 4.0×10⁻⁵ s⁻¹, at most 3.0×10⁻⁵ s⁻¹ and most preferably at most 2.9×10⁻⁵ s⁻¹, at most 2.0×10⁻⁵ s⁻¹, at most 1.5×10⁻⁵ s⁻¹, at most 1.0×10⁻⁵ s⁻¹, at most 5.0×10⁻⁶ s⁻¹, at most 2.0×10⁻⁶ s⁻¹, at most 1.0×10⁻⁶ s⁻¹, at most 5.0×10⁻⁷ s⁻¹, at most 2.0×10⁻⁷ s⁻¹, at most 1.0×10⁻⁷ s⁻¹, at most 1.0×10⁻⁸ s⁻¹, and at most 1.0×10⁻⁹ s⁻¹.

In a more preferred embodiment, the above bispecific antibody binding to the glycoprotein B (gB) of the HSV-1 and/or HSV-2 has the amino acid sequence of SEQ ID NO:27.

In general, bispecific antibodies are well-known in the art for decades and relates to an artificial protein that can simultaneously bind to two different types of antigen or two different epitopes on the same antigen.

Bispecific antibody molecules according to the invention are (monoclonal) bispecific antibodies that have binding specificities for at least two different sites or epitopes (which can be overlapping) and can be of any format. A wide variety of recombinant antibody formats have been developed in the recent past, e.g., bivalent, trivalent or tetravalent bispecific antibodies. Examples include the fusion of an IgG antibody format and single chain domains (for different formats see e.g., Coloma, M. J., et al., Nature Biotech 15 (1997), 159-163; WO 2001/077342; Morrison, S. L., Nature Biotech 25 (2007), 1233-1234; Holliger, P., et. al, Nature Biotech. 23 (2005), 1126-1136; Fischer, N., and Léger, O., Pathobiology 74 (2007), 3-14; Shen, J., et. al., J. Immunol. Methods 318 (2007), 65-74; Wu, C., et al., Nature Biotech. 25 (2007), 1290-1297). The bispecific antibody or fragment herein also includes bivalent, trivalent or tetravalent bispecific antibodies described in WO 2009/080251; WO 2009/080252; WO 2009/080253; WO 2009/080254; WO 2010/112193; WO 2010/115589; WO 2010/136172; WO 2010/145792; WO 2010/145793 and WO 2011/117330.

Accordingly, in the context of the third aspect of the present invention, “antibodies” of the present invention have two or more binding domains and are bispecific. That is, the antibodies may be bispecific even in cases where there are more than two binding domains (i.e., that the antibody is trivalent or multivalent). Bispecific antibodies of the invention include, for example, multivalent single chain antibodies, diabodies and triabodies, as well as antibodies having the constant domain structure of full-length antibodies to which further antigen-binding domains (e.g., single chain Fv, a VH domain and/or a VL domain, Fab, or (Fab)2,) are linked via one or more peptide-linkers. The antibodies can be full length from a single species, or be chimerized or humanized. For an antibody with more than two antigen binding domains, some binding domains may be identical, as long as the protein has binding domains for two different antigens.

As outlined, in certain embodiments, the bispecific antibody of the present invention comprises two main modules, i.e., a first binding domain (A) and a second binding domain (B). The arrangement of the first binding domain (A) and the second binding domain (B) within the bispecific antibody is not particularly limited. Accordingly, the first binding domain (A) and the second binding domain (B) may be placed at either end (i.e., at the N- or C-terminal end in case of the bispecific antibody). Thus, the bispecific antibody may have the arrangement of (A)-(B) or (B)-(A). Yet, preferably, the bispecific antibody has the arrangement (B)-(A).

The bispecific antibody according to the present invention may not only comprise the above two main modules, i.e., the first binding domain (A) and the second binding domain (B). Rather, it may be desirable that between the individual modules (a) linker moiety/moieties are placed which may, e.g., facilitate the construction of the construct.

The nature and the length of the linker is not particularly limited. In a preferred embodiment, the linker between the first binding domain (A) and the second binding domain (B) comprises one or more amino acids. These additional one or more amino acid(s) can comprise polypeptide chains of up to 1, 2, 3, 4, 5, 6, 7, 8, 9 or 10 amino acids, preferably of up to 20 amino acids or even more preferably of up to 30 amino acids.

Moreover, the bispecific antibody, in addition to the first binding domain (A) and the second binding domain (B), comprises in preferred embodiments, one or more additional amino acids which can be flanking or interspersed in relation to the first binding domain (A) and the second binding domain (B), respectively. Thus, the one or more additional amino acids may be added at the N- and/or C-terminal end of the first binding domain (A) and the second binding domain (B). The additional amino acid(s) comprise polypeptide chains of up to 1, 2, 3, 4, 5, 6, 7, 8, 9 or 10 amino acids, preferably of up to 20, 30, 40, 50, 60, 70, 80, 90 or 100 amino acids or even more preferably of up to 130, 150, 200, 300, 400 or 500 amino acids.

The bispecific antibody may be present in the form of a fusion protein, i.e., a protein which is formed by the expression of a hybrid gene made by combining at least two gene sequences. Typically, this is accomplished by cloning a cDNA into an expression vector in frame with an existing gene. Accordingly, the construct may be a fusion protein, i.e., a chimeric molecule which is formed by joining two or more polypeptides via a peptide bond between the amino terminus of one module and the carboxyl terminus of another molecule. In this way, the above first binding domain (A) and the second binding domain (B) are joined together in the form of a fusion protein. Once cloned in frame, the fusion protein is then recombinantly expressed by a corresponding nucleic acid sequence encoding said fusion protein.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g., Sambrook et al., Molecular Cloning, A laboratory manual, 2nd Ed, 1989).

Alternatively, at least one of the two modules of the bispecific antibody, preferably both modules, may also be covalently coupled by a chemical conjugate. Thus, the modules of the bispecific antibody may be chemically coupled in a covalent linkage.

The term “chemically coupled in a covalent linkage” relates to conjugation techniques which are well-known to the skilled person. Many methods for making covalent or non-covalent conjugates with proteins or peptides are known in the art and any such known method may be utilized. Without being bound to theory, a construct according to the present invention can be prepared by using a heterobifunctional cross-linker, such as N-succinyl 3-(2-pyridyldithio)propionate (SPDP). Yu et al., Int. J. Cancer 56: 244 (1994). General techniques for such conjugation are well-known in the art; see, for example, Wong, Chemistry of Protein Conjugation and Cross-linking (CRC Press 1991); Upeslacis et al., “Modification of Antibodies by Chemical Methods,” in Monoclonal Antibodies: Principles and Applications, Birch et al. (eds.), pages 187-230 (Wiley-Liss, Inc. 1995); Price, “Production and Characterization of Synthetic Peptide-Derived Antibodies,” in Monoclonal Antibodies: Production, Engineering and Clinical Application, Ritter et al. (eds.), pages 60-84 (Cambridge University Press 1995). Therefore, in view of the fact that methods for coupling moieties in a covalent linkage to each other, preferable to a protein/peptide are well-known to the person skilled in the art the examples provided herewith are not limiting. For an overview of methods for (covalently) coupling a dye to a protein reference is made, e.g., to the review article of Brinkley M. Bioconjug Chem. 1992 January-February; 3(1): 2-13 and to the article of Lopez-Jaramillo, et al., in chapter 16 entitled “Vinyl Sulfone: A Multi-Purpose Function in Proteomics” the book Biochemistry, Genetics and Molecular Biology “Integrative Proteomics” edited by Hon-Chiu Eastwood Leung, Subject editors: Tsz-Kwong Man and Ricardo J. Flores, ISBN 978-953-51-0070-6, Published: Feb. 24, 2012.

Thus, in one embodiment, both two modules (i.e., the first binding domain (A) and the second binding domain (B)) may individually be synthesized (either chemically or by recombinant technology), optionally purified and then chemically coupled in a covalent linkage.

In certain embodiments, an antibody according to the third aspect of the present invention is a multispecific antibody, e.g., a bispecific antibody. Multispecific antibodies are monoclonal antibodies that have binding specificities for at least two different sites.

Techniques for making multispecific antibodies, in particular, bispecific antibodies, include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies, in particular, bispecific antibodies, may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553; using “diabody” technology for making bispecific antibody fragments (see, e.g., Holliger, P., et al., Proc. Natl. Acad. Sci. USA 90 (1993) 6444-6448); and using single-chain Fv (sFv) dimers (see, e.g. Gruber, M., et al., J. Immunol. 152 (1994) 5368-5374); and preparing trispecific antibodies as described, e.g., in Tutt, A., et al., J. Immunol. 147 (1991) 60-69).

Preferred embodiments of the bispecific antibody binding to the glycoprotein B (gB) of the HSV-1 and/or HSV-2 in accordance with the third aspect of the present invention are described in more detail in the following.

In a preferred embodiment, in the context of the third aspect of the present invention, the present invention relates to a bispecific antibody binding to the glycoprotein B (gB) of the HSV-1 and/or HSV-2 as defined above, wherein
part (A) comprises the following framework regions:
an amino acid sequence with at least 70% sequence identity to each one of the amino acid residues shown in positions 1 to 25 (V_{H}FR1), 36 to 49 (V_{H}FR2), 67 to 98 (V_{H}FR3), 112 to 122 (V_{H}FR4) of SEQ ID NO: 7, 1 to 22 (V_{L}FR1), 34 to 48 (V_{L}FR2), 56 to 87 (V_{L}FR3), and 97 to 106 (VLFR4) of SEQ ID NO: 8; and
part (B) comprises the following framework regions:
an amino acid sequence with at least 70% sequence identity to the amino acid residues shown in positions 1 to 30 (V_{H}FR1), 38 to 51 (V_{H}FR2), 68 to 99 (V_{H}FR3), and 112 to 122 (V_{H}FR4) of SEQ ID NO: 17, 1 to 23 (V_{L}FR1), 41 to 55 (V_{L}FR2), 63 to 94 (V_{L}FR3), and 104 to 114 (V_{L}FR4) of SEQ ID NO: 18.

As regards this definition as well as preferred embodiments, the same applies, mutatis mutandis, to the bispecific antibody as has been set forth above in the context of the first aspect of the present invention (as regards part (A)) and the second aspect of the present invention (as regards part (B)), respectively, as defined above.

In a preferred embodiment, in the context of the third aspect of the present invention, the present invention relates to a bispecific antibody binding to the glycoprotein B (gB) of the HSV-1 and/or HSV-2 as defined above, wherein
part (A) comprises the V_{H} of SEQ ID NO:7 and the V_{L} of SEQ ID NO:8; and
part (B) comprises the V_{H} of SEQ ID NO:19 and the V_{L} of SEQ ID NO:20.

As regards this definition as well as preferred embodiments, the same applies, mutatis mutandis, to the bispecific antibody as has been set forth above in the context of the first aspect of the present invention (as regards part (A)) and the second aspect of the present invention (as regards part (B)), respectively, as defined above.

In a preferred embodiment, in the context of the third aspect of the present invention, the present invention relates to a bispecific antibody binding to the glycoprotein B (gB) of the HSV-1 and/or HSV-2 as defined above, wherein
part (A) recognizes the same epitope as said antibody, wherein said epitope is located at the contact amino acid residues D199, A203, K204, Y303, R304, K320, Q321, V322, D323, Y326, R335 and T337 of glycoprotein B of HSV-1 strain F and contact amino acids residues D191, A195, K196, Y295, R296, K312, Q313, V314, D315, Y318, R327 and T329 of glycoprotein B of HSV-2 strain G, respectively,
preferably,
wherein said epitope consists of the contact amino acid residues D199, A203, K204, Y303, R304, K320, Q321, V322, D323, Y326, R335 and T337 of glycoprotein B of HSV-1 strain F (SEQ ID NO:9) and of the contact amino acids residues D191, A195, K196, Y295, R296, K312, Q313, V314, D315, Y318, R327 and T329 of glycoprotein B of HSV-2 strain G (SEQ ID NO:40), respectively; and
part (B) recognizes the same epitope as said antibody, wherein said epitope is located at the amino acids Y301-E305 and H308, K320, D323, Y326, P339, T341, W356 and P358 of glycoprotein B of HSV-1 and corresponding sites Y293-E297 and H300, K312, D315, Y318, P331, T333, W348 and P350 of glycoprotein B of HSV-2,
preferably,
wherein said epitope consists of the contact amino acid residues Y301-E305 and H308, K320, D323, Y326, P339, T341, W356 and P358 of glycoprotein B of HSV-1 strain F (SEQ ID NO:9) and of the contact amino acids residues Y293-E297 and H300, K312, D315, Y318, P331, T333, W348 and P350 of glycoprotein B of HSV-2 strain G (SEQ ID NO:40), respectively.

In a particular embodiment, the epitope is determined by cryo-electron microscopy (Cryo-EM).

As regards this definition as well as preferred embodiments, the same applies, mutatis mutandis, to the bispecific antibody as has been set forth above in the context of the first aspect of the present invention (as regards part (A)) and the second aspect of the present invention (as regards part (B)), respectively, as defined above.

In a preferred embodiment, in the context of the third aspect of the present invention, the present invention relates to a bispecific antibody binding to the glycoprotein B (gB) of the HSV-1 and/or HSV-2 as defined above, wherein said bispecific antibody is a humanized or fully human antibody.

The term “humanized or fully human antibody” has already been defined above in the context of the first aspect of the present invention. As regards this definition as well as preferred embodiments of the term “humanized or fully human antibody”, the same applies, mutatis mutandis, to the bispecific antibody as has been set forth above in the context of the first aspect of the present invention as defined above.

In a preferred embodiment, in the context of the third aspect of the present invention, the present invention relates to a bispecific antibody binding to the glycoprotein B (gB) of the HSV-1 and/or HSV-2 as defined above, wherein said bispecific antibody is a full-length antibody. The term “full-length antibody” has already been defined above in the context of the first aspect of the present invention. As regards this definition as well as preferred embodiments of the term “full-length antibody”, the same applies, mutatis mutandis, to the bispecific antibody as has been set forth above in the context of the first aspect of the present invention as defined above.

In a preferred embodiment, in the context of the third aspect of the present invention, the present invention relates to a bispecific antibody binding to the glycoprotein B (gB) of the HSV-1 and/or HSV-2 as defined above, wherein said bispecific antibody is a human IgG1, an IgG2, an IgG2a, an IgG2b, an IgA1, an IgGA2, an IgG3, an IgG4, an IgA, an IgM, an IgD or an IgE antibody.

The terms “human IgG1, IgG2, IgG2a, IgG2b, IgA1, IgGA2, IgG3, IgG4, IgA, IgM, IgD and IgE antibody” have already been defined above in the context of the first aspect of the present invention. As regards this definition as well as preferred embodiments of the terms “human IgG1, IgG2, IgG2a, IgG2b, IgA1, IgGA2, IgG3, IgG4, IgA, IgM, IgD and IgE antibody”, the same applies, mutatis mutandis, to the bispecific antibody as has been set forth above in the context of the first aspect of the present invention as defined above.

In a preferred embodiment, in the context of the third aspect of the present invention, the present invention relates to a bispecific antibody binding to the glycoprotein B (gB) of the HSV-1 and/or HSV-2 as defined above, wherein said bispecific antibody is a F(ab)-, Fab′-SH-, Fv-, Fab′-, or a F(ab′)2-fragment.

The terms “F(ab)-, Fab′-SH-, Fv-, Fab′-, and F(ab′)2-fragment” have already been defined above in the context of the first aspect of the present invention. As regards this definition as well as preferred embodiments of the terms “F(ab)-, Fab′-SH-, Fv-, Fab′-, and F(ab′)2-fragment”, the same applies, mutatis mutandis, to the bispecific antibody as has been set forth above in the context of the first aspect of the present invention as defined above.

In a preferred embodiment, in the context of the third aspect of the present invention, the present invention relates to a bispecific antibody binding to the glycoprotein B (gB) of the HSV-1 and/or HSV-2 as defined above, wherein said bispecific antibody in a concentration of at 20 nM, preferably of at most 16 nM, more preferably of at most 13 nM, of at most 11 nM, of at most 9 nM, of at most 7 nM, of at most 6 nM, of at most 5 nM, and most preferably of at most 4 nM, is capable of neutralising a defined amount of HSV of 100 TCID50.

The term “capable of neutralising a defined amount of HSV of 100 TCID50” has already been defined above in the context of the first aspect of the present invention. As regards this definition as well as preferred embodiments of the term “capable of neutralising a defined amount of HSV of 100 TCID50”, the same applies, mutatis mutandis, to the bispecific antibody as has been set forth above in the context of the first aspect of the present invention as defined above.

In a preferred embodiment, in the context of the third aspect of the present invention, the present invention relates to a bispecific antibody binding to the glycoprotein B (gB) of the HSV-1 and/or HSV-2 as defined above, wherein said bispecific antibody is capable of inhibiting the spreading of HSV from an infected cell to an adjacent second non-infected cell (cell-to-cell spread).

The term “capable of inhibiting the spreading of HSV from an infected cell to an adjacent second non-infected cell (cell-to-cell spread)” has already been defined above in the context of the first aspect of the present invention. As regards this definition as well as preferred embodiments of the term “capable of inhibiting the spreading of HSV from an infected cell to an adjacent second non-infected cell (cell-to-cell spread)”, the same applies, mutatis mutandis, to the bispecific antibody as has been set forth above in the context of the first aspect of the present invention as defined above.

In a preferred embodiment, in the context of the third aspect of the present invention, the present invention relates to a bispecific antibody binding to the glycoprotein B (gB) of the HSV-1 and/or HSV-2 as defined above, wherein said bispecific antibody exerts its antiviral or neutralizing activity independent from antibody-dependent cellular cytotoxicity (ADCC) and/or complement-dependent cytotoxicity (CDC), preferably, wherein said antibody is capable of inhibiting cell-to-cell spread independent from antibody-dependent cellular cytotoxicity (ADCC) and/or complement-dependent cytotoxicity (CDC).

The term “capable of inhibiting cell-to-cell spread independent from antibody-dependent cellular cytotoxicity (ADCC) and/or complement-dependent cytotoxicity (CDC)” has already been defined above in the context of the first aspect of the present invention. As regards this definition as well as preferred embodiments of the term “capable of inhibiting cell-to-cell spread independent from antibody-dependent cellular cytotoxicity (ADCC) and/or complement-dependent cytotoxicity (CDC)”, the same applies, mutatis mutandis, to the bispecific antibody as has been set forth above in the context of the first aspect of the present invention as defined above.

In a preferred embodiment, in the context of the third aspect of the present invention, the present invention relates to a bispecific antibody binding to the glycoprotein B (gB) of the HSV-1 and/or HSV-2 as defined above, wherein said bispecific antibody is conjugated to an effector moiety, a therapeutic moiety, or a detectable label.

The term “conjugated to an effector moiety, a therapeutic moiety, or a detectable label” has already been defined above in the context of the first aspect of the present invention. As regards this definition as well as preferred embodiments of the term “conjugated to an effector moiety, a therapeutic moiety, or a detectable label”, the same applies, mutatis mutandis, to the bispecific antibody as has been set forth above in the context of the first aspect of the present invention as defined above.

In the following, the fourth aspect, is described in more detail.

Thus, as mentioned above, in a fourth aspect, the present invention relates to a trispecific antibody or an antigen-binding fragment thereof binding to the glycoprotein B (gB) of the HSV-1 and/or HSV-2 which comprises:

Further, in a fourth aspect, the present invention relates to a trispecific antibody or an antigen-binding fragment thereof binding to the glycoprotein B (gB) of the HSV-1 and/or HSV-2 which comprises:
(A) a first binding domain comprising:
   the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6; and
(B) a second binding domain comprising:
   the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16; and
(C) a third binding domain comprising:
   the complementarity determining regions V_{H}CDR1 comprising SEQ ID NO: 31, V_{H}CDR2 comprising SEQ ID NO: 32, V_{H}CDR3 comprising SEQ ID NO: 33, V_{L}CDR1 comprising SEQ ID NO: 34, V_{L}CDR2 comprising SEQ ID NO: 35, and V_{L}CDR3 comprising SEQ ID NO:36;
wherein said trispecific antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹; and wherein said trispecific antibody in a concentration of at 10 nM, preferably of at most 8 nM, more preferably of at most 6 nM, of at most 4 nM, of at most 2 nM, of at most 1 nM, of at most 0.9 nM, of at most 0.7 nM, and most preferably of at most 0.5 nM, is capable of neutralising a defined amount of HSV of 100 TCID50.

The sequences of part (A) of the trispecific antibody's first binding domain comprising: the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6 correspond to the CDR sequences of the antibody according to the first aspect of the present invention and have already described in detail in the contest of the first aspect of the present invention.

As regards this definition as well as preferred embodiments, the same applies, mutatis mutandis, to the trispecific antibody as has been set forth above in the context of the first aspect of the present invention as defined above.

The sequences of part (B) of the trispecific antibody's binding domain comprising: the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16 correspond to the CDR sequences of the antibody according to the second aspect (part (B) therein) of the present invention and have already described in detail in the contest of the second aspect (part (B) therein) of the present invention.

As regards this definition as well as preferred embodiments, the same applies, mutatis mutandis, to the trispecific antibody as has been set forth above in the context of the second aspect (part (B) therein) of the present invention as defined above.

As regards the trispecific antibody's capability of neutralizing a defined amount of HSV of 100 TCID50 in a concentration of at 10 nM, preferably of at most 8 nM, more preferably of at most 6 nM, of at most 4 nM, of at most 2 nM, of at most 1 nM, of at most 0.9 nM, of at most 0.7 nM, and most preferably of at most 0.5 nM as well as preferred embodiments, the same applies, mutatis mutandis, to the trispecific antibody as has been set forth above in the context of the first aspect of the present invention (as regards part (A)) and the second aspect of the present invention (as regards part (A)), respectively, as defined above.

As regards the trispecific antibody's capability of having a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹ as well as preferred embodiments, the same applies, mutatis mutandis, to the trispecific antibody as has been set forth above in the context of the first aspect of the present invention (as regards part (A)) and the second aspect of the present invention (as regards part (A)), respectively, as defined above.

Thus, in further preferred embodiments, said trispecific antibody has a low dissociation rate k_{dis} of at most 5.0×10⁻⁴ s⁻¹, preferably at most 4.0×10⁻⁴ s⁻¹, more preferably at most 3.0×10⁻⁴ s⁻¹, even more preferably at most 2.0×10⁻⁴ s⁻¹, at most 1.0×10⁻⁴ s⁻¹, at most 9.0×10⁻⁵ s⁻¹, at most 8.0×10⁻⁵ s⁻¹, at most 7.0×10⁻⁵ s⁻¹, at most 6.0×10⁻⁵ s⁻¹, at most 5.0×10⁻⁵ s⁻¹, at most 4.0×10⁻⁵ s⁻¹, at most 3.0×10⁻⁵ s⁻¹ and most preferably at most 2.9×10⁻⁵ s⁻¹, at most 2.0×10⁻⁵ s⁻¹, at most 1.5×10⁻⁵ s⁻¹, at most 1.0×10⁻⁵ s⁻¹, at most 5.0×10⁻⁶ s⁻¹, at most 2.0×10⁻⁶ s⁻¹, at most 1.0×10⁻⁶ s⁻¹, at most 5.0×10⁻⁷ s⁻¹, at most 2.0×10⁻⁷ s⁻¹, at most 1.0×10⁻⁷ s⁻¹, at most 1.0×10⁻⁸ s⁻¹, and at most 1.0×10⁻⁹ s⁻¹.

In a more preferred embodiment, the above trispecific antibody binding to the glycoprotein B (gB) of the HSV-1 and/or HSV-2 has the amino acid sequence of SEQ ID NO:30.

In general, trispecific antibodies are well-known in the art for decades and relates to an artificial protein that can simultaneously bind to three different types of antigen or three different epitopes on the same antigen.

Trispecific antibody molecules according to the invention are (monoclonal) bispecific antibodies that have binding specificities for at least three different sites or epitopes (which can be overlapping) and can be of any format. A wide variety of recombinant antibody formats have been developed in the recent past, e.g., trivalent or tetravalent bispecific antibodies. Examples include the fusion of an IgG antibody format and single chain domains (for different formats see e.g., Coloma, M. J., et al., Nature Biotech 15 (1997), 159-163; WO 2001/077342; Morrison, S. L., Nature Biotech 25 (2007), 1233-1234; Holliger, P., et. al, Nature Biotech. 23 (2005), 1126-1136; Fischer, N., and Leger, O., Pathobiology 74 (2007), 3-14; Shen, J., et. al., J. Immunol. Methods 318 (2007), 65-74; Wu, C., et al., Nature Biotech. 25 (2007), 1290-1297). The bispecific antibody or fragment herein also includes bivalent, trivalent or tetravalent bispecific antibodies described in WO 2009/080251; WO 2009/080252; WO 2009/080253; WO 2009/080254; WO 2010/112193; WO 2010/115589; WO 2010/136172; WO 2010/145792; WO 2010/145793 and WO 2011/117330.

Accordingly, in the context of the fourth aspect of the present invention, “antibodies” of the present invention have three or more binding domains and are trispecific. That is, the antibodies may be trispecific even in cases where there are more than three binding domains. Trispecific antibodies of the invention include, for example, multivalent single chain antibodies, diabodies and triabodies, as well as antibodies having the constant domain structure of full-length antibodies to which further antigen-binding domains (e.g., single chain Fv, a VH domain and/or a VL domain, Fab, or (Fab)2,) are linked via one or more peptide-linkers. The antibodies can be full length from a single species or be chimerized or humanized. For an antibody with more than two antigen binding domains, some binding domains may be identical, as long as the protein has binding domains for two different antigens.

In certain embodiments, an antibody according to the fourth aspect of the present invention is a multispecific antibody, e.g., a trispecific antibody. Multispecific antibodies are monoclonal antibodies that have binding specificities for at least three different sites.

Techniques for making multispecific antibodies, in particular, trispecific antibodies, include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies, in particular, bispecific antibodies, may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553; using “diabody” technology for making bispecific antibody fragments (see, e.g., Holliger, P., et al., Proc. Natl. Acad. Sci. USA 90 (1993) 6444-6448); and using single-chain Fv (sFv) dimers (see, e.g. Gruber, M., et al., J. Immunol. 152 (1994) 5368-5374); and preparing trispecific antibodies as described, e.g., in Tutt, A., et al., J. Immunol. 147 (1991) 60-69).

As outlined, in certain embodiments, the trispecific antibody of the present invention comprises three main modules, i.e., a first binding domain (A), a second binding domain (B) and a third binding domain (C).

The arrangement of the first binding domain (A) and the second binding domain (B) and the third binding domain (C) within the bispecific antibody is not particularly limited. Accordingly, the first binding domain (A), the second binding domain (B) and a third binding domain (C), respectively, may be placed at either end (i.e., at the N- or C-terminal end in case of the trispecific antibody) while the remaining binding domain is placed between the other two binding domains. Accordingly, module (C), (A) and (B), respectively, may be located between the other modules (B) and (A), (B) and (C), (C) and (B) and (C) and (A), respectively. Thus, the trispecific antibody may have the arrangement of (A)-(B)-(C), (A)-(C)-(B), (B)-(A)-(C), (B)-(C)-(A), (C)-(A)-(B) or (C)-(B)-(A). Yet, preferably, the bispecific antibody has the arrangement (B)-(A)-(C).

The trispecific antibody according to the present invention may not only comprise the above three main modules, i.e., the first binding domain (A) the second binding domain (B), and the third binding domain (C). Rather, it may be desirable that between two or three of the individual modules (a) linker moiety/moieties are placed which may, e.g., facilitate the construction of the construct.

The nature and the length of the linker is not particularly limited. In a preferred embodiment, the linker between the first binding domain (A) and/or the second binding domain (B) and/or the third binding domain (C) comprises one or more amino acids. These additional one or more amino acid(s) can comprise polypeptide chains of up to 1, 2, 3, 4, 5, 6, 7, 8, 9 or 10 amino acids, preferably of up to 20 amino acids or even more preferably of up to 30 amino acids. Moreover, the trispecific antibody, in addition to the first binding domain (A), the second binding domain (B) and the third binding domain (C) comprises in preferred embodiments, one or more additional amino acids which can be flanking or interspersed in relation to the first binding domain (A), the second binding domain (B), and the third binding domain (C), respectively. Thus, the one or more additional amino acids may be added at the N- and/or C-terminal end of the first binding domain (A) and/or the second binding domain (B) and/or the third binding domain (C). The additional amino acid(s) comprise polypeptide chains of up to 1, 2, 3, 4, 5, 6, 7, 8, 9 or 10 amino acids, preferably of up to 20, 30, 40, 50, 60, 70, 80, 90 or 100 amino acids or even more preferably of up to 130, 150, 200, 300, 400 or 500 amino acids.

The trispecific antibody may be present in the form of a fusion protein, i.e., a protein which is formed by the expression of a hybrid gene made by combining at least three gene sequences. Typically, this is accomplished by cloning a cDNA into an expression vector in frame with an existing gene. Accordingly, the construct may be a fusion protein, i.e., a chimeric molecule which is formed by joining two or more polypeptides via a peptide bond between the amino terminus of one module and the carboxyl terminus of another molecule. In this way, the above first binding domain (A), the second binding domain (B) and the third binding domain (C) are joined together in the form of a fusion protein. Once cloned in frame, the fusion protein is then recombinantly expressed by a corresponding nucleic acid sequence encoding said fusion protein.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g., Sambrook et al., Molecular Cloning, A laboratory manual, 2nd Ed, 1989).

Alternatively, at least one of the three modules of the trispecific antibody, preferably two or even more preferably three modules, may also be covalently coupled by a chemical conjugate. Thus, the modules of the trispecific antibody may be chemically coupled in a covalent linkage. The term “chemically coupled in a covalent linkage” has already been described above in the context of the bispecific antibody of the third aspect of the present invention. The same applies, mutatis mutandis, to the trispecific antibody of the fourth aspect of the present invention.

In one embodiment, all three modules (i.e., the first binding domain (A), the second binding domain (B) and the third binding domain (C)) may individually be synthesized (either chemically or by recombinant technology), optionally purified and then chemically coupled in a covalent linkage.

Thus, the trispecific antibody according to the present invention may be a trispecific antibody, wherein the first binding domain (A), the second binding domain (B) and the third binding domain (C) are chemically coupled in a covalent linkage. Alternatively, module (A) and (B) may be a fusion protein while module (C) is chemically coupled in a covalent linkage to said fusion protein comprising module (A) and (B). In another alternative, module (A) and (C) may be a fusion protein while module (B) is chemically coupled in a covalent linkage to said fusion protein comprising module (A) and (C). In another alternative, module (B) and (C) may be a fusion protein while module (A) is chemically coupled in a covalent linkage to said fusion protein comprising module (B) and (C).

It is also conceivable that two modules are in the form of a fusion protein while the third module is chemically coupled in a covalent linkage.

Preferred embodiments of the trispecific antibody binding to the glycoprotein B (gB) of the HSV-1 and/or HSV-2 in accordance with the fourth aspect of the present invention are described in more detail in the following.

In a preferred embodiment, in the context of the fourth aspect of the present invention, the present invention relates to a trispecific antibody binding to the glycoprotein B (gB) of the HSV-1 and/or HSV-2 as defined above, wherein
part (A) comprises the following framework regions:
an amino acid sequence with at least 70% sequence identity to each one of the amino acid residues shown in positions 1 to 25 (V_{H}FR1), 36 to 49 (V_{H}FR2), 67 to 98 (V_{H}FR3), 112 to 122 (V_{H}FR4) of SEQ ID NO: 7, 1 to 22 (V_{L}FR1), 34 to 48 (V_{L}FR2), 56 to 87 (V_{L}FR3), and 97 to 106 (VLFR4) of SEQ ID NO: 8; and
part (B) comprises the following framework regions:
an amino acid sequence with at least 70% sequence identity to the amino acid residues shown in positions 1 to 30 (V_{H}FR1), 38 to 51 (V_{H}FR2), 68 to 99 (V_{H}FR3), and 112 to 122 (V_{H}FR4) of SEQ ID NO: 17, 1 to 23 (V_{L}FR1), 41 to 55 (V_{L}FR2), 63 to 94 (V_{L}FR3), and 104 to 114 (VLFR4) of SEQ ID NO: 18; and
part (C) comprises the following framework regions:
an amino acid sequence with at least 70% sequence identity to the amino acid residues shown in positions 1 to 25 (V_{H}FR1), 36 to 49 (V_{H}FR2), 67 to 98 (V_{H}FR3), and 112 to 122 (V_{H}FR4) of SEQ ID NO: 37, 1 to 23 (V_{L}FR1), 35 to 49 (V_{L}FR2), 57 to 88 (V_{L}FR3), and 98 to 107 (VLFR4) of SEQ ID NO: 38.

As regards this definition as well as preferred embodiments, the same applies, mutatis mutandis, to the trispecific antibody as has been set forth above in the context of the first aspect of the present invention (as regards part (A)) and the second aspect of the present invention (as regards part (B)), respectively, as defined above.

In a preferred embodiment, in the context of the fourth aspect of the present invention, the present invention relates to a trispecific antibody binding to the glycoprotein B (gB) of the HSV-1 and/or HSV-2 as defined above, wherein
part (A) comprises the V_{H} of SEQ ID NO:7 and the V_{L} of SEQ ID NO:8; and
part (B) comprises the V_{H} of SEQ ID NO:19 and the V_{L} of SEQ ID NO:20; and
part (C) comprises the V_{H} of SEQ ID NO:37 and the V_{L} of SEQ ID NO:38.

As regards this definition as well as preferred embodiments, the same applies, mutatis mutandis, to the trispecific antibody as has been set forth above in the context of the first aspect of the present invention (as regards part (A)) and the second aspect of the present invention (as regards part (B)), respectively, as defined above.

In a preferred embodiment, in the context of the fourth aspect of the present invention, the present invention relates to a trispecific antibody binding to the glycoprotein B (gB) of the HSV-1 and/or HSV-2 as defined above, wherein said trispecific antibody is a humanized or fully human antibody.

The term “humanized or fully human antibody” has already been defined above in the context of the first aspect of the present invention. As regards this definition as well as preferred embodiments of the term “humanized or fully human antibody”, the same applies, mutatis mutandis, to the trispecific antibody as has been set forth above in the context of the first aspect of the present invention as defined above.

In a preferred embodiment, in the context of the fourth aspect of the present invention, the present invention relates to a trispecific antibody binding to the glycoprotein B (gB) of the HSV-1 and/or HSV-2 as defined above, wherein said trispecific antibody is a full-length antibody.

The term “full-length antibody” has already been defined above in the context of the first aspect of the present invention. As regards this definition as well as preferred embodiments of the term “full-length antibody”, the same applies, mutatis mutandis, to the trispecific antibody as has been set forth above in the context of the first aspect of the present invention as defined above.

In a preferred embodiment, in the context of the fourth aspect of the present invention, the present invention relates to a trispecific antibody binding to the glycoprotein B (gB) of the HSV-1 and/or HSV-2 as defined above, wherein said trispecific antibody is a human IgG1, an IgG2, an IgG2a, an IgG2b, an IgA1, an IgGA2, an IgG3, an IgG4, an IgA, an IgM, an IgD or an IgE antibody.

The terms “human IgG1, IgG2, IgG2a, IgG2b, IgA1, IgGA2, IgG3, IgG4, IgA, IgM, IgD and IgE antibody” have already been defined above in the context of the first aspect of the present invention. As regards this definition as well as preferred embodiments of the terms “human IgG1, IgG2, IgG2a, IgG2b, IgA1, IgGA2, IgG3, IgG4, IgA, IgM, IgD and IgE antibody”, the same applies, mutatis mutandis, to the trispecific antibody as has been set forth above in the context of the first aspect of the present invention as defined above.

In a preferred embodiment, in the context of the fourth aspect of the present invention, the present invention relates to a trispecific antibody binding to the glycoprotein B (gB) of the HSV-1 and/or HSV-2 as defined above, wherein said trispecific antibody is a F(ab)-, Fab′-SH-, Fv-, Fab′-, or a F(ab′)2-fragment.

The terms “F(ab)-, Fab′-SH-, Fv-, Fab′-, and F(ab′)2-fragment” have already been defined above in the context of the first aspect of the present invention. As regards this definition as well as preferred embodiments of the terms “F(ab)-, Fab′-SH-, Fv-, Fab′-, and F(ab′)2-fragment”, the same applies, mutatis mutandis, to the trispecific antibody as has been set forth above in the context of the first aspect of the present invention as defined above.

In a preferred embodiment, in the context of the fourth aspect of the present invention, the present invention relates to a trispecific antibody binding to the glycoprotein B (gB) of the HSV-1 and/or HSV-2 as defined above, wherein said trispecific antibody in a concentration of at 10 nM, preferably of at most 8 nM, more preferably of at most 6 nM, of at most 4 nM, of at most 2 nM, of at most 1 nM, of at most 0.9 nM, of at most 0.7 nM, and most preferably of at most 0.5 nM, is capable of neutralising a defined amount of HSV of 100 TCID50.

The term “capable of neutralising a defined amount of HSV of 100 TCID50” has already been defined above in the context of the first aspect of the present invention. As regards this definition as well as preferred embodiments of the term “capable of neutralising a defined amount of HSV of 100 TCID50”, the same applies, mutatis mutandis, to the trispecific antibody as has been set forth above in the context of the first aspect of the present invention as defined above.

In a preferred embodiment, in the context of the fourth aspect of the present invention, the present invention relates to a trispecific antibody binding to the glycoprotein B (gB) of the HSV-1 and/or HSV-2 as defined above, wherein said trispecific antibody is capable of inhibiting the spreading of HSV from an infected cell to an adjacent second non-infected cell (cell-to-cell spread).

The term “capable of inhibiting the spreading of HSV from an infected cell to an adjacent second non-infected cell (cell-to-cell spread)” has already been defined above in the context of the first aspect of the present invention. As regards this definition as well as preferred embodiments of the term “capable of inhibiting the spreading of HSV from an infected cell to an adjacent second non-infected cell (cell-to-cell spread)”, the same applies, mutatis mutandis, to the trispecific antibody as has been set forth above in the context of the first aspect of the present invention as defined above.

In a preferred embodiment, in the context of the fourth aspect of the present invention, the present invention relates to a trispecific antibody binding to the glycoprotein B (gB) of the HSV-1 and/or HSV-2 as defined above, wherein said trispecific antibody exerts its antiviral or neutralizing activity independent from antibody-dependent cellular cytotoxicity (ADCC) and/or complement-dependent cytotoxicity (CDC), preferably, wherein said antibody is capable of inhibiting cell-to-cell spread independent from antibody-dependent cellular cytotoxicity (ADCC) and/or complement-dependent cytotoxicity (CDC).

The term “capable of inhibiting cell-to-cell spread independent from antibody-dependent cellular cytotoxicity (ADCC) and/or complement-dependent cytotoxicity (CDC)” has already been defined above in the context of the first aspect of the present invention. As regards this definition as well as preferred embodiments of the term “capable of inhibiting cell-to-cell spread independent from antibody-dependent cellular cytotoxicity (ADCC) and/or complement-dependent cytotoxicity (CDC)”, the same applies, mutatis mutandis, to the trispecific antibody as has been set forth above in the context of the first aspect of the present invention as defined above.

In a preferred embodiment, in the context of the fourth aspect of the present invention, the present invention relates to a trispecific antibody binding to the glycoprotein B (gB) of the HSV-1 and/or HSV-2 as defined above, wherein said trispecific antibody is conjugated to an effector moiety, a therapeutic moiety, or a detectable label.

The term “conjugated to an effector moiety, a therapeutic moiety, or a detectable label” has already been defined above in the context of the first aspect of the present invention. As regards this definition as well as preferred embodiments of the term “conjugated to an effector moiety, a therapeutic moiety, or a detectable label”, the same applies, mutatis mutandis, to the trispecific antibody as has been set forth above in the context of the first aspect of the present invention as defined above.

The anti-HSV antibody or the antigen-binding fragment thereof according the first aspect of the present invention as defined above, the combination of the anti-HSV antibodies or antigen-binding fragments thereof according the second aspect of the present invention as defined above, the bispecific antibody according to the third aspect of the present invention as defined above as well as the trispecific antibody according to the fourth aspect of the present invention as defined above are particularly useful in medical settings.

Thus, in a preferred embodiment, the present invention relates to a pharmaceutical composition comprising an effective amount of the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, and at least one pharmaceutically acceptable excipient.

Hence, in a preferred embodiment, the present invention relates to the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, for use as a drug.

The term “treatment” and/or “prevention” and the like are used herein to generally mean obtaining a desired pharmacological and/or physiological effect. Accordingly, the treatment of the present invention may relate to the treatment of (acute) states of a certain disease but may also relate to the prophylactic treatment in terms of completely or partially preventing a disease or symptom thereof. Preferably, the term “treatment” is to be understood as being therapeutic in terms of partially or completely curing a disease and/or adverse effect and/or symptoms attributed to the disease. “Acute” in this respect means that the subject shows symptoms of the disease. In other words, the subject to be treated is in actual need of a treatment and the term “acute treatment” in the context of the present invention relates to the measures taken to actually treat the disease after the onset of the disease or the outbreak of the disease. The treatment may also be prophylactic or preventive treatment, i.e., measures taken for disease prevention, e.g., in order to prevent the infection and/or the onset of the disease.

The pharmaceutical composition or drug of the present invention may be administered via a large range of classes of forms of administration known to the skilled person. Administration may be systemically, locally, orally, through aerosols including but not limited to tablets, needle injection, the use of inhalators, creams, foams, gels, lotions and ointments.

Preferably, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is to be administered intravenously, topically, intradermally, subcutaneously, intra-cutanously, intramuscular an/or intrathecal. These routes of administration, i.e., via an intravenously, topically, intradermally, subcutaneously, intra-cutanously, intramuscular an/or intrathecal are known to the skilled person.

An excipient or carrier is an inactive substance formulated alongside the active ingredient, i.e., the antibody as described above of the present invention for the purpose of bulking-up formulations that contain potent active ingredients. Excipients are often referred to as “bulking agents,” “fillers,” or “diluents”. Bulking up allows convenient and accurate dispensation of a drug substance when producing a dosage form. They also can serve various therapeutic-enhancing purposes, such as facilitating drug absorption or solubility, or other pharmacokinetic considerations. Excipients can also be useful in the manufacturing process, to aid in the handling of the active substance concerned such as by facilitating powder flowability or non-stick properties, in addition to aiding in vitro stability such as prevention of denaturation over the expected shelf life. The selection of appropriate excipients also depends upon the route of administration and the dosage form, as well as the active ingredient and other factors.

Thus, the pharmaceutical composition or drug comprising the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, as described above may be in solid, liquid or gaseous form and may be, inter alia, in a form of (a) powder(s), (a) tablet(s), (a) solution(s) or (an) aerosol(s). It is preferred that said pharmaceutical composition optionally comprises a pharmaceutically acceptable carrier and/or diluent.

These pharmaceutical compositions can be administered to the subject at a suitable dose. Administration of the suitable compositions may be affected by different ways, e.g., by intravenous, intraperitoneal, subcutaneous, intramuscular, topical, intradermal, intranasal or intrabronchial administration. It is particularly preferred that said administration is carried out by injection and/or delivery, e.g., to a site in a lung artery or directly into the lung. The compositions of the invention may also be administered directly to the target site, e.g., by biolistic delivery to an external or internal target site, like the lung. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 1 μg to 10 mg units per kilogram of body weight per minute.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose, i.e., in “an effective amount” which can easily be determined by the skilled person by methods known in the art. The dosage regimen will be determined by the attending physician and clinical factors. As is well known in the medical arts, dosages for any one patient depends upon many factors, including the patient's or subject's size, body surface area, age, the particular compound to be administered, sex, time and route of administration, general health, and other drugs being administered concurrently.

Thus, preferably, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is/are included in an effective amount. The term “effective amount” refers to an amount sufficient to induce a detectable therapeutic response in the subject to which the pharmaceutical composition is to be administered. In accordance with the above, the content of the antibody/antibodies of the present invention in the pharmaceutical composition is not limited as far as it is useful for treatment as described above, but preferably contains 0.0000001-10% by weight per total composition. Further, the antibody/antibodies described herein is/are preferably employed in a carrier. Generally, an appropriate amount of a pharmaceutically acceptable salt is used in the carrier to render the composition isotonic. Examples of the carrier include but are not limited to saline, Ringer's solution and dextrose solution. Preferably, acceptable excipients, carriers, or stabilisers are non-toxic at the dosages and concentrations employed, including buffers such as citrate, phosphate, and other organic acids; salt-forming counter-ions, e.g. sodium and potassium; low molecular weight (>10 amino acid residues) polypeptides; proteins, e.g. serum albumin, or gelatine; hydrophilic polymers, e.g. polyvinylpyrrolidone; amino acids such as histidine, glutamine, lysine, asparagine, arginine, or glycine; carbohydrates including glucose, mannose, or dextrins; monosaccharides; disaccharides; other sugars, e.g. sucrose, mannitol, trehalose or sorbitol; chelating agents, e.g. EDTA; non-ionic surfactants, e.g. Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol). Suitable carriers and their formulations are described in greater detail in Remington's Pharmaceutical Sciences, 17th ed., 1985, Mack Publishing Co.

Therapeutic progress can be monitored by periodic assessment.

The pharmaceutical composition/drug of the invention may be in sterile aqueous or non-aqueous solutions, suspensions, and emulsions as well as creams and suppositories. Examples of non-aqueous solvents are propylene glycol, polyethylene glycol, vegetable oils such as olive oil, and organic esters such as ethyl oleate. Aqueous carriers include water, alcoholic/aqueous solutions, emulsions or suspensions, including saline and buffered media. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition. Said agents may be, e.g., polyoxyethylene sorbitan monolaurate, available on the market with the commercial name Tween, propylene glycol, EDTA, Citrate, Sucrose as well as other agents being suitable for the intended use of the pharmaceutical composition that are well-known to the person skilled in the art.

In accordance with this invention, the term “pharmaceutical composition” relates to a composition for administration to a patient, preferably a human patient.

As outlined, the second aspect of the present invention relates to a combination of two antibodies and/or antibody-binding fragments as defined above.

Accordingly, in particular, in the context of the second aspect of the present invention, the following applies.

The term “combination” in the context of the second aspect of the present invention to a combination of the following two components as follows (as well as the preferred embodiments as described herein-above in the context of the first and second aspect, respectively, of the present invention):
(A) an anti-HSV antibody or an antigen-binding fragment thereof as defined herein above in the context of the first aspect of the present invention, i.e., an anti-HSV antibody or an antigen-binding fragment thereof binding to the glycoprotein B (gB) of the HSV-1 and/or HSV-2, wherein said antibody comprises:
   the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6; and
(B) an anti-HSV antibody or an antigen-binding fragment thereof as defined herein above in the context of the second aspect of the present invention, i.e., an anti-HSV antibody or an antigen-binding fragment thereof binding to the glycoprotein B (gB) of the HSV-1 and/or HSV-2, wherein said antibody comprises:
   the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO: 16; wherein said antibody has a dissociation constant Kd of at most 40 nM, preferably at most 30 nM, more preferably at most 20 nM, even more preferably at most 15 nM, at most 13 nM and at most 10 nM as defined herein above in the context of the first aspect of the present invention.

In a preferred embodiment, a simultaneous application is envisaged. Yet, the combination also encompasses a subsequent application of the two components. Thus, one of these components may be administered before, simultaneously with or after the other one of the combination, or vice versa.

Accordingly, “in combination” as used herein does not restrict the timing between the administration of the two components. Thus, when the two components are not administered simultaneously with/concurrently, the administrations may be separated by 1 minute, 5 minutes, 15 minutes, 30 minutes, 45 minutes, 1 hour, 2 hours, 4 hours, 6 hours, 12 hours, 24 hours, 48 hours or 72 hours or by any suitable time differential readily determined by one of skill in art and/or described herein. In a preferred embodiment, when the two components are not administered simultaneously with/concurrently, the administrations may be separated by 1 hour, 2 hours, 4 hours, 6 hours, 12 hours, 24 hours, 48 hours or 72 hours or by any suitable time differential readily determined by one of skill in art and/or described herein.

In a further preferred embodiment, the present invention relates to the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, for use in a method for the prophylactical or therapeutical treatment of a disorder or disease selected from the group consisting of Herpes simplex labialis, Herpes simplex genitalis, chronic or disseminated cutaneous herpes simplex infection, Herpes gladiatorum, Eczema herpeticum, Herpes keratoconjunctivitis, Herpes neonatorum, Alzheimer disease (AD), HSV pneumonia, Bell's palsy, Herpes esophagitis, Herpesviral encephalitis and Herpesviral meningitis, Herpetic sycosis, Herpes with low, Herpes gingivostomatitis, presence of an oral herpes relapse or recidivism, presence of a genital herpes relapse or recidivism, eczema herpeticatum, herpes neonatorum, immune deficiency, immunocompromized patients, resistance against a virostatic agent, encephalitis, meningitis, meningoencephalitis, eye infections, and/or generalized HSV infections.

In more preferred embodiments, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is for use in the prophylactical or therapeutical treatment of an HSV-associated disease, wherein said disease is caused by HSV-1 or HSV-2, even more preferably wherein said HSV-associated disease is selected from the group consisting of Herpes simplex labialis, Herpes simplex genitalis, chronic or disseminated cutaneous herpes simplex infection, Herpes gladiatorum and Eczema herpeticum.

HSV infection may cause several distinct diseases. Common infection of the skin or mucosa may affect the face and mouth (orofacial herpes), genitalia (genital herpes), or hands (herpes whitlow). More serious disorders occur when the virus infects and damages the eye (herpes keratitis), or invades the central nervous system, damaging the brain (herpes encephalitis). Patients with immature or suppressed immune systems, such as newborns, transplant recipients, or AIDS patients are prone to severe complications from HSV infections. HSV-associated diseases also include herpes gladiatorum, Mollaret's meningitis, possibly Bell's palsy, disorders being associated with cognitive deficits of bipolar disorder, also known as manic depression, manic depressive disorder or bipolar affective disorder, and Alzheimer's disease. With regard to Alzheimer's disease, recent scientific publications demonstrated a striking localization of herpes simplex virus type 1 DNA within the beta-amyloid plaques, suggesting that this virus may be a cause of the plaques. In addition, serologic analysis correlated HSV-seropositivity with an increased risk for dementia or Alzheimer disease.

Finally, the use of the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is useful if the development of resistant strains against common chemotherapeutic virostatic agents is observed, e.g., during long-lasting prophylactical and therapeutical treatment of immunosuppressed patient. Thus, in a preferred embodiment, the HSV-associated disease is accompanied with one or more of the following features: presence of an oral herpes relapse or recidivism, presence of a genital herpes relapse or recidivism, eczema herpeticatum, herpes neonatorum, immune deficiency (immunocompromised patients), immunosuppression, encephalitis, meningitis, meningoencephalitis, eye infections, generalised HSV infections and/or resistance against a virostatic agent.

In a further preferred embodiment, the present invention relates to the anti-HSV antibody or the antigen-binding fragment thereof according to the first aspect of the present invention as defined above for use, the combination of the anti-HSV antibodies or antigen-binding fragments thereof according the second aspect of the present invention as defined above for use, the bispecific antibody according to the third aspect of the present invention as defined above for use, as well as the trispecific antibody according to the fourth aspect of the present invention as defined above for use, respectively, wherein said antibody is to be administered intravenously, topically, intradermally, subcutaneously, intra-cutaneously, intramuscular an/or intrathecal.

The invention also relates to method of treating or preventing a disorder or a disease as defined herein above in a subject, wherein the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is administered to a subject, preferably, in a therapeutically effective amount as defined above.

As regards the preferred embodiments of the method for treatment the same applies, mutatis mutandis, as has been set forth above in the context of the antibody or the pharmaceutical composition for use as defined above.

In the present invention, the subject is, in a preferred embodiment, a mammal such as a dog, cat, pig, cow, sheep, horse, rodent, e.g., rat, mouse, and guinea pig, or a primate, e.g., gorilla, chimpanzee, and human. In a most preferable embodiment, the subject is a human.

In a preferred embodiment, in the above-described medical settings, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, may be administered in combination with a virostatic agent.

Preferably, such a combination therapy exerts synergistic effects on the treatment in accordance with the present invention.

The term “combination” as used herein has been described above. As regards the preferred embodiments of such a combination therapy, the same applies, mutatis mutandis, as has been set forth above in the context of the pharmaceutical composition for use as defined above. Virostatic agents are well-known to the person skilled in the art and are commonly also referred to as antiviral drugs which are a class of medication used specifically for treating viral infections. Specific antivirals are used for specific viruses. Unlike most antibiotics, antiviral drugs do not destroy their target pathogen; instead they inhibit their development and/or infection and/or replication.

With respect to HSV infections, the skilled person is in a position to select an appropriate virostatic agent that is suitable to inhibit the virus' development as defined above in accordance with the present invention. As examples, virostatic agent may be selected from the group consisting of the drug classes of nucleoside analogues, pyrophosphate analogues, nucleotide analogues, amantadin derivatives and helicase-primase inhibitors. Thus, the present invention relates to the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, which is to be administered in combination with a virostatic agent selected from the group consisting of the drug classes of nucleoside analogues, pyrophosphate analogues, nucleotide analogues, and helicase-primase inhibitors.

Nucleoside analogues are known in the art and relate to molecules that act like nucleosides in DNA synthesis. They include a range of antiviral products used to prevent viral replication in infected cells. Once they are phosphorylated, they work as antimetabolites by being similar enough to nucleotides to be incorporated into growing DNA strands, but they act as chain terminators and stop viral DNA Polymerase. Nucleoside, nucleotide and pyrophosphate analogues in general are known to inhibit viral nucleic acid synthesis to block viral replication. Nucleoside, nucleotide analogues are antimetabolite drugs. Pyrophosphate analogues (e.g. Foscarnet) structurally mimic the anion pyrophosphate and exert antiviral activity by a selective inhibition of the pyrophosphate binding site on virus-specific DNA polymerases at concentrations that do not affect cellular DNA polymerases. Nucleotide and pyrophosphate analogues do not require an initial activation (phosphorylation) by thymidine kinases or other kinases before taken up into cells. Helicase-primase inhibitors are non-nucleosidic inhibitors that target the viral helicase-primase.

Preferably, commonly known and approved virostatic agents may be used as summarized in the following. As a nucleoside analogue a compound selected from the group consisting of Acyclovir, Penciclovir, Valacyclovir and Famaciclovir may exemplarily be mentioned and used in the combination therapy described above. As a pyrophosphate analogue Foscarnet may be used. As a nucleotide analogue Cidofovir may be used. As a helicase-primase inhibitor Pritelivir is exemplarily mentioned. As an amantadine derivative, Tromantandin may be used. Acyclovir, also known as acycloguanosine (ACV) or 2-Amino-9-(2-hydroxyethoxymethyl)-3H-purin-6-on, is a guanosine analogue antiviral drug, marketed under trade names such as, ACERPES®, Acic®, Aciclobeta®, AcicloCT®, Aciclostad®, Aciclovir, Acic®, Ophtal®, Acivir®, AciVision, Acyclovir®, Aviral®, Cyclovir, Helvevir®, Herpex, Supraviran®, Virucalm®, Virupos® Virzin, Zoliparin®, Zovir, and Zovirax®.

Penciclovir (2-amino-9-[4-hydroxy-3-(hydroxymethyl)butyl]-6,9-dihydro-3H-purin-6-on) is a guanine analogue antiviral drug, marketed under trade names such as Denavir and Fenistil.

Famciclovir (2-[(acetyloxy)methyl]-4-(2-amino-9H-purin-9-yl)butyl acetate) is a prodrug of penciclovir with improved oral bioavailability.

Foscarnet is the conjugate base of the chemical compound with the formula HO₂CPO₃H₂ and is marketed under the trade names Foscavir® and Triapten®.

Valacyclovir, also known as (S)-2-[(2-amino-6-oxo-6,9-dihydro-3H-purin-9-yl)methoxy]ethyl-2-amino-3-methylbutanoate, is a prodrug of the guanosine analogue antiviral drug ACV marketed under the name e.g. Valtrex®.

Cidovovir (CDV), also known as (S)-1-[3-hydroxy-2-(phosphonylmethoxypropyl)]cytosine, is a nucleotide analogue antiviral drug marketed under the name Visitde®.

Pritelevir is a thiazolylamide, also known as AIC-316, or BAY 57-1293, is a helicase-primase inhibitor currently in clinical phase II trials for treatment of genital HSV-2 infections.

The local therapeutic drug Tromantandin (Viru-Merz Serol Gel) is explicitly used for local treatment of HSV skin infections. Tromantandin is an amantadin derivative. Griffin U.S. Pat. No. 4,351,847 discloses that an amantadine derivative is effective against herpes simplex virus.

In more preferred embodiments, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is/are to be administered topically.

Thus, in more preferred embodiments, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is for use in the prophylactical or therapeutical treatment an HSV-associated disease, wherein said disease is caused by HSV-1 or HSV-2, even more preferably wherein said HSV-associated disease is selected from the group consisting of Herpes simplex labialis, Herpes simplex genitalis, chronic or disseminated cutaneous herpes simplex infection, Herpes gladiatorum and Eczema herpeticum, wherein said antibody/antibodies is/are to be topically administered. In further preferred embodiments, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is for use in treating an acute infection of mucosal or epidermal tissue in a subject caused by HSV-1 or HSV-2 selected from the group consisting of Herpes simplex labialis, Herpes simplex genitalis, chronic or disseminated cutaneous herpes simplex infection, Herpes gladiatorum and Eczema herpeticum, wherein said antibody is to be topically administered.

As described in WO 2015/197763 (A1), it has surprisingly been demonstrated in that the topical administration of a humanized anti-HSV antibody in an acute infection of the tissue of the lips upon HSV-infection rapidly eliminates the infection within 24 hours while the local spreading of the Herpes infection via cell-to-cell spread is prevented, thereby avoiding the generation of lesions.

Not only but, in particular, relevant in the context of the more specific embodiment regarding the topical administration, the infections of mucosal or epidermal tissue are infections selected from the group consisting of Herpes simplex labialis, Herpes simplex genitalis, chronic or disseminated cutaneous herpes simplex infection, Herpes gladiatorum and Eczema herpeticum are well known to the person skilled in the art and represent well-defined diseases. Herpes simplex labialis (also called cold sores, herpes simplex labialis, recurrent herpes labialis, or orolabial herpes) is a type of herpes simplex occurring on the lip, i.e. an infection by herpes simplex virus (HSV). An outbreak typically causes small blisters or sores on or around the mouth commonly known as cold sores or fever blisters. The sores typically heal within 2 to 3 weeks, but the herpes virus remains dormant in the facial nerves, following orofacial infection, periodically reactivating (in symptomatic people) to create sores in the same area of the mouth or face at the site of the original infection. Cold sore has a rate of frequency that varies from rare episodes to 12 or more recurrences per year. People with the condition typically experience one to three attacks annually. The frequency and severity of outbreaks generally decreases over time.

Herpes simplex genitalis (or genital herpes) is a genital infection caused by the herpes simplex virus. A 1998 study indicated it was the most common sexually transmitted infection by the number of cases. Most individuals carrying herpes are unaware they have been infected and many will never suffer an outbreak, which involves blisters similar to cold sores. While there is no cure for herpes, over time symptoms are increasingly mild and outbreaks are decreasingly frequent. HSV has been classified into two distinct categories, HSV-1 and HSV-2. Although genital herpes was previously caused primarily by HSV-2, genital HSV-1 infections are increasing and now cause up to 80% of infections. When symptomatic, the typical manifestation of a primary HSV-1 or HSV-2 genital infection is clusters of genital sores consisting of inflamed papules and vesicles on the outer surface of the genitals, resembling cold sores. These usually appear 4-7 days after sexual exposure to HSV for the first time. Genital HSV-1 infection recurs at rate of about one sixth of that of genital HSV-2.

Chronic or disseminated cutaneous herpes simplex infections are known which are not restricted to labial or genital tract. Mostly, immunodeficient patients are affected with this disease like, e.g., patients with Hypogammaglobulinema or patients with cutaneous T-cell lymphomas. Chronic cutaneous herpes simplex is a distinctive clinical presentation of the herpes simplex virus (HSV) in a compromised host. This infection can be defined as chronically active destructive skin lesions that potentially may progress into the disseminated (systemic) form. While most HSV infections display episodes that show healing in one or two weeks, the lesions of chronic cutaneous herpes simplex have an indolent course that may last for several months. Chronic cutaneous herpes simplex, which is common in immunosuppressed patients, is characterized by atypical, chronic, and persistent lesions, which complicate and delay the diagnosis. This may lead to death caused by associated complications. It is of vital importance that when evaluating chronic ulcers of long duration, especially in children, the possibility of a chronic herpes simplex virus infection be considered.

Herpes gladiatorum is a herpes skin infection that occurs in adolescence among wrestlers but it is also common in other contact sports. It usually occurs on the head, most commonly the jaw area, the neck, chest, face, stomach, and legs.

Eczema herpeticum, also known as a form of Kaposi varicelliform eruption caused by viral infection, usually with the herpes simplex virus (HSV), is an extensive cutaneous vesicular eruption that arises from pre-existing skin disease, usually atopic dermatitis (AD). Children with AD have a higher risk of developing eczema herpeticum, in which HSV type 1 (HSV-1) is the most common pathogen. Eczema herpeticum can be severe, progressing to disseminated infection and death if untreated.

Accordingly, in preferred embodiments, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is to be topically applied to infected mucosal or epidermal tissue of the lips, genitals, nose, ears, eyes, fingers, toes and/or skin areas throughout the body, preferably on the head, the jaw area, neck, chest, face, stomach and/or legs.

Moreover, in further preferred embodiments, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is to be topically applied to areas surrounding the infected mucosal or epidermal tissue.

Not only but, in particular relevant in the context of the more specific embodiment regarding the topical administration, preferably, the treatment relates to the treatment of an acute infection.

The term “treatment” and the like are used herein to generally mean obtaining a desired pharmacological and/or physiological effect. Yet, in the context of a topical administration, it is preferred that the treatment relates to the treatment of acute infections and, accordingly, excludes that the effect may be prophylactic in terms of completely or partially preventing a disease or symptom thereof. Rather, in this context, the term “treatment” is to be understood as being therapeutic in terms of partially or completely curing a disease and/or adverse effect and/or symptoms attributed to the disease of an acute HSV infection as defined above. Hence, the treatment of the present invention relates to the treatment of acute infections. “Acute” in this respect means that the subject shows symptoms of the disease. In other words, the subject to be treated is in actual need of a treatment and the term “acute treatment” in the context of the present invention relates to the measures taken to actually treat the disease after the onset or the breakout of the disease. The term “acute” as referred to in the context of the present invention is opposed to a prophylactic treatment or preventive treatment, i.e., measures taken for disease prevention, e.g., in order to prevent the infection and/or the onset/outbreak of the disease. More specifically, prophylactic treatment may be understood in a way that it prevents attachment of free virus particles (from outside the body) to target cells and in turn prevents virus replication. In contrast, at an acute infection (which could be a primary or a recurrent infection) progeny virus have been raced upon HSV replication. Thus, the “acute treatment” referred to in the present invention does explicitly not relate to prophylactic or preventive treatment of an infection caused by HSV-1 or HSV-2.

Mucosal tissue that may display an acute infection refers to tissues of the mucous membranes which are linings of mostly endodermal origin, covered in epithelium, which are involved in absorption and secretion. They line cavities that are exposed to the external environment and internal organs. They are at several places contiguous with skin: e.g., at the nostrils, the lips of the mouth, the eyelids, the ears, the genital area, and the anus.

Epidermal tissue that may display an acute infection refers to tissues of the epidermis, i.e., the outermost layers of cells in the skin, which together with the dermis forms the cutis. The epidermis is a stratified squamous epithelium composed of proliferating basal and differentiated suprabasal keratinocytes which acts as the body's major barrier against an inhospitable environment, by preventing pathogens from entering, making the skin a natural barrier to infection. It also regulates the amount of water released from the body into the atmosphere through transepidermal water loss.

Topical administration in accordance with the present invention relates to a medication or application or administration that is applied to body surfaces such as the skin or mucous membranes to treat the infection referred to above via a large range of classes of forms of administration, including but not limited to creams, foams, gels, lotions and ointments. In a preferred embodiment, topical administration is understood to be epicutaneous, meaning that the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, is applied directly to the skin. Without being bound by theory and to provide some further non-limiting examples, topical application may also be inhalational, such as asthma medications, or applied to the surface of tissues other than the skin, such as eye drops applied to the conjunctiva, or ear drops placed in the ear, or medications applied to the surface of a tooth. As a route of administration, topical administration are contrasted with enteral (in the digestive tract) and intravascular/intravenous (injected into the circulatory system). In its broadest sense, a topical effect may be understood in a way that it relates to, in the pharmacodynamic sense, a local, rather than systemic, target for a medication.

The mode of topical administration in accordance with the present invention, i.e., the medication, pharmaceutical composition or application or administration that is applied to body surfaces such as the skin or mucous membranes to treat the infection of acute infection of mucosal or epidermal tissue in a subject caused by HSV-1 or HSV-2 selected from the group consisting of Herpes simplex labialis, Herpes simplex genitalis, chronic or disseminated cutaneous herpes simplex infection, Herpes gladiatorum and Eczema herpeticum is not particularly limited and the skilled person knows many forms and preparations that may be suitable for topical administration. Without being bound by theory and without being limiting, the following examples are given. There are many general classes, with no clear dividing line between similar formulations suitable for topical medication. As an example, a topical solution may be used. Topical solutions are generally of low viscosity and often use water or alcohol in the base.

As another example, a lotion may be used to administer the anti-HSV antibody topically. Lotions are similar to solutions but are thicker and tend to be more emollient in nature than solution. They are usually an oil mixed with water, and more often than not have less alcohol than solutions.

As another example, a cream may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically. A cream is usually an emulsion of oil and water in approximately equal proportions. It penetrates the stratum corneum outer layer of skin well. Cream is thicker than lotion, and maintains its shape when removed from its container. It tends to be moderate in moisturizing tendency.

As another example, an ointment may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically. An ointment is commonly a homogeneous, viscous, semi-solid preparation, most commonly a greasy, thick oil (oil 80%-water 20%) with a high viscosity, that is intended for external application to the skin or mucous membranes. Ointments have a Water number that defines the maximum amount of water that it can contain. They may be used as emollients or for the application the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, in accordance with the present invention to the skin for protective, therapeutic, or prophylactic purposes and where a degree of occlusion is desired. The vehicle of an ointment is known as the ointment base. The choice of a base depends upon the clinical indication for the ointment and is appropriately chosen based on the person skilled in the art's knowledge. Different types of ointment bases may be hydrocarbon bases, e.g. hard paraffin, soft paraffin, microcrystalline wax and ceresine; absorption bases, e.g. wool fat, beeswax; water soluble bases, e.g. macrogols 200, 300, 400; emulsifying bases, e.g. emulsifying wax, cetrimide; vegetable oils, e.g. olive oil, coconut oil, sesame oil, almond oil and peanut oil. Commonly, the medicament, is dispersed in the base, and later they get divided after the drug penetration into the living cells of skin. Ointments are commonly formulated using hydrophobic, hydrophilic, or water-emulsifying bases to provide preparations that are immiscible, miscible, or emulsifiable with skin secretions. They can also be derived from hydrocarbon (fatty), absorption, water-removable, or water-soluble bases.

As another example, a gel may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically. Gels are usually thicker than a solution. Gels are often a semisolid emulsion in an alcohol base. Some will melt at body temperature. Gel tends to be cellulose cut with alcohol or acetone.

As another example, a foam may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically.

As another example, a transdermal patch may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically. Transdermal patches can be a very precise time released method of delivering a drug. The release of the active component from a transdermal delivery system (patch) may be controlled by diffusion through the adhesive which covers the whole patch, by diffusion through a membrane which may only have adhesive on the patch rim or drug release may be controlled by release from a polymer matrix.

As another example, a powder may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically. Powder is either the pure drug by itself (talcum powder), or is made of the drug mixed in a carrier such as corn starch or corn cob powder (Zeosorb AF—miconazole powder).

As another example, a solid form may be used to administer the anti-HSV antibody topically. Thus, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, may be placed in a solid form. Examples are deodorant, antiperspirants, astringents, and hemostatic agents. In a preferred embodiment, in particular in the context of the topical administration of the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, in the treatment of an acute infection of mucosal or epidermal tissue caused by HSV-1 or HSV-2 of Herpes simplex genitalis, the anti-HSV antibody may be administered in the form of a suppository. A suppository is a drug delivery system that in the context of the treatment of Herpes simplex genitalis comprises the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, and may be is inserted into the vagina (i.e., in the form of a vaginal suppository), where it dissolves or melts and releases the anti-HSV antibody and, accordingly serves to deliver locally the anti-HSV-antibody.

As another example, a vaporizing device may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically. Thus, the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, may be applied as an ointment or gel, and reach the mucous membrane via vaporization.

As another example, a paste may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically. Paste combines three agents—oil, water, and powder. It is an ointment in which a powder is suspended.

As a final, non-limiting example, a tincture may be used to administer the anti-HSV antibody or the antigen-binding fragment thereof according to the first aspect of the present invention as defined above, the combination of the anti-HSV antibodies or antigen-binding fragments thereof according the second aspect of the present invention as defined above, the bispecific antibody according to the third aspect of the present invention as defined above, as well as the trispecific antibody according to the fourth aspect of the present invention as defined above, respectively, topically. A tincture is a skin preparation that has a high percentage of alcohol.

Other aspects and advantages of the invention will be described in the following examples, which are given for purposes of illustration and not by way of limitation. Each publication, patent, patent application or other document cited in this application is hereby incorporated by reference in its entirety.

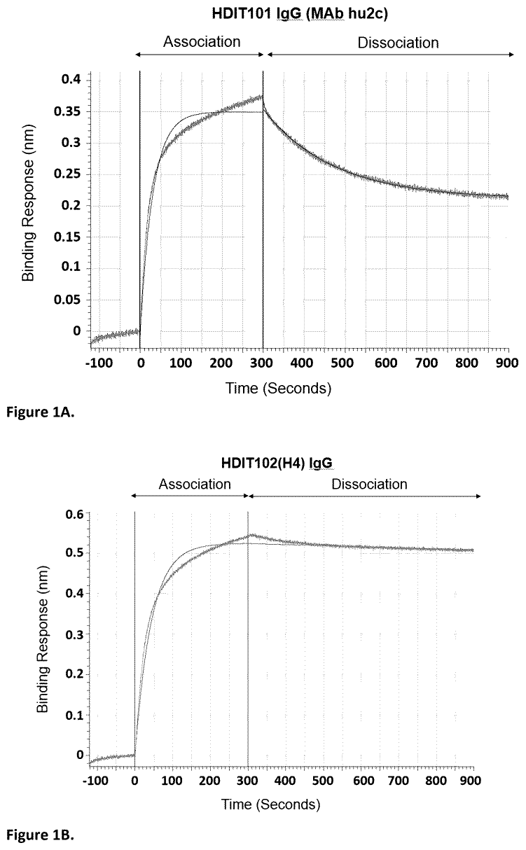

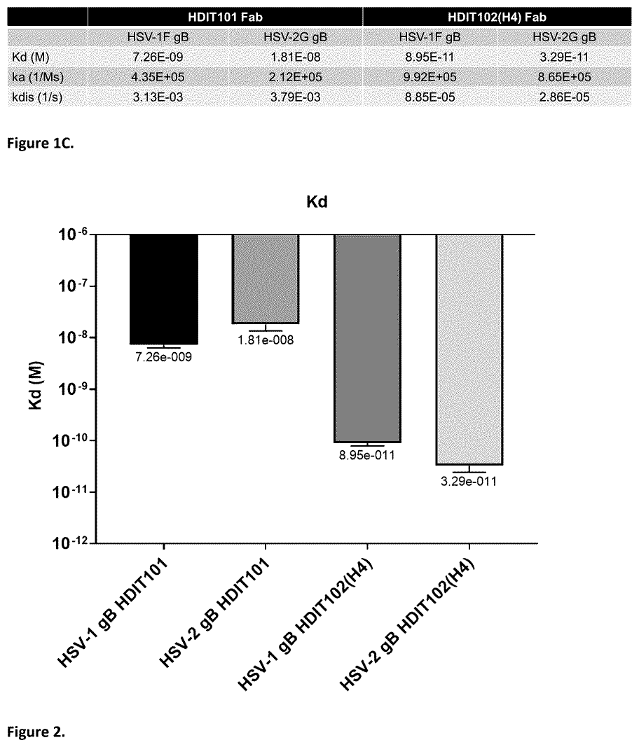

FIG. 1. Bio-layer interferometry (BLI) analysis of recombinant HSV gB interaction with HDIT101 or HDIT102(H4).
Octet sensorgrams demonstrating binding of HSV gB-specific IgGs HDIT101 (MAb hu2c) (as a control) (A) or HDIT102(H4) (B) to immobilized wild type recombinant glycoprotein B of HSV-1F. Biotinylated gB was immobilized on streptavidin (SA) biosensor tips and incubated with 100 nM of HDIT101 or HDIT102(H4) Fab. (C) Measurements of association (ka) and dissociation (kdis) rates and calculation of binding affinities (Kd) using a dilution series of HDIT101 or HDIT102(H4) Fab on HSV-1 or HSV-2 recombinant gB.

FIG. 2. Dissociation constant (Kd) determinations using bio-layer interferometry.
Binding affinity of HDIT101 or HDIT102(H4) Fab to recombinant gB from HSV-1 or HSV-2 was determined using bio-layer interferometry (Octet). Kd values were determined by Kd=kdis/ka.

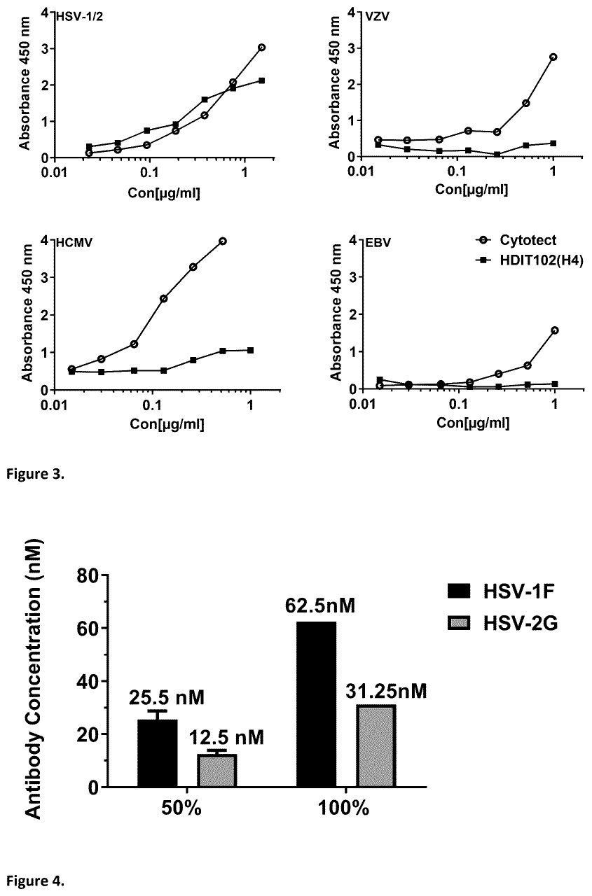

FIG. 3. Specific recognition of HSV-1F or HSV-2G gB by HDIT102(H4).
Binding of a dilution series of HDIT102(H4) at several concentrations to HSV-1/2, VZV, HCMV and EBV antigens was analysed by ELISA using microplates coated with respective viral antigens (Enzygnost, Siemens). Absorbance at 450 nm was measured. Anti-HSV-ELISA does not discriminate between detection of anti-HSV-1 and anti-HSV-2 IgGs. Binding was detected with an HRP-conjugated anti-human gamma Fc-specific IgG. Cytotect (anti-CMV polyclonal antibody preparation) was used as a positive control for all.

FIG. 4. In vitro neutralization of cell-free HSV-1F or HSV-2G by HDIT102(H4).
The antibody concentration required for reducing virus-induced cytopathic effect (CPE) by 100% or 50% was determined by an endpoint dilution assay. Serial dilutions of antibodies were incubated with 100 TCID50 of HSV-1F or HSV-2G for 1 h at 37° C. in cell culture medium. The antibody virus inoculum was applied to Vero cell monolayers grown in microtiter plates and cytopathic effect (CPE) was scored after 72 h of incubation at 37° C. Means and error bars, showing standard deviation of mean, were calculated based on three independent experiments (biological replicates on three different days). For 100% neutralization experiments resulted in identical EC50s, hence no error bars can be shown.

FIG. 5. HDIT102(H4) protects NOD/SCID mice from a lethal infection with HSV-1
(A) Therapeutic activity of HDIT102(H4) was evaluated by infecting (intravaginal inoculation with 5.0×10⁵TCID50) eight-week old female NOD/SCID mice (N=9 for treatment group and N=6 for control). Then, 4 h post infection the mice were treated i.p. with 600 μg, 300 μg or 150 μg of HDIT102(H4). Mice were monitored daily for 55 days post infection for body weight loss, symptoms of HSV infection, such as hair loss, redness, swelling and lesions of the vagina as well as survival. The differences between survival curves were calculated using Logrank Mantel Cox test, ****p-value<0.0001 (B) Viral genome copy numbers in the vaginal swabs obtained on day 1, 3 and 6 post infection, were assessed with qPCR. Mean values are shown from two technical replicates and error bars are standard deviation of the means.

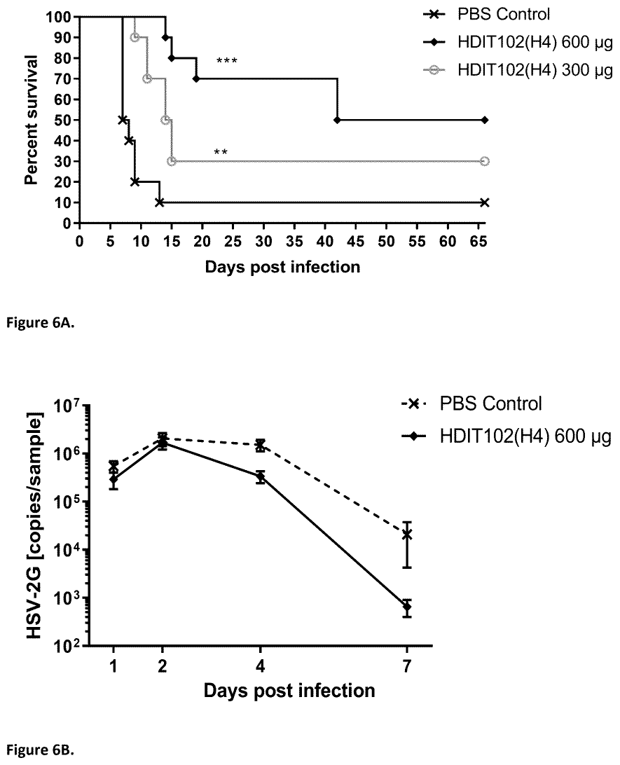

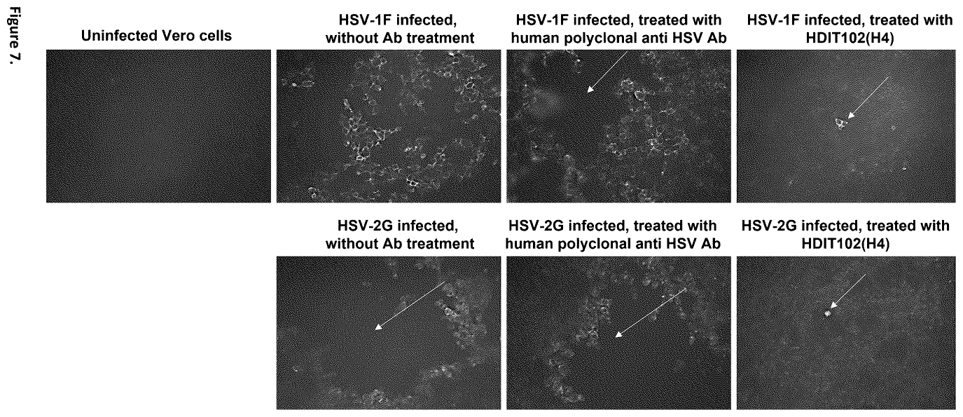

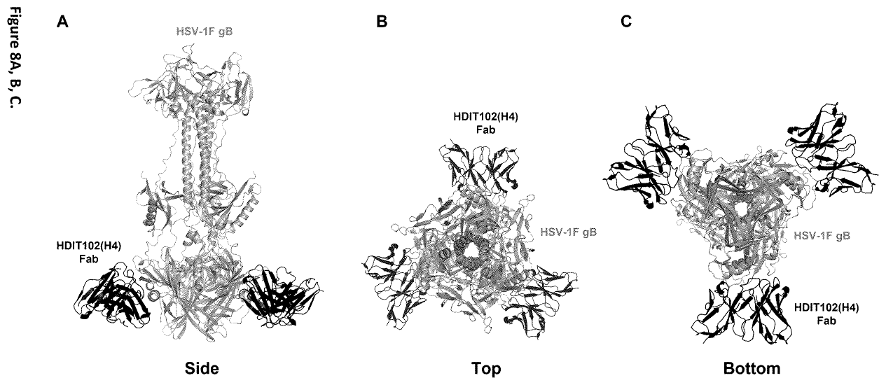

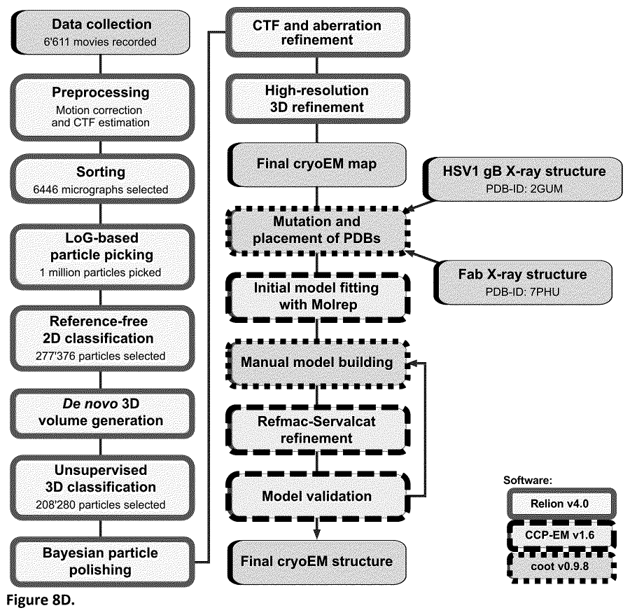

FIG. 6. Survival of HDIT102(H4) treated immunocompetent Balb/c mice after infection with HSV-2G.
(A) HDIT102(H4) protection in-vivo from lethal HSV-2G infection was examined using eight-week-old immunocompetent Balb/c mice. The mice were infected with the lethal dose HSV-2G (5.0×10⁴ TCID50) intravaginally and 4 h later 600 μg or 300 μg of HDIT102(H4) (10 mice per group) were injected intraperitoneally, while the control group received PBS. The statistical differences between survival curves were calculated using Logrank Mantel Cox test, **p-value<0.05, ***p-value<0.001. (B) Vaginal swabs were obtained on day 1, 2, 4 and 7 after infection and HSV-2G copy numbers were measured by qPCR. Mean are values from two technical replicates and error bars represent standard deviation of the mean.

FIG. 7. Inhibition of HSV-1F or HSV-2G cell-to-cell spread by HDIT102(H4).
Fluorescence microscopy images of Vero cells infected with HSV-1F or HSV-2G, treated with either HDIT102(H4), or human polyclonal anti-HSV antibody or left untreated are shown. The cells were infected with HSV-1F or HSV-2G and incubated with 500 nM (75 μg) of test antibody for 48 h, washed, stained with anti HSV-FITC (5 μg/ml) and Hoechst (1:5000) and then fixed with 5% paraformaldehyde before imaging (20×, inverted microscope, Leica). Results are showing one representative image per condition. Arrows are showing the plaques or in case of HDIT102(H4) the initially infected cells.

FIG. 8. Cryo-electron microscopy resolved co-structure of HDIT102(H4) Fab bound to HSV-1F gB.
Cryo-electron microscopy solved structure of HDIT102(H4) Fab (black) bound to recombinantly produced trimeric HSV-1F gB (grey) at a resolution of ~3.44 Å. Side (A), top (B) and bottom (C) view of the trimeric gB with three Fab molecules bound are shown. (D) Single particle Cryo-EM data processing workflow. A graphical representation of the processing steps is shown. Calculations were performed using either Relion (Kimanius, Dong et al., 2021, Biochem J, Vol. 478 (24)), CCP-EM (Nicholls, Tykac et al., 2018, Acta Crystallogr D Struct Biol, Vol. 74 (Pt 6)) or coot (Casanal, Lohkamp et al., 2020, Protein Sci, Vol. 29 (4)) software.

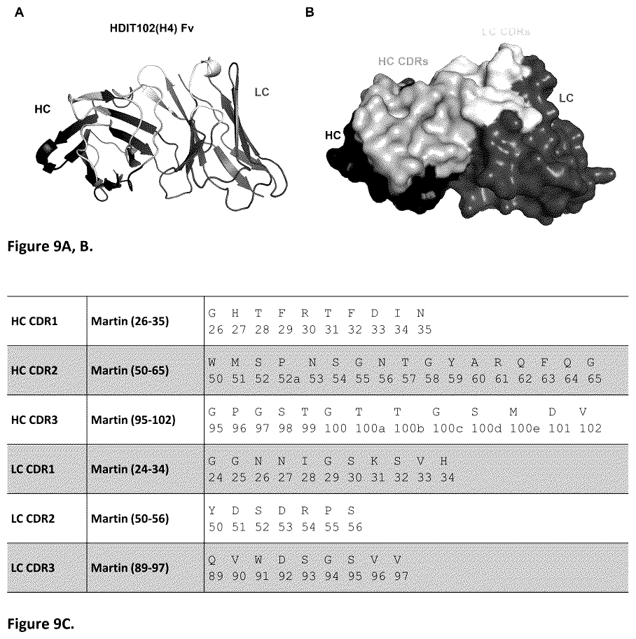

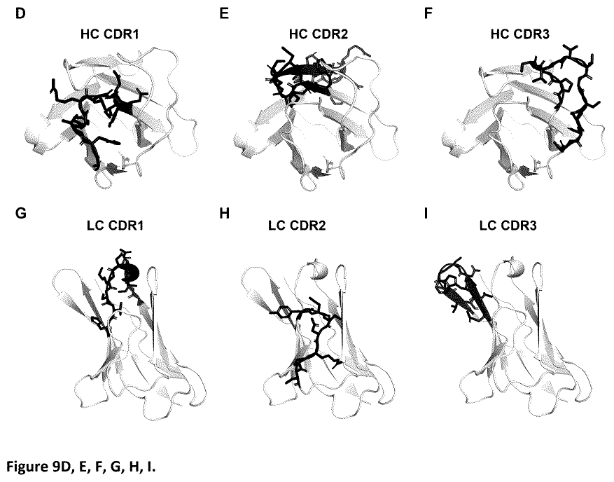

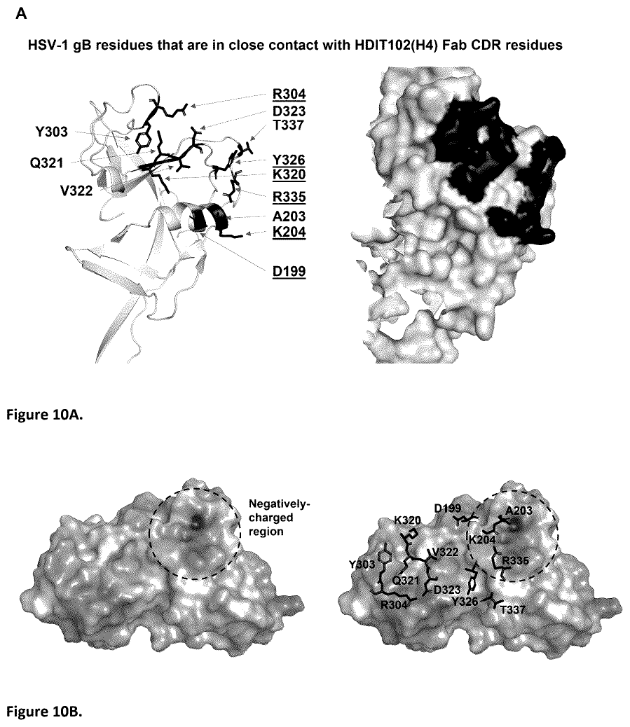

FIG. 9. Structural analysis of the HDIT102(H4) variable fragment and CDRs
Cartoon (A) and surface structure (B) of the HDIT102(H4) variable fragment (Fv) derived from Cryo-EM co-structure of HDIT102(H4) bound to HSV-1F gB depicting heavy chain (HC) and light chain (LC) and complementary-determining regions (CDRs). (C) Sequences of the HDIT102(H4) CDRs of HC and LC and the respective numbering according to the Martin numbering scheme. (D) Location of CDR1-defining residues in HC. (E) Location of CDR2-defining residues in HC. (F) Location of CDR3-defining residues in HC. (G) Location of CDR1-defining residues in LC. (H) Location of CDR2-defining residues in LC. (1) Location of CDR3-defining residues in LC.

FIG. 10. Structural analysis of the HDIT102(H4) epitope in HSV-1 gB with critical residues.
(A) Cartoon and surface structural models of a region of HSV-1F gB protein (light grey) solved in Cryo-EM analysis with gB amino acid residues in black that are in close proximity to CDR residues of HDIT102(H4) to mediate either polar or non-polar interactions. (B) Electrostatic map of HDIT102(H4) Fv with HSV-1F gB residues in black that are in close proximity for mediating interaction. Positively charged HSV-1F gB residues K204 and R335 are in proximity to a negatively charged region of the HDIT102(H4) Fv (circle).

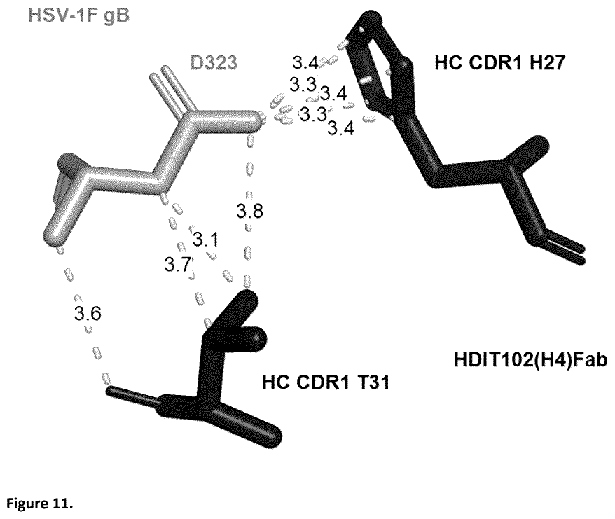

FIG. 11. Analysis of HSV-1 gB residue D323 interactions with HDIT102(H4) CDR residues.
Derived from Cryo-EM structure of HDIT102(H4) Fab in complex with recombinantly produced HSV-1F gB. HSV-1 gB D323 is in proximity to form contacts with HDIT102(H4) HC CDR1 H27 and HC CDR1 T31.

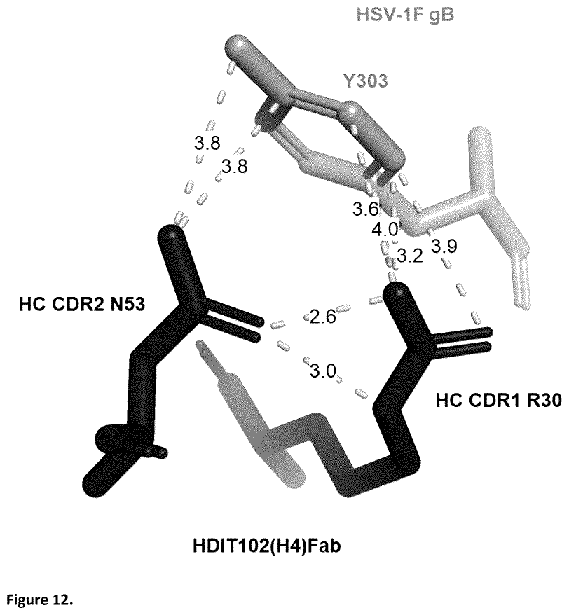

FIG. 12. Analysis of HSV-1 gB residue Y303 interactions with HDIT102(H4) CDR residues
Derived from Cryo-EM structure of HDIT102(H4) Fab in complex with recombinantly produced HSV-1F gB. HSV-1 gBY303 is in proximity to form contacts with HDIT102(H4) HC CDR1 R30 and HC CDR2 N53.

FIG. 13. Analysis of HSV-1 gB residue R304 interactions with HDIT102(H4) CDR residues.
Derived from Cryo-EM structure of HDIT102(H4) Fab in complex with recombinantly produced HSV-1F gB. HSV-1 gB R304 is in proximity to form contact with HDIT102(H4) HC CDR1 H27.

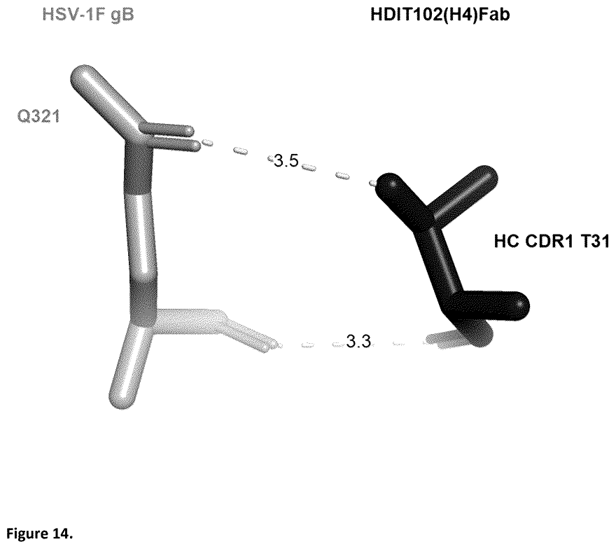

FIG. 14. Analysis of HSV-1 gB residue Q321 interactions with HDIT102(H4) CDR residues.
Derived from Cryo-EM structure of HDIT102(H4) Fab in complex with recombinantly produced HSV-1F gB. HSV-1 gB Q321 is in proximity to form contacts with HDIT102(H4) HC CDR1 T31.

FIG. 15. Analysis of HSV-1 gB residue V322 interactions with HDIT102(H4) CDR residues.
Derived from Cryo-EM structure of HDIT102(H4) Fab in complex with recombinantly produced HSV-1F gB. HSV-1 gB V322 is in proximity to form contacts with HDIT102(H4) HC CDR3 T99.

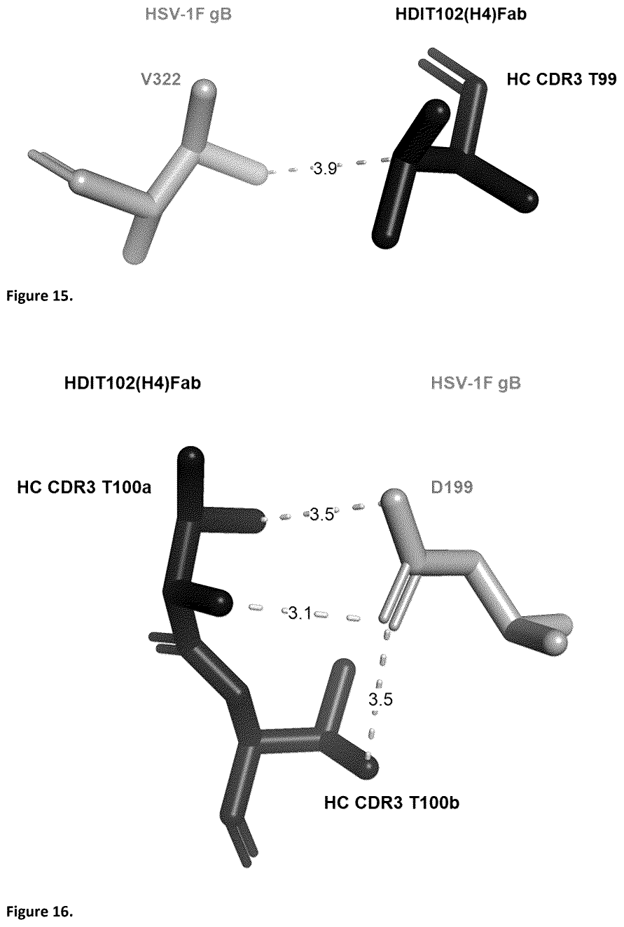

FIG. 16. Analysis of HSV-1 gB residue D199 interactions with HDIT102(H4) CDR residues.
Derived from Cryo-EM structure of HDIT102(H4) Fab in complex with recombinantly produced HSV-1F gB. HSV-1 gB D199 is in proximity to form polar contacts with HDIT102(H4) HC CDR3 T100a and T100b.

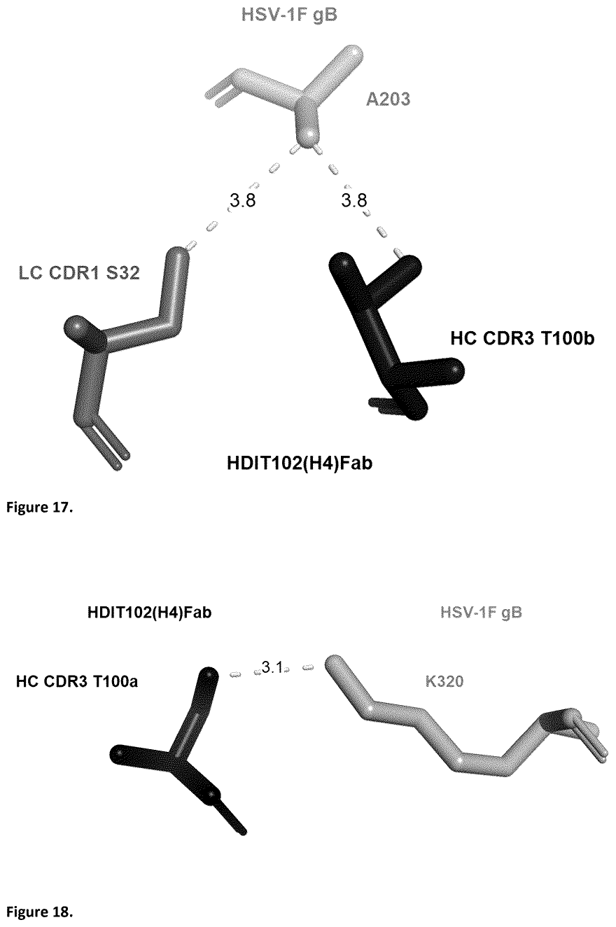

FIG. 17. Analysis of HSV-1 gB residue A203 interactions with HDIT102(H4) CDR residues.
Derived from Cryo-EM structure of HDIT102(H4) Fab in complex with recombinantly produced HSV-1F gB. HSV-1 gB A203 is in proximity to form non-polar contacts with HDIT102(H4) HC CDR3 T100b and LC CDR1 S32.

FIG. 18. Analysis of HSV-1 gB residue K320 interactions with HDIT102(H4) CDR residues.
Derived from Cryo-EM structure of HDIT102(H4) Fab in complex with recombinantly produced HSV-1F gB. HSV-1 gB K320 is, when in a specific rotamer conformation, in proximity to form polar contacts with HDIT102(H4) HC CDR3 T100b and LCCDR1 S32.

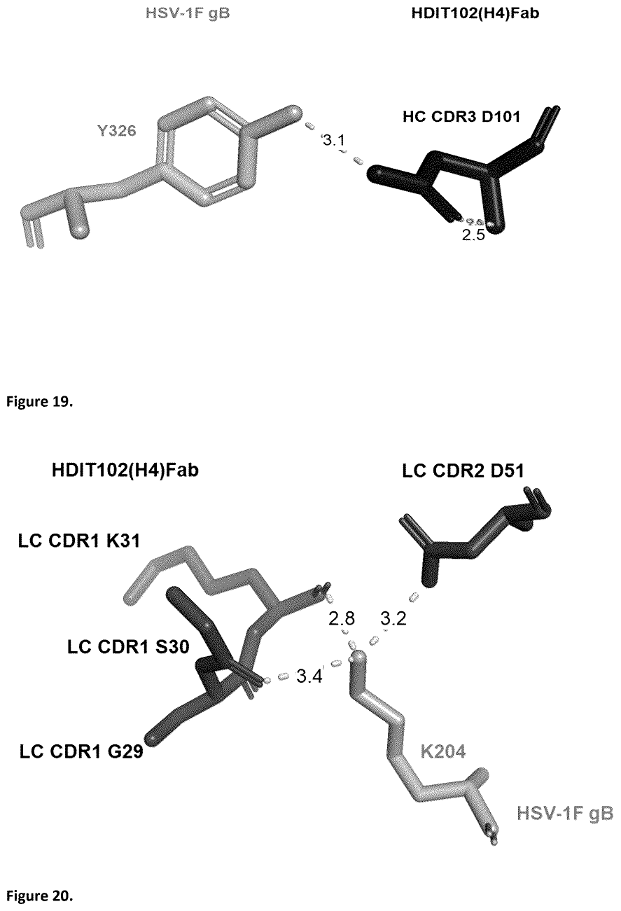

FIG. 19. Analysis of HSV-1 gB residue Y326 interactions with HDIT102(H4) CDR residues.
Derived from Cryo-EM structure of HDIT102(H4) Fab in complex with recombinantly produced HSV-1F gB. HSV-1 gB Y326 is in proximity to form polar contacts with HDIT102(H4) HC CDR3 D101.

FIG. 20. Analysis of HSV-1 gB residue K204 interactions with HDIT102(H4) CDR residues.
Derived from Cryo-EM structure of HDIT102(H4) Fab in complex with recombinantly produced HSV-1F gB. HSV-1 gB K320 is in proximity to form polar contacts with HDIT102(H4) LC CDR2 D51 and peptide backbone at LC CDR1 G29, S30 and K31.

FIG. 21. Analysis of HSV-1 gB residue R335 interactions with HDIT102(H4) CDR residues.
Derived from Cryo-EM structure of HDIT102(H4) Fab in complex with recombinantly produced HSV-1F gB. HSV-1 gB R335 is in proximity to form polar contact with HDIT102(H4) LC CDR2 D53 and non-polar contact with LC CDR2 Y50.

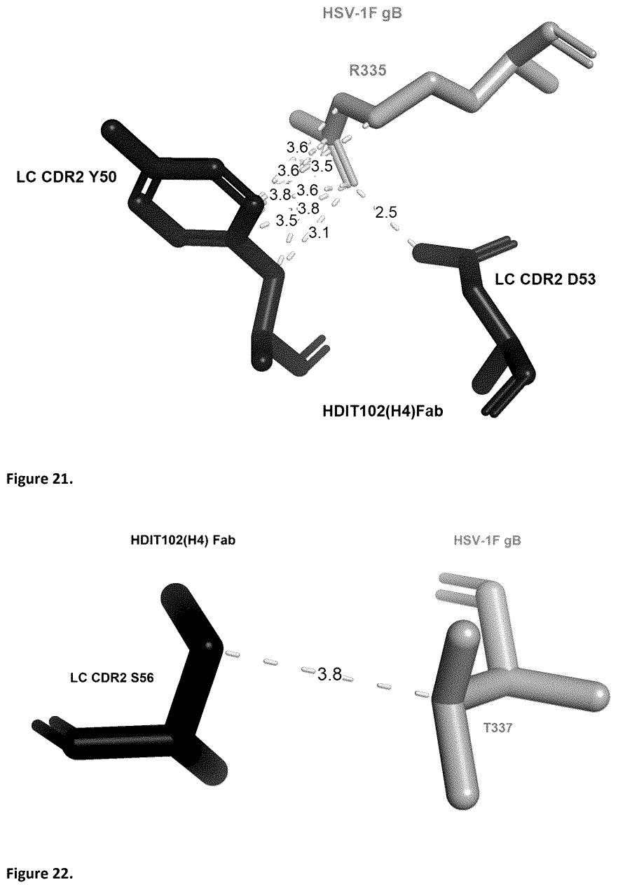

FIG. 22. Analysis of HSV-1 gB residue T337 interactions with HDIT102(H4) CDR residues.
Derived from Cryo-EM structure of HDIT102(H4) Fab in complex with recombinantly produced HSV-1F gB. HSV-1 gB T337 is in proximity to form contact with HDIT102(H4) LC CDR2 S56.

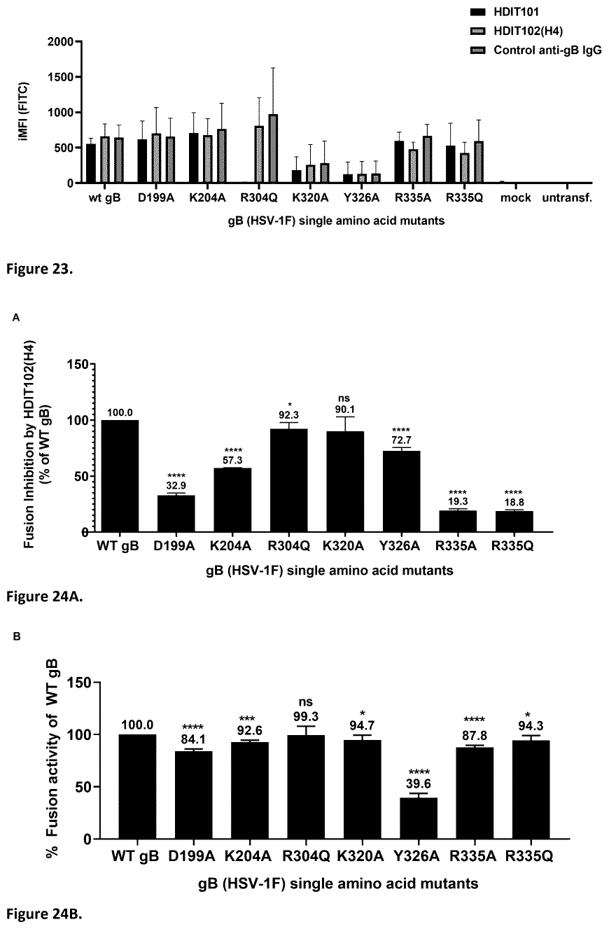

FIG. 23. Alanine scanning mutational analysis of epitope residues in HSV-1F gB and analysis of HDIT102(H4) binding.
Structural information from Cryo-EM analyses was used to choose specific HSV-1F gB amino acid residues in close proximity to HDIT102(H4) CDR residues and interrogate them for their contribution to HDIT102(H4) binding by substitution to alanine. In case of R304 substitution to Q304 was tested as this is the HDIT101-resistance change evolving in vitro under suboptimal HDIT101 concentrations. In case of R335, both A335 and Q335 were tested as R335Q is the observed HDIT102(H4)-resistant escape mutant evolving in vitro under suboptimal HDIT102(H4) concentration. Plasmids encoding wild type HSV-1F gB or indicated single amino acid point mutants were transfected into HEK293T cells, which were stained two days later with HDIT102(H4), HDIT101 or an anti-gB control IgG or without primary antibody. Secondary staining was performed with a FITC-labelled anti-human Fc antibody. To include information on possible differences in cell surface presentation of the mutant gB proteins, the integrated mean fluorescent intensity (iMFI) was calculated by multiplying percentage of HDIT102(H4)-positively stained cells with the geometric MFI of positively stained cells.

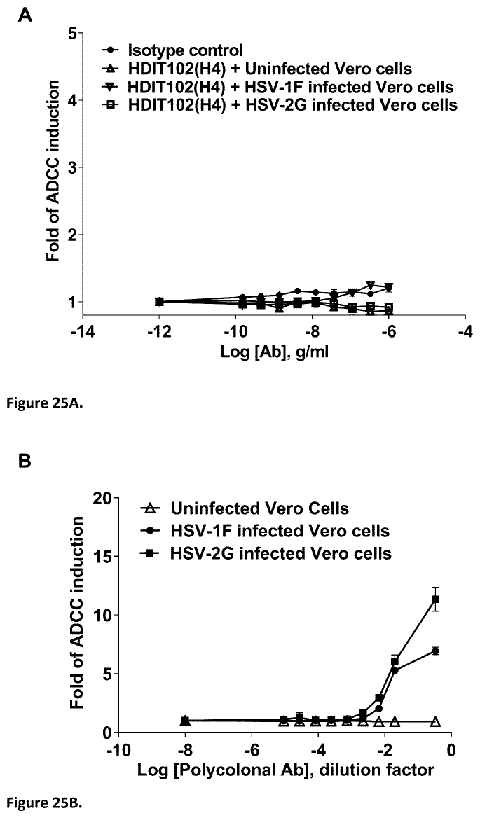

FIG. 24. Alanine scanning mutational analysis of epitope residues in HSV-1F gB and analysis of HDIT102(H4) induced inhibition of cell-cell fusion.
(A) Specific HSV-1F gB amino acid residues in close proximity to HDIT102(H4) CDR residues in the Cryo-EM co-structure were interrogated for their contribution to HDIT102(H4)-mediated inhibition of gB-induced cell-cell fusion by substitution to alanine. Plasmids encoding wild type HSV-1F gB or single amino acid point mutants were co-transfected into a 1:1 mix of HEK293T-GFP and HEK293T-E2C fluorescent reporter cells, together with plasmids encoding HSV-1F gD, gH and gL. Transfected cells were incubated at 37° C. and 5% CO₂ for 5 hours before HDIT102(H4) (75 μg/ml) was added. In control samples no antibody was added. Fusion of cells was judged by the presence of GFP+E2C+ double positive cells by flow cytometry two days later. Changes in fusion inhibition by HDIT102(H4) were calculated comparing HDIT102(H4) treated with untreated cells for each mutant and normalized to wild type gB (WT gB) mean values are shown for at least three independent biological replicates (n=3). Statistical analyses were performed using unpaired t-tests. (B) Fusion activity as combined indicator of cell surface expression and fusogenicity of gB mutants was analysed as in (A) by comparison to wild type gB (WT gB) in the absence of any antibody. Mean values of three independent biological replicates are shown with error bars indicating standard deviation (n=3). Statistical analyses were performed using unpaired t-tests. Mutants that had reduced sensitivity to HDIT102(H4)-mediated fusion inhibition showed also reduced fusion activity as compared to wild type gB possibly indicating reduced fitness of potential HDIT102(H4)-resistant mutant viruses.

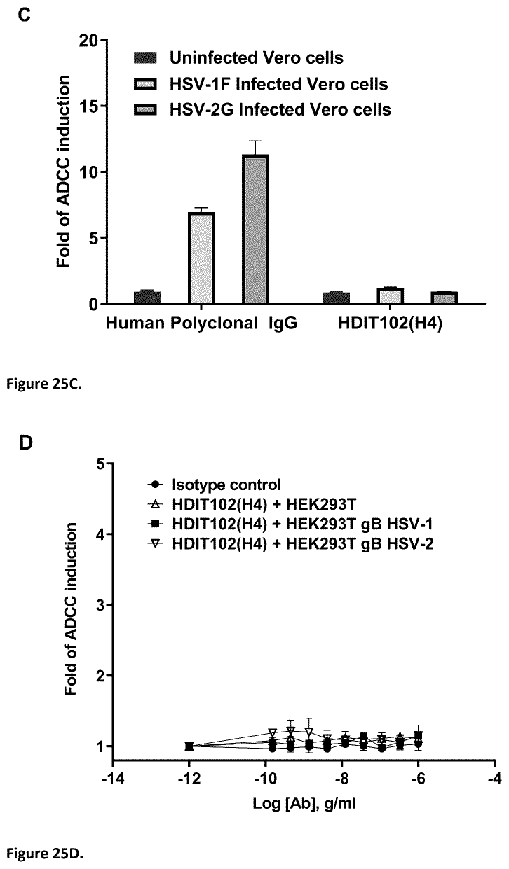

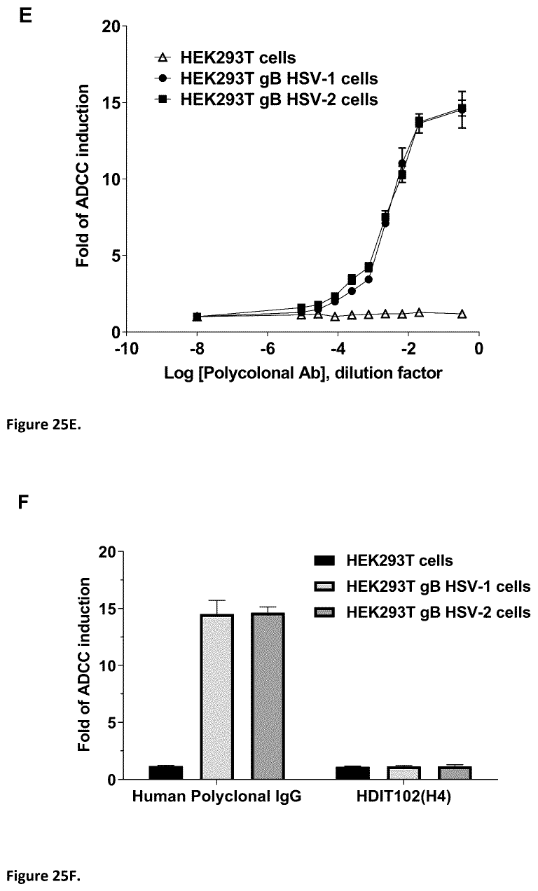

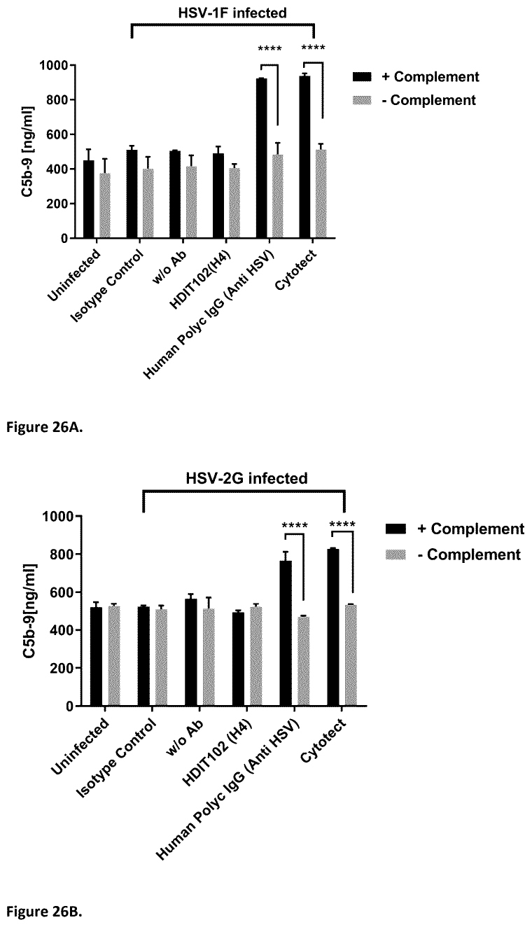

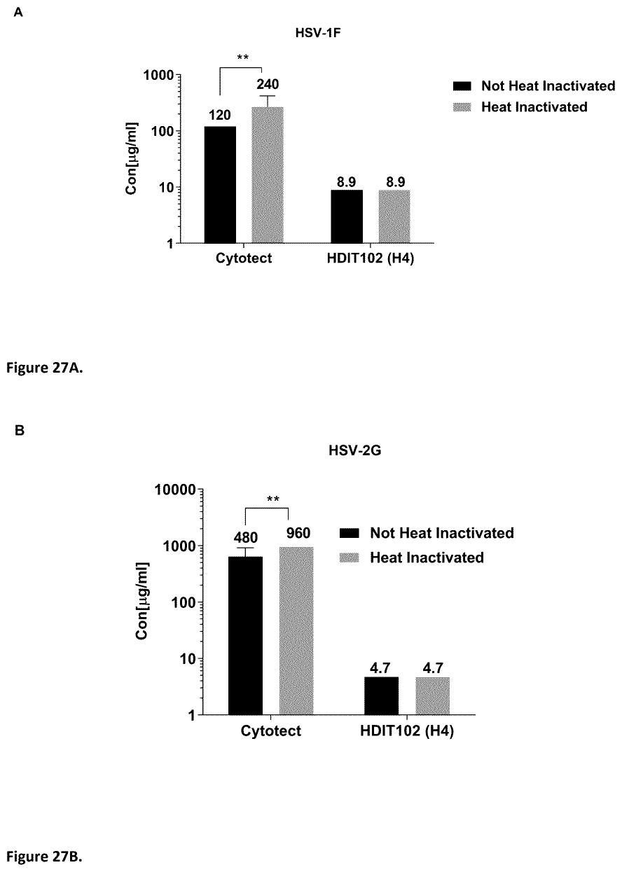

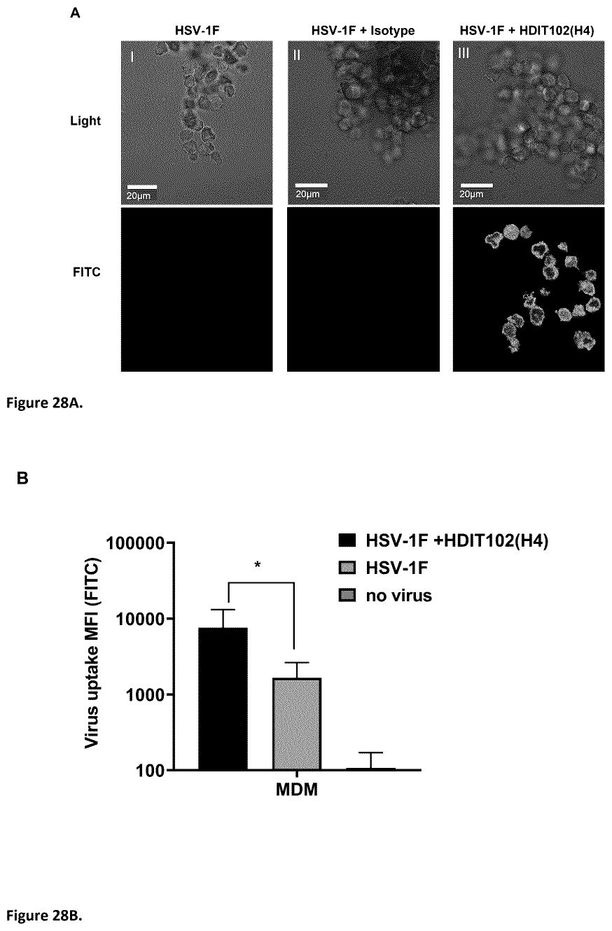

FIG. 25. HDIT102(H4) does not elicit ADCC on HSV-1F or HSV-2G infected Vero cells or HEK293T cells ectopically expressing gB.
Different dilutions of HDIT102(H4) or human polyclonal serum containing anti-HSV IgGs were added to HSV-1F or HSV-2G infected target cells, followed by incubation with effector Jurkat cells (Promega #G7010) stably expressing FcγRIIIA receptor, V158 (high affinity) variant and the NFAT-luciferase reporter at an effector-to-target cell ratio of 6:1 for 6 h at 37° C. Vero cells (African green monkey kidney cells) expressing viral glycoproteins on the cell surface 18 h after infection or HSV-1 gB or HSV-2 gB expressing HEK293T cells were used as target cells. ADCC was quantified by luminescence readout from luciferase activity upon NFAT pathway activation and calculated as fold of induction=RLU (induced-background)/RLU (no antibody-background). Data were fitted to a 4PL curve using GraphPad Prism Vers. 7.02. (A) ADCC activity of anti-gB antibody HDIT102(H4) or isotype control on Vero cells infected with either HSV-1F or HSV-2G or uninfected as control. Mean of three technical replicates is shown. (B) Same as in A, however using human polyclonal antibody as control. (C) Summary of A and B (Fold of ADCC induction in highest concentration of antibodies). (D) ADCC activity of anti-gB antibody HDIT102(H4) on parental HEK293T or lentiviral vector transduced HEK293T ectopically expressing either HSV-1F gB or HSV-2G gB. Mean of three technical replicates is shown. (E) Same as in D, however using human polyclonal antibody as control. (F) Summary of D and E (Fold of ADCC induction in highest concentration of antibodies).

FIG. 26. HDIT102(H4) does not induce complement dependent cytotoxicity (quantitative evaluation of the terminal C5b-9 complement complex).
An ELISA based assay was employed to measure the levels of C5b-9 complex as terminal complement complex to quantify the complement activation by HDIT102(H4). HSV-1F (A) or HSV-2G (B) infected or uninfected Vero cells were incubated with heat-inactivated or with not heat-inactivated IgG-depleted human serum in the presence or absence of test antibody (either HDIT102(H4), human polyclonal IgG, or Cytotect as positive controls, or unrelated isotype IgG as negative control). The supernatants were collected and subjected for an ELISA assay using plates coated with an antiC5b-9 antibody. The differences were calculated by two-way ANOVA, error bars represent standard deviation of the mean for two technical replicates, ****p-value<0.0001).

FIG. 27. HDIT102(H4) neutralizes cell-free HSV-1 or HSV-2 independently of the complement system.
Vero cells were incubated with 100 TCID50 of HSV-1F (A) or HSV-2G (B) in the presence of a dilution series of either HDIT102(H4) or as control Cytotect (human polyclonal IgG preparation). Neutralization activity was tested with not heat inactivated (complement present) or with heat inactivated (complement not present) serum using IgG-depleted human serum. The resulting half maximal effective doses of antibodies to neutralize viral infection were determined 48 h after infection. (TCID50 based assay, the differences were calculated by one-way ANOVA, error bars represent standard deviation of the mean of three technical replicates, **p-value<0.005). In case of absence of error bars, the results of the replicates were identical.

FIG. 28. Antibody-dependent cell phagocytosis (ADCP) mediated by HDIT102(H4).
(A) ADCP was analysed using a microscopy-based assay in which monocytic THP-1 cells were co-incubated for 20 h with HSV-1F in presence of neutralizing amounts of HDIT102(H4) or isotype control. Afterwards, the cells were harvested, stained with a FITC-conjugated anti-human Fc antibody and spun onto glass slides for imaging. Visualization of opsonophagocytic uptake by THP-1 cells was performed using light and fluorescence confocal microscope imaging with 63-fold magnification. I) THP-1 cells incubated with HSV-1F in the absence of antibody. II) THP-1 cells incubated with isotype control (anti-hen egg lysozyme, IgG1)+HSV-1F. III) THP-1 cells incubated with HDIT102(H4)+HSV-1F. The images are representative of two independent experiments with 3 technical replicates each. Same results were observed with HSV-2G (data not shown). (B) Primary human monocyte derived macrophages (MDMs) from four independent healthy donors were incubated with ATTO488 NHS-ester labelled HSV-1F in the presence or absence of HDIT102(H4). The phagocytic uptake of HSV-1F immune complexes with HDIT102(H4) was then determined by measuring virus-ATTO488 positive MDMs via flow cytometry. The statistical differences between groups were calculated using One-way ANOVA. *p-value=0.02.

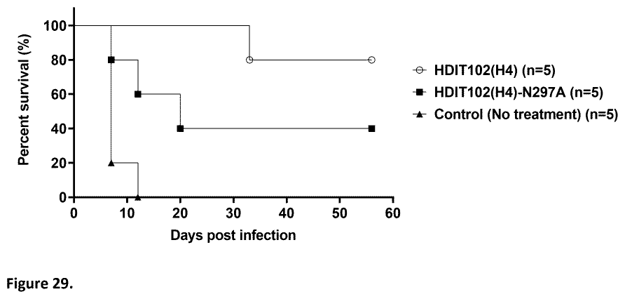

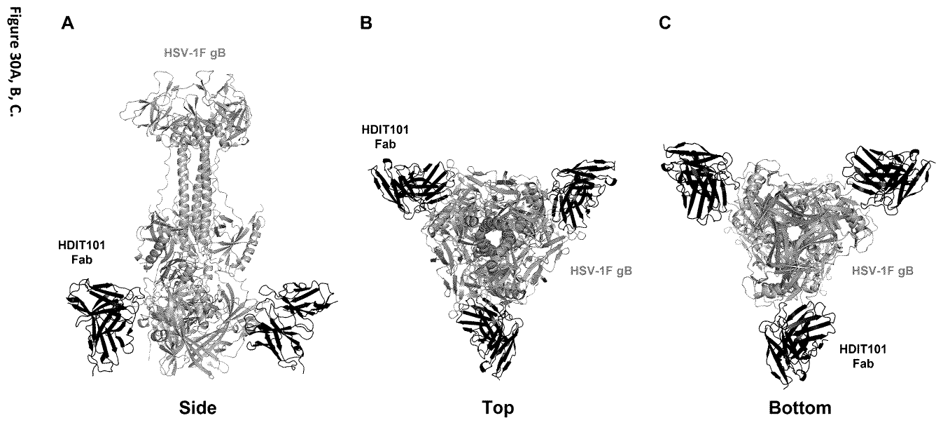

FIG. 29. HDIT102(H4) Fc part plays a pivotal role in rescuing Balb/c mice from a lethal HSV-2 infection.
Eight-week-old immunocompetent female Balb/c mice (5 animals/group) were infected intravaginally with HSV-2G using a lethal dose of 5×10e4 TCID50 and 1 h post infection 600 μg of HDIT102(H4) or HDIT102(H4)-N297A mutant were injected intraperitoneally, while the control group received no treatment. The statistical differences between survival curves were calculated using Logrank Mantel Cox test. P-value=0.15 (HDIT102(H4) vs. HDIT102(H4)-N297A). P-value=0.0016 (HDIT102(H4) vs. untreated). P-value=0.03 (HDIT102(H4)-N297A vs. untreated).

FIG. 30. Cryo-electron microscopy solved co-structure of HDIT101 bound to HSV-1F gB.
Cryo-electron microscopy solved structure of HDIT101 Fab (black) bound to recombinantly produced trimeric HSV-1F gB (grey) at a resolution of ~3.27 Å. Side (A), top (B) and bottom (C) view of the trimeric gB with three HDIT101 Fab molecules bound are shown. (D) Single particle Cryo-EM data processing workflow. A graphical representation of the processing steps is shown. Calculations were performed using either Relion (Kimanius, Dong et al., 2021, Biochem J, Vol. 478 (24)), CCP-EM (Nicholls, Tykac et al., 2018, Acta Crystallogr D Struct Biol, Vol. 74 (Pt 6)) or coot (Casanal, Lohkamp et al., 2020, Protein Sci, Vol. 29 (4)) software.

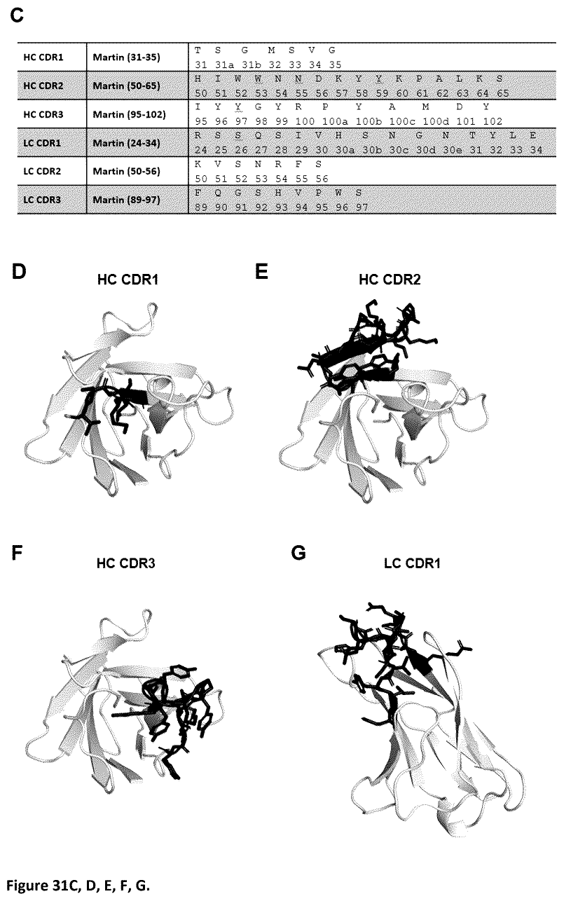

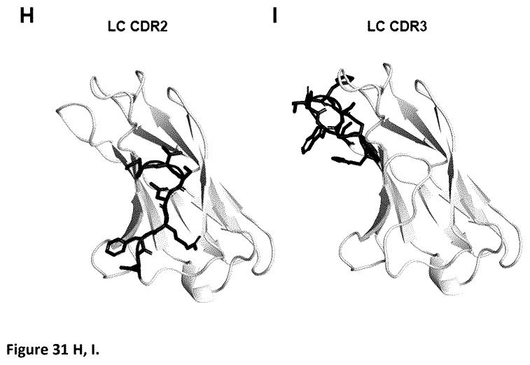

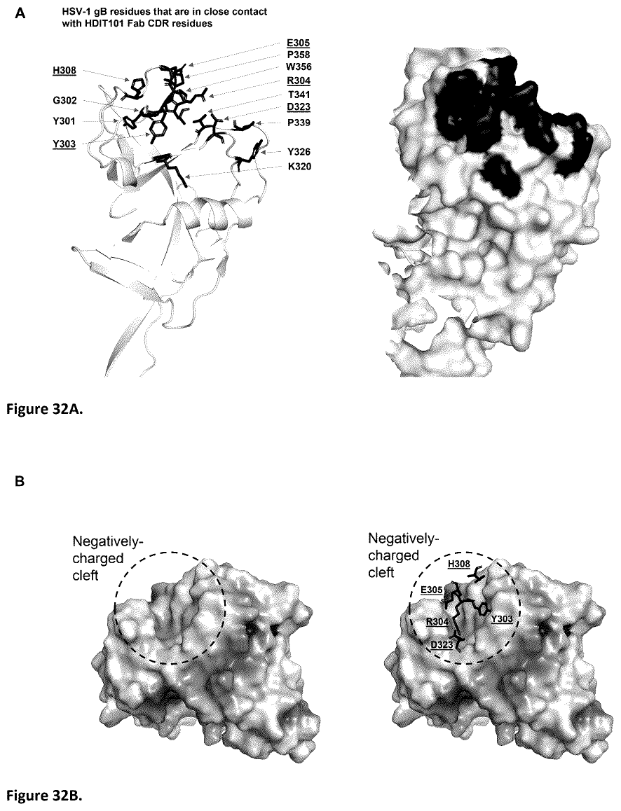

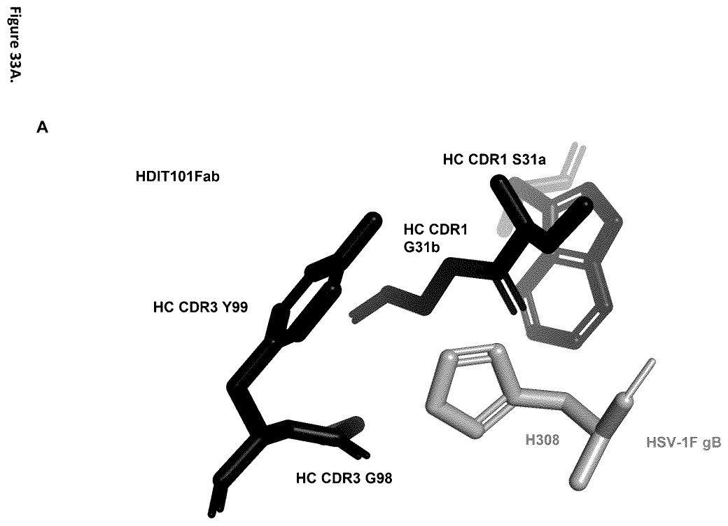

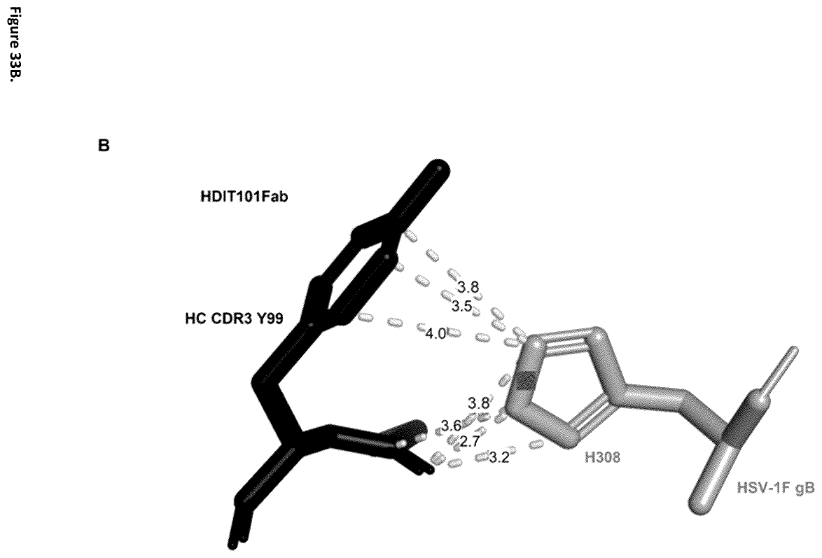

FIG. 31. Structural analysis of the HDIT101 variable fragment and CDRs.
Cartoon (A) and surface structure (B) of the HDIT101 variable fragment (Fv) derived from Cryo-EM co-structure of HDIT101 bound to HSV-1F gB depicting heavy chain (HC) and light chain (LC) and complementary determining regions (CDRs). (C) Sequences of the HDIT101 CDRs of HC and LC and the respective numbering according to the Martin numbering scheme. (D) Location of CDR1-defining residues in HC. (E) Location of CDR2-defining residues in HC. (F) Location of CDR3-defining residues in HC. (G) Location of CDR1-defining residues in LC. (H) Location of CDR2-defining residues in LC. (1) Location of CDR3-defining residues in LC.

FIG. 32. Structural analysis of the HDIT101 epitope in HSV-1 gB with critical residues.
(A) Cartoon and surface structural models of a region of HSV-1F gB protein (light grey) solved in Cryo-EM analysis with gB amino acid residues in black that are in close proximity to CDR residues of HDIT101 to mediate either polar or non-polar interactions. (B) Electrostatic map of HDIT101 Fv with key HSV-1F gB residues in black that are in close proximity for mediating interaction. Key residues of HSV-1F gB, i.e. Y303, R304, E305 and D323 are embedded in a cleft of the HDIT101 Fv (circle) mediating polar and non-polar interactions.

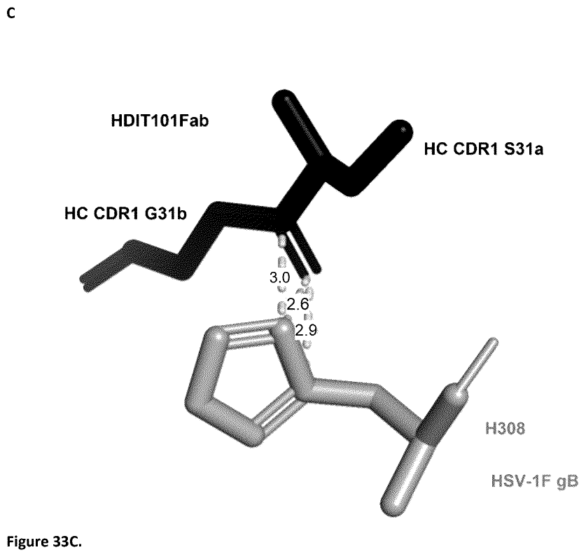

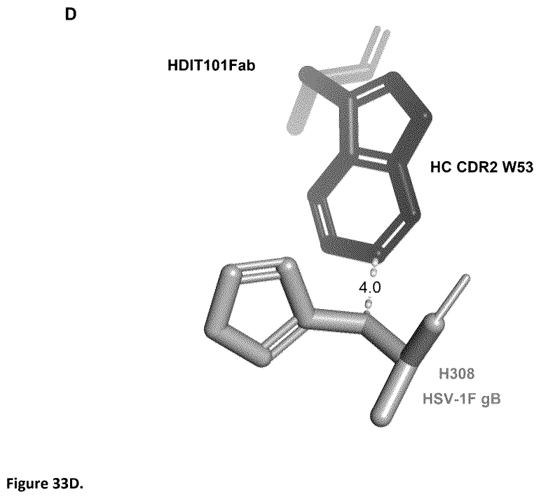

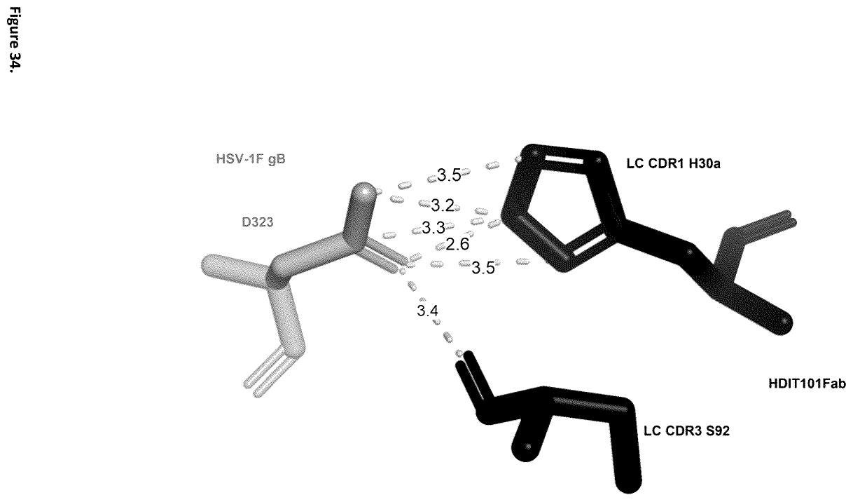

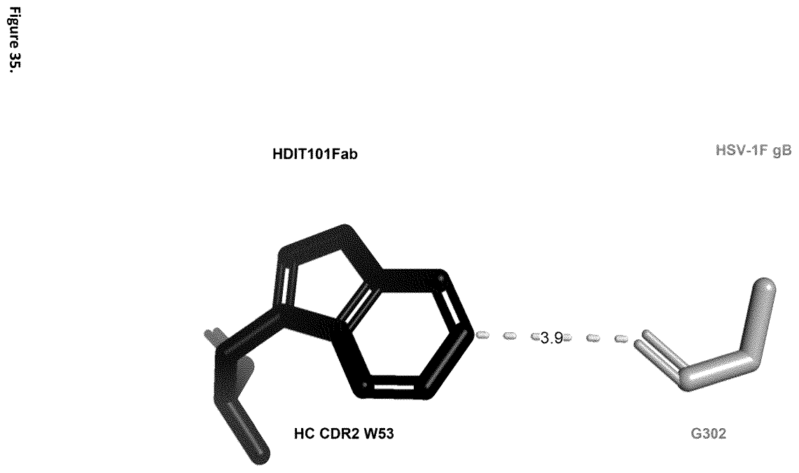

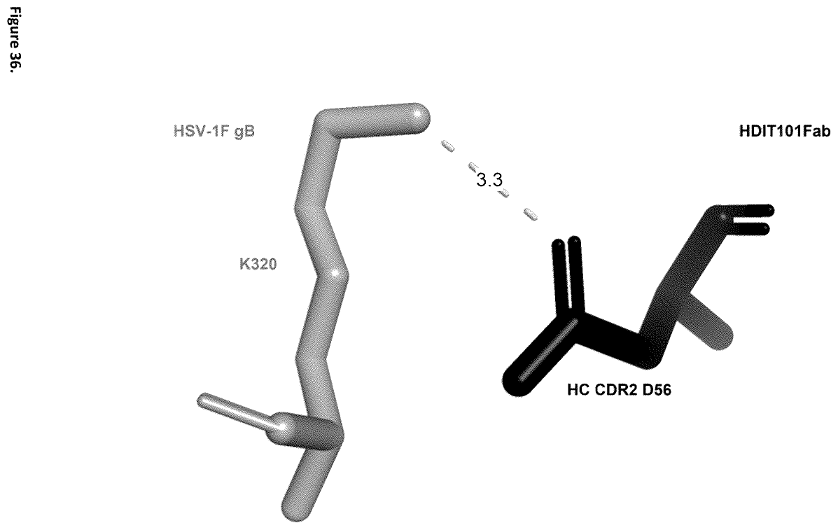

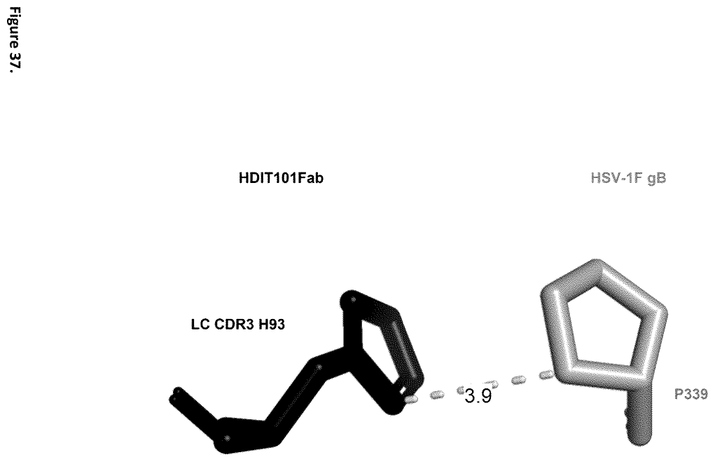

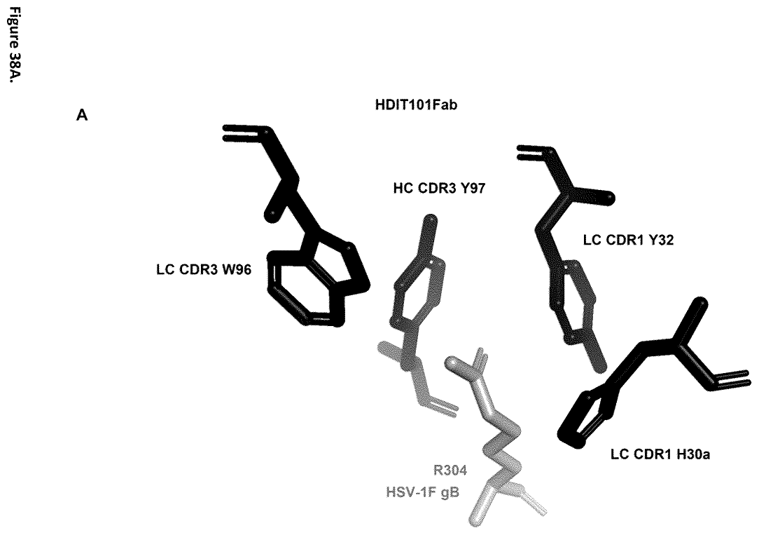

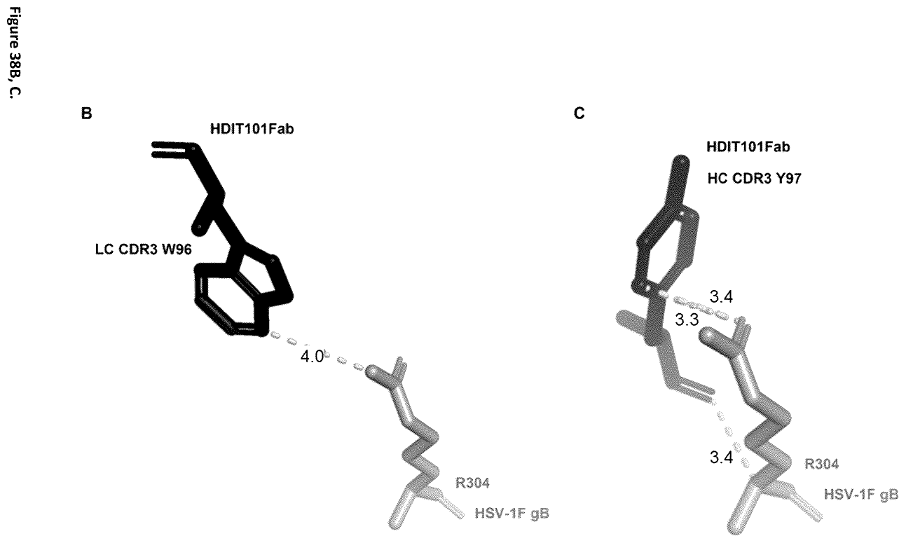

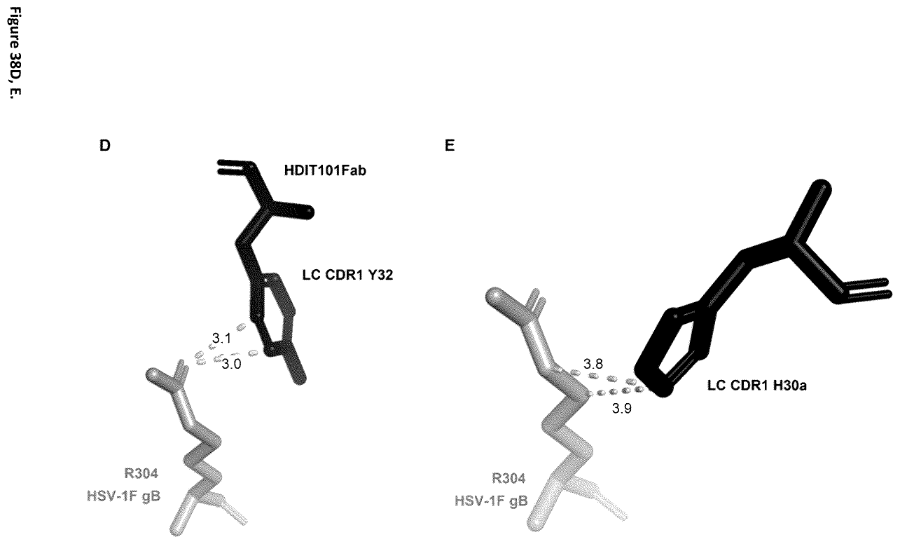

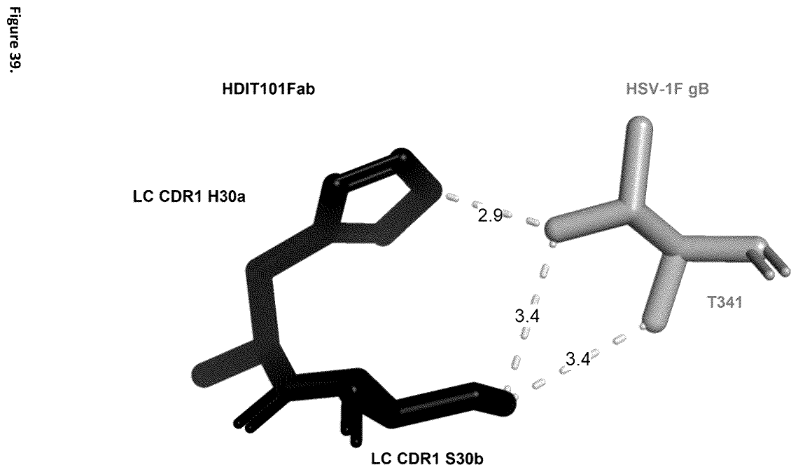

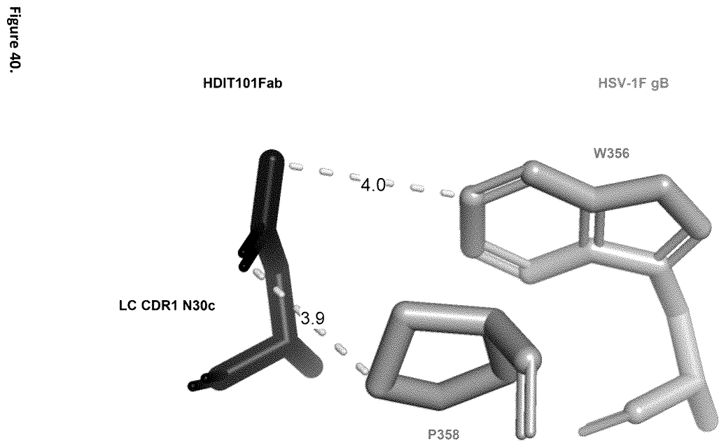

FIG. 33. Analysis of HSV-1 gB residue H308 interactions with HDIT101 CDR residues.
Derived from Cryo-EM structure of HDIT101 Fab in complex with recombinantly produced HSV-1F gB. HSV-1 gB H308 is in proximity to form contacts with HDIT101 HC CDR3 Y99, HC CDR1 G31b and HC CDR2 W53 and the peptide backbone.

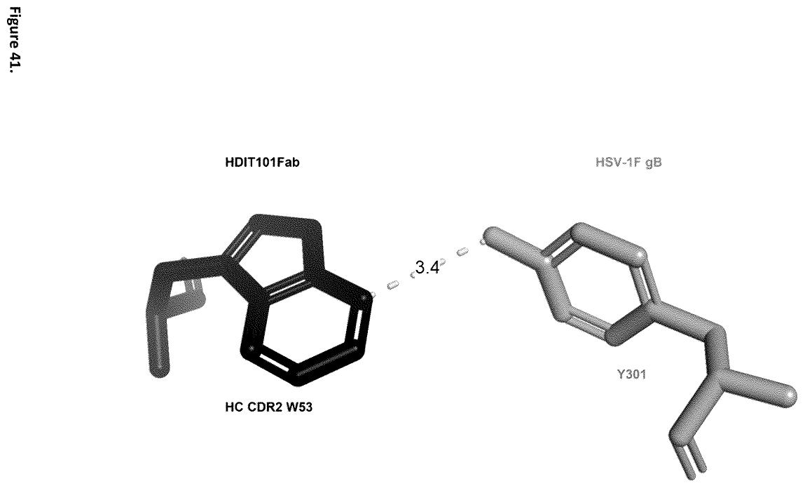

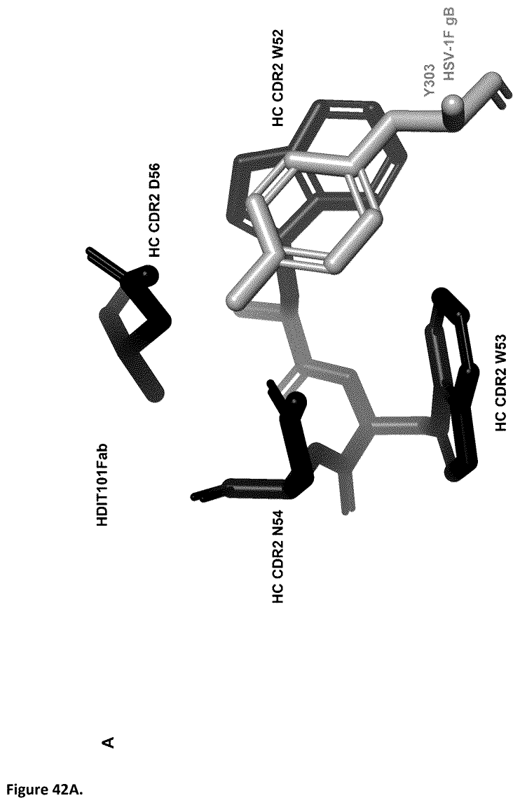

FIG. 34. Analysis of HSV-1 gB residue D323 interactions with HDIT101 CDR residues.
Derived from Cryo-EM structure of HDIT101 Fab in complex with recombinantly produced HSV-1F gB. HSV-1 gB D323 is in proximity to form contacts with HDIT101 LC CDR1 H30a and the peptide backbone at LC CDR3 S92.

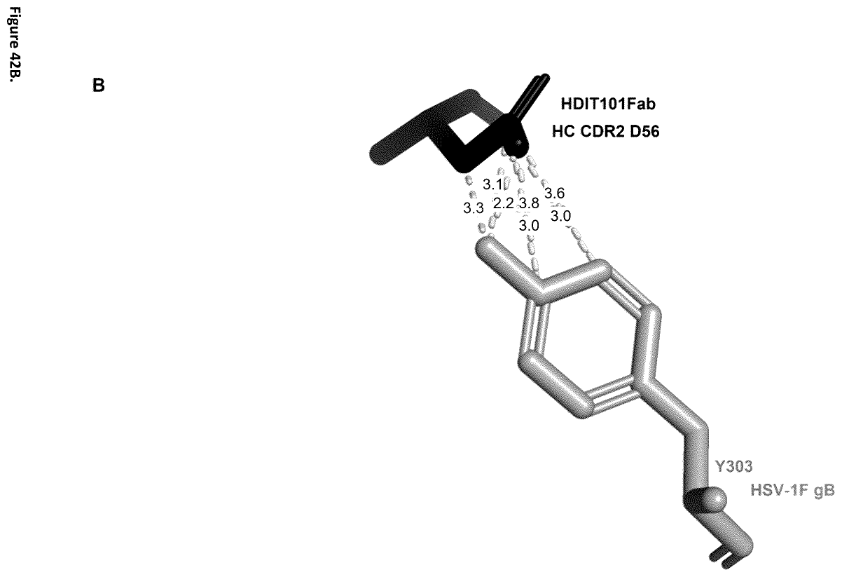

FIG. 35. Analysis of HSV-1 gB residue G302 interactions with HDIT101 CDR residues.
Derived from Cryo-EM structure of HDIT101 Fab in complex with recombinantly produced HSV-1F gB. HSV-1 gB G302 peptide backbone is in proximity to form contact with HC CDR2 W53.

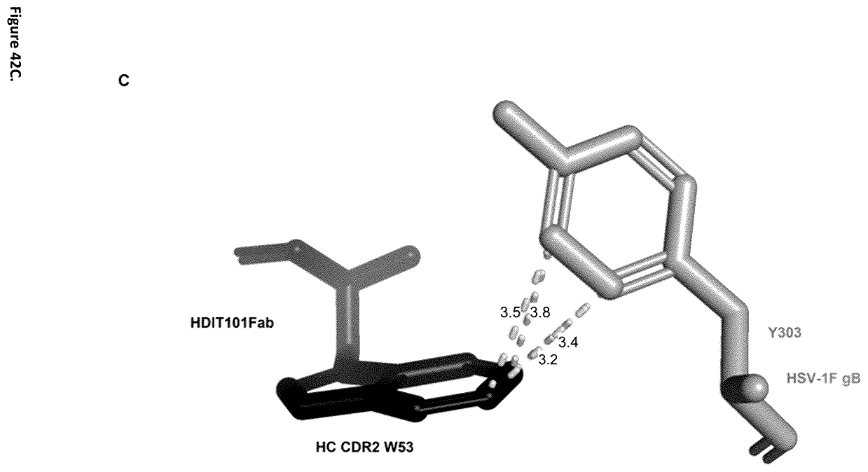

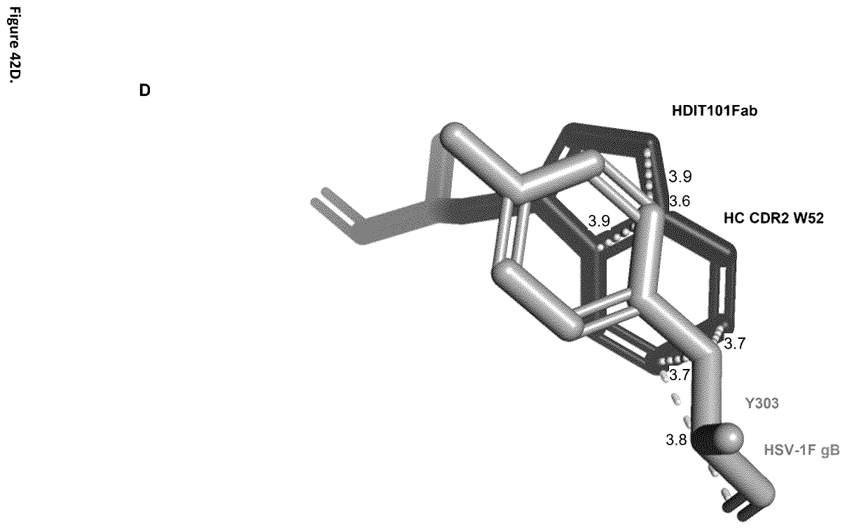

FIG. 36. Analysis of HSV-1 gB residue K320 interactions with HDIT101 CDR residues.
Derived from Cryo-EM structure of HDIT101 Fab in complex with recombinantly produced HSV-1F gB. HSV-1 gB K320 is in proximity to form polar contact with HDIT101 HC CDR2 D56.

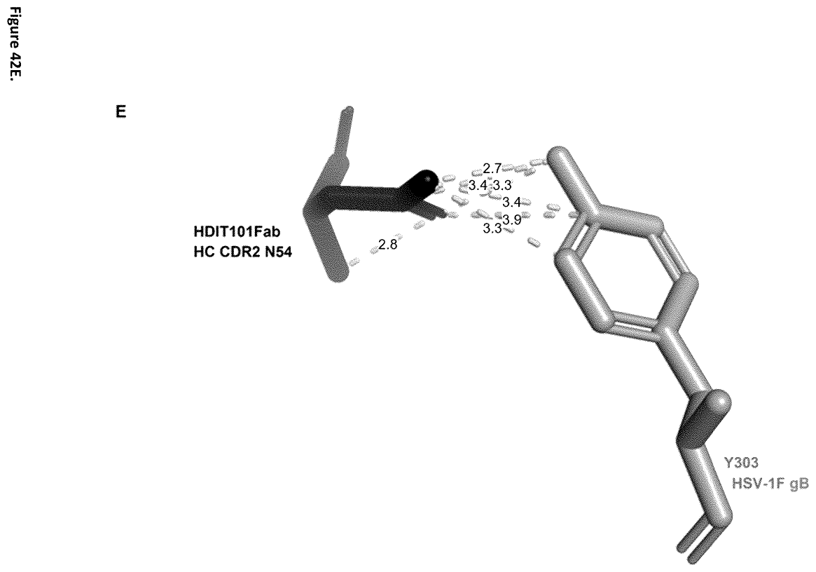

FIG. 37. Analysis of HSV-1 gB residue P339 interactions with HDIT101 CDR residues.
Derived from Cryo-EM structure of HDIT101 Fab in complex with recombinantly produced HSV-1F gB. HSV-1 gB P339 is in proximity to form contact with HDIT101 LC CDR3 H93.

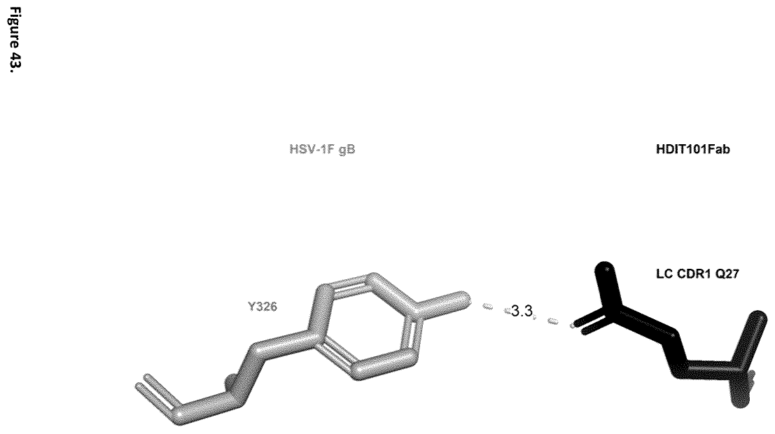

FIG. 38. Analysis of HSV-1 gB residue R304 interactions with HDIT101 CDR residues.
Derived from Cryo-EM structure of HDIT101 Fab in complex with recombinantly produced HSV-1F gB. HSV-1 gB R304 is in proximity to form contacts with HDIT101 LC CDR1 H30a, LC CDR1 Y32, LC CDR3 W96 and HC CDR3 Y97.

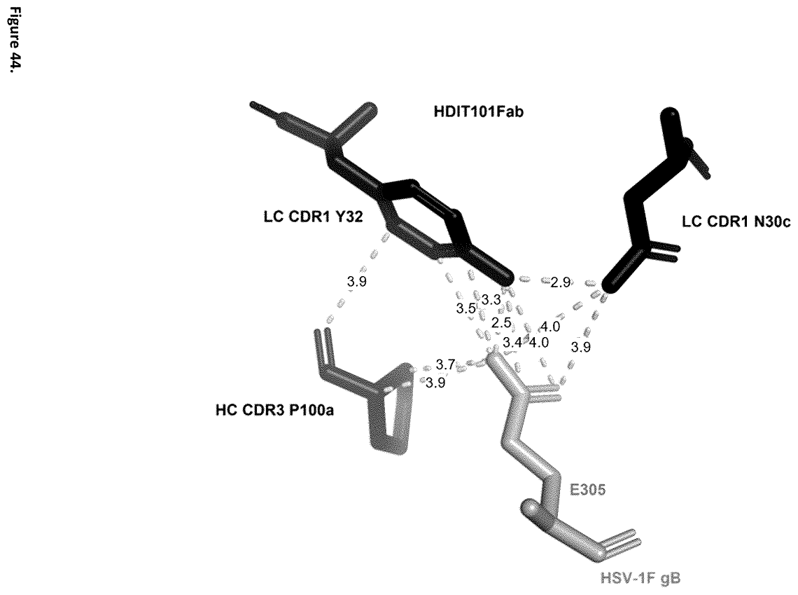

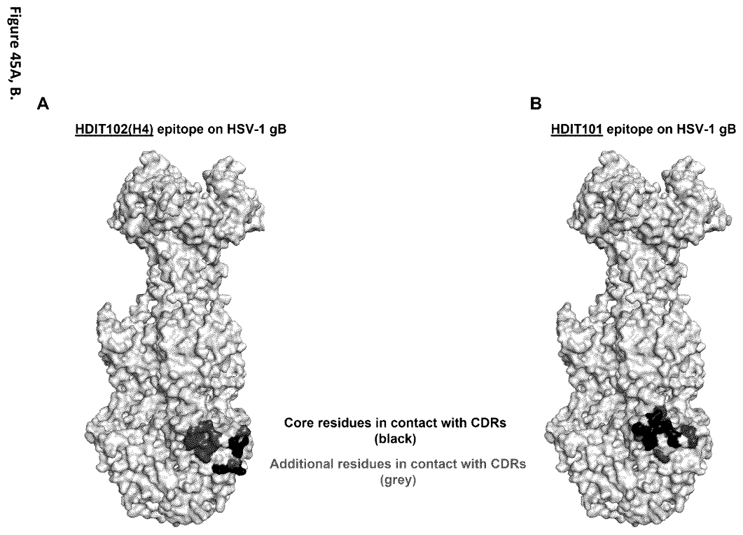

FIG. 39. Analysis of HSV-1 gB residue T341 interactions with HDIT101 CDR residues.
Derived from Cryo-EM structure of HDIT101 Fab in complex with recombinantly produced HSV-1F gB. HSV-1 gB T341 is in proximity to form contacts with HDIT101 LC CDR1 H30a and LC CDR1 S30b.

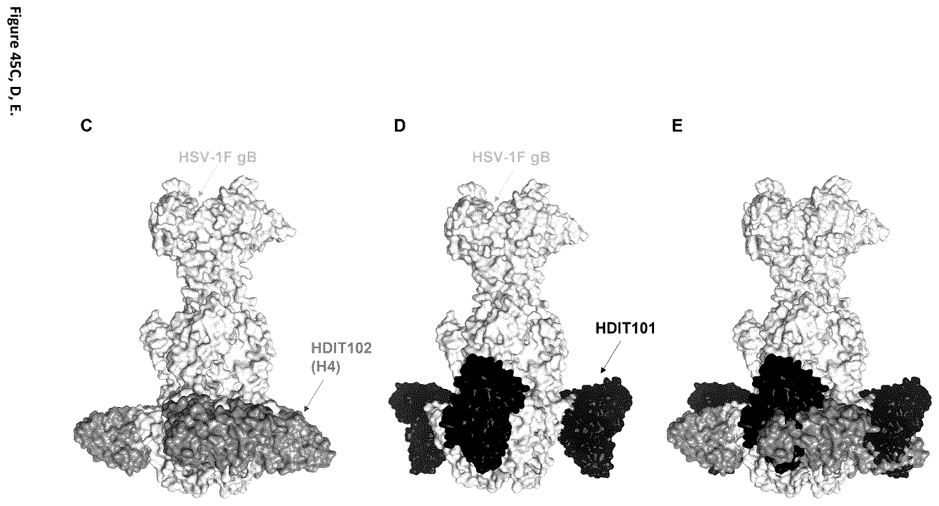

FIG. 40. Analysis of HSV-1 gB residue W356 and P358 interactions with HDIT101 CDR residues
Derived from Cryo-EM structure of HDIT101 Fab in complex with recombinantly produced HSV-1F gB. HSV-1 gB W356 and P358 are in proximity to form contacts with HDIT101 LC CDR1 N30c.

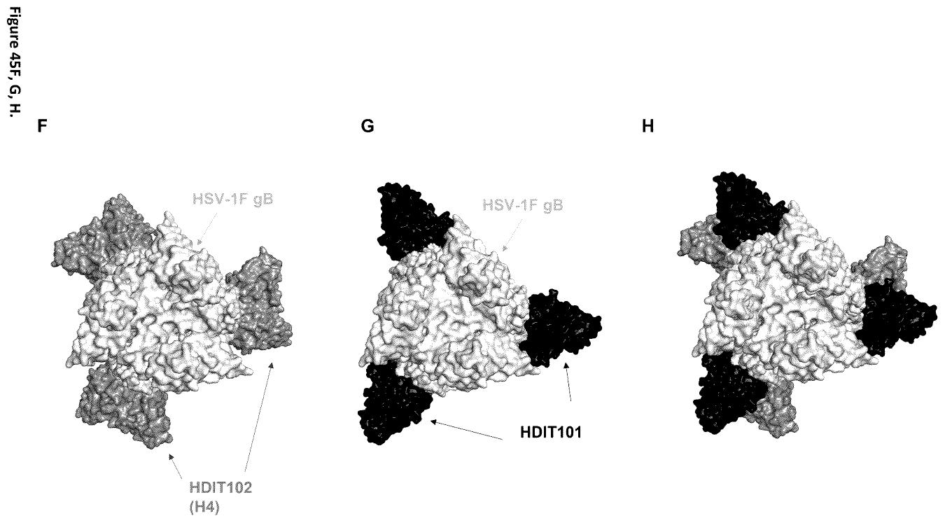

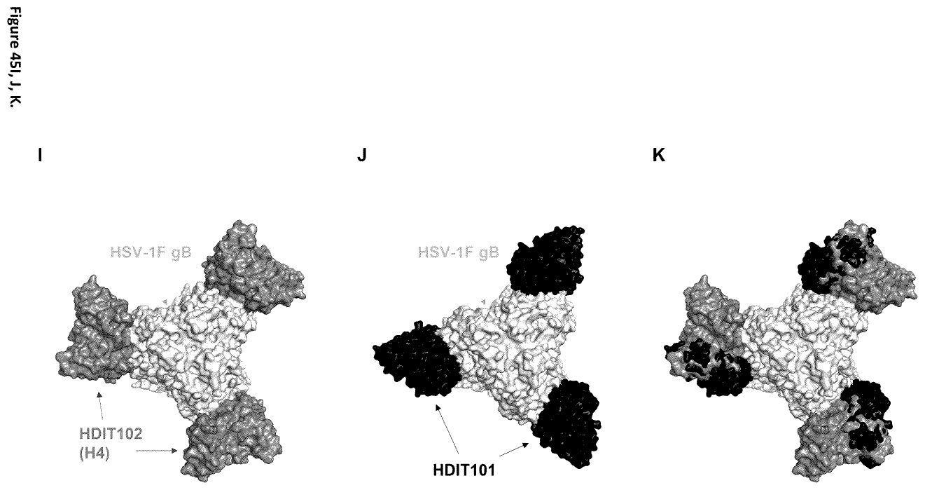

FIG. 41. Analysis of HSV-1 gB residue Y301 interactions with HDIT101 CDR residues.
Derived from Cryo-EM structure of HDIT101 Fab in complex with recombinantly produced HSV-1F gB. HSV-1 gB Y301 is in proximity to form contact with HDIT101 HC CDR2 W53.

FIG. 42. Analysis of HSV-1 gB residue Y303 interactions with HDIT101 CDR residues.
Derived from Cryo-EM structure of HDIT101 Fab in complex with recombinantly produced HSV-1F gB. HSV-1 gB Y303 is in proximity to form non-polar contacts with HDIT101 HC CDR2 W52 and HDIT101 HC CDR2 W53 as well as polar contacts with HC CDR2 N54 and HC CDR2 D56.

FIG. 43. Analysis of HSV-1 gB residue Y326 interactions with HDIT101 CDR residues.
Derived from Cryo-EM structure of HDIT101 Fab in complex with recombinantly produced HSV-1F gB. HSV-1 gB Y326 is in proximity to form polar contact with LC CDR1 Q27.

FIG. 44. Analysis of HSV-1 gB residue E305 interactions with HDIT101 CDR residues.
Derived from Cryo-EM structure of HDIT101 Fab in complex with recombinantly produced HSV-1F gB. HSV-1 gB E305 is in proximity to form contacts with LC CDR1 N30c, LC CDR1 Y32 and HCCDR3 P100a.

FIG. 45. Comparison of HDIT101 and HDIT102(H4) Fab binding and overlap of epitopes on the HSV-1F gB trimeric structure.
(A) Post-fusion conformation of HSV-1F gB trimer derived from the Cryo-EM co-structure with HDIT102(H4) Fab. Critical residues for HDIT102(H4) binding are indicated in black and further residues in close proximity to CDR amino acid residues are indicated in grey. (B) Post-fusion conformation of HSV-1F gB trimer derived from the Cryo-EM co-structure with HDIT101 Fab. Critical residues for HDIT101 binding are indicated in black and further residues in close proximity to CDR amino acid residues are indicated in grey. (C-E) Superimposition of HSV-1F gB trimer in post-fusion conformation side view with bound HDIT101 (black) and HDIT102(H4) (grey) Fab demonstrates steric hindrance of simultaneous binding of both Fabs to one gB protomer. (F-H) top view of superimposed structures. (I-K) bottom view of superimposed structures. (L) Side view of schematic illustration demonstrating perpendicular orientation of HDIT101 and HDIT102(H4) IgG when bound to gB. (M) Top view of schematic illustration demonstrating perpendicular orientation of HDIT101 and HDIT102(H4) IgG when bound to gB.

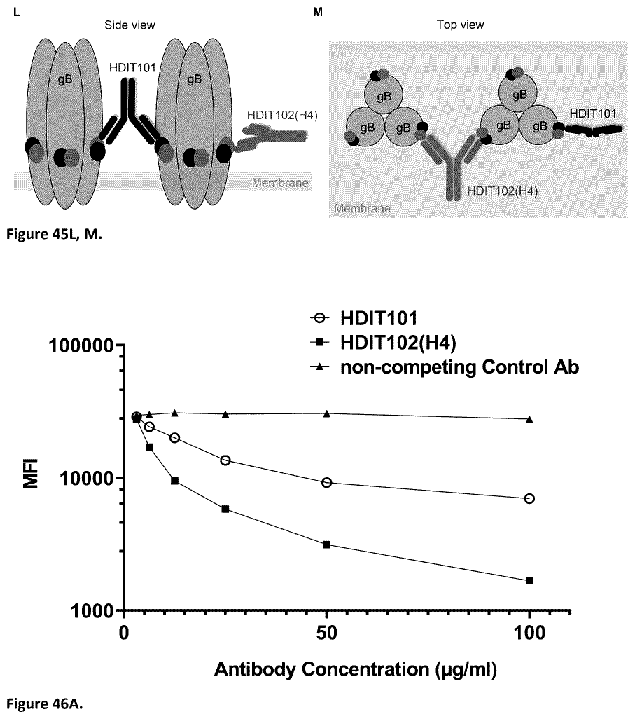

FIG. 46. HDIT102(H4) and HDIT101 are competing for binding to gB ectopically expressed on HEK293T cells and recombinant HSV-1 gB.
(A) A FACS-based assay was performed to test competition in binding of HDIT101 and HDIT102(H4) to gB expressed on cells. 10 μg/mL of a murine version of HDIT101 called MAb2c were incubated with HEK293T cells expressing HSV-1F gB in combination with a serial dilution of HDIT101, HDIT102(H4) or a none competing control antibody and then an anti-murine IgG-APC was used to detect bound murine MAb2c antibody on the target cells. Both, HDIT101 and HDIT102(H4) competed for binding to ectopically expressed gB on target cells with the murine MAb2c, while the control antibody did not. (B) 2D-image reconstructions from Cryo-EM analyses of recombinant HSV-1F gB mixed with 1:1 molar ratio of HDIT101 Fab and HDIT102(H4) Fab. gB trimers with heterogenic binding of two HDIT102(H4) Fabs and one HDIT101 Fab to the gB trimer. (C) Homogenic HDIT102(H4) binding to gB trimers. Heterogenic was nicely separable from homogenic binding due to perpendicular orientation of HDIT101 and HDIT102(H4) Fabs when bound to gB. Simultaneous binding of HDIT101 and HDIT102(H4) Fab to one gB protomer was not observed.

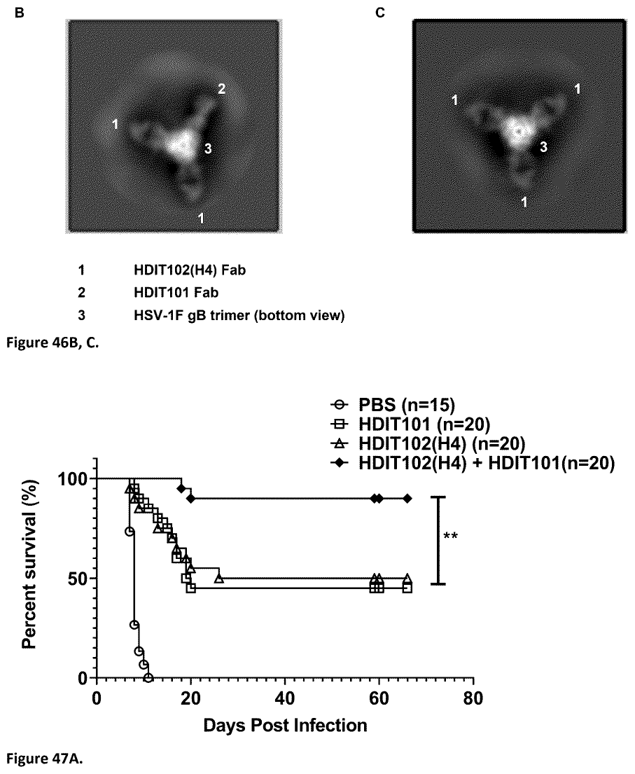

FIG. 47. A combination therapy of HDIT101 with HDIT102(H4) demonstrates synergistic effects in vivo promoting enhanced survival compared to monotherapy after infection with a lethal dose of HSV-2G.
Eight-week-old Balb/c mice were infected with a lethal dose of HSV-2G (5.0×10⁴ TCID50). HDIT102(H4) was mixed with HDIT101 at equimolar ratio (combination therapy) and injected at a final total IgG dose of 300 μg intraperitoneally. HDIT101 or HDIT102(H4) alone (monotherapy) were injected intraperitoneally at the same dose (300 g). 20 mice per treatment group and 15 mice for the control arm were used in total. Survival and clinical symptoms were scored for a period of 60 days. (A) Survival curves are shown. The statistical differences between survival curves were calculated using Logrank Mantel Cox test. **P-value=0.0069. (B) The differences between cumulative combined clinical scores were analysed using Kolmogorov-Smirnow test. ****P-value<0.0001).

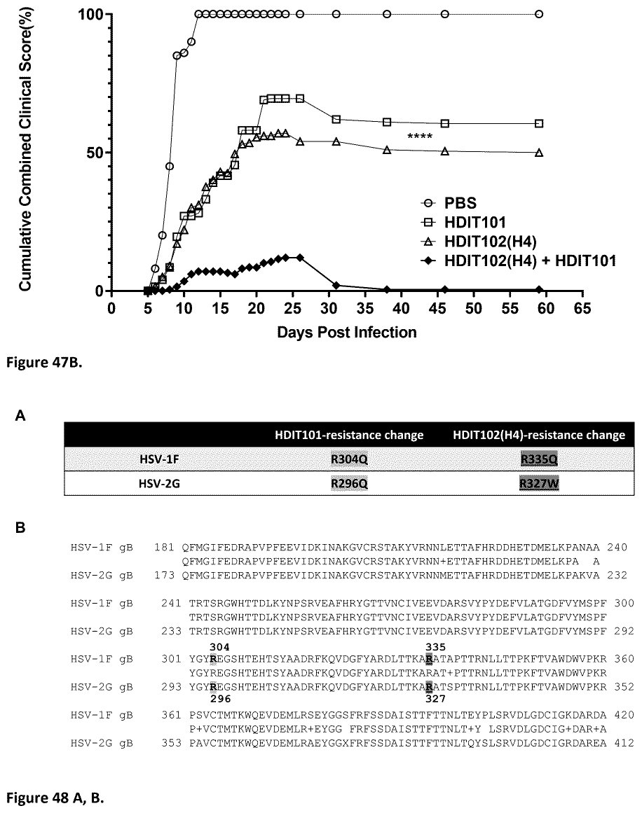

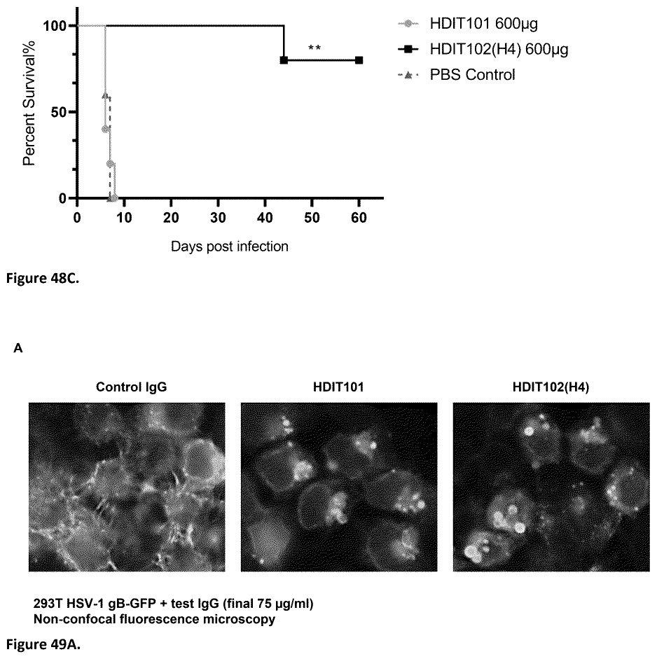

FIG. 48. HDIT102(H4) neutralizes in vitro generated HDIT101-resistant mutant HSV-2G in vivo.
(A) Sequencing analysis of escape mutants which were generated by serial passages of HSV-1F or HSV-2G on Vero cells in the presence of suboptimal doses of neutralizing gB(HSV)-specific monoclonal antibodies showed single mutations leading to amino acid substitution in the gB protein sequence at indicated positions. HDIT101 suboptimal doses let to emergence of HSV-1F-R304Q (HSV-1FHDIT101R) and HSV-2G-R296Q (HSV-2GHDIT101R), while HDIT102(H4) suboptimal doses let to HSV-1F-R335Q and HSV-2G-R327W. (B) Alignment of HSV-1F and HSV-2G gB amino acid sequences (SEQ ID NO:39 and SEQ ID NO:40, respectively) in the HDIT101 and HDIT102(H4) binding regions demonstrates strong conservation in the epitope regions. HSV-1F R304 corresponds to HSV-2G R296 and HSV-1F R335 corresponds to HSV-2G R327. (C) Eight-week-old Balb/c mice were intravaginally infected with a lethal dose of HSV-2GHDIT101R (1.0×10⁵ TCID50) carrying gB R296Q HDIT101-resistance change. HDIT102(H4)—as well as HDIT101-mediated protection was examined in vivo after lethal HSV-2GHDIT101R infection. The mice were infected with the lethal dose and one hour later 600 g of antibodies were injected intraperitoneally, while the control group received PBS (5 mice per group). The statistical differences between survival curves were calculated using Logrank Mantel Cox test. **P-value<0.0015.

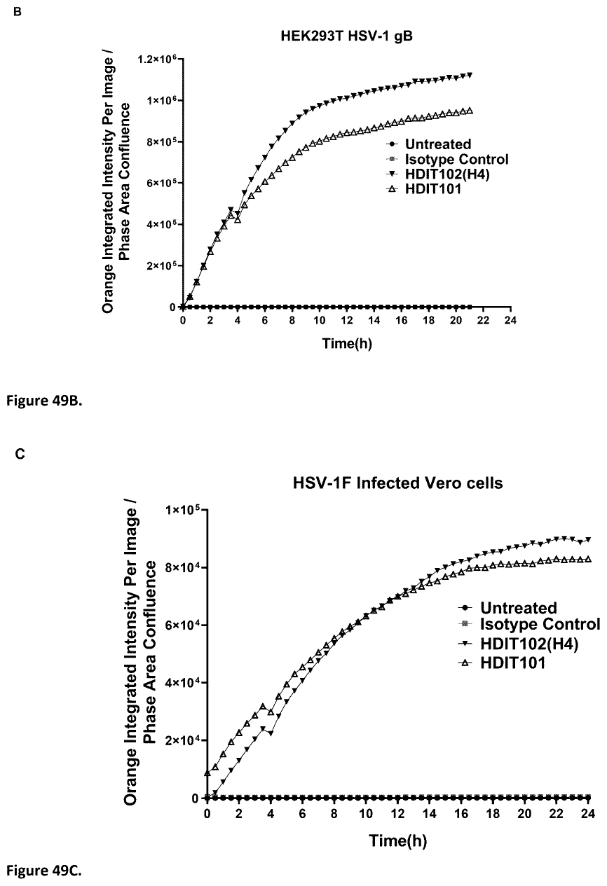

FIG. 49. HDIT101 as well as HDIT102(H4) induce the internalization of gB from the cell surface of gB-expressing as well as HSV-1 infected cells
(A) 293T cells ectopically expressing carboxyterminal GFP-tagged HSV-1F gB were analysed by fluorescence microscopy after incubation with either 75 μg/ml HDIT101 or HDIT102(H4) or with a control IgG (anti-CD22) treatment overnight. Accumulation of intracellular aggregates were documented. (B) HEK293T cells ectopically expressing gB from HSV-1F or (C) HSV-1F infected Vero cells were incubated with labelled HDIT102(H4) or HDIT101 (IncuCyte® Fabfluor-pH Antibody Labeling Dye, Sartorius) and monitored using an Incucyte system (Sartorius) in three technical replicates at intervals of 30 minutes for 20-24 hours. The amount of phagocytosed virus was normalized to the cell count in the imaged area. Untreated and labeled isotype IgG treated cells were included as a negative controls.

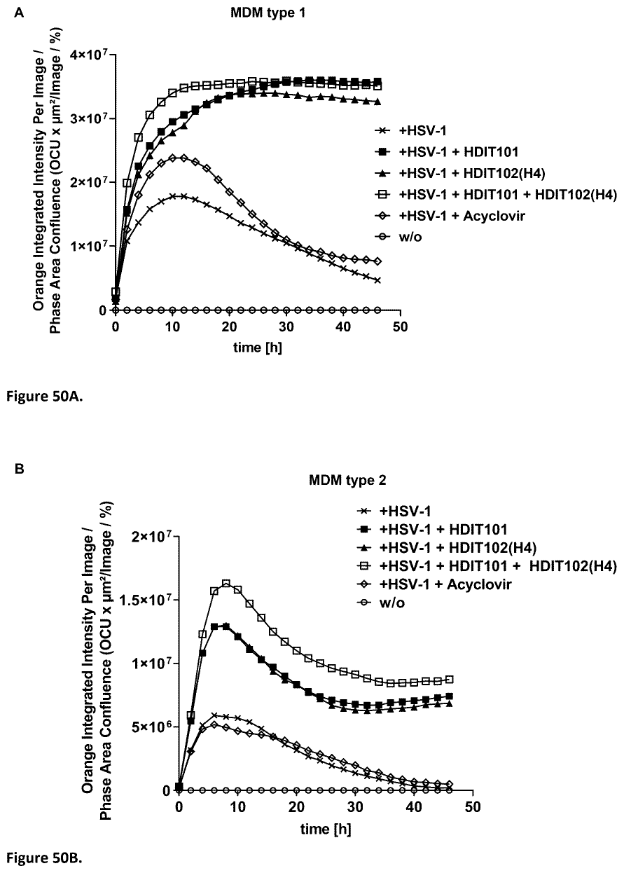

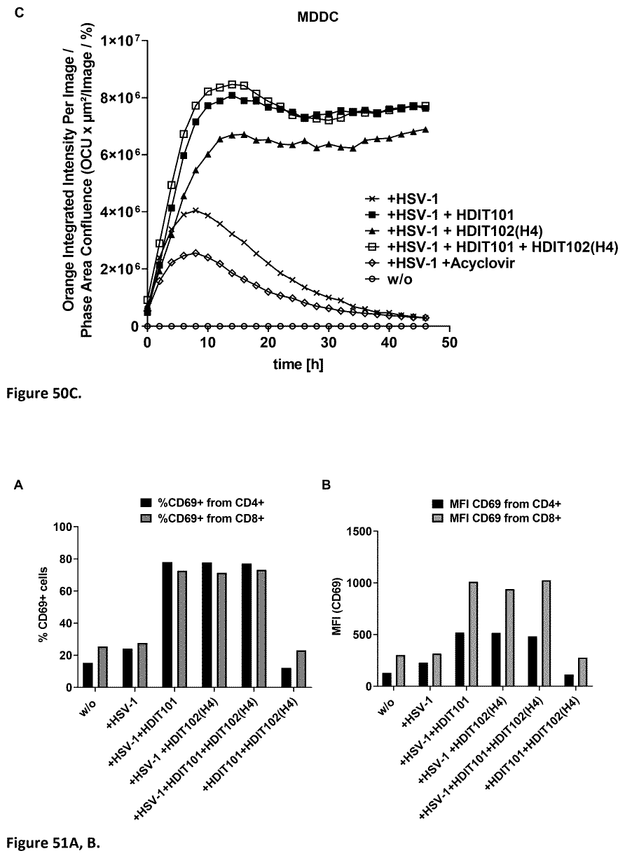

FIG. 50. HDIT102(H4) induces phagocytosis in MDM type 1 or type 2 and in MDDCs individually and in combination with HDIT101.
Monocytes were isolated from PBMCs by MACS CD14+ MicroBead separation (Miltenyi Biotec) according to the manufacturer instruction. The cells were differentiated for one week by adding (A) GM-CSF (80 ng/ml) (MDM type 1 cells), (B) M-CSF (50 ng/ml) (MDM type 2 cells), or (C) GM-CSF (80 ng/ml)+interleukin 4 (IL-4) (20 ng/ml) (MDDC). The differentiated cells were then exposed to HSV-1 labeled with a pH-sensitive dye (IncuCyte pHrodo Orange Cell Labeling Dye, Sartorius) at an MOI of 10 with or without the addition of HDIT101 and/or HDIT102(H4) antibody at a total concentration of 150 μg/ml or Acyclovir (50 μg/ml). Three technical replicates each were then monitored using an Incucyte system (Sartorius) at intervals of 1 h. The amount of virus taken up as measured by dye fluorescence was normalized to the cell count in the imaged area.

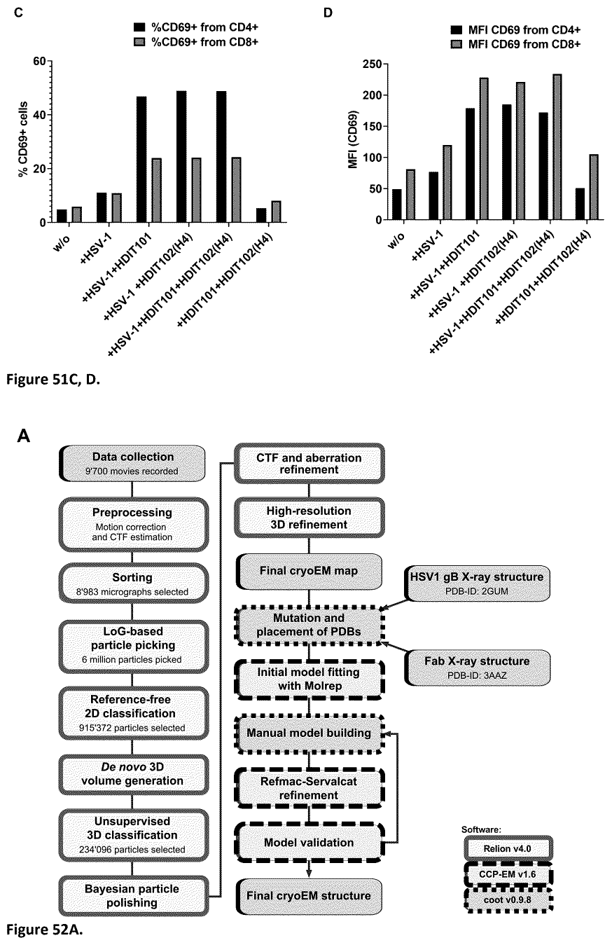

FIG. 51. Activation of autologous T cells upon HDIT102-induced phagocytosis of virus by MDMs individually and in combination with HDIT101.
Autologous T-cell activation was measured after exposure to MDMs from two independent HSV-seropositive healthy donors (donor 1, (A) and (B), donor 2, (C) and (D)) that were pre-incubated for 24 hours with HSV-1F (MOI 10) in the presence or absence of neutralizing amounts of HDIT102(H4) or HDIT101 or combination of both. Analysis was done by flow cytometry using BV785-labeled anti-CD69 IgG (Biolegend) as T-cell activation marker, APC-labeled anti-CD4 (Biolegend) and BV605-labeled anti-CD8 (Biolegend) 24 hours after addition of T-cells to the pre-incubated MDMs. Percentage of CD69-positive cells ((A) and (C)) as well as mean fluorescent intensity (CD69 MFI) ((B) and (D)) were measured and analysed separately for CD4+ as well as CD8+ T-cells.
(A) Sequencing analysis of escape mutants which were generated by serial passages of HSV-1F or HSV-2G on Vero cells in the presence of suboptimal doses of neutralizing gB(HSV)-specific monoclonal antibodies showed single mutations leading to amino acid substitution in the gB protein sequence at indicated positions. HDIT101 suboptimal doses let to emergence of HSV-1F-R304Q (HSV-1FHDIT101R) and HSV-2G-R296Q (HSV-2GHDIT101R), while HDIT102(H4) suboptimal doses let to HSV-1F-R335Q and HSV-2G-R327W. (B) Alignment of HSV-1F and HSV-2G gB amino acid sequences in the HDIT101 and HDIT102(H4) binding regions demonstrates strong conservation in the epitope regions. HSV-1F R304 corresponds to HSV-2G R296 and HSV-1F R335 corresponds to HSV-2G R327.

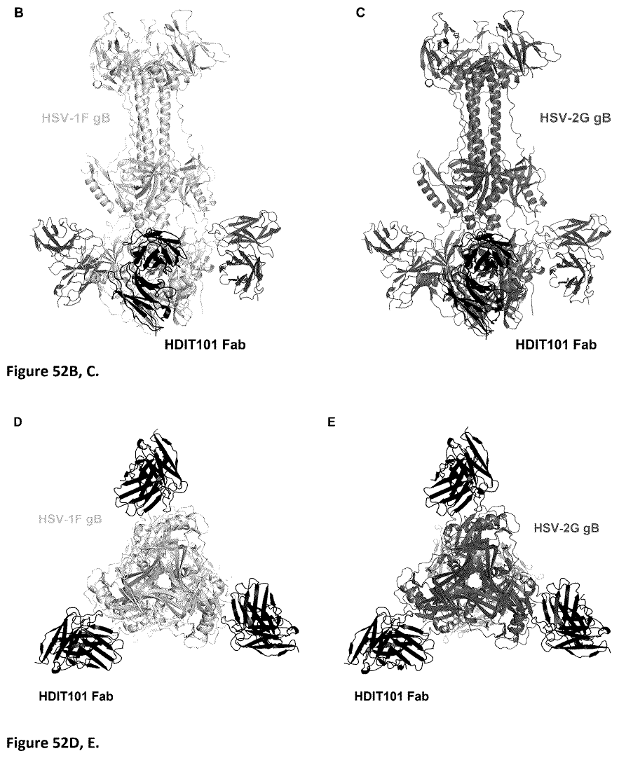

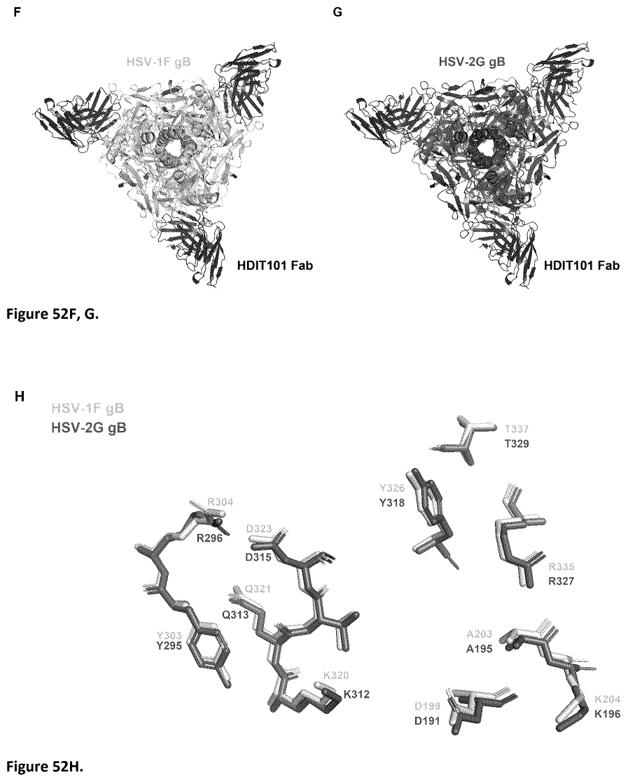

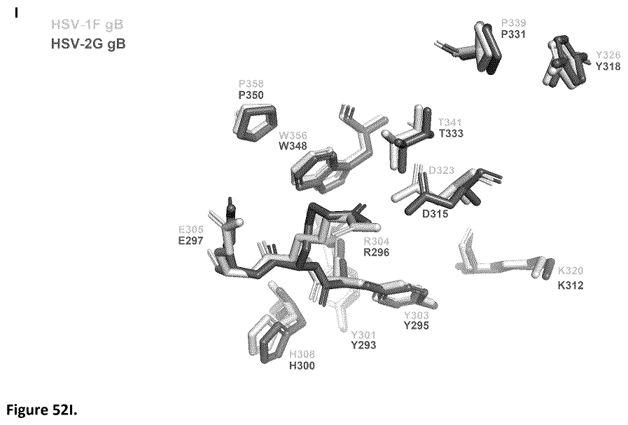

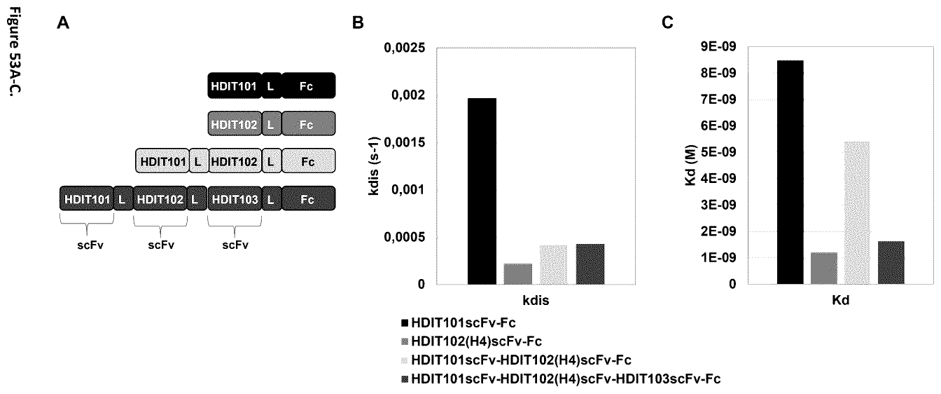

FIG. 52. Cryo-EM solved co-structure of HDIT101 bound to HSV-2G gB.
(A) Single particle Cryo-EM data processing workflow. A graphical representation of the processing steps is shown. Calculations were performed using either Relion (Kimanius, Dong et al., 2021, Biochem J, Vol. 478 (24)), CCP-EM (Nicholls, Tykac et al., 2018, Acta Crystallogr D Struct Biol, Vol. 74 (Pt 6)) or coot (Casanal, Lohkamp et al., 2020, Protein Sci, Vol. 29 (4)) software. (B) Cryo-electron microscopy solved structure of HDIT101 Fab (black) bound to recombinantly produced trimeric HSV-1F gB (light grey) at a resolution of ~3.27 Å. (C) Cryo-electron microscopy solved structure of HDIT101 Fab (black) bound to recombinantly produced trimeric HSV-2G gB (dark grey) at a resolution of ~3.45 Å. (D) and (E) bottom view, (F) and (G) top view. (H) Superimposition of HSV-1F gB (light grey) and HSV-2G gB (dark grey) structures derived from HDIT101 Fab-bound Cryo-EM gB structures showing orientation of HDIT102(H4) binding residues, indicating identical orientation and structural arrangement. (1) Superimposition of HSV-1F gB (light grey) and HSV-2G gB (dark grey) structures derived from HDIT101 Fab-bound Cryo-EM gB structures showing orientation of HDIT101 binding residues, indicating identical orientation and structural arrangement.

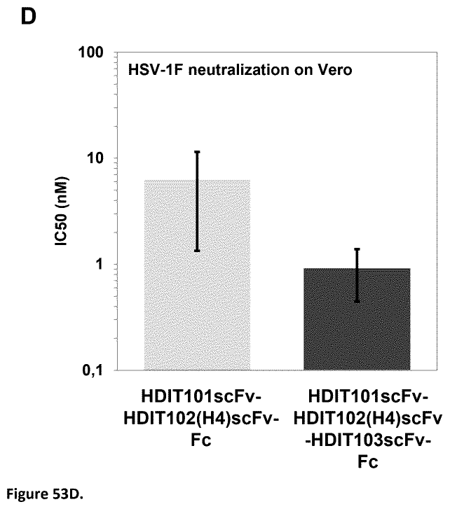

FIG. 53. Bi- or Tri-specific scFv-Fc constructs with gB-targeting domains have combined advantages properties.
(A) Schematic presentation of scFv-Fc fusion constructs. (B) Measurement of dissociation rates of scFv-Fc fusions constructs using bio-layer interferometry. (C) Kd measurements for scFv-Fc constructs using bio-layer interferometry. (D) Inhibitory concentration 50 (IC50) determination bi- or tri-specific scFv-Fc fusion construct to neutralize 100 TCID50 of HSV-1F on Vero cells.

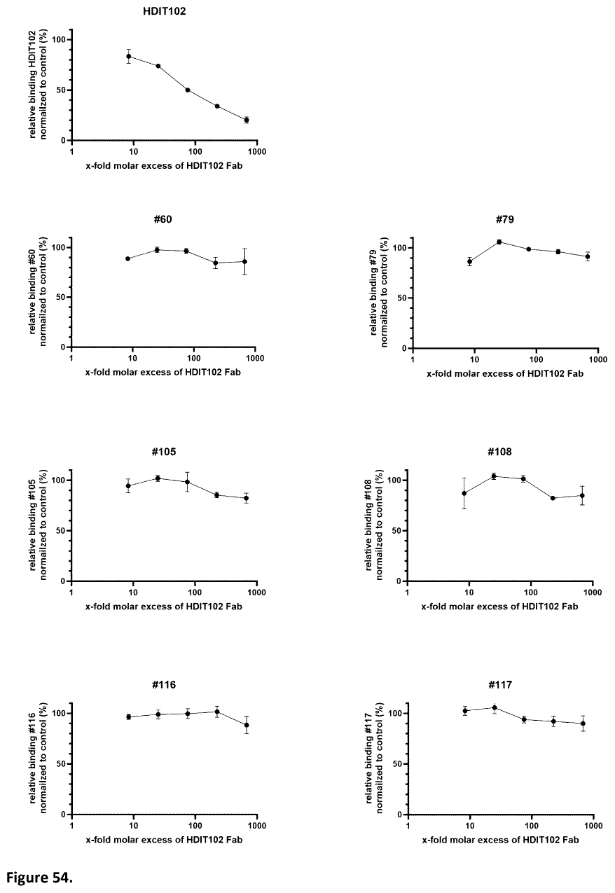

FIG. 54. HDIT102(H4) recognizes a specific epitope that is not recognized by other antibodies.
Competitive binding towards HSV gB of HDIT102 and various anti-gB antibodies isolated from memory B-cells #60, #79, #116, #117, #105, #108 was performed by ELISA. The binding of the respective antibodies was detected with an anti-human Fc-HRP conjugate and the chromogenic substrate TMB in the absence (control) or in the presence of an increasing molar excess of HDIT102 Fab. Shown is the relative binding in % normalized to the control.

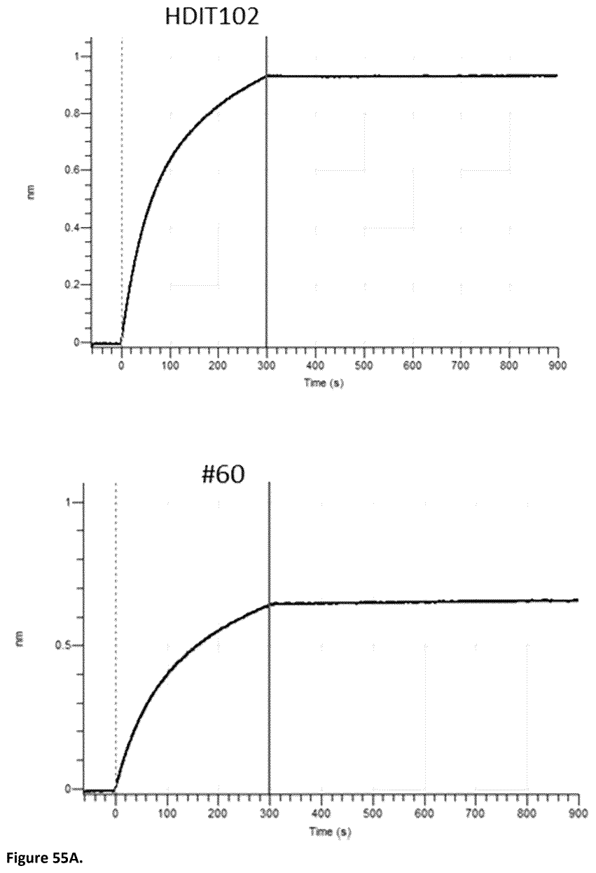

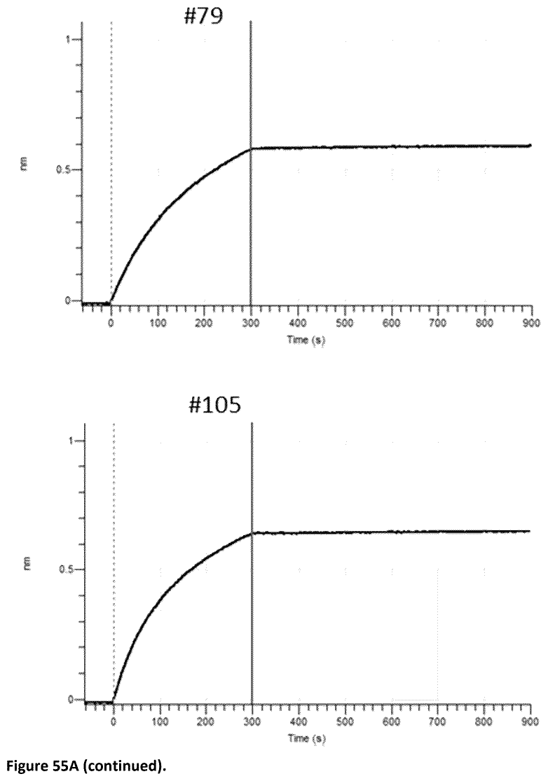

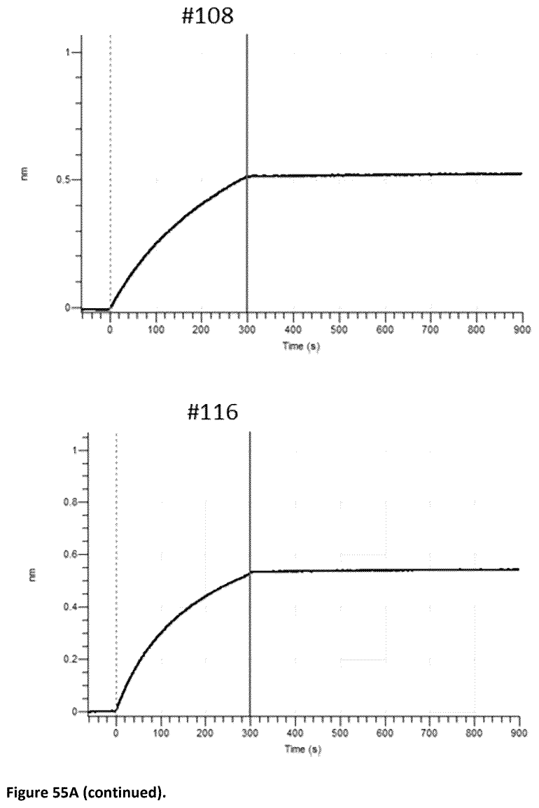

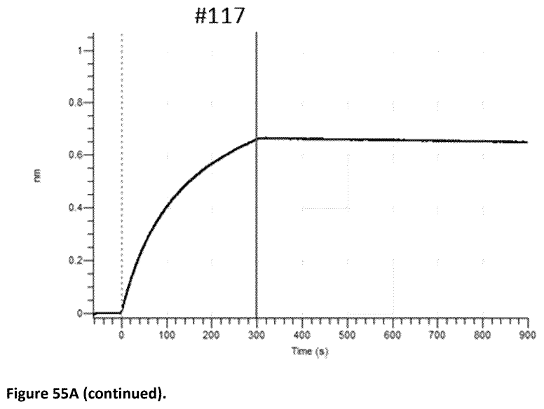

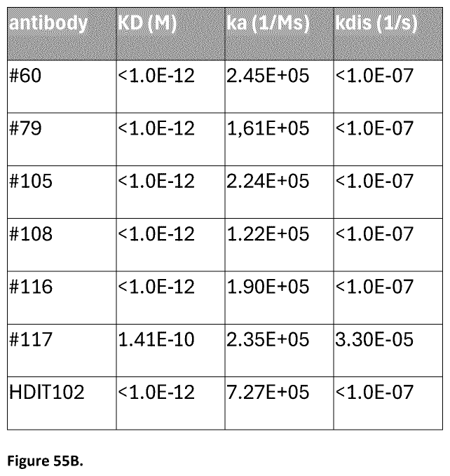

FIG. 55. Bio-layer interferometry (BLI) analysis of recombinant HSV gB interaction with HDIT102(H4) and other antibodies.
(A) Octet sensorgrams demonstrating the association and dissociation of HSV neutralizing antibodies to immobilized wild type recombinant glycoprotein B of HSV immobilized on streptavidin (SA) biosensor tips. (B) Measurements of association (ka) and dissociation (k_{dis}) rates and calculation of binding affinities (KD) using a dilution series of HDIT102(H4) or antibodies #60, #79, #105, #108 #116, #117, respectively on HSV-2 recombinant gB.

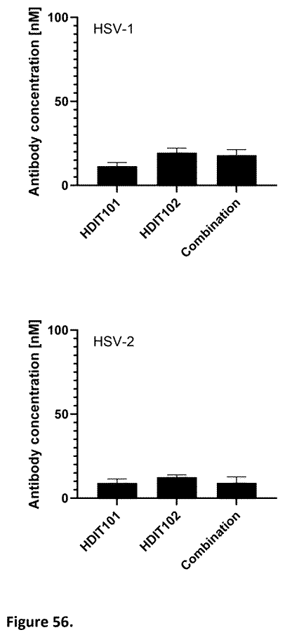

FIG. 56. In vitro neutralization of cell-free HSV-1F or HSV-2G by HDIT102(H4), HDIT101 and a combination thereof.
The antibody concentration required for reducing virus-induced cytopathic effect (CPE) by 50% was determined by an endpoint dilution assay. Serial dilutions of antibodies were incubated with 100 TCID50 of HSV-1F or HSV-2G for 1 h at 37° C. in cell culture medium. The antibody virus inoculum was applied to Vero cell monolayers grown in microtiter plates and cytopathic effect (CPE) was scored after 72 h of incubation at 37° C. Means and error bars, showing standard deviation of mean, were calculated based on three independent experiments.

### EXAMPLES

### Example 1: Generation of a Fully Human Anti-gB Antibody from a scFv Library

To generate human monoclonal antibodies targeting herpes simplex virus with therapeutic potential, the human lymph node derived antibody library (LYNDAL) (phage display derived from the B cell repertoire of head and neck cancer patients) was screened against recombinant insect cell-derived trimeric glycoprotein B of HSV-1 (KOS). Lymph nodes were sampled from patients with squamous cell head and neck carcinoma. Antibody coding regions were obtained by amplifying relevant regions from lymph node-derived B cell mRNA and cloned as individual scFv phage display libraries. Finally, scFv libraries from donors with target-specific immune response were combined for subsequent selection with recombinant gB protein and obtained gB-binding candidates were further characterized.

As the first characterizing assay the obtained gB-specific scFvs were subjected for the analysis of binding to gB. In this assay binding specificity of scFvs targeting glycoprotein B of HSV-1 strain F or HSV-2 strain G were assessed by flow cytometry of infected Vero cells. Twenty-two out of 34 scFvs showed relatively high reactivity towards both HSV-1 or HSV-2 infected cells (including the HDIT102(H4) scFv). The neutralizing potency of selected scFvs was evaluated by a standard plaque reduction assay. Eight scFvs showed more than 10% HSV-1 neutralizing activity at a concentration of 2 μM. When scFvs were cross-linked using an anti-myc tag specific IgG, several cross-linked scFvs showed largely augmented neutralizing potency and HDIT102(H4) had highest neutralization potential and was hence used for further development. To do this the scFv was sequenced and the obtained coding regions for light and heavy chains were codon-optimized and cloned into a human IgG1 backbone. The respective fully human IgG was named HDIT102(H4).

### Example 2: Production of Recombinant HSV-1/2 gB and Bio-Layer Interferometry Analysis Interaction with HDIT101 or HDIT102(H4) Fab Fragments

To analyse HDIT102(H4) binding to HSV-1 gB, the interaction between Fab fragments of the test antibodies HDIT102(H4) or HDIT101 and recombinant HSV-1 gB was explored using bio-layer interferometry (BLI) with an Octet machine (Sartorius).

HSV-1 and HSV-2 gB recombinant protein was produced as follows. The codon-optimized DNA sequence encoding for HSV-1F gB ectodomain (aa 30-729; UniProtKB P06436.1) or HSV-2 gB ectodomain (aa 22-724; GenBank: QAU10948.1) including a BM40 signal peptide and a C-terminal double Strep-tag was cloned into a pCAGGS mammalian expression vector. HSV gB recombinant protein was then transiently expressed in HEK293-E6 suspension cells cultured in F17 medium (ThermoFisher) supplemented with 0.1% Kolliphor (Sigma) and 4 mM Glutamine. HEK293-E6 cells were transfected with PeiMax (Polysciences) at a cell density of 1.5-2.0×10⁶ cells/ml with 1 μg plasmid and 2 μg PeiMax/ml culture media. Twenty-four hours after the transfection Tryptone N1 feeder (Organi Technie) was added to the cultures. On day 5 after the transfection the supernatant was harvested by two centrifugation steps, first 1200 rpm to remove the cells and then 3600 rpm to remove cell debris. Next, the pH of the supernatant was adjusted by the addition of 1 ml 2 M Tris buffer pH 9/100 ml supernatant. Afterwards the gB protein was purified by Strep-Tactin XT (IBA, Germany) gravity flow purification according to the manufacturer protocol. The Strep-tag of gB protein was then removed by thrombin digestion (Serva) and dialyzed against 50 mM Tris, 150 mM NaCl, pH 8. The thrombin digested gB protein was then purified three times by Strep-Tactin XT affinity chromatography to deplete the sample from any remaining Strep-tagged gB protein. Next, the gB protein was concentrated with Amicon spin columns (cut-off 30 k) and was further purified with a Superdex 200 10/300 GL SEC column and a Äkta Pure FPLC. The peek fractions were pooled and concentrated with Amicon spin columns. HDIT101 Fab was generated by papain digest and subsequent purification, while H4 Fab was generated recombinantly by transfection of light and truncated heavy chain encoding plasmids into 293T-E6 cells. HSV gB recombinant protein was biotinylated (NHS-PEG4-Biotin (Thermo Fischer Scientific, A39259)) in ratio 3:1 for 30 min at room temperature and afterwards, the residual biotin was removed by using desalting columns and centrifugation at 1000 g for 2 min (Zeba Spin Desalting Columns; 7K MWCO, 2 ml (Thermo Scientific UE285726). An initial loading scout was performed to find out the best biosensor loading concentration. The streptavidin biosensors (of the Octet) were loaded with different concentrations of biotinylated gB and the absorption kinetics of the test antibody Fab fragments HDIT102(H4) or HDIT101 were measured. The optimal gB concentration for loading the biosensor was determined with 5 μg/ml. Biotinylated gB (wt) [5 μg/ml] was used to load the biosensor and the binding kinetics of antibody Fab fragments (100 nM) against immobilized gB were analyzed using global 1:1 Fit model. To determine the association rate (ka), the dissociation rate (kdis) and Kd, a dilution series of Fab fragments was analysed.

The association rates (ka) for HDIT101 and HDIT102(H4) IgG were measured and demonstrated comparable association kinetics for both IgG antibodies (approximately 5.0×105 to 7.0×10⁵ M⁻¹s⁻¹). However, and quite strikingly, HDIT102(H4) IgG had no measurable dissociation rate (kdis), while kdis for HDIT101 IgG could be determined (approximately 1.1×10⁻³ s⁻¹) (FIG. 1A, B). Because of the lack of measurable kdis for HDIT102(H4) full IgG binding to recombinant HSV gB the binding constant Kd could not be determined. Therefore binding of the respective Fab fragments was analysed. HDIT102(H4) Fab had increased association rates (ka) to HSV-1F gB (9.92×10⁵ M⁻¹s⁻¹) as well as to HSV-2G gB (8.65×10⁵ M⁻¹s⁻¹) as compared to HDIT101 Fab (HSV-1F gB, 4.35×10⁵ M⁻¹s⁻¹ and HSV-2G gB, 2.12×10⁵ M⁻¹s⁻¹) (FIG. 1C). In addition, as seen for the HDIT102(H4) IgG, dissociation rates for HDIT102(H4) Fab were dramatically decreased as compared to HDIT101, for HSV-1F (HDIT102(H4) vs. HDIT101, 8.85×10⁻⁵ s⁻¹ vs. 3.13×10⁻³ s⁻¹) as well as for HSV-2G (HDIT102(H4) vs. HDIT101, 2.86×10⁻⁵ s⁻¹ vs. 3.79×10⁻³ s⁻¹). This results in substantially decreased Kd for HDIT102(H4) binding to HSV-1 gB (HDIT102(H4) vs. HDIT101, 8.95E-11 M vs. 7.26E-09 M) or HSV-2 gB (HDIT102(H4) vs. H DIT101, 3.29×10⁻¹¹ M vs. 1.81×10⁻⁸ M).

The data demonstrate that HDIT102(H4) has a substantially decreased dissociation rate (Kdis) as compared to HDIT101 resulting in dramatically improved Kd for binding to both, HSV-1F gB or HSV-2G gB (FIG. 2). The therapeutic potential is hence expected to be improved.

### Example 3: Cross-Reactivity Analyses for HDIT102(H4)

To test for HDIT102(H4) cross-reactivity with other common members of the Herpesviridae family, commercial Enzygnost anti-HSV/VZV/CMV/EBV IgG ELISA kits were used. First, the antibody-containing samples were applied to special 96-well plates coated with the target antigens (HSV/VZV/CMV/EBV infected cell lysate). No differentiation between HSV-1 and HSV-2 can be done with this test. After an incubation period of 1 hour at 37° C., samples were washed three times with 200 μl washing buffer. The bound antibodies were detected with specific secondary antibody (Rabbit anti human Fcγ IgG-HRP conjugated polyclonal antibody (Jackson ImmunoResearch)). Except for the above-mentioned modification for secondary antibody, the test was carried out according to the manufacturer's manual. Quantification of the bound antibodies was carried out with an absorbance/fluorescence plate reader (Tecan) at wavelength of 450 nm and 650 nm was set as a reference wavelength. As a positive control human Cytomegalovirus (HCMV) polyclonal immune globulin Cytotect® was used, which also demonstrated binding to HSV, EBV and VZV antigens while HDIT102(H4) did not show binding to VZV/CMV/EBV antigens (FIG. 3).

### Example 4: Neutralization of Cell-Free HSV-1 and HSV-2 by HDIT102(H4)

To examine the antiviral activity of HDIT102(H4) towards cell-free virus, different antibody dilutions were incubated with a constant viral dose (100 TCID50 HSV-1F or HSV-2G) for 1 h at 37° C. The antibody-virus mixtures were applied to 80-90% confluent Vero cells in 96-well plates (2.0×10⁴ cells per well) in a volume of 100 μl per well. As a control, Vero cells were infected with a viral dose of 100 TCID50 without prior incubation with neutralizing antibody. The extent of the cytopathic effect was examined by light microscopy three days after infection. The neutralization titre was determined to be the highest antibody dilution at which the virus was completely neutralized and the formation of a CPE in the inoculated cell cultures was completely prevented.

The neutralization titer is determined to be the highest antibody dilution at which the virus is still completely neutralized and the formation of a CPE in the inoculated cell cultures is completely prevented. In addition, the neutralizing antibody concentration at which 50% of the cell culture wells are protected from infection (IC50) are calculated using the 50% neutralization titer formula (Krawczyk, Krauss et al., 2011, J Virol, Vol. 85 (4)): T=x+((b/10)·x), T=neutralizing antibody titer at which 50% of the infected cell cultures are protected from infection. x=Lowest antibody dilution for which at least 50% of cell cultures are infected. b=number of infected cell cultures, which at dilution x exceed the 50% infection rate.

Inhibitory concentrations for neutralizing HSV-1F by HDIT102(H4) were 25.5 nM and 62.5 nM for 50% and 100% neutralization, respectively, while inhibitory concentrations for HSV-2G were 12.5 nM and 31.25 nM, for 50% (IC50) and 100% neutralization, respectively (FIG. 4).

### Example 5: Protection of Immunodeficient NOD/SCID Mice from a Lethal HSV-1 Infection by HDIT102(H4)

In vivo efficacies of HDIT102(H4) was investigated in HSV-1F infected NOD.CB17-PrkdcSCID/NCrHsd mice. One week prior to the experiment, 6-week-old female NOD/SCID mice (weight 16-19 grams) were purchased from ENVIGO and one week prior to the viral inoculation, they were pre-treated subcutaneously with medroxyprogesteron (long-acting progestin Depo-Clinovir, prepared at 25 mg/ml in PBS, 100 μl per mouse). On the day of intravaginal HSV-1F inoculation, the mice were anesthetized using isoflurane. During the short anaesthesia, the vaginal mucosa was cleaned from the vaginal secretions by using a sterile ESwab following infection by intravaginal inoculation of HSV-1F (10 μl of HSV-1F stock containing 5.0×10⁵TCID50) to the vaginal mucosa using a pipette. Afterwards, a small amount of Epiglu tissue adhesive was applied on the surface to avoid inoculum flow out. The glue was lost within 1 day after inoculation. The efficiency of HDIT102(H4) in protecting mice from HSV-1F induced death was assessed by intraperitoneal administration of different HDIT102(H4) antibody doses: 600 μg (~30 mg/kg), 300 μg (~15 mg/kg) or 150 μg (~15 mg/kg), all in 100 μl PBS, (N=9), 4 hours post-infection. The control group was treated with PBS (N=6). The experimental animals were regularly inspected for weight loss and the occurrence of perineal hair loss (HL), redness of skin (R) and swelling (S) and neurologic symptoms (e.g. limb paralysis, gastrointestinal track blockage) over an observation period of 55 days. Visible inspection was graded from slight to severe symptoms accordingly +/++/+++. Viral shedding was checked on days 1, 3 and 6 post-inoculation by taking vaginal swabs in 200 μl PBS for subsequent qPCR. Experimental animals were sacrificed in case of severe signs of neurological disease (e.g. herpes encephalitis) or visible lesions to prevent undue suffering. All experiments were done in line with ethical approval.

All mice in the control group died of the HSV-1 infection by day 10. Statistically significant enhanced survival was observed with increasing doses of HDIT102(H4). Doses of 150 μg or 300 μg resulted in partial but significant protection. A dose of 600 μg (~30 mg/kg) of HDIT102(H4) rescued ~50% of immunodeficient mice from death in the observation period (FIG. 5A).

To analyse absolute copy numbers of HSV-1 in vaginal swabs of infected mice, a quantitative polymerase chain reaction (qPCR), using a Roche Light Cycler 480 with prima QUANT 2×qPCR-SYBR-Green-Blue-Master MIX was performed. In principle, qPCR works by measuring the fluorescence signal (SYBR Green) of amplified DNA in each sample after each PCR cycle and comparing the signal to a reference standard with known DNA copy numbers. For this purpose, viral DNA from vaginal swabs were isolated using the QiaAmp DNA Blood Kit according to the manufacturer's recommendation for samples with low DNA yield (adding 5-10 μg of ultrapure salmon's sperm DNA (Invitrogen) as carrier DNA) and eluted in 50 μl ddH₂O. For preparation of standards, solutions with serial dilution of known copy numbers of target DNA were prepared. Briefly, the target region spanning the primers used in qPCR was amplified by PCR and cloned into pJet vector. The plasmid was amplified in bacteria, isolated, and the DNA concentration was determined by spectrometry. The DNA was diluted to 2.0×10′ copies/μl and further by using 10-fold dilution steps down to 2 copies/μl. The qPCR primers were described previously (Kaeman and Whitley, Journal Infectious Diseases, 1995 April; 171(4):857-63. doi: 10.1093/infdis/171.4.857.) and are specific for a 179 bp sequence in the HSV polymerase gene (UL30), which is conserved between HSV-1 and HSV-2, hence can be used for quantification of both viruses. The assay was performed using a Roche LightCycler480 with the following 2-step program: Initial denaturation at 95° C. for 120 s, followed by 45 cycles of denaturation at 95° C. for 15 s, annealing and elongation at 65° C. for 30 s and 60 s final elongation. Prior to each run, 5 μl of standard or sample was mixed with 15 μl of a mix containing 10 μl of 2× master mix and specific primers (5′ATCAACTTCGACTGGCCCTT3′ (SEQ ID NO:25) and 5′CCGTACATGTCGATGTTCAC 3′ (SEQ ID NO:26), to reach a final concentration of 300 nM each). Enzymes, Buffer and SYBR Green dye is provided in the 2× master mix. Absolute quantification was performed using the “abs quanti/2nd derivative Max” method using the Light Cycler 1.5 Software. By comparing the cycle, in which a defined fluorescence signal was reached in each individual sample with the standard the exact copy numbers of HSV genomes in every sample was calculated. All samples were run in duplicates and samples with a lower than 2-fold variation between the individual wells were considered valid. Data was analysed using Microsoft Excel and plotted using Graph Pad Prism.

The swabbing data demonstrated that HSV-1 shedding was significantly reduced at day 6 post-treatment with 150 μg, 300 μg, or 600 μg HDIT102(H4) in a dose response manner as compared to control treated animals (FIG. 5B). The data proposes that HDIT102(H4) could possibly be used to protect immunocompromised patients from a severe HSV infections.

### Example 6: Protection of Immunocompetent Balb/c Mice from a Lethal HSV-2 Infection by HDIT102(H4)

In vivo efficacy of HDIT102(H4) was investigated in HSV-2G infected Balb/cOlaHsd mice. One week prior to the experiment, 6-week-old Female mice Balb/c (weight 16-19 grams) were purchased from ENVIGO and one week prior to the viral inoculation, they were pre-treated subcutaneously with medroxyprogesteron (longacting progestin Depo-Clinovir, prepared at 25 mg/ml in PBS, 100 μl per mouse). On the day of intravaginal inoculation, the experimental animals were anesthetized using isoflurane. During the short anaesthesia, the vaginal mucosa was cleaned from the vaginal secretions by using a sterile ESwab and the experimental animals were infected by intravaginal inoculation of 10 μl of herpes virus stock (containing 5.0×10⁴ TCID50 HSV-2G) to the vaginal mucosa using a pipette. Afterwards, a small amount of Epiglu tissue adhesive was applied on the surface to glue the vagina (avoids inoculum to flow out). Normally the glue was lost within 1 day after inoculation. The efficiency of HDIT102(H4) in protecting mice from HSV-2G induced death was assessed by intraperitoneal administration of different HDIT102(H4) antibody doses: 600 μg (~30 mg/kg), 300 μg (~15 mg/kg) or 150 μg (~15 mg/kg), all in 100 μl PBS, (N=10), 4 hours post-infection. The control group was treated with PBS(N=10). The experimental animals were regularly inspected for weight loss and the occurrence of perineal hair loss (HL), redness (R) and swelling (S) and neurological symptoms (hind limb paralysis, gastrointestinal track blockage) over a 65 days of observation period. Visible inspection was graded from slight to severe symptoms accordingly +/++/+++. Viral shedding was checked on days 1, 2, 4 and 7 post-inoculation by taking vaginal swabs in 200 μl PBS for subsequent qPCR. Experimental animals were sacrificed in case of severe signs of neurological symptoms, e.g., herpes encephalitis or limb paralysis or severe lesions to prevent undue suffering. All experiments were done in line with ethical approval.

To analyse absolute copy numbers of HSV-2G in vaginal swabs of infected mice, a quantitative polymerase chain reaction (qPCR), using a Roche Light Cycler 480 with prima QUANT 2×qPCR-SYBR-Green-Blue-Master MIX was performed and analysed as described above for HSV-1.

When immunocompetent Balb/c mice were infected with HSV-2 at a lethal dose, 90% (9/10) of control animals died of the infection within 15 days. In contrast 80% (8/10) of mice treated with 600 μg HDIT102(H4) were alive at this time point. The difference between control group and HDIT102(H4) treated groups was significant within the observation period (FIG. 6A). Analysis of vaginal swabs showed a reduction of viral copy numbers at day 4 which increased to more than one order of magnitude by day 7 post infection in HDIT102(H4) treated versus control mice, indicating that HDIT102(H4) treatment reduced virus shedding (FIG. 6B).

### Example 7: Cell-to-Cell Spread of HSV is Inhibited by HDIT102(H4)

The potential of HDIT102(H4) to inhibit the “cell-to-cell spread” of HSV-1 in cultivated Vero cells was investigated by immuno-fluorescence microscopy. For this purpose, initially 2.0×10⁵ Vero cells were seeded in each well of a 4-well chamber slide. The following day, the culture medium was discarded, and the cells were inoculated with a viral load of 200 TCID50 in a volume of 500 μl DMEM per well. Four hours after infection, the supernatant containing virus was discarded, unbound virus particles were removed by washing one time with 500 μl PBS, and the cells were incubated with 500 μl culture medium containing 10% FCS and 500 nM of HDIT102(H4). As a control for inhibition of cell to cell spread, human polyclonal anti-HSV antibodies (Enzygnost) was used with a dilution of 1:20 in culture medium. After two days of incubation, the medium was discarded. The cells were washed once with PBS and then fixed with 5% paraformaldehyde solution and washed again with PBS. Subsequently, HSV-infected cells were stained using a FITC-conjugated anti-HSV antibody. After 1 hour, the supernatant was discarded, and cells were washed with PBS to remove excess antibodies. Afterwards, the cells were subjected to staining with Hoechst, DNA staining, for 15 minutes and then fixed with 5% paraformaldehyde for 15 minutes. The evaluation was carried out by fluorescence microscopy (20×, Inverted microscope, Leica).

When infected cells were incubated with HDIT102(H4), no cell-to-cell spread was observed. This indicates that HDIT102(H4) can effectively abrogate cell-to-cell spread at clinically achievable concentrations.

### Example 8: Identification of the HDIT102(H4) Epitope on Recombinant HSV-1 gB Protein by Solving the Structure at a Resolution of 3.44 Å Using Cryo-EM

To determine the HDIT102(H4) epitope on HSV gB, Cryo-EM was performed on a mix of recombinant HSV-1 gB and HDIT102(H4) Fab and the co-structure was solved at a resolution of 3.44 Å (FIG. 8A-C).

Cryo-EM grid preparation and data collection was done as follows. HSV-1 gB and HDIT102(H4) Fab were mixed in a ratio of 1 to 3.5. A 4 μl aliquot of the mixture was adsorbed onto glow-discharged Quantifoil Cu-R2/1-300 mesh holey carbon-coated grids (Quantifoil, Germany), blotted with Whatman 1 filter paper and vitrified into liquid ethane at −180° C. using a Leica EM GP2 plunger (Leica microsystems, Austria) operated at 10° C. and 85% humidity. Data of HSV-1 gB and HDIT102(H4)-Fab complex was acquired on a Titan Krios G1 TEM (ThermoFisher, USA) operated at 300 kV and equipped with a Gatan Energy Filter (Ametek, USA) and a K2 Summit direct electron detector (Ametek, USA). Micrograph movies of 40 frames were recorded in counting mode at a magnification of 165,000× (pixel size 0.82 Å) with a dose of 1.15 e⁻/Å²/frame, resulting in a total accumulated dose on the specimen level of approximately 46 e⁻/Å² per exposure.

Data processing and model building was done as follows. All image processing steps were performed with Relion v4.0 (Kimanius, Dong et al., 2021, Biochem J, Vol. 478 (24)). Dose weighting and motion correction of dose-fractionated and gain-corrected movies were performed using Relion's implementation of the UCSF motioncor2 program. Contrast transfer function (CTF) parameters were estimated using ctffind 4.1.14 (Rohou and Grigorieff, 2015, J Struct Biol, Vol. 192 (2)). Micrographs displaying strong drift, astigmatism greater than 1000 Å and maximum CTF resolution worse than 8 Å were excluded from further processing. A total of 1 million particles were picked using the Laplacian-of-Gaussian (LoG) filter in Relion 4.0 (Kimanius, Dong et al., 2021, Biochem J, Vol. 478 (24)). The particle dataset was cleaned through three rounds of reference-free 2D classification resulting in 277′376 particles. Relion's Stochastic Gradient Desecnt (SGD) algorithm was used to generate a de novo 3D initial model from the 2D particles. The particle dataset was further cleaned through three rounds of unsupervised 3D classification. The remaining 208′280 particles were subjected to Bayesian particle polishing, CTF and aberration refinement, and a final high-resolution 3D refinement, which resulted in a final map with an overall resolution of 3.44 Å according to the gold standard Fourier shell correlation (FSC) at FSC=0.143.

The HSV-1 gB X-ray structure (PDB-ID: 2GUM) was manually mutated at positions T313S, Q443L and V553A and placed into the final map using coot (Casanal, Lohkamp et al., 2020, Protein Sci, Vol. 29 (4)). For the HDIT102(H4) Fab, the crystal structure of a humanized recombinant Fabfragment (PDB-ID 7PHU) was mutated in coot (Casanal, Lohkamp et al., 2020, Protein Sci, Vol. 29 (4)) based on a sequence alignment generated by Needle EMBOSS (Rice, Longden et al., 2000, Trends Genet, Vol. 16 (6)). Three HDIT102(H4) Fabs were placed into the final map using coot (Casanal, Lohkamp et al., 2020, Protein Sci, Vol. 29 (4)). Molrep of the CCP-EM software suite v1.6 (Nicholls, Tykac et al., 2018, Acta Crystallogr D Struct Biol, Vol. 74 (Pt 6)) was used for the initial fitting of HSV-1 gB and the three HDIT102(H4) Fabs into the final map. The final protein model was obtained by several iterations of manual model building in coot (Casanal, Lohkamp et al., 2020, Protein Sci, Vol. 29 (4)), Refmac-Servalcat refinement and model validation in the CCP-EM software suite v1.6 (Nicholls, Tykac et al., 2018, Acta Crystallogr D Struct Biol, Vol. 74 (Pt 6)). Data collection, refinement and validation statistics are summarized in Table 2 and the data processing workflow in FIG. 8D.

TABLE 2
Cryo-EM data collection HDIT102(H4) Fab on HSV-1F
gB, refinement and validation statistics.

| Data collection | |
|---|---|
| Microscope | Titan |
| Voltage (kV) | 300 |
| Magnification | 165′000 |
| Pixel size (Å) | 0.82 |
| Collection software | Serial EM |
| Defocus range (μm) | −1.0 to −2.0 |
| Movies recorded | 6′611 |
| Frames per movie | 40 |
| Exposure rate (e⁻/Å2/frame) | 1.15 |
| Exposure time (s) | 4 |
| Cumulative exposure (e⁻/Å2) | 46 |
| Refinement | |
| Software | Relion v4.0 |
| Initial particle images (no.) | 1′059′496 |
| Final particle images (no.) | 208′280 |
| Symmetry imposed | C₁ |
| 0.143 FSC half map (Å) | 3.44 |
| Map sharpening B-factor | −74.05 |
| (Å²) | |
| Model building and validation | |
| Software | CCP-EM v1.6 |
| | coot v0.9.8 |
| MolProbity score | 1.37 |
| All-atom clashscore | 6.77 |
| Rotamer | |
| Favored (%) | 98.92 |
| Outliers (%) | 0 |
| Ramachandran | |
| Favored (%) | 97.68 |
| Outliers (%) | 0 |
| CaBLAM outliers (%) | 1.30 |
| Cβ outliers (%) | 0.10 |
| FSC 0.5 average (%) | 80.11 |

The structural organisation of the Fv domain of the HDIT102(H4) Fab is shown in FIG. 9A, B. Definition of the individual CDRs of HDIT102(H4) was done using the Martin numbering scheme (Norman, R. A., F. Ambrosetti, A. Bonvin, L. J. Colwell, S. Kelm, S. Kumar and K. Krawczyk (2020). “Computational approaches to therapeutic antibody design: established methods and emerging trends.” Brief Bioinform 21(5): 1549-1567) and numbering is used in the detailed analysis of interacting residues between gB and HDIT102(H4) (FIG. 9C). Structural arrangement of the CDRs of HDIT102(H4) heavy and light chain are shown in (FIG. 9D-1). Analysis of the HSV-1F gB residues that were identified to be in close proximity within a distance of 4 Å to residues of the HDIT102(H4) CDRs revealed in total 12 residues: D199, A203, K204, Y303, R304, Q321, V322, D323, K320, Y326, R335 and T337, which are located in two close patches on the gB surface structure (FIG. 10A). Analysis of the electrostatic charge of surface exposed residues in the HDIT102(H4) Fab revealed a negatively charged region that was in close proximity to positively charged HSV-1F gB residues K204 and R335 (FIG. 10B). R335 and surrounding residues were in particular of interest, since in vitro HSV-1 evolved the amino acid substitution R335Q when replicated under suboptimal concentrations of HDIT102(H4), conferring resistance to HDIT102(H4)-mediated neutralization. The gB residues in close proximity to CDR residues were further analysed. HSV-1 gB D323 is in proximity to form contacts with HDIT102(H4) HC CDR1 H27 and HC CDR1 T31 (FIG. 11). HSV-1 gB Y303 is in proximity to form contacts with HDIT102(H4) HC CDR1 R30 and HC CDR2 N53 (FIG. 12). HSV-1 gB R304 is in proximity to form contact with HDIT102(H4) HC CDR1 H27 (FIG. 13). HSV-1 gB Q321 is in proximity to form contacts with HDIT102(H4) HC CDR1 T31 (FIG. 14). HSV-1 gB V322 is in proximity to form contacts with HDIT102(H4) HC CDR3 T99 (FIG. 15). HSV-1 gB D199 is in proximity to form polar contacts with HDIT102(H4) HC CDR3 T100a and T100b (FIG. 16). HSV-1 gB A203 is in proximity to form non-polar contacts with HDIT102(H4) HC CDR3 T100b and LC CDR1 S32 (FIG. 17). HSV-1 gB K320 is, when in a specific rotamer conformation, in proximity to form polar contacts with HDIT102(H4) HC CDR3 T100b and LC CDR1 S32 (FIG. 18). HSV-1 gB Y326 is in proximity to form polar contacts with HDIT102(H4) HC CDR3 D101 (FIG. 19). HSV-1 gB K320 is in proximity to form polar contacts with HDIT102(H4) LC CDR2 D51 and peptide backbone at LC CDR1 G29, S30 and K31 (FIG. 20). HSV-1 gB R335 is in proximity to form polar contact with HDIT102(H4) LC CDR2 D53 and non-polar contact with LC CDR2 Y50 (FIG. 21). Finally, HSV-1 gB T337 is in proximity to form contact with HDIT102(H4) LC CDR2 S56 (FIG. 22).

### Example 9: Alanine Scanning Mutational Analysis of HDIT102(H4) Epitope Residues in HSV-1F gB

Structural information from Cryo-EM analyses was used to choose specific HSV-1F gB amino acid residues in close proximity to HDIT102(H4) CDR residues and interrogate them for their contribution to HDIT102(H4) binding by substitution to alanine (using site-directed mutagenesis (SDM)). 1.5×10¹ HEK293T cells were seeded per well in a 6 well plate one day before transfection (using 2 mL DMEM+10% FCS+P/S per well). 2 μg of plasmid encoding HSV-1gB wild type or selected gB mutants was prepared in a 1.5 mL tube and was mixed thoroughly (by vortexing) with 300 μL of PEI solution (300 μL OPTIMEM with 8 μL PEI (4 μg PEI per 1 μg DNA)) and incubated at room temperature for 15 min. Then the medium of prepared cells in the 6-well plate was carefully removed and 1 mL fresh DMEM (10% FCS+P/S) was added. Afterwards, the transfection mixture was added dropwise to HEK293T cells in 6-well plate and the plates were stored at 37° C. incubator 5% CO2. After 12-18 hours the transfection media was changed to 2 mL DMEM+10% FCS+P/S per well. Two days after transfection, the HEK293T cells were washed with PBS and detached through scraping tool or pipetting (no trypsin). The harvested cells were pelleted in 1.5 mL tubes (300×g for 5 min) and resuspend in fresh PBS and subjected for staining with different primary antibodies (HDIT102(H4), HDIT101 or control anti-gB IgG) staining for 45 min at room temperature. Then the cells were pelleted at 300×g for 5 min, the supernatant was discarded and cells were washed with 200 μl PBS, followed by secondary antibody staining with anti-human Fcγ IgG, polyclonal FITC, Jackson ImmunoResearch in 100 μL PBS, 45 min at RT, with no light exposure. At the end of the incubation the cells were pelleted and washed 2 times with PBS and taken up in 500 μl PBS. Following this, the mean fluorescence intensity (MFI) was determined for each sample by flow cytometry. To include information on possible differences in cell surface presentation of the mutant gB proteins, the integrated mean fluorescent intensity (iMFI) was calculated by multiplying percentage of HDIT102(H4)-positively stained cells with the geometric MFI of positively stained cells.

While all analysed mutants showed comparable expression on cells as compared to wild type gB, mutants gB K320A or Y326A were reduced in cell surface expression, indicating a loss of function of these mutants. Slight reduction in binding of HDIT102(H4) as compared to wild type gB was observed for R335A or R335Q mutants, however the magnitude of loss of binding was by far not as obvious as compared to HDIT101 staining of HDIT101-resistance mutant gB R304Q, used here as control (FIG. 23). The data indicate that HDIT102(H4) is able to bind to the analysed gB mutants, however for some this binding may be reduced in affinity as compared to the wild type gB.

### Example 10: Alanine Scanning—Analysis of HDIT102(H4) Induced Inhibition of Cell-Cell Fusion

To analyse the impact of HDIT102(H4)-induced inhibition of gB-mediated membrane fusion, cell-cell-fusion experiments were performed in the presence or absence of HDIT102(H4) using wild type or mutant gB constructs. Plasmids encoding wild type HSV-1F gB or single amino acid point mutants were co-transfected into a 1:1 mix of HEK293T-GFP/HEK293T-E2C fluorescent reporter cells (seeded with cell density of 1.0×10⁶ (total) per well in 6-well plate one day prior to the transfection), together with plasmids encoding HSV-1F gD, gH and gL (Mock transfection includes all plasmid except gB producing plasmids, 0.5 μg per plasmid was prepared and mixed with transfection medium including 150 μl 0.9% saline (NaCl) with 8 μL PEImax (4 μg PEI per 1 μg DNA) and thoroughly mixed by vortexing then incubated 15 minutes at room temperature, then the medium of prepared cells in the 6-well plate was carefully removed and 1 mL fresh DMEM (10% FCS+P/S) was added. Afterwards, the transfection mixture was added dropwise to the cells in 6-well plate and the plates were stored at 37° C. incubator 5% CO2 for 5 hours before HDIT102(H4) was added (1 ml DMEM+FCS+P/S containing HDIT102(H4) 75 μg/ml). In control samples no antibody was added. Two days later, the supernatants were removed, and the cells were washed with 1 ml PBS and detached using 0.5 ml trypsin (37° C.) and taken up in 5001 PBS. Fusion of cells was judged by the presence of GFP+E2C+ double positive cells by flow cytometry. Fold-change in fusion inhibition by HDIT102(H4) was calculated comparing HDIT102(H4) treated with untreated cells for each mutant and fold changes were normalized to fold-change for wild type gB.

Inhibition of gB-mediated fusion by HDIT102(H4) was only marginally reduced for gB mutants R304Q (92.3% of WT) or K320A (90.1% of WT) and moderately reduced for mutant Y326A (72.7% of WT). In contrast, for mutants D199A, K204A, R335A or R335Q, inhibition of fusion by HDIT102(H4) was significantly reduced to 32.9% of WT, 57.3% of WT, 19.3% of WT and 18.8% of WT, respectively (FIG. 24A). This indicates that these residues are critical for HDIT102(H4)-induced inhibition of gB-mediated membrane fusion, i.e., are important residues of the HDIT102(H4) epitope in gB. When fusion activity, as a combination of cell surface exposure and fusogenicity, of these mutants was analysed and compared to wild type gB, mutants that showed reduced sensitivity to HDIT102(H4)-induced inhibition, had reduced fusion activity (D199, 84.1% of WT; K204A, 92.6% of WT; R335A, 87.8% of WT; R335Q, 94.3% of WT), suggesting that viruses that would evolve escape mutations leading to these amino acid changes, may have fitness deficits (FIG. 24B). This is in contrast to HDIT101-resistance mutant R304Q, which had almost identical fusion activity as compared to wild type gB, suggesting superiority of HDIT102(H4) over HDIT101 in preventing emergence of resistance mutants.

### Example 11: HDIT102(H4) is not Inducing Measurable Antibody Dependent Cellular Cytotoxicity (ADCC) Upon Binding to its Target

ADCC is a defence mechanism of the immune system whereby immune effector cells lyse the target cells via an antibody-dependent process. In this assay, antibodies bind to gB that is expressed on the surface of infected Vero cells and the Fc effector portion of gB-bound antibodies binds to Fcγ receptors on the cell surface of effector cells (engineered Jurkat cells stably expressing the FcγRIIIa receptor, V158 (high affinity) variant, and an NFAT response element driving expression of firefly luciferase (Promega) after antibody binding to FcR occurs. Subsequently, the antibodies' biological activity in activating ADCC can be quantified by measuring the luciferase produced as a result of FcγRIIIa receptor binding and NFAT activation. In brief, Vero cells were plated and the day after infected at MOI of 1 with HSV-1F or HSV-2G. Twenty hours after infection, infected Vero cells were harvested and distributed in white flat bottom 96-well plates (1.25×10⁴ cells per well) and incubated 6 hours together with effector cells (effector: target ratio=6:1) and a serial dilution of HDIT102(H4). Uninfected Vero cells were included as a negative control. Raji cells incubated with Rituximab served as positive control for the assay (data not shown). In addition, polyclonal anti-HSV antibody served as positive control. Subsequently luciferase substrate was added to the plates and incubated 15 minutes. Afterwards the luminescence intensity was measured using a plate reader. Triplicate reads were performed. Plate Background was determined by calculating the average RLU (relative light unit) from control wells (empty). Fold of ADCC induction was calculated accordingly: Fold of ADCC induction=RLU (induced-background)/RLU (no antibody control-background) and data was graphed as fold of ADCC induction versus log 10 antibody concentration. In addition to infected Vero cells, HEK293T cells stably transduced to express gB from HSV-1 or HSV-2 were used to determine ADCC induction.

Neither in HSV-1F infected, nor in HSV-2G infected Vero cells, HDIT102(H4) activated ADCC (FIG. 25A). In contrast, polyclonal anti-HSV antibody activated ADCC in HSV-1F or HSV-2G infected cells, but not in uninfected control cells (FIG. 25B). Summary of the data from infected Vero cells is shown in FIG. 25C. Similar results were observed in HEK293T cells ectopically expressing the target protein gB from HSV-1 or HSV-2 FIG. 25D-F.

### Example 12: HDIT102(H4) is not Inducing Measurable Complement-Dependent Cellular Cytotoxicity (CDC) Upon Binding to its Target

For the quantitative determination of complement activation, human terminal complement complex C5b-9 (TCC C5b-9) concentration was measured by following the protocol of a commercial kit (Human terminal complement complex C5b-9 ELISA, Blue Gene for life science). The assay is based on a sandwich enzyme immunoassay technique. Antibodies specific for TCC C5b-9 are pre-coated on a microplate. TCC C5b-9 in the samples or standards is bound by the immobilized antibody. After removing any unbound substances, a biotin-conjugated antibody specific for TCC C5b-9 is added to the wells. After washing, avidin conjugated Horseradish Peroxidase (HRP) is added. Following a wash to remove any unbound avidin-enzyme reagent, a substrate solution is added to the wells and colour develops in proportion to the amount of TCC C5b-9 bound in the initial step. The colour development was stopped, and the intensity of the colour was measured with an absorbance/fluorescence plate reader (Tecan) at wavelength of 450 nm.

It was tested, whether binding of HDIT102(H4) to its target, activates complement, i.e. C5b-9 secretion. Cytotect® (Biotest) (human polyclonal anti CMV antibodies) and polyclonal anti-HSV antibody were used as positive controls. Uninfected cells and an unrelated isotype IgG were used as negative controls. Complement activation was measured by detecting C5b-9 in the media after incubation as indicated. When HSV-1F infected Vero cells, but not uninfected cells, were incubated with HDIT102(H4), no detectable increase in C5b-9 concentration was observed as compared to negative controls. In contrast, C5b-9 concentration increased in the presence of not-heat inactivated serum, but not in the presence of heat-inactivated serum, when polyclonal anti-HSV antibody or Cytotect® was used (FIG. 26A). Similar results were also seen for HSV-2 infected cells (FIG. 26B).

### Example 13: HDIT102(H4) Efficiently Neutralizes HSV-1 or HSV-2 Cell-Free Virus Independently of Presence of Complement

To clarify the question, whether antibody neutralization potency of HDIT102(H4) is dependent on complement or not, a TCID50-based neutralization assay was performed. In this assay, DMEM was supplemented with 10% heat-inactivated or not heat-inactivated IgG-depleted human serum as complement source. The human polyclonal IgG preparation targeting human cytomegalovirus (Cytotect) was used as a positive control. HDIT102(H4) neutralization potencies for HSV-1F or HSV-2G were compared in the presence of complement and heat inactivated complement. Vero cells were incubated with 100 TCID50 of HSV-1F (FIG. 27A) or HSV-2G (FIG. 27B) in the presence of a dilution series of either HDIT102(H4) or control. The extent of the cytopathic effect was examined by light microscopy three days after infection. The neutralization titre was determined to be the highest antibody dilution at which the virus was completely neutralized and the formation of a CPE in the inoculated cell cultures was completely prevented. (The differences were calculated by one-way ANOVA, error bars represent standard deviation of the mean of three technical replicates, **p-value<0.005). In case of absence of error bars, the results of the replicates were identical. The results show that HDIT102(H4) neutralizes HSV-1F or HSV-2G with similar efficiency regardless of presence or absence of complement.

### Example 14: HDIT102(H4) Mediates Antibody-Dependent Cellular Phagocytosis (ADCP) of HSV-1 and HSV-2

Antibody-dependent cellular phagocytosis (ADCP) is a mechanism by which professional phagocytes, such as macrophages and neutrophils contribute to antitumor or antimicrobial (e.g. antiviral) potency of monoclonal antibodies via the engagement of Fcγ receptors by antibody-opsonized material. A microscopy-based assay was used to define the phagocytic activity of HDIT102(H4). The assay was optimized with undifferentiated THP-1 cells, a human monocytic cell line derived from an acute monocytic leukaemia patient. For this purpose, 100 TCID50 of HSV-1F or HSV-2G were prepared in RPMI with 10% IgG depleted human serum and mixed with 500 nM of HDIT102(H4). The antibody-virus mixtures were incubated 1 hour at 37° C. Afterwards 1.0×10¹ THP-1 cells per sample were washed with PBS and incubated with samples at 37° C. for 18 hours (total volume of mixture 500 μl, 250 μl of virus+antibody with 250 μl cells). After 18 hours of incubation, THP-1 cells were harvested into 1.5 ml tubes and centrifuged 5 min at 300×g at room temperature and subsequently washed once with PBS. The THP-1 cells were resuspended in 200 μl of fixation buffer (Perm/Fix Kit, BD) and incubated 30 min at room temperature. Fixed THP-1 cells were washed twice with 200 μl of 1×permeabilization buffer (Perm/Fix Kit, BD) and resuspended in 200 μl of 1×permeabilization buffer (containing conjugated antibody or primary antibody) and incubated for 45 min at room temperature and protected from light. Afterwards, the samples were pelleted by centrifugation as above and washed once with 1×permeabilization buffer and stained with Hoechst (diluted in 1×permeabilization buffer) for 3 min at RT. Samples were washed with PBS and resuspended in 20 μl of water. Samples were mounted with a drop of Mowiol on microscopy glass slides. Twenty-four hours later microscopic analysis was performed. All centrifugation steps were carried out at RT and 300×g, for 5 minutes. For detecting virus which underwent phagocytosis a FITC conjugated anti-HSV antibody was used.

In the presence of HDIT102(H4), but not an irrelevant IgG isotype control, THP-1 cells phagocytosed HSV-1F as indicated by increased FITC signal after staining (FIG. 28A).

To test whether phagocytosis was also mediated in primary cells, human CD14-positive monocyte derived macrophages (MDMs) from four independent healthy donors were generated. Frozen PBMC from healthy donors were thawed and pelleted (400×g, 5 minutes, 4° C.) and the supernatant was discarded. The cells were resuspended in 5 ml cold 1×PBS (PBS+2 mM EDTA: 50 ml PBS+200 μl 0.5 M EDTA) and again pelleted (400 g, 5 minutes, 4° C.). The supernatant was discarded. The cell pellet was resuspended in 80 μL of MACS buffer (PBS, 0.5% BSA, 2 mM EDTA, sterile filtered 0.22 μm) per 1.0×10⁷ total cells followed by adding 20 μL of CD14 MicroBeads (ultrapure beads, Miltenyi) per 1.0×10⁷ total cells (mixed well and incubated for 15 minutes in the refrigerator (2-8° C.)). The cells were washed by adding 1-2 mL of buffer per 1.0×10⁷ cells and centrifugation at 300×g for 10 minutes. The supernatant was discarded, and the cells were resuspended up to 1.0×10⁸ cells in 500 μL of MACS buffer and were subjected to magnetic separation with LS columns. A collection tube was placed under the LS column (50 ml tube) for flow through and the column (wings to the front) were positioned in the magnetic field of a suitable MACS separator and was rinsed with the appropriate amount of buffer (3 mL MACS buffer), afterwards the cell suspension was applied onto the column. Then the column was washed 3 times with 3 ml buffer. The flow through tube was replaced with the fresh tube and the magnetically labelled cells were flushed out by pipetting 5 ml of the buffer onto the column firmly pushing the plunger into the column. The eluted cells were pelleted at 400×g for 5 minutes and counted and seeded with GM-CSF media (RPMI+10% FCS+P/S with GM-CSF (80 ng/ml final), 50.000 monocytes per well of 96-well plate flat bottom, in 150 μl final volume). Seven days later, LPS+INFγ (each 20 ng/ml final) were added in 10 μl directly into each well and cells were incubated overnight. The required amount of virus (MOI:10) was stained with ATTO Dye-NHS (1:100) for 1 hour at RT and then to quench excess amount of dye, same volume of Tris-HCL (1M, pH8.0) was added to the stained virus sample and incubated 30 minutes at RT. Then an amount of labelled HSV1-F corresponding to MOI10 (incubated 30 minutes at RT+/−test antibody: HDIT102(H4, final concentration of [150 μg/ml]) was added into the wells containing the MDMs and cells were incubated for 48 hours at 37° C. The supernatants were discarded and MDMs were detached using trypsin (incubated with 80 μl of warm trypsin in each well of 96 well plates at 37° C. for 8-10 minutes and harsh pipetting) and taken up in 100 μl of RPMI with 10% FCS and pelleted at 400×g for 3 minutes. The pellets were resuspended in 200 μl PBS. Followed by live-dead staining with ZOMBIE Aqua DYE (Biolegend) (1:500 diluted in PBS, 100 μl, 15 minutes RT, protected from light). After the incubation time, 100 μl PBS containing 2% FCS was added and the cells were pelleted for 3 minutes at 400×g and the supernatant was discarded. The washing step was repeated with 200 μl PBS containing 2% FCS. Afterwards the cells were resuspended in 50 μl PBS containing 0.25 μl Fc block (Human TruStain FcX, Biolegend) per well, and Fc-receptors were blocked for 5 minutes at RT. Afterwards the cells were stained by adding 50 μl PBS containing anti-CD86 BV421 (Biolegend) and incubated for 15 minutes at 4° C. and washed 2 times (200 μl PBS, 3 min 400×g). Thereafter, the cells were fixed for 15 minutes with PFA (100 μl, 4% PFA in PBS), washed one time with PBS and, resuspended in 150 μl PBS+FCS. The phagocytic uptake of labelled HSV-1F immune complexes opsonized with HDIT102(H4) was then determined by measuring virus-ATT0488 positive MDMs via flow cytometry. The statistical differences between groups were calculated using One-way ANOVA. *p-value=0.0061.

MDM showed a substantially increased uptake of HSV-1F in the presence of HDIT102(H4) (FIG. 28B). Conclusively, the data show that HDIT102(H4) can mediate ADCP of viral particles by MDMs.

### Example 15: HDIT102(H4) Fc Part Plays a Pivotal Role in Rescuing Balb/c Mice from a Lethal HSV-2 Infection

In vivo efficacy of the HDIT102(H4) Fc mutant N297A was investigated in an HSV-2 infection Balb/cOlaHsd mouse model. N297 is normally glycosylated and glycosylation is required for efficient interaction with Fcγ-receptors. Amino acid substitution of this site to alanine reduces the interaction with Fcγ-receptors and subsequent ADCP. One week prior to the experiment, 6-week-old female mice Balb/c (weight: 16-19 grams) were purchased from ENVIGO and one week prior to virus inoculation pre-treated subcutaneously with medroxyprogesteron (longacting progestin Depo-Clinovir, prepared at 25 mg/ml in PBS and 100 μl per mouse). On the day of intravaginal virus inoculation, the experimental animals were anesthetized by isoflurane. During the short anaesthesia, the vaginal mucosa was cleaned from the vaginal secretions by using a sterile ESwab and the experimental animals were infected by intravaginal inoculation of 101 of herpes virus stock (containing 5.0×10⁴ TCID50 HSV-2G) to the vaginal mucosa using a pipette. Afterwards, a small amount of Epiglu tissue adhesive was applied on the surface to glue the vagina (avoids inoculum to flow out). Normally the glue was lost within 1 day after inoculation. The efficiency of HDIT102(H4)-N297A in protecting mice from a lethal HSV-2G infection was assessed by the intraperitoneal administration of 600 μg HDIT102(H4)-N297A or HDIT102(H4) (~30 mg/kg) in 1001 PBS (N=5) one hour post-exposure. The control group was treated with PBS (N=5). The experimental animals were regularly inspected for weight loss and the occurrence of perineal hair loss (HL), redness (R) and swelling (S) and neurologic damage (e.g. hind limb paralysis, gastrointestinal track blockage) over an observation period of 60 days. Visible inspection was graded from slight to severe symptoms accordingly +/++/+++. Experimental animals were sacrificed in case of severe signs of herpes encephalitis, or paralysis or occurrence of severe lesions to prevent undue suffering. All experiments were done in line with ethical approval.

All control animals died of the infection within 12 days. While HDIT102(H4) treated animals were readily protected from the lethal HSV-2G infection, HDIT102(H4)-N297A treated animals were substantially less protected and at the end of the observation phase only 2/5 (40%) of HDIT102(H4)-N297A treated mice survived, while 4/5 (80%) of HDIT102(H4)-treated mice survived (FIG. 29). The data show that glycosylation of HDIT102(H4) at N297 is a likely pre-requisite necessary for function and suggest that interaction with Fc-receptors and ADCP is essential for efficient therapeutic effects.

### Example 16: Cryo-Electron (Cryo-EM) Microscopy Solved Co-Structure of HDIT101 Bound to HSV-1F gB

A combination of HDIT101 with HDIT102(H4) may be a beneficial strategy to overcome possible emergence of resistant viruses. To identify the specific epitope that HDIT101 binds, Cryo-EM analyses were performed with recombinant HSV-1F gB and HDIT101 Fab, using a similar approach as described in Example 8. The structure of HDIT101 Fab bound to HSV-1F gB was solved at a resolution of 3.27 Å (FIG. 30A-C).

Cryo-EM grid preparation and data collection was done as follows: HSV-1 gB and HDIT101 Fab were mixed in a ratio of 1 to 3.5. A 4 W aliquot of the mixture was adsorbed onto glow-discharged Quantifoil Cu-R1.2/1.3-300 mesh holey carbon-coated grids (Quantifoil, Germany), blotted with Whatman 1 filter paper and vitrified into liquid ethane at −180° C. using a Leica EM GP2 plunger (Leica microsystems, Austria) operated at 10° C. and 85% humidity. Data of HSV-1-gB and HDIT101-Fab complex was acquired on a Glacios TEM (ThermoFisher) operated at 200 kV and equipped with a Quantum K3 direct electron detector (Gatan). Micrograph movies of 40 frames were recorded in counting mode at a magnification of 45,000× (pixel size 0.878 Å) with a dose of 1.25 e⁻/Å²/frame, resulting in a total accumulated dose on the specimen level of approximately 50 e⁻/Å² per exposure. For data processing and model building the following was done: All image processing steps were performed with Relion v4.0 (Kimanius, Dong et al., 2021, Biochem J, Vol. 478 (24)). Dose weighting and motion correction of dose-fractionated and gain-corrected movies were performed using Relion's implementation of the UCSF motioncor2 program. Contrast transfer function (CTF) parameters were estimated using ctffind 4.1.14 (Rohou and Grigorieff, 2015, J Struct Biol, Vol. 192 (2)). Micrographs displaying strong drift, astigmatism greater than 1000 Å and maximum CTF resolution worse than 8 Å were excluded from further processing. A total of 3 million particles were picked using the Laplacian-of-Gaussian (LoG) filter in Relion 4.0 (Kimanius, Dong et al., 2021, Biochem J, Vol. 478 (24)). The particle dataset was cleaned through three rounds of reference-free 2D classification resulting in 714′565 particles. Relion's Stochastic Gradient Desecnt (SGD) algorithm was used to generate a de novo 3D initial model from the 2D particles. The particle dataset was further cleaned through three rounds of unsupervised 3D classification. The remaining 233′330 particles were subjected to Bayesian particle polishing, CTF and aberration refinement, and a final high-resolution 3D refinement, which resulted in a final map with an overall resolution of 3.27 Å according to the gold standard Fourier shell correlation (FSC) at FSC=0.143. The HSV1 gB X-ray structure (PDB-ID: 2GUM) was manually mutated at positions T313S, Q443L and V553A and placed into the final map using coot (Casanal, Lohkamp et al., 2020, Protein Sci, Vol. 29 (4)). For the HDIT101 Fab, the crystal structure of a humanized recombinant Fab fragment of a murine antibody (PDB-ID 3AAZ) was mutated in coot (Casanal, Lohkamp et al., 2020, Protein Sci, Vol. 29 (4)) based on a sequence alignment generated by Needle EMBOSS (Rice, Longden et al., 2000, Trends Genet, Vol. 16 (6)). Three HDIT101 Fabs were placed into the final map using coot (Casanal, Lohkamp et al., 2020, Protein Sci, Vol. 29 (4)). Molrep of the CCP-EM software suite v1.6 (Nicholls, Tykac et al., 2018, Acta Crystallogr D Struct Biol, Vol. 74 (Pt 6)) was used for the initial fitting of HSV1 gB and the three HDIT101 Fabs into the final map. The final protein model was obtained by several iterations of manual model building in coot (Casanal, Lohkamp et al., 2020, Protein Sci, Vol. 29 (4)), Refmac-Servalcat refinement and model validation in the CCP-EM software suite v1.6 (Nicholls, Tykac et al., 2018, Acta Crystallogr D Struct Biol, Vol. 74 (Pt 6)). Data collection, refinement and validation statistics are summarized in Table 3 and the data processing workflow is shown in FIG. 30D.

TABLE 3
Cryo-EM data collection HDIT101 Fab on HSV-1F
gB, refinement and validation statistics.

| Data collection | |
|---|---|
| Microscope | Glacios |
| Voltage (kV) | 200 |
| Magnification | 45′000 |
| Pixel size (Å) | 0.878 |
| Collection software | Serial EM |
| Defocus range (μm) | −1.0 to −2.0 |
| Movies recorded | 5′815 |
| Frames per movie | 40 |
| Exposure rate (e⁻/Å2/frame) | 1.25 |
| Exposure time (s) | 4 |
| Cumulative exposure (e⁻/Å2) | 50 |
| Refinement | |
| Software | Relion v4.0 |
| Initial particle images (no.) | 2′993′934 |
| Final particle images (no.) | 233′330 |
| Symmetry imposed | C₁ |
| 0.143 FSC half map (Å) | 3.27 |
| Map sharpening B-factor | −50.38 |
| (Å²) | |
| Model building and validation | |
| Software | CCP-EM v1.6 |
| | coot v0.9.8 |
| MolProbity score | 1.71 |
| All-atom clashscore | 12.28 |
| Rotamer | |
| Favored (%) | 97.98 |
| Outliers (%) | 0.19 |
| Ramachandran | |
| Favored (%) | 97.44 |
| Outliers (%) | 0 |
| CaBLAM outliers (%) | 1.09 |
| Cβ outliers (%) | 0 |
| FSC 0.5 average (%) | 78.62 |

The structural organisation of the Fv domain of the HDIT101 Fab is shown in FIG. 31A, B. Definition of the individual CDRs of HDIT101 was done using the Martin numbering scheme (Norman, R. A., F. Ambrosetti, A. Bonvin, L. J. Colwell, S. Kelm, S. Kumar and K. Krawczyk (2020). “Computational approaches to therapeutic antibody design: established methods and emerging trends.” Brief Bioinform 21(5): 1549-1567) and numbering is used in the detailed analysis of interacting residues between gB and HDIT101 (FIG. 31C). Structural arrangement of the CDRs of HDIT101 heavy and light chain are shown in (FIG. 31D-1). Analysis of the HSV-1FgB residues that were identified to be in close proximity within a distance of 4 Å to residues of the HDIT101 CDRs revealed in total 13 residues: Y301, G302, Y303, R304, E305, H308, K320, D323, Y326, P339, T341, W356 and P358, which are located in a patch on the gB surface structure (FIG. 32A). Analysis of the electrostatic charge of surface exposed residues in the HDIT101 Fab revealed a negatively charged cleft that was in close proximity to positively charged HSV-1F gB residue R304 (FIG. 32B). R304 and surrounding residues were in particular of interest, since in vitro HSV-1 evolved the amino acid substitution R304Q when replicated under suboptimal concentrations of HDIT101, conferring resistance to HDIT101-mediated neutralization. The gB residues in close proximity to CDR residues were further analysed.

HSV-1 gB H308 is in proximity to form contacts with HDIT101 HC CDR3 Y99, HC CDR1 G31b and HC CDR2 W53 and the peptide backbone (FIG. 33). HSV-1 gB D323 is in proximity to form contacts with HDIT101 LC CDR1 H30a and the peptide backbone at LC CDR3 S92 (FIG. 34). HSV-1 gB G302 peptide backbone is in proximity to form contact with HC CDR2 W53 (FIG. 35). HSV-1 gB K320 is in proximity to form polar contact with HDIT101 HC CDR2 D56 (FIG. 36). HSV-1 gB P339 is in proximity to form contact with HDIT101 LC CDR3 H93 (FIG. 37). HSV-1 gB R304 is in proximity to form contacts with HDIT101 LC CDR1 H30a, LC CDR1 Y32, LC CDR3 W96 and HC CDR3 Y97 (FIG. 38). HSV-1 gB T341 is in proximity to form contacts with HDIT101 LC CDR1 H30a and LC CDR1 S30b (FIG. 39). HSV-1 gB W356 and P358 are in proximity to form contacts with HDIT101 LC CDR1 N30c (FIG. 40). HSV-1 gB Y301 is in proximity to form contact with HDIT101 HC CDR2 W53 (FIG. 41). HSV-1 gB Y303 is in proximity to form non-polar contacts with HDIT101 HC CDR2 W52 and HDIT101 HC CDR2 W53 as well as polar contacts with HC CDR2 N54 and HC CDR2 D56 (FIG. 42). HSV-1 gB Y326 is in proximity to form polar contact with LC CDR1 Q27 (FIG. 43). Finally, HSV-1 gB E305 is in proximity to form contacts with LC CDR1 N30c, LC CDR1 Y32 and HC CDR3 P100a (FIG. 44).

### Example 17: Comparison of HDIT101 and HDIT102(H4) Fab Binding Regions Shows Overlap of Epitopes on the HSV-1 gB Trimeric Structure

The analysis of the HDIT101 (FIG. 45A) and HDIT102(H4) (FIG. 45B) epitopes on HSV-1 gB using the solved Cryo-EM structures with bound respective Fab fragment showed that both epitopes overlapped. The structures of HSV-1F gB bound by HDIT101 Fab or HDIT102(H4) Fab were superimposed and viewed from the side (FIG. 45C-E), bottom (FIG. 45F-H), or top (FIG. 45I-K). Superimposition of HSV-1F gB trimer in post-fusion conformation with bound HDIT101 (black) or HDIT102(H4) (grey) Fab demonstrates steric hindrance of simultaneous binding of both Fabs to one gB protomer. Both Fabs bind in a perpendicular orientation to another, suggesting that in case of the full IgG binding, these may also be organized in a perpendicular orientation with Fc domains pointing into orthogonal different orientation (FIG. 45L, H).

### Example 18: HDIT102(H4) and HDIT101 are Competing for Binding to HEK293T Cells Ectopically Expressing HSV-1 gB

A FACS-based assay was performed to test the competition of HDIT101 and HDIT102(H4) binding to gB expressed on cells. HEK293T ectopically expressing HSV-1 gB were grown in culture for one week before conducting the experiment. On the day of the experiment the cells were detached via scraping method and were washed with PBS (300×g 7 minutes). The cell pellets were incubated with antibody mixture for 45 min at room temperature on a shaker. Antibody mixture contained 10 μg/mL of a murine version of HDIT101 called MAb2c in combination with a serial dilution of HDIT101, HDIT102(H4) or a none competing control antibody. After incubation the cells were washed and an anti-murine IgG-APC was added to detect bound murine MAb2c antibody on the target cells and were incubated 30 min at room temperature on a shaker. The cells were washed again and subjected to FACS measurement. Both, HDIT101 and HDIT102(H4) competed for binding to gB expressing target cells with the murine MAb2c, while the control antibody did not (FIG. 46A). When we analysed 2D-image reconstructions from Cryo-EM analyses of recombinant HSV-1F gB mixed with 1:1 molar ratio of HDIT101 Fab and HDIT102(H4) Fab, we observed different classes: a) heterogenic gB-Fab immune complexes consisting of two HDIT102(H4) Fab molecules and one HDIT101 Fab molecule bound to the trimeric gB complex (FIG. 46B), as well as b) homogenic gB-Fab immune complexes with only HDIT102(H4) Fab bound (FIG. 46C), easily distinguishable by the different orientations of the Fabs. Simultaneous binding of HDIT101 and HDIT102(H4) Fab to one gB protomer was not observed.

### Example 19: a Combination Therapy of HDIT101 with HDIT102(H4) Demonstrates Synergistic Effects In Vivo Promoting Enhanced Survival Compared to Monotherapy after Infection with a Lethal Dose of HSV-2

One week prior to the experiment, 6-week-old female Balb/c mice (weight: 16-19 grams) were purchased from ENVIGO company and one week prior to the viral inoculation, they were pre-treated subcutaneously with medroxyprogesteron (longacting progestin Depo-Clinovir, prepared at 25 mg/ml in PBS and 100 μl per mouse). On the day of intravaginal inoculation, the experimental animals were anesthetized by isoflurane. During the short anaesthesia, the vaginal mucosa was cleaned from the vaginal secretions using a sterile ESwab and the mice were infected by intravaginal inoculation of the respective herpesvirus by applying 10 of herpes virus stock (containing 5.0×10⁴TCID50 HSV-2G) to the vaginal mucosa using a pipette. Afterwards, a small amount of Epiglu tissue adhesive was applied on the surface to prevent inoculum flow out. Normally the glue was lost within 1 day after inoculation. The efficiency of HDIT102(H4) in combination with HDIT101 in protecting mice from HSV-2 induced death was assessed by intraperitoneal administration of HDIT102(H4), HDIT101 (monotherapy) or HDIT102(H4)+HDIT101 (combination therapy) at a final total dose of 300 μg in 100 μl PBS per mouse (20 mice per treatment group and 15 mice for the control arm) one hour post exposure. The mice were regularly inspected for weight loss and the occurrence of perineal hair loss (HL), redness (R) and swelling (S) and neurological symptoms (e.g., hind limb paralysis, gastrointestinal track blockage) over a 60 days of observation period. Visible inspection was graded from slight to severe symptoms accordingly +/++/+++. Experimental animals were sacrificed in case of severe signs of herpes encephalitis, paralysis or visible severe lesions to prevent undue suffering. The statistical differences between survival curves were calculated using Logrank Mantel Cox test. **P-value=0.0069. The differences between cumulative combined clinical scores were analysed using Kolmogorov-Smirnow test. ****P-value<0.0001). All experiments were done in line with ethical approval.

All mice of the control group died of the HSV-2 infection by day 11 post infection. While the monotherapy with either HDIT101 or HDIT102(H4) at a dose of 300 μg rescued 9/20 (45%) or 10/20 (50%) of animals, respectively, within the observation period, the combination of both antibodies (150+150 g) let to the survival of 18/20 mice (90%) (FIG. 47A). The cumulative combined clinical scores demonstrated a significant improvement in clinical symptoms when the HDIT101+HDIT102(H4) combination therapy was used, as compared to the monotherapies (FIG. 47B).

### Example 20: HDIT102(H4) Neutralizes In Vitro Generated HDIT101-Resistant Mutant HSV-1 In Vivo and In Vitro

To address the question whether propagation of HSV-1 or HSV-2 in cultured cells in the presence of neutralizing antibody HDIT101 results in evolutionary adaptations that mediates resistance to antibodies and whether HDIT101-resistant mutants could be neutralized by HDIT102(H4), an in vitro assay was established to generate HDIT101-resistant mutants. In brief, confluent Vero cells (4.0×10⁵ cells/ml) were infected with HSV-1F or HSV-2G at MOI 0.01 and passaged in the presence of increasing concentration of HDIT101 in multiple rounds. After each round of propagation, virus was harvested, and an aliquot was used to inoculate fresh Vero cells. After four rounds of passaging the virus, harvested virus was characterized using HDIT101 neutralization assay (TCID50-based) to test for resistance against HDIT101. Isolated total DNA from infected cells was subjected to PCR sequencing to identify possible mutations in the gB coding region. We observed the emergence of HSV-1F R304Q (HSV-1FHDIT101R) and HSV-2G R296Q (HSV-2GHDIT101R) HDIT101-resistance mutants. Similar experiments with HDIT102(H4) let to the in vitro emergence of resistant mutants HSV-1F R335Q and HSV-2G R327W (FIG. 48A, B). FIG. 48B shows an alignment of HSV-1F and HSV-2G gB amino acid sequences (SEQ ID NO:39 and SEQ ID NO:40, respectively). From the alignment of HSV-1F and HSV-2G gB amino acid sequences, a strong conservation in the HDIT101 and HDIT102(H4) binding regions in the epitope regions can be derived. Despite several attempts to grow mutants in vitro that would escape both antibodies, HDIT101 and HDIT102(H4), we failed, suggesting that double resistant mutants do not evolve, possibly due to loss of viral fitness. In addition to the observed synergistic effects in vivo, this underlines that a combination of both antibodies may be beneficial over monotherapy as an effective novel anti-HSV therapy.

A TCID50 based assay was performed to evaluate the neuralization efficiency of antibodies against HDIT101-resistant mutant HSV-1F or HSV-2G in vitro. HDIT102(H4) and HDIT101 were used in their concentration to efficiently neutralize wild type virus (HDIT102(H4): 61.5 nM and 31.25 nM for HSV-1FHDIT101R and HSV-2GHDIT101R, respectively and HDIT101: 31.25 nM for both HSV-1FHDIT101R and HSV-2GHDIT101R). The results of these assays are summarized in Table 4. HDIT101-resistant HSV-1F or HSV-2G were both equally neutralized by HDIT102(H4) as their parental wild type strains.

TABLE 4
Sensitivity of HDIT101-resistant mutant
viruses to HDIT102(H4) neutralization.

| | HSV-1FHDIT101R | HSF-2GHDIT101R |
|---|---|---|
| HDIT102(H4) | + | + |
| HDIT101 | − | − |

TCID50 based assay was performed to evaluate the neutralization efficiency of antibodies against resistant HSV. HDIT102(H4) and HDIT101 were used in their concentration to efficiently neutralize wild type virus (HDIT102(H4): 61.5 nM and 31.25 nM for HSV-1FHDIT101R and HSV-2GHDIT101R, respectively and HDIT101: 31.25 nM for both HSV-1FHDIT101R and HSV-2GHDIT101R). + indicates sensitive to antibody-mediated neutralization, − indicates insensitive to antibody-mediated neutralization.

To test HDIT102(H4) neutralization efficiency against in vitro generated HDIT101-resistant mutant HSV-1F in vivo, a Balb/c model was employed. One week prior to the experiment, six-week-old female mice Balb/c (weight: 16-19 grams) were purchased from ENVIGO company and one week prior to the viral inoculation, they were pre-treated subcutaneously with medroxyprogesteron (longacting progestin Depo-Clinovir, prepared at 25 mg/ml in PBS and 100 μl per mouse). On the day of intravaginal inoculation, the experimental animals were anesthetized by isoflurane. During the short anaesthesia, the vaginal mucosa was cleaned from the vaginal secretions by using a sterile ESwab and the experimental animals were infected by intravaginal inoculation of the respective herpesvirus by applying 10 μl virus stock (containing a lethal dose of HSV-2GHDIT101R (1.0×10⁵ TCID50) carrying gB R296Q HDIT101-resistance change) to the vaginal mucosa using a pipette. Afterwards, a small amount of Epiglu tissue adhesive was applied on the surface to glue the vagina to avoids inoculum flow out. Normally the glue was lost within 1 day after inoculation. The efficiency of HDIT102(H4) in protecting mice from HSV-2GHDIT101R infection was assessed by intraperitoneal administration of HDIT102(H4) (N=5) one hour post exposure. HDIT101 was used as a control (N=5) and PBS control was also included (N=5). The experimental animals were regularly inspected for weight loss and the occurrence of perineal hair loss (HL), redness (R) and swelling (S) and neurological damage (e.g. hind limb paralysis, gastrointestinal track blockage) over an observation period of 60 days. Visible inspection was graded from slight to severe symptoms accordingly +/++/+++. Experimental animals were sacrificed in case of severe signs of herpes encephalitis, paralysis or visible severe lesions to prevent undue suffering. The statistical differences between survival curves were calculated using Logrank Mantel Cox test. **P-value<0.0015. All experiments were done in line with ethical approval.

All mice of the control group died within 10 days post virus infection and treatment. Also, all HDIT101 treated animals died of the lethal infection by HSV-2GHDIT101R carrying HDIT101-resistance change R296Q. In contrast 4/5 (80%) of HDIT102(H4)-treated animals infected with HSV-2GHDIT101R survived, demonstrating that HDIT102(H4) can efficiently neutralize HDIT101-resistant virus in vivo (FIG. 48C)

### Example 21: HDIT101 and HDIT102(H4) Induce the Internalization of gB from the Cell Surface of gB-Expressing as Well as HSV-1 Infected Cells

293T cells expressing carboxyterminal GFP-tagged HSV-1 gB were analysed by fluorescence microscopy after incubation with either 75 μg/ml HDIT101 or HDIT102(H4) or with a control IgG (anti-CD22 huRFB4) treatment overnight. Both, HDIT101, as well as HDIT102(H4) induced the aggregation of gB within the cells in large vesicle-like structures (FIG. 49A). Intracellular aggregation of transmembrane proteins after antibody binding could lead to enhanced proteasomal degradation and presentation of antigen peptides on major histocompatibility complexes (MHC). Induction of gB internalization by HDIT101 or HDIT102(H4) treatment was also analysed in HSV-infected cells and cells ectopically expressing the untagged target gB protein using an Incucyte machine (Sartorius) and labelling the antibody with a pH sensitive dye, to demonstrate uptake and transit of gB after antibody binding into the endosomal compartment. In this assay HEK293T HSV-1 gB were cultured in DMEM complete media at 37° C. and 25000 cells in 50 μl per well were seeded into a 96-well flat bottom microplate one day before the measurement. Next day, the test antibodies were labelled with Incucyte® Fabfluor-pH antibody labelling dye in target cell growth media in an amber tube to protect from light (at 2× of the final concentration of 4 g/mL of test antibody or the Fabfluor-pH antibody labelling Dye, A 1:3 molar ratio of test antibody is recommended, incubated at 37° C. for 15 minutes protected from light). Afterwards, the plate was removed from the incubator and 50 μl of 2× labelled antibody and control solutions were added to designated wells. Bubbles were removed and the plate was placed immediately back in the Incucyte® Live-Cell Analysis System and scanning was started (in the Incucyte® integrated software, it was scheduled to image every 15-30 min, depending on the speed of the specific antibody internalization). In principle the pH-sensitive dye-based system exploits the acidic environment of the lysosomes to quantify internalization of the labelled antibody. As Fabfluor labelled HDIT101 or HDIT102(H4) reside in the neutral extracellular solution (pH 7.4), they interact with cell surface-expressed gB inducing internalization. Once in the endo-lysosomal pathway, the antigen-antibody complex enters an acidic environment (pH 4.5-5.5) and a substantial increase in fluorescence was observed (FIG. 49B). This was not the case for an isotype control antibody labelled with pH-sensitive dye. The assay was repeated with HSV-1F infected Vero cells in the exact same way, the only difference was that Vero cells were infected with MOI 5 of HSV-1F 20 hours before adding the labelled test antibodies (FIG. 49C). Importantly, binding of gB expressed on the cell surface of HSV-infected cells also let to the internalization. This observation can fully explain the ability of HDIT102(H4) to mediated cell-to-cell spread. In a natural infection HSV gB cycles to the cell surface and back to the endoplasmic reticulum-Golgi compartment before it gets incorporated into the viral membrane. HDIT102(H4) binding to cell-surface exposed gB and subsequent internalization of the gB-bound HDIT102(H4) blocks cell-to-cell spread by neutralizing the virus already at the intracellular compartment in which gB gets incorporated into the viral membrane. This may also lead to enhanced aggregation and lysosomal or proteasomal degradation, followed by enhanced MHC-presentation of viral peptides.

### Example 22: HDIT102(H4) Induces Phagocytosis in MDM Type 1 or Type 2 and in MDDCs Individually and in Combination with HDIT101

To answer the question of whether a combination of HDIT101 with HDIT102(H4) enhances the uptake of HSV-immune complexes into primary antigen-presenting cells (APCs) monocytes were isolated from PBMCs by MACS CD14+ MicroBead separation (Miltenyi Biotec) according to the manufacturer instruction and differentiated for one week into by adding GM-CSF (80 ng/ml) to generate monocyte-derived macrophages (MDM) of type 1, M-CSF (50 ng/ml) to generate MDMs of type 2, or GM-CSF (80 ng/ml)+interleukin 4 (IL-4) (20 ng/ml) to generate monocyte-derived dendritic cells (MDDC). The differentiated cells were then exposed to HSV-1 labeled with a pH-sensitive dye (IncuCyte pHrodo Orange Cell Labeling Dye, Sartorius) at an MOI of 10 in the presence or absence of HDIT101 and/or HDIT102(H4) antibody at a total concentration of 150 μg/ml or Acyclovir (50 μg/ml). Three technical replicates were then monitored using an Incucyte system (Sartorius) at intervals of one hour. The amount of virus taken up as measured by dye fluorescence was normalized to the cell count in the imaged area.

HDIT102(H4) induced phagocytosis of HSV particles in MDM type 1 (FIG. 50A) or type 2 (FIG. 50B), as well as in MDDCs (FIG. 50C). The efficiency of ADCP was similar for HDIT101 and HDIT102(H4). As expected, a combination of HDIT101 and HDIT102(H4) showed efficient ADCP in all tested primary cell types (FIG. 50A-C). In all cases, HSV-1 alone or in combination with Acyclovir did lead to reduced uptake of and a decrease of antigen-positive APCs over time, while APCs that had taken up HSV as an immune complex via ADCP showed prolonged persistence of Ag-positive APCs. This demonstrates not only that HDIT102(H4) leads to better APC-induced phagocytosis of HSV, but also indicates that APC that have taken up HSV-HDIT101 and/or HDIT102(H4) immune complexes with HSV may be more efficient in activating T cells.

### Example 23: Activation of Autologous T Cells Upon HDIT102(H4)-Induced Phagocytosis of Virus by MDMs Individually and in Combination with HDIT101

Autologous T-cell activation was measured after exposure of HSV-HDIT101 and/or HDIT102(H4) immune complexes to MDMs from two independent HSV-seropositive healthy donors. MDMs were pre-incubated for 24 hours with HSV-1F (MOI 10) in the presence or absence of neutralizing amounts of HDIT102(H4) and/or HDIT101. Analysis was done by flow cytometry using BV785-labeled anti-CD69 IgG (Biolegend) as T-cell activation marker, APC-labeled anti-CD4 (Biolegend) and BV605-labeled anti-CD8 (Biolegend) 24 hours after addition of T-cells to the pre-incubated MDMs. Percentage of CD69-positive cells as well as mean fluorescent intensity (CD69 MFI) were measured and analysed separately for CD4+ as well as CD8+ T-cells. The data indicate that when MDMs were preincubated with HSV-immune complexes containing HDIT101, HDIT102(H4) or the combination HDIT101+HDIT102(H4), T-cells were substantially more activated (FIG. 51A-D). Activation was seen for both, CD4-positive as well as CD8-positive cells. In the absence of anti-HSV antibodies, or in the absence of virus, T-cell activation was not substantially induced as compared to untreated controls (FIG. 51A-D).

### Example 24: Comparison of HDIT101-Fab Bound HSV-2 gB Cryo-EM Structure at a Resolution of 3.45 Å with the HSV-1 gB Structure

To analyse possible differences in the structural binding of HDIT101 Fab to HSV-2 gB, recombinant HSV-2G gB ectodomain was generated as described for HSV-1 gB and Cryo-EM analysis was done of the HDIT101 Fab bound complex. The structural complex was solved at a resolution of 3.45 Å.

For Cryo-EM grid preparation and data collection, the following steps were performed, recombinant HSV-2G gB ectodomain and HDIT101 Fab were mixed in a ratio of 1 to 3.5. A 4 μl aliquot of the mixture was adsorbed onto glow-discharged Quantifoil Au-R2/1-300 mesh holey carbon-coated grids (Quantifoil, Germany), blotted with Whatman 1 filter paper and vitrified into liquid ethane at −180° C. using a Leica EM GP2 plunger (Leica microsystems, Austria) operated at 10° C. and 85% humidity. Data of HSV-2 gB and HDIT101-Fab complex was acquired on a Glacios TEM (ThermoFisher) operated at 200 kV and equipped with a Quantum K3 direct electron detector (Gatan). Micrograph movies of 40 frames were recorded in counting mode at a magnification of 45,000× (pixel size 0.878 Å) with a dose of 1.325 e⁻/Å²/frame, resulting in a total accumulated dose on the specimen level of approximately 53 e⁻/Å² per exposure. For data processing and model building, all image processing steps were performed with Relion v4.0 (Kimanius, Dong et al., 2021, Biochem J, Vol. 478 (24)). Dose weighting and motion correction of dose-fractionated and gain-corrected movies were performed using Relion's implementation of the UCSF motioncor2 program. Contrast transfer function (CTF) parameters were estimated using ctffind 4.1.14 (Rohou and Grigorieff, 2015, J Struct Biol, Vol. 192 (2)). Micrographs displaying strong drift, astigmatism greater than 1000 Å and maximum CTF resolution worse than 8 Å were excluded from further processing. A total of 6 million particles were picked using the Laplacian-of-Gaussian (LoG) filter in Relion 4.0 (Kimanius, Dong et al., 2021, Biochem J, Vol. 478 (24)). The particle dataset was cleaned through five rounds of reference-free 2D classification resulting in 915′372 particles. Relion's Stochastic Gradient Desecnt (SGD) algorithm was used to generate a de novo 3D initial model from the 2D particles. The particle dataset was further cleaned through three rounds of unsupervised 3D classification. The remaining 234′096 particles were subjected to Bayesian particle polishing, CTF and aberration refinement, and a final high-resolution 3D refinement, which resulted in a final map with an overall resolution of 3.45 Å according to the gold standard Fourier shell correlation (FSC) at FSC=0.143. The HSV-1 gB X-ray structure (PDB-ID: 2GUM) was manually mutated according to a sequence alignment with sequence QAU10436.1 (UniProt entry A0A410TI43) and placed into the final map using coot (Casanal, Lohkamp et al., 2020, Protein Sci, Vol. 29 (4)). For the HDIT101 Fab, the crystal structure of a humanized recombinant Fab fragment of a murine antibody (PDB-ID 3AAZ) was mutated in coot (Casanal, Lohkamp et al., 2020, Protein Sci, Vol. 29 (4)) based on a sequence alignment generated by Needle EMBOSS (Rice, Longden et al., 2000, Trends Genet, Vol. 16 (6)). Three HDIT101 Fabs were placed into the final map using coot (Casanal, Lohkamp et al., 2020, Protein Sci, Vol. 29 (4)). Molrep of the CCP-EM software suite v1.6 (Nicholls, Tykac et al., 2018, Acta Crystallogr D Struct Biol, Vol. 74 (Pt 6)) was used for the initial fitting of HSV-1 gB and the three HDIT101 Fabs into the final map. The final protein model was obtained by several iterations of manual model building in coot (Casanal, Lohkamp et al., 2020, Protein Sci, Vol. 29 (4)), Refmac-Servalcat refinement and model validation in the CCP-EM software suite v1.6 (Nicholls, Tykac et al., 2018, Acta Crystallogr D Struct Biol, Vol. 74 (Pt 6)). Data collection, refinement and validation statistics are summarized in Table 3 and the data processing workflow in (FIG. 52A). Overall structures of HDIT101 Fab bound to HSV-1F versus HSV-2G gB ectodomains did not reveal any differences (FIG. 52B-G). When HDIT102(H4)-specific binding residues in gB were superimposed for HSV-1 and HSV-2 gB derived from the Cryo-EM structures with bound HDIT101 Fab, no difference in the orientation of the amino acids was observed (FIG. 52H). The same is true for HDIT101-specific binding residues in gB (FIG. 52I). The data indicate that there are no major differences observed on the structural level in the HDIT101 or HDIT102(H4) binding residues between recombinant HSV-1 and HSV-2 gB.

TABLE 5
Cryo-EM data collection HDIT101 Fab on HSV-2G
gB, refinement and validation statistics.

| Data collection | |
|---|---|
| Microscope | Glacios |
| Voltage (kV) | 200 |
| Magnification | 45′000 |
| Pixel size (Å) | 0.878 |
| Collection software | Serial EM |
| Defocus range (μm) | −1.0 to −2.0 |
| Movies recorded | 9′700 |
| Frames per movie | 40 |
| Exposure rate (e⁻/Å2/frame) | 1.325 |
| Exposure time (s) | 3.2 |
| Cumulative exposure (e⁻/Å2) | 53 |
| Refinement | |
| Software | Relion v4.0 |
| Initial particle images (no.) | 6′289′845 |
| Final particle images (no.) | 234′096 |
| Symmetry imposed | C₁ |
| 0.143 FSC half map (Å) | 3.45 |
| Map sharpening B-factor | −79.24 |
| (Å²) | |
| Model building and validation | |
| Software | CCP-EM v1.6 |
| | coot v0.9.8 |
| MolProbity score | 1.63 |
| All-atom clashscore | 10.74 |
| Rotamer | |
| Favored (%) | 98.08 |
| Outliers (%) | 0 |
| Ramachandran | |
| Favored (%) | 97.64 |
| Outliers (%) | 0 |
| CaBLAM outliers (%) | 1.1 |
| Cβ outliers (%) | 0 |
| FSC 0.5 average (%) | 79.99 |

### Example 25: Generation of Bispecific Tandem scFv-Fc Constructs with HDIT101 and HDIT102(H4) Targeting Domains as Well as Trispecific scFv Constructs with HDIT101, HDIT102(H4) and HDIT103 Targeting Domains

To combine the specificities and improved characteristics (synergistic effects) seen in the combination therapy of HDIT101 and HDIT102(H4) in the mouse model, as well as to generate an antiviral molecule to which resistance development is more difficult, a bispecific molecule comprising the HDIT101 and HDIT102(H4) target binding moieties was designed. In tandem combined scFv domains of HDIT101 and HDIT102(H4) separated by a linker and carboxy-terminally fused to a human IgG1 Fc (SEQ ID NO:27) were encoded in an expression vector (FIG. 53A). In parallel, monospecific HDIT101scFv-Fc and HDIT102(H4)scFv-Fc constructs were generated (SEQ ID NO:28 and SEQ ID NO:29, respectively).

In addition, a trispecific construct was generated, in which a third gB-targeting scFv domain derived from HDIT103 was encoded in tandem (SEQ ID NO:30). HDIT103 targets a different site to HDIT101 or HDIT102(H4) in gB by interaction with the VH (SEQ ID NO:37) and VL (SEQ ID NO:38) domains that comprise the CDR sequences HC CDR1 ((SEQ ID NO:31), HC CDR2 (SEQ ID NO:32), HC CDR3 (SEQ ID NO:33), LC CDR1 (SEQ ID NO:34), LC CDR2 (SEQ ID NO:35) and LC CDR3 (SEQ ID NO:36).

These vectors were transfected into HEK293T-E6 cells to produce proteins. Concentrations in supernatants were determined using bio-layer interferometry analyses with Octet. Binding affinities to recombinant gB were determined by bio-layer interferometry analysis (Octet) demonstrating for HDIT102(H4)scFv-Fc as well as the bi- and tri-specific constructs strong binding with a measurable dissociation rate (kdis), which was approximately 10-fold lower than kdis for HDIT101scFv-Fc and an approximately eight-fold lower Kd (FIG. 53B, C). When neutralization capacity was analysed for the bi- and tri-specific tandem-scFv-Fc proteins towards cell-free HSV-1F infecting Vero cells, the concentration to neutralize 50% of 100 TCID50 HSV-1 was determined with 6.4 nM and 0.9 nM, respectively (FIG. 53D), hence demonstrating improved neutralization efficiency of the bi- or tri-specific constructs.

### Example 26: The Antibody of the Present Invention Exhibits Surprising Properties Over Antibodies Disclosed in the Prior Art

The antibody of the present invention, i.e., the HDIT102 (H4) antibody, is a fully human IgG1 antibody, whose variable domains were isolated from an scFv phage display library utilizing HSV-experienced B cell repertoires, through targeted selection against gB of HSV-1. HDIT102 (H4) has unique properties for therapeutic use, as it potently neutralizes HSV-1 and HSV-2 without activating ADCC or CDC, thus avoiding non-specific toxicities in patients.

These properties are unique for the antibodies of the present invention, in particular, vis-à-vis those described in WO 2023/003951. WO 2023/003951 recently described some anti-HSV gB antibodies with neutralizing activity isolated from seropositive human subjects (see, e.g., FIG. 5A and FIG. 5B of WO 2023/003951).

The anti-gB antibodies of WO 2023/003951 were isolated from reactive memory B-cells from HSV-2 seropositve human subjects. This document describes that its antibodies had neutralizing capability but, in contrast to HDIT102 (H4), exhibit the activity of inducing antibody-dependent cellular cytotoxicity (ADCC). These anti-gB antibodies were shown to have strong (mAbs 59, 60, 79,116,117), moderate (mAbs 105, 108, 121), and weak (mAbs 97, 107, 11) neutralizing properties. WO 2023/003951 shows that the strongly and moderately neutralizing gB mAbs compete with each other and thus recognize similar or overlapping epitopes (see [00213] and FIG. 7 of WO 2023/003951).

In the following, comparative experiments are described wherein the antibody of the present invention (i.e., the HDIT102 (H4) antibody) is compared with the above-mentioned anti-gB antibodies of WO 2023/003951.

In the comparative experiments, representative antibodies of the neutralizing antibodies of WO 2023/003951 have been chosen. The above “59” and “121” antibody have not been tested because the “121” antibody is the weakest candidate of the above moderate neutralizing antibodies (see the IC50 value of WO 2023/003951's FIG. 5B). Moreover, the “59” antibody has a similar IC50 value as the “79” antibody.

In a first assay, it has been tested whether the antibodies disclosed in WO 2023/003951 bind to different antibodies as the antibody of the present invention.

As outlined in the following, it has been shown that the strong neutralizing antibody HDIT102 (H4) clearly binds a different epitope than the antibodies with strong and moderate neutralizing properties described in WO 2023/003951 as shown by competitive binding assays using enzyme-linked immunosorbent assay (ELISA).

For ELISA the mAbs were used as purified scFv-Fc and added in 3-fold serial dilutions (3 nM-0,037 nM) either in the absence or presence of 8- to 675-fold molar excess of Fab HDIT102 (H4) to HSV-2 gB coated microtiter plates. After 1.5 h at room temperature wells were washed and subsequently incubated for 1 h with anti-human Fc-HRP conjugate (1:10,000). After a final wash, bound scFv-Fc was detected using TMB chromogenic substrate and signal was detected using a Tecan plate reader. As shown in FIG. 54, the Fab HDIT102 displaces the scFv-Fc HDIT102 from its binding with increasing molar excess, as both compete for the same epitope. In contrast HDIT102 did not compete the antibodies recognizing similar or overlapping epitopes from WO 2023/003951, exemplarily shown for the strong neutralizing antibodies (60, 79, 116, 117) and the moderate neutralizing antibodies (105, 108), even when present at 675-fold molar excess (FIG. 54).

In a second assay, the binding characteristics of the antibody of the present invention vis-h-vis the binding characteristics of the above-cited antibodies of WO 2023/003951 have been tested.

More specifically, the binding characteristics of HDIT102 and of antibodies described in WO 2023/003951 were compared using biolayer-interferometry (Octet, Sartorius). In contrast to the antibodies of WO 2023/003951, HDIT102 (H4) is characterized by unique binding properties, as HDIT102 IgG binds very quickly to the epitope and remains bound. The association rate (ka) of the bivalent HDIT102 is 7.0×10⁵ M⁻¹s⁻¹ (FIG. 1B). Accordingly, the antibody of the present invention has superior properties because all the antibodies of WO 2023/003951 showed slower association rates (FIGS. 55A and 55B). As a result thereof, it can be concluded that also the dissociation constant KD of the antibody of the present invention is superior over the prior art antibodies because the dissociation constant (KD) is determined in terms of the association rate (ka) and the dissociation rate (kdis) of the binding interaction and is mathematically given by: KD=kdis/ka. Thus, the KD value provides an indication of the binding affinity by comparing how quickly the complex forms versus how quickly it dissociates. A lower KD (resulting from a high ka and/or low kdis) indicates a higher affinity, whereas a higher KD (resulting from a low ka and/or high kdis) indicates a lower affinity. The higher association rate of HDIT102 as compared to antibodies 60, 79, 105, 108 and 116 results therefore in a superior affinity of HDIT102 although the dissociation rates are similar. Antibody 117 exhibits a short binding duration with the antigen, as indicated by the high kdis, resulting in a low affinity (KD 1.41×10⁻¹⁰ M).

These superior binding properties of HDIT102 to HSV gB translate into improved therapeutic properties. Cell-to-cell spread inhibition is likely the main way of viral spread in vivo and it was proposed for HSV-1 that individuals with higher levels of cell-to-cell spread inhibiting antibodies may have fewer orolabial recurrences, suggesting that this characteristic may be advantageous when developing a monoclonal antibody therapy against HSV. Cell-to-cell spread of HSV has been considered as viral immune evasion mechanism. HSV gB and other HSV glycoproteins have been proposed to be transported to the cell membrane and re-imported via endosomes in a Rab-dependent way before becoming incorporated into the viral membrane within the trans-Golgi network. The data provided suggest that HDIT102 binds rapidly to gB exposed at the cell-surface of infected cells and is co-internalized and transported on the natural gB trafficking way, hence interact with gB before becoming incorporated into the viral membrane, blocking spreading of newly produced progeny viruses.

### Example 27: Neutralization of Cell-Free HSV-1 and HSV-2 by HDIT102(H4), HDIT101 and a Combination Thereof

As a combination therapy of HDIT101 with HDIT102(H4) demonstrated synergistic effects in vivo (FIG. 47A) we examined the antiviral activity of HDIT102 in combination with HDIT101 towards cell-free virus in vitro. Neutralization capacities of different antibody dilutions were determined for HSV-1F or HSV-2G. As control, Vero cells were infected without prior incubation of virus with antibody. The highest antibody concentration preventing the viral cytopathic effect (CPE) to 50% relative to the control were determined three days after infection and considered the endpoint. The combination of both antibodies in a 1:1 ratio maintained neutralization efficiency comparable to that of a single antibody treatment. The combination prevented virus-induced CPE of HSV-1 by 50% at a concentration of 18 nM and of HSV-2 at a concentration of 9.3 nM (FIG. 56).

### REFERENCES

1. James, C., et al., Herpes simplex virus: global infection prevalence and incidence estimates, 2016. Bull World Health Organ, 2020. 98(5): p. 315-329.
2. Zhu, S. and A. Viejo-Borbolla, Pathogenesis and virulence of herpes simplex virus. Virulence, 2021. 12(1): p. 2670-2702.
3. Looker, K. J., et al., Effect of HSV-2 infection on subsequent HIV acquisition: an updated systematic review and meta-analysis. Lancet Infect Dis, 2017. 17(12): p. 1303-1316.
4. Looker, K. J., et al., First estimates of the global and regional incidence of neonatal herpes infection. Lancet Glob Health, 2017. 5(3): p. e300-e309.
5. Spruance, S. L., et al., Acyclovir cream for treatment of herpes simplex labialis: results of two randomized, double-blind, vehicle-controlled, multicenter clinical trials. Antimicrob Agents Chemother, 2002. 46(7): p. 2238-43.
6. Brown, Z. A., et al., Neonatal herpes simplex virus infection in relation to asymptomatic maternal infection at the time of labor. N Engl J Med, 1991. 324(18): p. 1247-52.
7. Belshe, R. B., et al., Efficacy results of a trial of a herpes simplex vaccine. N Engl J Med, 2012. 366(1): p. 34-43.
8. Belshe, R. B., et al., Neutralizing Antibody Kinetics and Immune Protection Against Herpes Simplex Virus 1 Genital Disease in Vaccinated Women. J Infect Dis, 2023. 227(4): p. 522-527.
9. Corey, L., et al., Recombinant glycoprotein vaccine for the prevention of genital HSV-2 infection: two randomized controlled trials. Chiron HSV Vaccine Study Group. JAMA, 1999. 282(4): p. 331-40.
10. Bernstein, D. I., et al., Therapeutic Vaccine for Genital Herpes Simplex Virus-2 Infection: Findings From a Randomized Trial. J Infect Dis, 2017. 215(6): p. 856-864.
11. Van Wagoner, N., et al., Effects of Different Doses of GEN-003, a Therapeutic Vaccine for Genital Herpes Simplex Virus-2, on Viral Shedding and Lesions: Results of a Randomized Placebo-Controlled Trial. J Infect Dis, 2018. 218(12): p. 1890-1899.
12. Flechtner, J. B., et al., Immune responses elicited by the GEN-003 candidate HSV-2 therapeutic vaccine in a randomized controlled dose-ranging phase 1/2a trial. Vaccine, 2016. 34(44): p. 5314-5320.
13. Bernstein, D. I., et al., Therapeutic HSV-2 vaccine decreases recurrent virus shedding and recurrent genital herpes disease. Vaccine, 2019. 37(26): p. 3443-3450.
14. Dropulic, L. K., et al., A Randomized, Double-Blinded, Placebo-Controlled, Phase 1 Study of a Replication-Defective Herpes Simplex Virus (HSV) Type 2 Vaccine, HSV529, in Adults With or Without HSV Infection. J Infect Dis, 2019. 220(6): p. 990-1000.
15. Wang, K., et al., Serum and Cervicovaginal Fluid Antibody Profiling in Herpes Simplex Virus-Seronegative Recipients of the HSV529 Vaccine. J Infect Dis, 2021. 224(9): p. 1509-1519.
16. Mikloska, Z., P. P. Sanna, and A. L. Cunningham, Neutralizing antibodies inhibit axonal spread of herpes simplex virus type 1 to epidermal cells in vitro. J Virol, 1999. 73(7): p. 5934-44.
17. Backes, I. M., D. A. Leib, and M. E. Ackerman, Monoclonal antibody therapy of herpes simplex virus: An opportunity to decrease congenital and perinatal infections. Front Immunol, 2022. 13: p. 959603.
18. Petro, C. D., et al., HSV-2 DeltagD elicits FcgammaR-effector antibodies that protect against clinical isolates. JCI Insight, 2016. 1(12).
19. Burn Aschner, C., et al., HVEM signaling promotes protective antibody-dependent cellular cytotoxicity (ADCC) vaccine responses to herpes simplex viruses. Sci Immunol, 2020. 5(50).
20. Lee, C. C., et al., Structural basis for the antibody neutralization of herpes simplex virus. Acta Crystallogr D Biol Crystallogr, 2013. 69(Pt 10): p. 1935-45.
21. Sanna, P. P., et al., Protection of nude mice by passive immunization with a type-common human recombinant monoclonal antibody against HSV. Virology, 1996. 215(1): p. 101-6.
22. Politch, J. A., et al., Safety, acceptability, and pharmacokinetics of a monoclonal antibody-based vaginal multipurpose prevention film (MB66): A Phase I randomized trial. PLoS Med, 2021. 18(2): p. e1003495.
23. Zeitlin, L., et al., Topically applied human recombinant monoclonal IgG1 antibody and its Fab and F(ab′)2 fragments protect mice from vaginal transmission of HSV-2. Virology, 1996. 225(1): p. 213-5.
24. Eis-Hubinger, A. M., K. Mohr, and K. E. Schneweis, Different mechanisms of protection by monoclonal and polyclonal antibodies during the course of herpes simplex virus infection. Intervirology, 1991. 32(6): p. 351-60.
25. Eis-Hubinger, A. M., D. S. Schmidt, and K. E. Schneweis, Anti-glycoprotein B monoclonal antibody protects T cell-depleted mice against herpes simplex virus infection by inhibition of virus replication at the inoculated mucous membranes. J Gen Virol, 1993. 74 (Pt 3): p. 379-85.
26. Krawczyk, A., et al., Impact of valency of a glycoprotein B-specific monoclonal antibody on neutralization of herpes simplex virus. J Virol, 2011. 85(4): p. 1793-803.
27. Krawczyk, A., et al., Overcoming drug-resistant herpes simplex virus (HSV) infection by a humanized antibody. Proc Natl Acad Sci USA, 2013. 110(17): p. 6760-5.
28. Krawczyk, A., et al., Prevention of herpes simplex virus induced stromal keratitis by a glycoprotein B-specific monoclonal antibody. PLoS One, 2015. 10(1): p. e0116800.
29. Silke Heilingloh, C., et al., Herpes Simplex Virus Type 2 Is More Difficult to Neutralize by Antibodies Than Herpes Simplex Virus Type 1. Vaccines (Basel), 2020. 8(3).
30. Alt, M., et al., Cell-to-cell spread inhibiting antibodies constitute a correlate of protection against herpes simplex virus type 1 reactivations: A retrospective study. Front Immunol, 2023. 14: p. 1143870.
31. Arkatkar, T., et al., Memory T cells possess an innate-like function in local protection from mucosal infection. J Clin Invest, 2023.
32. Blank, A., et al., First-in-human, randomized, double-blind, placebo-controlled, dose escalation trial of the anti-herpes simplex virus monoclonal antibody HDIT101 in healthy volunteers. Clin Transl Sci, 2022. 15(10): p. 2366-2377.
33. Kovacech, B., et al., Monoclonal antibodies targeting two immunodominant epitopes on the Spike protein neutralize emerging SARS-CoV-2 variants of concern. EBioMedicine, 2022. 76: p. 103818.
34. Baum, A., et al., Antibody cocktail to SARS-CoV-2 spike protein prevents rapid mutational escape seen with individual antibodies. Science, 2020. 369(6506): p. 1014-1018.
35. Pal, P., et al., Development of a highly protective combination monoclonal antibody therapy against Chikungunya virus. PLoS Pathog, 2013. 9(4): p. e1003312.
36. Mankowski, M. C., et al., Synergistic anti-HCV broadly neutralizing human monoclonal antibodies with independent mechanisms. Proc Natl Acad Sci USA, 2018. 115(1): p. E82-E91.
37. Julg, B., et al., Safety and antiviral activity of triple combination broadly neutralizing monoclonal antibody therapy against HIV-1: a phase 1 clinical trial. Nat Med, 2022. 28(6): p. 1288-1296.
38. Sneller, M. C., et al., Combination anti-HIV antibodies provide sustained virological suppression. Nature, 2022. 606(7913): p. 375-381.
39. Corraliza-Gorjon, I., et al., New Strategies Using Antibody Combinations to Increase Cancer Treatment Effectiveness. Front Immunol, 2017. 8: p. 1804.

1. An anti-HSV antibody or an antigen-binding fragment thereof binding to the glycoprotein B (gB) of HSV-1 and/or HSV-2, wherein said antibody comprises:
the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6;
wherein said antibody or antigen-binding fragment has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹,
preferably, wherein said antibody or antigen-binding fragment thereof is capable of neutralizing HSV.

2. The anti-HSV antibody or the antigen-binding fragment thereof according to claim 1, wherein said antibody comprises the following framework regions:
an amino acid sequence with at least 70% sequence identity to each one of the amino acid residues shown in positions 1 to 25 (V_{H}FR1), 36 to 49 (V_{H}FR2), 67 to 98 (V_{H}FR3), 112 to 122 (V_{H}FR4) of SEQ ID NO: 7, 1 to 22 (V_{L}FR1), 34 to 48 (V_{L}FR2), 56 to 87 (V_{L}FR3), and 97 to 106 (V_{L}FR4) of SEQ ID NO: 8.

3. The anti-HSV antibody or the antigen-binding fragment thereof according to claim 1, wherein said antibody comprises the VH of SEQ ID NO:7 and the VL of SEQ ID NO:8.

4. The anti-HSV antibody or antigen-binding fragment thereof which recognizes the same epitope as said antibody according to any one of claim 1, wherein
(a) said epitope is located at the contact amino acid residues D199, A203, K204, Y303, R304, K320, Q321, V322, D323, Y326, R335 and T337 of glycoprotein B of HSV-1 strain F and contact amino acids residues D191, A195, K196, Y295, R296, K312, Q313, V314, D315, Y318, R327 and T329 of glycoprotein B of HSV-2 strain G, respectively;
(b) preferably, wherein said epitope consists of the contact amino acid residues D199, A203, K204, Y303, R304, K320, Q321, V322, D323, Y326, R335 and T337 of glycoprotein B of HSV-1 strain F (SEQ ID NO:9) and of the contact amino acids residues D191, A195, K196, Y295, R296, K312, Q313, V314, D315, Y318, R327 and T329 of glycoprotein B of HSV-2 strain G (SEQ ID NO:40), respectively; and/or
(c) wherein said epitope of (a) or (b) is determined by cryo-electron microscopy (Cryo-EM).

5. (canceled)

6. The anti-HSV antibody or the antigen-binding fragment thereof according to claim 1, wherein said anti-HSV antibody or antigen-binding fragment thereof:
(a) is a humanized or fully human antibody,
(b) is a full-length antibody;
(c) is a human IgG1, an IgG2, an IaG2a, an IgG2b, an IgA1, an IgGA2, an IgG3, an IgG4, an IgA, an IgM, an IgD or an IgE antibody;
(d) is a human F(ab)-, Fab′-SH-, Fv-, Fab′-, or a F(ab′)2-fragment;
(e) has a dissociation constant Kd of at most 10 nM, preferably at most 8 nM, more preferably at most 4 nM, even more preferably at most 2 nM, at most 1 nM, at most 0.8 nM, at most 0.4 nM, at most 0.2 nM, at most 0.1 nM, at most 0.09 nM, at most 0.08 nM, at most 0.07 nM, at most 0.06 nM, al most 0.05 nM, at most 0.04 nM and most preferably at most 0.03 nM; and/or
(f) is in a concentration of at most 20 nM, preferably of at most 16 nM, more preferably of at most 12 nM, of at most 10 nM, of at most 8 nM, of at most 6 nM, and most preferably of at most 4 nM is capable of neutralising a defined amount of HSV of 100 TCID50;
(g) is capable of inhibiting the spreading of HSV from an infected cell to an adjacent second non-infected cell (cell-to-cell spread); and/or
(h) exerts its antiviral or neutralizing activity independent from antibody-dependent cellular cytotoxicity (ADCC) and/or complement-dependent cytotoxicity (CDC), preferably, wherein said antibody is capable of inhibiting cell-to-cell spread independent from antibody-dependent cellular cytotoxicity (ADCC) and/or complement-dependent cytotoxicity (CDC).

7. (canceled)

8. (canceled)

9. (canceled)

10. (canceled)

11. (canceled)

12. (canceled)

13. (canceled)

14. The anti-HSV antibody or the antigen-binding fragment thereof according to claim 1, wherein said antibody is conjugated to an effector moiety, a therapeutic moiety, or a detectable label.

15. The anti-HSV antibody or an antigen-binding fragment thereof of claim 1 combined with a second anti-HSV antibody or an antigen-binding fragment thereof recognizing/binding to gB of the HSV-1 and/or HSV-2 (“second gB antibody”, wherein said second gB antibody comprises:
the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO: 16;
wherein said second gB antibody has a dissociation constant Kd of at most 40 nM, preferably at most 30 nM, more preferably at most 20 nM, even more preferably at most 15 nM, at most 13 nM and at most 10 nM.

16. The anti-HSV antibody or the antigen-binding fragment thereof according to claim 15, wherein said second gB antibody comprises the following framework regions:
an amino acid sequence with at least 70% sequence identity to the amino acid residues shown in positions 1 to 30 (V_{H}FR1), 38 to 51 (V_{H}FR2), 68 to 99 (V_{H}FR3), and 112 to 122 (V_{H}FR4) of SEQ ID NO: 17, 1 to 23 (V_{L}FR1), 41 to 55 (V_{L}FR2), 63 to 94 (V_{L}FR3), and 104 to 114 (V_{L}FR4) of SEQ ID NO: 18.

17. The anti-HSV antibody or the antigen-binding fragment thereof according to claim 15, wherein said second gB antibody comprises the V_{H} of SEQ ID NO:19 and the V_{L} of SEQ ID NO:20.

18. The anti-HSV antibody or the antigen-binding fragment thereof according to claim 15, wherein said second gB antibody is an antibody that recognizes the same epitope as said second gB antibody, wherein (a) said epitope is located at the amino acids Y301-E305 and H308, K320, D323, Y326, P339, T341, W356 and P358 of glycoprotein B of HSV-1 and corresponding sites Y293-E297 and H300, K312, D315, Y318, P331, T333, W348 and P350 of glycoprotein B of HSV-2;
(b) preferably, wherein said epitope consists of the contact amino acid residues Y301-E305 and H308, K320, D323, Y326, P339, T341, W356 and P358 of glycoprotein B of HSV-1 strain F (SEQ ID NO:9) and of the contact amino acids residues Y293-E297 and H300, K312, D315, Y318, P331, T333, W348 and P350 of glycoprotein B of HSV-2 strain G (SEQ ID NO:40), respectively; and/or
(c) wherein said epitope of (a) or (b) is determined by cryo-electron microscopy (Cryo-EM).

19. (canceled)

20. The anti-HSV antibody or the antigen-binding fragment thereof according to claim 15, wherein said second gB antibody or antigen-binding fragment thereof:
(a) is a humanized or fully human antibody;
(b) is a full-length antibody;
(c) is a human IgG1, an IgG2, an IgG2a, an IgG2b, an IgA1, an IgGA2, an IgG3, an IgG4, an IgA, an IgM, an IgD or an IgE antibody;
(d) is a F(ab)-, Fab′-SH-, Fv-, Fab′-, or a F(ab′)2-fragment,
(e) has a dissociation constant Kd of at most 10 nM, preferably at most 8 nM, more preferably at most 4 nM, even more preferably at most 2 nM, at most 1 nM, at most 0.8 nM, al most 0.4 nM, at most 0.2 nM, at most 0.1 nM, at most 0.09 nM, at most 0.08 nM, at most 0.07 nM, at most 0.06 nM, at most 0.05 nM, at most 0.04 nM and most preferably at most 0.03 nM:
(f) is in a concentration of at most 20 nM, preferably of at most 16 nM, more preferably of al most 12 nM, of at most 10 nM, of at most 8 nM, of al most 6 nM, and most preferably of at most 4 nM is capable of neutralising a defined amount of HSV of 100 TCID50;
(q) is capable of inhibiting the spreading of HSV from an infected cell to an adjacent second non-infected cell (cell-to-cell spread); and/or
(h) exerts its antiviral or neutralizing activity independent from antibody-dependent cellular cytotoxicity (ADCC) and/or complement-dependent cytotoxicity (CDC) preferably, wherein said antibody is capable of inhibiting cell-to-cell spread independent from antibody-dependent cellular cytotoxicity (ADCC) and/or complement-dependent cytotoxicity (CDC).

21. (canceled)

22. (canceled)

23. (canceled)

24. (canceled)

25. (canceled)

26. (canceled)

27. The anti-HSV antibody or the antigen-binding fragment thereof according to claim 15, wherein said second gB antibody is conjugated to an effector moiety, a therapeutic moiety, or a detectable label.

28. A pharmaceutical composition comprising an effective amount of the anti-HSV antibody or the antigen-binding fragment thereof according to claim 1 and at least one pharmaceutically acceptable excipient.

29. (canceled)

30. A method of treating a patient in need thereof, wherein said method comprises administering the anti-HSV antibody or the antigen-binding fragment thereof according to claim 1 either prophylactically or therapeutically to a patient, wherein said patient is suffering from Herpes simplex labialis, Herpes simplex genitalis, chronic or disseminated cutaneous herpes simplex infection, Herpes gladiatorum, Eczema herpeticum, Herpes keratoconjunctivitis, Herpes neonatorum, Alzheimer disease (AD), HSV pneumonia, Bell's palsy, Herpes esophagitis, Herpesviral encephalitis and Herpesviral meningitis, Herpetic sycosis, Herpes withlow, Herpes gingivostomatitis, presence of an oral herpes relapse or recidivism, presence of a genital herpes relapse or recidivism, eczema herpeticatum, herpes neonatorum, immune deficiency, immunocompromized patients, resistance against a virostatic agent, encephalitis, meningitis, meningoencephalitis, eye infections, and/or generalized HSV infections.

31. The method of claim 30, wherein said antibody is to be administered intravenously, topically, intradermally, subcutaneously, intra-cutaneously, intramuscular and/or intrathecal.

32. A bispecific antibody or an antigen-binding fragment thereof binding to the glycoprotein B (gB) of the HSV-1 and/or HSV-2 which comprises:
(A) a first binding domain comprising:
the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6; and
(B) a second binding domain comprising:
the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16;
wherein said bispecific antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹.

33. A trispecific antibody or an antigen-binding fragment thereof binding to the glycoprotein B (gB) of the HSV-1 and/or HSV-2 which comprises:
(A) a first binding domain comprising:
the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6; and
(B) a second binding domain comprising:
the complementarity determining regions V_{H}CDR1 comprising SEQ ID NO: 11, V_{H}CDR2 comprising SEQ ID NO: 12, V_{H}CDR3 comprising SEQ ID NO: 13, V_{L}CDR1 comprising SEQ ID NO: 14, V_{L}CDR2 comprising SEQ ID NO: 15, and V_{L}CDR3 comprising SEQ ID NO:16; and
(C) a third binding domain comprising:
the complementarity determining regions V_{H}CDR1 comprising SEQ ID NO: 31, V_{H}CDR2 comprising SEQ ID NO: 32, V_{H}CDR3 comprising SEQ ID NO: 33, V_{L}CDR1 comprising SEQ ID NO: 34, V_{L}CDR2 comprising SEQ ID NO: 35, and V_{L}CDR3 comprising SEQ ID NO:36;
wherein said trispecific antibody has a low dissociation rate k_{dis} of at most 5.0×10⁻⁴ s⁻¹, preferably at most 1.0×10⁻⁴ s⁻¹, at most 5.0×10⁻⁵ s⁻¹, and most preferably at most 2.9×10⁻⁵ s⁻¹;
and wherein said trispecific antibody in a concentration of at most 10 nM, preferably of at most 8 nM, more preferably of at most 6 nM, of at most 4 nM, of at most 2 nM, of at most 1 nM, of at most 0.9 nM, of at most 0.7 nM, and most preferably of at most 0.5 nM, is capable of neutralising a defined amount of HSV of 100 TCID50.

34. A method of treating a patient in need thereof, wherein said method comprises administering the anti-HSV antibody or the antigen-binding fragment thereof according to claim 15, wherein said anti-HSV antibody or the antigen-binding fragment thereof and said second gB antibody are administered either prophylactically or therapeutically to a patient, wherein said patient is suffering from Herpes simplex labialis, Herpes simplex genitalis, chronic or disseminated cutaneous herpes simplex infection, Herpes gladiatorum, Eczema herpeticum, Herpes keratoconjunctivitis, Herpes neonatorum, Alzheimer disease (AD), HSV pneumonia, Bell's palsy, Herpes esophagitis, Herpesviral encephalitis and Herpesviral meningitis, Herpetic sycosis, Herpes withlow, Herpes gingivostomatitis, presence of an oral herpes relapse or recidivism, presence of a genital herpes relapse or recidivism, eczema herpeticatum, herpes neonatorum, immune deficiency, immunocompromized patients, resistance against a virostatic agent, encephalitis, meningitis, meningoencephalitis, eye infections, and/or generalized HSV infections.

35. The method of claim 34, wherein said anti-HSV antibody or the antigen-binding fragment thereof and said second gB antibody are administered separately.

36. The method of claim 34, wherein the said anti-HSV antibody or the antigen-binding fragment thereof and said second gB antibody are administered as a bispecific or trispecific antibody.


SEQUENCE LISTING (40 sequences)

SEQ ID NO: 1 - protein, 10 aa, synthetic construct
  source 1..10: note = CDR1 HC
GHTFRTFDIN

SEQ ID NO: 2 - protein, 17 aa, synthetic construct
  source 1..17: note = CDR2 HC
WMSPNSGNTGYARQFQG

SEQ ID NO: 3 - protein, 13 aa, synthetic construct
  source 1..13: note = CDR3 HC
GPGSTGTTGSMDV

SEQ ID NO: 4 - protein, 11 aa, synthetic construct
  source 1..11: note = CDR1 LC
GGNNIGSKSVH

SEQ ID NO: 5 - protein, 7 aa, synthetic construct
  source 1..7: note = CDR2 LC
YDSDRPS

SEQ ID NO: 6 - protein, 9 aa, synthetic construct
  source 1..9: note = CDR3 LC
QVWDSGSVV

SEQ ID NO: 7 - protein, 452 aa, synthetic construct
  source 1..452: note = variable heavy chain-CH1-Hinge-CH2- CH3
EVQLVQSGAEVKTPGASVRVSCKASGHTFRTFDINWVRQAAGQGLEWMGWMSPNSGNTGYARQFQGRVTMTRNISANTAYMELRGLRFDDTAVYYCARGPGSTGTTGSMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 212 aa, synthetic construct
  source 1..212: note = variable light chain-Constant lambda3
QAGLTQPPSVSVAPGKTARISCGGNNIGSKSVHWYQQKPGQAPVLVIYYDSDRPSGIPERFSGSNSGNTATLTISRVEAGDEADYYCQVWDSGSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 903 aa, unidentified
  source 1..903: note = HSV1 strain F
MRQGAARGCRWFVVWALLGLTLGVLVASAAPSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 10 - protein, 904 aa, unidentified
  source 1..904: note = HSV1 strain KOS
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 11 - protein, 7 aa, synthetic construct
  source 1..7: note = CDR1 HC
TSGMSVG

SEQ ID NO: 12 - protein, 16 aa, synthetic construct
  source 1..16: note = CDR2 HC
HIWWNNDKYYKPALKS

SEQ ID NO: 13 - protein, 12 aa, synthetic construct
  source 1..12: note = CDR3 HC
IYYGYRPYAMDY

SEQ ID NO: 14 - protein, 16 aa, synthetic construct
  source 1..16: note = CDR1 LC
RSSQSIVHSNGNTYLE

SEQ ID NO: 15 - protein, 7 aa, synthetic construct
  source 1..7: note = CDR2 LC
KVSNRFS

SEQ ID NO: 16 - protein, 9 aa, synthetic construct
  source 1..9: note = CDR3 LC
FQGSHVPWS

SEQ ID NO: 17 - protein, 122 aa, synthetic construct
  source 1..122: note = human germline VH framework
  VARIANT 100..107: note = X can be any naturally occurring amino acid
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYYCARXXXXXXXXYFDYWGQGTLVTVSS

SEQ ID NO: 18 - protein, 114 aa, synthetic construct
  source 1..114: note = human germline VL framework
DIVMTQTPLSLPVTPGEPASISCRSSQSLLDSDDGNTYLEWYLQKPGQSPQLLIYTLSYRASGVPDRFSGSGSGTDFTLKISRVEAEDVGVYYCMQRIEFPWTFGQGTKVEIKR

SEQ ID NO: 19 - protein, 122 aa, synthetic construct
  source 1..122: note = VH of the humanized mAb hu2c antibody
QVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSS

SEQ ID NO: 20 - protein, 113 aa, synthetic construct
  source 1..113: note = VL of the humanized mAb hu2c antibody
DIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKVEIKR

SEQ ID NO: 21 - protein, 904 aa, unidentified
  source 1..904: note = HSV1 strain gC-39-R6
MHQGAPSWGRRWFVVWALLGLTLGVLVASAAPSSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQHHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 22 - protein, 904 aa, unidentified
  source 1..904: note = HSV2 strain HG52
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINTKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 23 - protein, 904 aa, unidentified
  source 1..904: note = HSV2 strain 333
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVTCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLGVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 24 - protein, 901 aa, unidentified
  source 1..901: note = HSV2 strain MMA
MRGGGLICALVVGALVAAVASAAPAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKAQATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 25 - DNA, 20 bp, synthetic construct
  source 1..20: mol_type = other DNA; note = primer
atcaacttcgactggccctt

SEQ ID NO: 26 - DNA, 20 bp, synthetic construct
  source 1..20: mol_type = other DNA; note = primer
ccgtacatgtcgatgttcac

SEQ ID NO: 27 - protein, 754 aa, synthetic construct
  source 1..754: note = HDIT101scFv-HDIT102(H4)scFv-Fc
MAQVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSSGGGGSGGGGSGGSAQDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKLEIKRTRGGGGSGGGGSGGGGSGGGGSMAEVQLVQSGAEVKTPGASVRVSCKASGHTFRTFDINWVRQAAGQGLEWMGWMSPNSGNTGYARQFQGRVTMTRNISANTAYMELRGLRFDDTAVYYCARGPGSTGTTGSMDVWGQGTTVTVSSGGGGSGGGGSGGSAQQAGLTQPPSVSVAPGKTARISCGGNNIGSKSVHWYQQKPGQAPVLVIYYDSDRPSGIPERFSGSNSGNTATLTISRVEAGDEADYYCQVWDSGSVLFGGGTKLTVLTRSGAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 487 aa, synthetic construct
  source 1..487: note = HDIT101scFv-Fc
MAQVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSSGGGGSGGGGSGGSAQDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKLEIKRTRSGAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 480 aa, synthetic construct
  source 1..480: note = HDIT102(H4)scFv-Fc
MAEVQLVQSGAEVKTPGASVRVSCKASGHTFRTFDINWVRQAAGQGLEWMGWMSPNSGNTGYARQFQGRVTMTRNISANTAYMELRGLRFDDTAVYYCARGPGSTGTTGSMDVWGQGTTVTVSSGGGGSGGGGSGGSAQQAGLTQPPSVSVAPGKTARISCGGNNIGSKSVHWYQQKPGQAPVLVIYYDSDRPSGIPERFSGSNSGNTATLTISRVEAGDEADYYCQVWDSGSVLFGGGTKLTVLTRSGAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 1024 aa, synthetic construct
  source 1..1024: note = HDIT101scFv-HDIT102(H4)scFv-HDIT103scFv-Fc
MAQVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSSGGGGSGGGGSGGSAQDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKLEIKRTRSGGGGSGGGGSGGGGSGGGGSMAEVQLVQSGAEVKTPGASVRVSCKASGHTFRTFDINWVRQAAGQGLEWMGWMSPNSGNTGYARQFQGRVTMTRNISANTAYMELRGLRFDDTAVYYCARGPGSTGTTGSMDVWGQGTTVTVSSGGGGSGGGGSGGSAQQAGLTQPPSVSVAPGKTARISCGGNNIGSKSVHWYQQKPGQAPVLVIYYDSDRPSGIPERFSGSNSGNTATLTISRVEAGDEADYYCQVWDSGSVLFGGGTKLTVLTRSGGGGSGGGGSGGGGSGGGGSMAEVQLLESGGGLVKPEGSLRLSCAASGFTFSSYAMNWVRQAPGKGLEWVSSISANGLRTYYADSVKGRFTISRDNSGNTLHLQMNSLRAEDTAIYYCAKHSRMATRDDPMDVWGQGTTVTVSSGGGGSGGGGSGGSAQEIVLTQSPGTLSLAPGEEATFSCRASESVSRSLAWYQQKPGQAPRLLIYDASSRATGIPDRFSGSVSETDFTLTISSLEPEDFAVYFCQQYGHSPWTFGQGTKVEIKTRSGAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 10 aa, synthetic construct
  source 1..10: note = HDIT103 CDR1 HC
GFTFSSYAMN

SEQ ID NO: 32 - protein, 17 aa, synthetic construct
  source 1..17: note = HDIT103 CDR2 HC
SISANGLRTYYADSVKG

SEQ ID NO: 33 - protein, 13 aa, synthetic construct
  source 1..13: note = HDIT103 CDR3 HC
HSRMATRDDPMDV

SEQ ID NO: 34 - protein, 11 aa, synthetic construct
  source 1..11: note = HDIT103 CDR1 LC
RASESVSRSLA

SEQ ID NO: 35 - protein, 7 aa, synthetic construct
  source 1..7: note = HDIT103 CDR2 LC
DASSRAT

SEQ ID NO: 36 - protein, 9 aa, synthetic construct
  source 1..9: note = HDIT103 CDR3 LC
QQYGHSPWT

SEQ ID NO: 37 - protein, 452 aa, synthetic construct
  source 1..452: note = HDIT103 variable heavy chain-CH1-Hinge-CH2- CH3
EVQLLESGGGLVKPEGSLRLSCAASGFTFSSYAMNWVRQAPGKGLEWVSSISANGLRTYYADSVKGRFTISRDNSGNTLHLQMNSLRAEDTAIYYCAKHSRMATRDDPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
  source 1..214: note = HDIT103 variable light chain-Constant kappa
EIVLTQSPGTLSLAPGEEATFSCRASESVSRSLAWYQQKPGQAPRLLIYDASSRATGIPDRFSGSVSETDFTLTISSLEPEDFAVYFCQQYGHSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 240 aa, synthetic construct
  source 1..240: note = HSV-1F gB amino acids 181-420
QFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDA

SEQ ID NO: 40 - protein, 240 aa, synthetic construct
  source 1..240: note = HSV-2G gB amino acids 173-412
QFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGXFRFSSDAISTTFTTNLTQYSLSRVDLGDCIGRDAREA